SEQUENCE LISTING (109 sequences)

SEQ ID NO: 1 - protein, 380 aa, Homo sapiens
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 2 - protein, 385 aa, Mus sp.
MEDLRPMATCPVLQKETLFRTGVHAYRIPALLYLKKQKTLLAFAEKRASKTDEHAELIVLRRGSYNEATNRVKWQPEEVVTQAQLEGHRSMNPCPLYDKQTKTLFLFFIAVPGRVSEHHQLHTKVNVTRLCCVSSTDHGRTWSPIQDLTETTIGSTHQEWATFAVGPGHCLQLRNPAGSLLVPAYAYRKLHPAQKPTPFAFCFISLDHGHTWKLGNFVAENSLECQVAEVGTGAQRMVYLNARSFLGARVQAQSPNDGLDFQDNRVVSKLVEPPHGCHGSVVAFHNPISKPHALDTWLLYTHPTDSRNRTNLGVYLNQMPLDPTAWSEPTLLAMGICAYSDLQNMGQGPDGSPQFGCLYESGNYEEIIFLIFTLKQAFPTVFDAQ

SEQ ID NO: 3 - protein, 5 aa, Mus sp.
EDLRP

SEQ ID NO: 4 - protein, 6 aa, Mus sp.
MEDLRP

SEQ ID NO: 5 - protein, 380 aa, synthetic construct
  REGION 1..380: note = Description of Artificial Sequence: Synthetic polypeptide
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGLLYEANDYEEIVFLMFTLKQAFPAEYLPQ

SEQ ID NO: 6 - protein, 386 aa, synthetic construct
  REGION 1..386: note = Description of Artificial Sequence: Synthetic polypeptide
MEDLRPMASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 7 - protein, 364 aa, Homo sapiens
ENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISV

SEQ ID NO: 8 - protein, 428 aa, Homo sapiens
MEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 9 - protein, 461 aa, Homo sapiens
MRPADLPPRPMEESPASSSAPTETEEPGSSAEVMEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 10 - protein, 484 aa, Homo sapiens
MGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 11 - protein, 496 aa, Homo sapiens
MMSSAAFPRWLSMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 12 - protein, 5 aa, Homo sapiens
MASLP

SEQ ID NO: 13 - protein, 4 aa, Homo sapiens
ASLP

SEQ ID NO: 14 - protein, 8 aa, synthetic construct
  REGION 1..8: note = Description of Artificial Sequence: Synthetic peptide
TVEKSVVF

SEQ ID NO: 15 - protein, 12 aa, Homo sapiens
GDYDAPTHQVQW

SEQ ID NO: 16 - protein, 8 aa, synthetic construct
  REGION 1..8: note = Description of Artificial Sequence: Synthetic peptide
SMDQGSTW

SEQ ID NO: 17 - protein, 8 aa, synthetic construct
  REGION 1..8: note = Description of Artificial Sequence: Synthetic peptide
STDGGKTW

SEQ ID NO: 18 - protein, 8 aa, Homo sapiens
PRPPAPEA

SEQ ID NO: 19 - protein, 8 aa, synthetic construct
  REGION 1..8: note = Description of Artificial Sequence: Synthetic peptide
QTPLEAAC

SEQ ID NO: 20 - protein, 9 aa, Homo sapiens
NPRPPAPEA

SEQ ID NO: 21 - protein, 7 aa, synthetic construct
  REGION 1..7: note = Description of Artificial Sequence: Synthetic peptide
SQNDGES

SEQ ID NO: 22 - protein, 7 aa, synthetic construct
  REGION 1..7: note = Description of Artificial Sequence: Synthetic peptide
LSHSLST

SEQ ID NO: 23 - DNA, 1092 bp, Homo sapiens
  source 1..1092: mol_type = other DNA
gagaacgactttggactggtgcagcctctggtcaccatggaacagctgctgtgggtttccggcagacagatcggcagcgtggacaccttcagaatccctctgatcaccgccacacctagaggcaccctgctggcctttgccgaggccagaaagatgagcagctctgacgagggcgccaagtttattgccctgaggcggtctatggaccagggctctacatggtcccctaccgccttcatcgtgaacgatggcgacgtgcccgatggcctgaatctgggagctgtggtgtccgatgtggaaaccggcgtggtgttcctgttctacagcctgtgtgcccacaaggccggttgtcaggtggccagcacaatgctcgtgtggtccaaggacgacggcgtgtcctggtctacccctagaaacctgagcctggacatcggcaccgaagtgtttgctccaggacctggctctggcatccagaagcagagagagcccagaaagggcagactgatcgtgtgtggccacggcacccttgagagagatggcgttttctgcctgctgagcgacgatcatggcgcctcttggagatacggcagcggagtgtctggaatcccttacggccagcctaagcaagagaacgatttcaaccccgacgagtgccagccttacgagctgcctgatggcagcgtcgtgatcaacgcccggaaccagaacaactaccactgccactgccggatcgtgctgagaagctacgacgcctgcgataccctgcggcctagagatgtgaccttcgatcctgagctggtggaccctgttgttgccgctggtgccgtcgtgacatctagcggcatcgtgttcttcagcaaccctgctcaccccgagttcagagtgaatctgaccctgcggtggtccttcagcaatggcacaagctggcggaaagaaaccgtgcagctttggcctggacctagcggctactcttctctggctacactggaaggcagcatggacggcgaagaacaggcccctcagctgtacgtgctgtacgagaagggcagaaaccactacaccgagagcatcagcgtggccaagatcagcgtt

SEQ ID NO: 24 - DNA, 1140 bp, Homo sapiens
  source 1..1140: mol_type = other DNA
atggccagcctgcctgtgctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgcccctacacatcaggtgcagtggcaggctcaagaggtggtggctcaggctagactggacggccacagatctatgaacccctgtcctctgtacgatgcccagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccatccagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagctgtgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 25 - DNA, 1284 bp, Homo sapiens
  source 1..1284: mol_type = other DNA
atggaggaagtgaccacctgtagcttcaacagccctctgttccggcaagaggacgaccggggcatcacctacagaatccctgctctgctgtacatccctcctacacacacctttctggccttcgccgagaagcggagcaccagacgagatgaagatgccctgcacctggtgctgagaagaggcctgagaatcggacagctggtgcagtggggacctctgaagcctctgatggaagccacactgcccggccacagaaccatgaatccttgtcctgtgtgggagcagaaaagcggctgcgtgttcctgttcttcatctgcgtgcggggccacgtgaccgagagacagcaaatcgtgtccggcagaaacgccgccagactgtgcttcatctacagccaggatgccggctgctcttggagcgaagttcgggatctgaccgaagaagtgatcggcagcgagctgaagcactgggccacatttgctgttggccctggccacggaatccagctgcaatctggcagactggtcatccccgcctacacctactatatccccagctggttcttctgcttccaactgccttgcaagacccggcctcacagcctgatgatctacagcgacgatctgggcgtgacatggcaccacggcagactgatcagacccatggtcaccgtggaatgcgaggtggccgaagtgacaggcagagctggacaccctgtgctgtactgctctgccagaacacccaaccggtgtagagccgaggctctgtctacagatcacggcgagggctttcagagactggccctctctagacagctgtgcgaacctcctcatggctgtcagggcagcgtggtgtccttcagacctctggaaatccctcaccggtgccaggacagcagctctaaggatgcccctaccatccagcagtctagccctggcagcagcctgagactggaagaggaagccggaacacctagcgagagctggctgctgtactctcaccccaccagcagaaagcagagagtggacctgggcatctacctgaatcagacccctctggaagccgcctgttggagcagaccttggattctgcactgtggcccttgcggctactctgatctggccgctctggaagaagagggcctgttcggctgcctgtttgagtgcggcacaaagcaagagtgcgagcagatcgccttccggctgttcacccacagagagatcctgagccatctgcagggcgactgcacaagcccaggcagaaatcccagccagttcaagagcaac

SEQ ID NO: 26 - DNA, 1452 bp, Homo sapiens
  source 1..1452: mol_type = other DNA
atgggcgtgcccagaacacccagcagaaccgtgctgttcgagagagagaggaccggcctgacctacagagtgccttctctgctgcctgtgcctcctggacctacactgctggccttcgtggaacagagactgagccccgatgattctcacgcccacagactggtgctgagaagaggaacactggctggcggctctgttagatggggagcactgcatgtgctgggcacagctgctcttgccgagcacagatccatgaatccctgtcctgtgcacgacgccggaaccggcacagtgtttctgttctttatcgccgtgctgggccacacacctgaggccgttcaaattgccaccggcagaaatgccgccagactgtgttgtgtggcctccagagatgccggcctgtcttggggatctgccagagatctgaccgaggaagccattggcggagccgttcaggattgggccacatttgctgttggacctggacacggcgtgcagctgccaagtggtagactgctggtgcctgcctacacatacagagtggatcggagagagtgcttcggaaagatctgccggacaagccctcacagcttcgccttctactccgacgatcacggccggacttggagatgtggtggcctggtgcctaatctgagaagcggcgaatgtcaactggccgccgttgatggtggacaggctggcagcttcctgtactgcaacgccagatctcctctgggctctagagtgcaggccctgtctaccgatgagggcaccagttttctgcccgccgaaagagttgcctctctgcctgaaacagcctggggctgtcagggctctatcgtgggatttcctgctcctgctccaaacagaccccgggacgattcttggagtgtcggccctggatctccactgcagcctccattgcttggaccaggcgttcacgagccacctgaagaggctgccgttgatcctagaggcggacaagttcctggcggcccttttagcagactgcagccaagaggcgacggccctagacaacctggaccaagacctggcgtcagcggagatgttggctcttggacactggccctgcctatgccttttgccgctcctcctcagtctcctacctggctgctgtactctcaccctgttggcagacgggccagactgcacatgggcatcagactgtctcagagccctctggaccccagaagctggacagagccttgggtcatctatgagggccctagcggctacagcgatctggcctctattggcccagctcctgaaggcggactggtgttcgcttgtctgtatgagagcggcgccagaaccagctacgacgagatcagcttctgcaccttcagcctgcgcgaggtgctggaaaatgtgcccgcctctcctaagcctcctaacctgggcgataagcctagaggctgttgctggccatct

SEQ ID NO: 27 - protein, 47 aa, Homo sapiens
MTGERPSTALPDRRWGPRILGFWGGCRVWVFAAIFLLLSLAASWSKA

SEQ ID NO: 28 - protein, 22 aa, synthetic construct
  REGION 1..22: note = Description of Artificial Sequence: Synthetic peptide
MDMRVPAQLLGLLLLWLPGARC

SEQ ID NO: 29 - protein, 4 aa, Homo sapiens
YGTL

SEQ ID NO: 30 - protein, 382 aa, Salmonella typhimurium
MTVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYN

SEQ ID NO: 31 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 227 aa, synthetic construct
  REGION 1..227: note = Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 33 - protein, 232 aa, synthetic construct
  REGION 1..232: note = Description of Artificial Sequence: Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1383 bp, Homo sapiens
  source 1..1383: mol_type = other DNA
atgagacctgcggacctgcccccgcgccccatggaagaatccccggcgtccagctctgccccgacagagacggaggagccggggtccagtgcagaggtcatggaagaagtgacaacatgctccttcaacagccctctgttccggcaggaagatgacagagggattacctaccggatcccagccctgctctacataccccccacccacaccttcctggcctttgcagagaagcgttctacgaggagagatgaggatgctctccacctggtgctgaggcgagggttgaggattgggcagttggtacagtgggggcccctgaagccactgatggaagccacactaccggggcatcggaccatgaacccctgtcctgtatgggagcagaagagtggttgtgtgttcctgttcttcatctgtgtgcggggccatgtcacagagcgtcaacagattgtgtcaggcaggaatgctgcccgcctttgcttcatctacagtcaggatgctggatgttcatggagtgaggtgagggacttgactgaggaggtcattggctcagagctgaagcactgggccacatttgctgtgggcccaggtcatggcatccagctgcagtcagggagactggtcatccctgcgtatacctactacatcccttcctggttcttttgcttccagctaccatgtaaaaccaggcctcattctctgatgatctacagtgatgacctaggggtcacatggcaccatggtagactcattaggcccatggttacagtagaatgtgaagtggcagaggtgactgggagggctggccaccctgtgctatattgcagtgcccggacaccaaacaggtgccgggcagaggcgctcagcactgaccatggtgaaggctttcagagactggccctgagtcgacagctctgtgagcccccacatggttgccaagggagtgtggtaagtttccggcccctggagatcccacataggtgccaggactctagcagcaaagatgcacccaccattcagcagagctctccaggcagttcactgaggctggaggaggaagctggaacaccgtcagaatcatggctcttgtactcacacccaaccagtaggaaacagagggttgacctaggtatctatctcaaccagacccccttggaggctgcctgctggtcccgcccctggatcttgcactgtgggccctgtggctactctgatctggctgctctggaggaggagggcttgtttgggtgtttgtttgaatgtgggaccaagcaagagtgtgagcagattgccttccgcctgtttacacaccgggagatcctgagtcacctgcagggggactgcaccagccctggtaggaacccaagccaattcaaaagcaat

SEQ ID NO: 35 - DNA, 1488 bp, Homo sapiens
  source 1..1488: mol_type = other DNA
atgatgagctctgcagccttcccaaggtggctgagcatgggggtccctcgtaccccttcacggacagtgctcttcgagcgggagaggacgggcctgacctaccgcgtgccctcgctgctccccgtgccccccgggcccaccctgctggcctttgtggagcagcggctcagccctgacgactcccacgcccaccgcctggtgctgaggaggggcacgctggccgggggctccgtgcggtggggtgccctgcacgtgctggggacagcagccctggcggagcaccggtccatgaacccctgccctgtgcacgatgctggcacgggcaccgtcttcctcttcttcatcgcggtgctgggccacacgcctgaggccgtgcagatcgccacgggaaggaacgccgcgcgcctctgctgtgtggccagccgtgacgccggcctctcgtggggcagcgcccgggacctcaccgaggaggccatcggtggtgccgtgcaggactgggccacattcgctgtgggtcccggccacggtgtgcagctgccctcaggccgcctgctggtacccgcctacacctaccgcgtggaccgccgagagtgttttggcaagatctgccggaccagccctcactccttcgccttctacagcgatgaccacggccgcacctggcgctgtggaggcctcgtgcccaacctgcgctcaggcgagtgccagctggcagcggtggacggtgggcaggccggcagcttcctctactgcaatgcccggagcccactgggcagccgtgtgcaggcgctcagcactgacgagggcacctccttcctgcccgcagagcgcgtggcttccctgcccgagactgcctggggctgccagggcagcatcgtgggcttcccagcccccgcccccaacaggccacgggatgacagttggtcagtgggccccgggagtcccctccagcctccactcctcggtcctggagtccacgaacccccagaggaggctgctgtagacccccgtggaggccaggtgcctggtgggcccttcagccgtctgcagcctcggggggatggccccaggcagcctggccccaggcctggggtcagtggggatgtggggtcctggaccctggcactccccatgccctttgctgccccgccccagagccccacgtggctgctgtactcccacccagtggggcgcagggctcggctacacatgggtatccgcctgagccagtccccgctggacccgcgcagctggacagagccctgggtgatctacgagggccccagcggctactccgacctggcgtccatcgggccggcccctgaggggggcctggtttttgcctgcctgtacgagagcggggccaggacctcctatgatgagatttccttttgtacattctccctgcgtgaggtcctggagaacgtgcccgccagccccaaaccgcccaaccttggggacaagcctcgggggtgctgctggccctcc

SEQ ID NO: 36 - protein, 379 aa, synthetic construct
  REGION 1..379: note = Description of Artificial Sequence: Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 37 - protein, 379 aa, synthetic construct
  REGION 1..379: note = Description of Artificial Sequence: Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 38 - protein, 380 aa, synthetic construct
  REGION 1..380: note = Description of Artificial Sequence: Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 39 - protein, 380 aa, synthetic construct
  REGION 1..380: note = Description of Artificial Sequence: Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 40 - protein, 450 aa, synthetic construct
  REGION 1..450: note = Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 213 aa, synthetic construct
  REGION 1..213: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 242 aa, synthetic construct
  REGION 1..242: note = Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 43 - protein, 873 aa, synthetic construct
  REGION 1..873: note = Description of Artificial Sequence: Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 44 - protein, 873 aa, synthetic construct
  REGION 1..873: note = Description of Artificial Sequence: Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 45 - protein, 874 aa, synthetic construct
  REGION 1..874: note = Description of Artificial Sequence: Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 46 - protein, 874 aa, synthetic construct
  REGION 1..874: note = Description of Artificial Sequence: Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 47 - protein, 874 aa, synthetic construct
  REGION 1..874: note = Description of Artificial Sequence: Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 48 - protein, 874 aa, synthetic construct
  REGION 1..874: note = Description of Artificial Sequence: Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 49 - protein, 213 aa, synthetic construct; identical to SEQ ID NO: 41
  REGION 1..213: note = Description of Artificial Sequence: Synthetic polypeptide

SEQ ID NO: 50 - protein, 450 aa, synthetic construct; identical to SEQ ID NO: 40
  REGION 1..450: note = Description of Artificial Sequence: Synthetic polypeptide

SEQ ID NO: 51 - protein, 616 aa, synthetic construct
  REGION 1..616: note = Description of Artificial Sequence: Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 52 - protein, 616 aa, synthetic construct
  REGION 1..616: note = Description of Artificial Sequence: Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 53 - protein, 617 aa, synthetic construct
  REGION 1..617: note = Description of Artificial Sequence: Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 54 - protein, 617 aa, synthetic construct
  REGION 1..617: note = Description of Artificial Sequence: Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 55 - protein, 618 aa, synthetic construct
  REGION 1..618: note = Description of Artificial Sequence: Synthetic polypeptide
TVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYNGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 56 - DNA, 1854 bp, synthetic construct
  misc_feature 1..1854: note = Description of Artificial Sequence: Synthetic polynucleotide
  source 1..1854: mol_type = other DNA
acagtggaaaagtccgtggtgttcaaggccgagggcgagcacttcaccgaccagaaaggcaataccatcgtcggctctggcagcggcggcaccaccaagtactttagaatccccgccatgtgcaccaccagcaagggcaccattgtggtgttcgccgacgccagacacaacaccgccagcgatcagagcttcatcgataccgctgccgccagatctaccgatggcggcaagacctggaacaagaagatcgccatctacaacgaccgcgtgaacagcaagctgagcagagtgatggaccctacctgcatcgtggccaacatccagggcagagaaaccatcctggtcatggtcggaaagtggaacaacaacgataagacctggggcgcctacagagacaaggcccctgataccgattgggacctcgtgctgtacaagagcaccgatgacggcgtgaccttcagcaaggtggaaacaaacatccacgacatcgtgaccaagaacggcaccatctctgccatgctcggcggcgttggatctggcctgcaactgaatgatggcaagctggtgttccccgtgcagatggtccgaacaaagaatatcaccaccgtgctgaataccagcttcatctacagcaccgacggcatcacatggtccctgcctagcggctactgtgaaggctttggcagcgagaacaacatcatcgagttcaacgccagcctggtcaacaacatccggaacagcggcctgcggagaagcttcgagacaaaggacttcggaaagacgtggaccgagtttcctccaatggacaagaaggtggacaaccggaaccacggcgtgcagggcagcacaatcacaatccctagcggcaacaaactggtggccgctcactctagcgcccagaacaagaacaacgactacaccagaagcgacatcagcctgtacgcccacaacctgtacagcggcgaagtgaagctgatcgacgacttctaccccaaagtgggcaatgccagcggagccggctacagctgtctgagctaccggaaaaatgtggacaaagaaaccctgtacgtggtgtacgaggccaacggcagcatcgagtttcaggacctgagcagacatctgcccgtgatcaagagctacaacggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 57 - protein, 450 aa, synthetic construct; identical to SEQ ID NO: 40
  REGION 1..450: note = Description of Artificial Sequence: Synthetic polypeptide

SEQ ID NO: 58 - DNA, 1350 bp, synthetic construct
  misc_feature 1..1350: note = Description of Artificial Sequence: Synthetic polynucleotide
  source 1..1350: mol_type = other DNA
gaggtgcagctggttgaatctggcggaggactggttcagcctggcggatctctgagactgtcttgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgttctagatggggaggcgacggcttctacgccatggattattggggccagggcaccctggtcaccgtttcttctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 59 - protein, 846 aa, synthetic construct
  REGION 1..846: note = Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYN

SEQ ID NO: 60 - DNA, 2538 bp, synthetic construct
  misc_feature 1..2538: note = Description of Artificial Sequence: Synthetic polynucleotide
  source 1..2538: mol_type = other DNA
gaggtgcagctggttgaatctggcggaggactggttcagcctggcggatctctgagactgtcttgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgttctagatggggaggcgacggcttctacgccatggattattggggccagggcaccctggtcaccgtttcttctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggtggcggaggatctggcggaggtggaagcggcggaggcggatctacagtggaaaagtccgtggtgttcaaggccgagggcgagcacttcaccgaccagaaaggcaataccatcgtcggctctggcagcggcggcaccaccaagtactttagaatccccgccatgtgcaccaccagcaagggcaccattgtggtgttcgccgacgccagacacaacaccgccagcgatcagagcttcatcgataccgctgccgccagaagtacagacggcggcaagacctggaacaagaagatcgccatctacaacgaccgcgtgaacagcaagctgagcagagtgatggaccctacctgcatcgtggccaacatccagggcagagaaaccatcctggtcatggtcggaaagtggaacaacaacgataagacctggggcgcctacagagacaaggcccctgataccgattgggacctcgtgctgtataagagcaccgacgacggcgtgaccttcagcaaggtggaaacaaacatccacgacatcgtgaccaagaacggcaccatctctgccatgctcggcggcgttggatctggcctgcaactgaatgatggcaagctggtgttccccgtgcagatggtccgaacaaagaacatcaccaccgtgctgaataccagcttcatctactccaccgacggcatcacatggtccctgcctagcggctactgtgaaggctttggcagcgagaacaacatcatcgagttcaacgccagcctggtcaacaacatccggaacagcggcctgcggagaagcttcgagacaaaggacttcggaaagacgtggaccgagtttcctccaatggacaagaaggtggacaaccggaaccacggcgtgcagggcagcacaatcacaatccctagcggcaacaaactggtggccgctcactctagcgcccagaacaagaacaacgattacaccagaagcgacatcagcctgtacgcccacaacctgtactccggcgaagtgaagctgatcgacgacttctaccccaaagtgggcaatgccagcggagccggctacagctgtctgagctaccggaaaaatgtggacaaagaaaccctgtacgtggtgtacgaggccaacggcagcatcgagtttcaggacctgagcagacatctgcccgtgatcaagagctacaat

SEQ ID NO: 61 - protein, 618 aa, synthetic construct
  REGION 1..618: note = Description of Artificial Sequence: Synthetic polypeptide
TVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMVPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSVNNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYNGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 62 - DNA, 1854 bp, synthetic construct
  misc_feature 1..1854: note = Description of Artificial Sequence: Synthetic polynucleotide
  source 1..1854: mol_type = other DNA
acagtggaaaagtccgtggtgttcaaggccgagggcgagcacttcaccgaccagaaaggcaataccatcgtcggctctggcagcggcggcaccaccaagtactttagaatccccgccatgtgcaccaccagcaagggcaccattgtggtgttcgccgacgccagacacaacaccgccagcgatcagagcttcatcgataccgctgccgccagaagtacagacggcggcaagacctggaacaagaagatcgccatctacaacgaccgcgtgaacagcaagctgagcagagtgatggtccctacctgcatcgtggccaacatccagggcagagaaaccatcctggtcatggtcggaaagtggaacaacaacgataagacctggggcgcctacagagacaaggcccctgataccgattgggacctcgtgctgtataagagcaccgacgacggcgtgaccttcagcaaggtggaaacaaacatccacgacatcgtgaccaagaacggcaccatctctgccatgctcggcggcgttggatctggcctgcaactgaatgatggcaagctggtgttccccgtgcagatggtccgaacaaagaacatcaccaccgtgctgaataccagcttcatctactccaccgacggcatcacatggtccctgcctagcggctactgtgaaggctttggcagcgtgaacaacatcatcgagttcaacgccagcctggtcaacaacatccggaacagcggcctgcggagaagcttcgagacaaaggacttcggaaagacgtggaccgagtttcctccaatggacaagaaggtggacaaccggaaccacggcgtgcagggcagcacaatcacaatccctagcggcaacaaactggtggccgctcactctagcgcccagaacaagaacaacgattacaccagaagcgacatcagcctgtacgcccacaacctgtactccggcgaagtgaagctgatcgacgacttctaccccaaagtgggcaatgccagcggagccggctacagctgtctgagctaccggaaaaatgtggacaaagaaaccctgtacgtggtgtacgaggccaacggcagcatcgagtttcaggacctgagcagacatctgcccgtgatcaagagctacaatggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 63 - protein, 386 aa, synthetic construct; identical to SEQ ID NO: 6
  REGION 1..386: note = Description of Artificial Sequence: Synthetic polypeptide

SEQ ID NO: 64 - DNA, 1158 bp, synthetic construct
  misc_feature 1..1158: note = Description of Artificial Sequence: Synthetic polynucleotide
  source 1..1158: mol_type = other DNA
atggaagatctcaggcccatggcatctctgcctgtgctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgcccctacacatcaggtgcagtggcaggctcaagaggtggtggctcaggctagactggacggccacagatctatgaacccctgtcctctgtacgatgcccagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccatccagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagctgtgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 65 - protein, 873 aa, synthetic construct; identical to SEQ ID NO: 43
  REGION 1..873: note = Description of Artificial Sequence: Synthetic polypeptide

SEQ ID NO: 66 - DNA, 2619 bp, synthetic construct
  misc_feature 1..2619: note = Description of Artificial Sequence: Synthetic polynucleotide
  source 1..2619: mol_type = other DNA
gcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgcccctacacatcaggtgcagtggcaggctcaagaggtggtggctcaggctagactggacggccacagatctatgaacccctgtcctctgtacgatgcccagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagctgtgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggaggcggaggatctggcggaggtggaagtggcggaggcggatctgaggtgcagctggttgaatctggcggaggactggttcagcctggcggatctctgagactgtcttgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgttctagatggggaggcgacggcttctacgccatggattattggggccagggcaccctggtcaccgtttcttctggcggaggaggatctggcggaggcggaagtggcggaggcggatctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgccagctttctgtactccggcgtgcccagcagattcagcggctctagaagcggcaccgacttcaccctgaccataagcagtctgcagcccgaggacttcgccacctactactgtcagcagcactacaccacacctccaacctttggccagggcaccaaggtggaaatcaag

SEQ ID NO: 67 - protein, 214 aa, synthetic construct
  REGION 1..214: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - DNA, 642 bp, synthetic construct
  misc_feature 1..642: note = Description of Artificial Sequence: Synthetic polynucleotide
  source 1..642: mol_type = other DNA
gacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgccagctttctgtactccggcgtgcccagcagattcagcggctctagaagcggcaccgacttcaccctgaccataagcagtctgcagcccgaggacttcgccacctactactgtcagcagcactacaccacacctccaacctttggccagggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 69 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 380 aa, synthetic construct
  REGION 1..380: note = Description of Artificial Sequence: Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 71 - protein, 380 aa, synthetic construct
  REGION 1..380: note = Description of Artificial Sequence: Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 72 - protein, 380 aa, synthetic construct
  REGION 1..380: note = Description of Artificial Sequence: Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 73 - protein, 380 aa, synthetic construct
  REGION 1..380: note = Description of Artificial Sequence: Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 74 - protein, 874 aa, synthetic construct
  REGION 1..874: note = Description of Artificial Sequence: Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 75 - protein, 874 aa, synthetic construct
  REGION 1..874: note = Description of Artificial Sequence: Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 76 - protein, 617 aa, synthetic construct
  REGION 1..617: note = Description of Artificial Sequence: Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 77 - protein, 617 aa, synthetic construct
  REGION 1..617: note = Description of Artificial Sequence: Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 78 - protein, 617 aa, synthetic construct
  REGION 1..617: note = Description of Artificial Sequence: Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 79 - protein, 617 aa, synthetic construct
  REGION 1..617: note = Description of Artificial Sequence: Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 80 - DNA, 1143 bp, Salmonella typhimurium
  source 1..1143: mol_type = other DNA
acagtggaaaagtccgtggtgttcaaggccgagggcgagcacttcaccgaccagaaaggcaataccatcgtcggctctggcagcggcggcaccaccaagtactttagaatccccgccatgtgcaccaccagcaagggcaccattgtggtgttcgccgacgccagacacaacaccgccagcgatcagagcttcatcgataccgctgccgccagatctaccgatggcggcaagacctggaacaagaagatcgccatctacaacgaccgcgtgaacagcaagctgagcagagtgatggaccctacctgcatcgtggccaacatccagggcagagaaaccatcctggtcatggtcggaaagtggaacaacaacgataagacctggggcgcctacagagacaaggcccctgataccgattgggacctcgtgctgtacaagagcaccgatgacggcgtgaccttcagcaaggtggaaacaaacatccacgacatcgtgaccaagaacggcaccatctctgccatgctcggcggcgttggatctggcctgcaactgaatgatggcaagctggtgttccccgtgcagatggtccgaacaaagaatatcaccaccgtgctgaataccagcttcatctacagcaccgacggcatcacatggtccctgcctagcggctactgtgaaggctttggcagcgagaacaacatcatcgagttcaacgccagcctggtcaacaacatccggaacagcggcctgcggagaagcttcgagacaaaggacttcggaaagacgtggaccgagtttcctccaatggacaagaaggtggacaaccggaaccacggcgtgcagggcagcacaatcacaatccctagcggcaacaaactggtggccgctcactctagcgcccagaacaagaacaacgactacaccagaagcgacatcagcctgtacgcccacaacctgtacagcggcgaagtgaagctgatcgacgacttctaccccaaagtgggcaatgccagcggagccggctacagctgtctgagctaccggaaaaatgtggacaaagaaaccctgtacgtggtgtacgaggccaacggcagcatcgagtttcaggacctgagcagacatctgcccgtgatcaagagctacaac

SEQ ID NO: 81 - protein, 781 aa, Vibrio cholera
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 82 - DNA, 2424 bp, Vibrio cholera
  source 1..2424: mol_type = other DNA
ttgtcaatcaagatgacttcacaacgaagaagagcatcgattcacaaggaaacagattctaatataaagggagtagatatgcgtttcaaaaacgtaaagaaaaccgctttaatgcttgcaatgttcggtatggcgacaagctcaaacgccgcactttttgactataacgcaacgggtgacactgagtttgacagtccagccaaacagggatggatgcaagacaacacgaataatggcagcggcgttttaaccaatgcagatggaatgcccgcttggttggtgcaaggtattggagggagagctcaatggacatattctctctctactaatcaacatgcccaagcatcaagtttcggttggcgaatgacgacagaaatgaaagtgctcagtggtggaatgatcacaaactactacgccaacggcactcagcgtgtcttacccatcatttcattagatagcagtggtaacttagttgttgagtttgaagggcaaactggacgcaccgttttggcaaccggcacagcagcaacggaatatcataaatttgaattggtattccttcctggaagtaacccatccgctagcttttacttcgatggcaaactcattcgtgacaacatccagccgactgcatcaaaacaaaatatgatcgtatgggggaatggctcatcaaatacggatggtgtcgccgcttatcgtgatattaagtttgaaattcaaggcgacgtcatcttcagaggcccagaccgtataccgtccattgtagcaagtagcgtaacaccaggggtggtaaccgcatttgcagagaaacgtgtggggggaggagatcccggtgctctgagtaataccaatgacataatcactcgtacctcacgagatggcggtataacttgggataccgagctcaacctcactgagcaaatcaatgtcagtgatgagtttgatttctccgatcctcggcctatctatgatccttcctccaatacggttcttgtctcttatgctcgatggccgaccgatgccgctcaaaacggagatcgaataaaaccatggatgccaaacggtattttttacagcgtctatgatgttgcatcagggaactggcaagcgcctatcgatgttaccgatcaggtgaaagaacgcagtttccaaatcgctggttggggtggttcagagctgtatcgccgaaataccagcctaaatagccagcaagactggcaatcaaacgctaagatccgaattgttgatggtgcagcgaaccagatacaagttgccgatggtagccgaaaatatgttgtcacactgagtattgatgaatcaggtggtctagtcgctaatctaaacggtgttagtgctccgattatcctgcaatctgaacacgcaaaggtacactctttccatgactacgaacttcaatattcggcgttaaaccacaccacaacgttattcgtggatggtcagcaaatcacaacttgggctggcgaagtatcgcaggagaacaacattcagtttggtaatgcggatgcccaaattgacggcagactgcatgtgcaaaaaattgttctcacacagcaaggccataacctcgtggagtttgatgctttctatttagcacagcaaacccctgaagtagagaaagaccttgaaaagcttggttggacaaaaattaaaacgggcaacaccatgagtttgtatggaaatgccagtgtcaacccaggaccgggtcatggcatcacccttactcgacaacaaaatatcagtggcagccaaaacggccgcttgatctacccagcgattgtgcttgatcgtttcttcttgaacgtcatgtctatttacagtgatgatggcggttcaaactggcaaaccggttcaacactccctatcccctttcgctggaagagttcgagtatcctagaaactctcgaacctagtgaagctgatatggttgaactccaaaacggtgatctactccttactgcacgccttgattttaaccaaatcgttaatggtgtgaactatagcccacgccagcaatttttgagtaaagatggtggaatcacgtggagcctacttgaggctaacaacgctaacgtctttagcaatatcagtactggtaccgttgatgcttctattactcggttcgagcaaagtgacggtagccatttcttactctttactaacccacaaggaaaccctgcggggacaaatggcaggcaaaatctaggcttatggtttagcttcgatgaaggggtgacatggaaaggaccaattcaacttgttaatggtgcatcggcatattctgatatttatcaattggattcggaaaatgcgattgtcattgttgaaacggataattcaaatatgcgaattcttcgtatgcctatcacattgctaaaacagaagctgaccttatcgcaaaactaa

SEQ ID NO: 83 - protein, 409 aa, Mus sp.
MVGADPTRPRGPLSYWAGRRGQGLAAIFLLLVSAAESEARAEDDFSLVQPLVTMEQLLWVSGKQIGSVDTFRIPLITATPRGTLLAFAEARKKSASDEGAKFIAMRRSTDQGSTWSSTAFIVDDGEASDGLNLGAVVNDVDTGIVFLIYTLCAHKVNCQVASTMLVWSKDDGISWSPPRNLSVDIGTEMFAPGPGSGIQKQREPGKGRLIVCGHGTLERDGVFCLLSDDHGASWHYGTGVSGIPFGQPKHDHDFNPDECQPYELPDGSVIINARNQNNYHCRCRIVLRSYDACDTLRPRDVTFDPELVDPVVAAGALATSSGIVFFSNPAHPEFRVNLTLRWSFSNGTSWLKERVQVWPGPSGYSSLTALENSTDGKKQPPQLFVLYEKGLNRYTESISMVKISVYGTL

SEQ ID NO: 84 - protein, 393 aa, Mus sp.
MTVQPSPWFSDLRPMATCPVLQKETLFRTGVHAYRIPALLYLKKQKTLLAFAEKRASKTDEHAELIVLRRGSYNEATNRVKWQPEEVVTQAQLEGHRSMNPCPLYDKQTKTLFLFFIAVPGRVSEHHQLHTKVNVTRLCCVSSTDHGRTWSPIQDLTETTIGSTHQEWATFAVGPGHCLQLRNPAGSLLVPAYAYRKLHPAQKPTPFAFCFISLDHGHTWKLGNFVAENSLECQVAEVGTGAQRMVYLNARSFLGARVQAQSPNDGLDFQDNRVVSKLVEPPHGCHGSVVAFHNPISKPHALDTWLLYTHPTDSRNRTNLGVYLNQMPLDPTAWSEPTLLAMGICAYSDLQNMGQGPDGSPQFGCLYESGNYEEIIFLIFTLKQAFPTVFDAQ

SEQ ID NO: 85 - protein, 418 aa, Mus sp.
MEEVPPYSLSSTLFQQEEQSGVTYRIPALLYLPPTHTFLAFAEKRTSVRDEDAACLVLRRGLMKGRSVQWGPQRLLMEATLPGHRTMNPCPVWEKNTGRVYLFFICVRGHVTERCQIVWGKNAARLCFLCSEDAGCSWGEVKDLTEEVIGSEVKRWATFAVGPGHGIQLHSGRLIIPAYAYYVSRWFLCFACSVKPHSLMIYSDDFGVTWHHGKFIEPQVTGECQVAEVAGTAGNPVLYCSARTPSRFRAEAFSTDSGGCFQKPTLNPQLHEPRTGCQGSVVSFRPLKMPNTYQDSIGKGAPATQKCPLLDSPLEVEKGAETPSATWLLYSHPTSKRKRINLGIYYNRNPLEVNCWSRPWILNRGPSGYSDLAVVEEQDLVACLFECGEKNEYERIDFCLFSDHEVLSCEDCTSPSSD

SEQ ID NO: 86 - protein, 501 aa, Mus sp.
METAGAPFCFHVDSLVPCSYWKVMGPTRVPRRTVLFQRERTGLTYRVPALLCVPPRPTLLAFAEQRLSPDDSHAHRLVLRRGTLTRGSVRWGTLSVLETAVLEEHRSMNPCPVLDEHSGTIFLFFIAVLGHTPEAVQIATGKNAARLCCVTSCDAGLTWGSVRDLTEEAIGAALQDWATFAVGPGHGVQLRSGRLLVPAYTYHVDRRECFGKICWTSPHSLAFYSDDHGISWHCGGLVPNLRSGECQLAAVDGDFLYCNARSPLGNRVQALSADEGTSFLPGELVPTLAETARGCQGSIVGFLAPPSIEPQDDRWTGSPRNTPHSPCFNLRVQESSGEGARGLLERWMPRLPLCYPQSRSPENHGLEPGSDGDKTSWTPECPMSSDSMLQSPTWLLYSHPAGRRARLHMGIYLSRSPLDPHSWTEPWVIYEGPSGYSDLAFLGPMPGASLVFACLFESGTRTSYEDISFCLFSLADVLENVPTGLEMLSLRDKAQGHCWPS

SEQ ID NO: 87 - DNA, 3850 bp, Mus sp.
  source 1..3850: mol_type = other DNA
gggtcacatgctgatggactaattggagtcgcggcagcgcgggctgcggcccccaaggggaggggtcggagtgacgtgcgcgcttttaaagggccgaggtcagctgacggcttgccaccggtgaccagttcctggacagggatcgccgggagctatggtgggggcagacccgaccagaccccggggaccgctgagctattgggcgggccgtcggggtcaggggctcgcagcgatcttcctgctcctggtgtccgcggcggaatccgaggccagggcagaggatgacttcagcctggtgcagccgctggtgaccatggagcagctgctgtgggtgagcgggaagcagatcggctctgtagacactttccgcatcccgctcatcacagccacccctcggggcacgctcctggccttcgctgaggccaggaaaaaatctgcatccgatgagggggccaagttcatcgccatgaggaggtccacggaccagggtagcacgtggtcctctacagccttcatcgtagacgatggggaggcctccgatggcctgaacctgggcgctgtggtgaacgatgtagacacagggatagtgttccttatctataccctctgtgctcacaaggtcaactgccaggtggcctctaccatgttggtttggagtaaggacgacggcatttcctggagcccaccccggaatctctctgtggatattggcacagagatgtttgcccctggacctggctcaggcattcagaaacagcgggagcctgggaagggccggctcattgtgtgtggacacgggacgctggagcgagatggggtcttctgtctcctcagtgatgaccacggtgcctcctggcactacggcactggagtgagcggcattccctttggccagcccaaacacgatcacgatttcaaccccgacgagtgccagccctacgagcttccagatggctcggtcatcatcaacgcccggaaccagaataactaccattgccgctgcaggatcgtcctccgcagctatgacgcctgtgacaccctcaggccccgggatgtgaccttcgaccctgagctcgtggaccctgtggtagctgcaggagcactagccaccagctccggcattgtcttcttctccaatccagcccaccctgagttccgagtgaacctgaccctgcgctggagtttcagcaatggtacatcctggcagaaggagagggtccaggtgtggccgggacccagcggctactcgtccctgacagccctggaaaacagcacggatggaaagaagcagcccccgcagctgttcgttctgtacgagaaaggcctgaaccggtacaccgagagcatctccatggtcaaaatcagcgtctacggcacgctctgagccccgtgcccaaaggacaccaagtcctggtcgctgacttcacagctctctggaccatctgcagagggtgcctgaaacacagctcttcctctgaactctgaccttttgcaacttctcatcaacagggaagtctcttcgttatgacttaacacccagcttcctctcggggcaggaagtccctccgtcaccaagagcacttttttccagtatgctggggatggcccctgtccattctcttccaggacaacggagctgtgcctttctgggacaggatgggggaggggctccccctggagagatgaacagatacgaactcagggaactgagaaggcccggtgtcctagggtacaaaggcaggtactagatgtgattgctgaaagtccccagggcagagtgtcctttcagagcaaggataagcacacctacgtgtgcacctttgattatttatgaatcgaaatatttgtaacttaaaatttttgatgcagaaaaagcgtttgtggagtctgtggttctgtctgctcacgccttcccaattgcctcctggagagacaggaaggcagctggaagaggagccgatgtacttactgggaagcagaaacccctagattccatcctggctgctgctgtttgcaagtgtcaaagatgggggggcgtgtttatattttatatttctaagatggggtggcataggaaatagggaacagatgtgtaaaaccagatgggaaggacagtctgtgagaaaggagcaagcagttgctgcaggtgtgggagagcaaagcccttctccacgtggaaagagcccagatggacgctaagcatgttgggcacctgtaaccccgcactcgctggactgacggtgtagctcagtggtggagctagtacttggaacgcctaagactctgggttcagtccttgggggggggggtatgtgtttattgagaggaaggtgtacgtactgtaggtcagaggacagcttactggagttgtctctctccttcacgctgtgagtcctgtggaatgacctcaggtgtcagagttgggggcaggtgcctttgccagctgagccatcttgctgtctctgctttaatttaaaaaaaaaaaaaaaaaagaatattaaggtctgagggattcgggctgcgttcatttcaattagagggtcatatttcttttgacatttcttctctaagaaatgttaagatcatttgttctgtgtgatagaggtatagctccattgtatgtcagcagtgagggatcctgtgcattttatccagagtttgtacggtgttctaggggctgctagtgcagcccagtgctaaacacttcagcatgcacaaggcctcaatcagtgcatgcatgtgcacacacacacagacacacacgtacacactgacacaggtacacaaatacacactggcccacatgtacacatcgactcacaggtacacagacccactttgacacacatatacacagacacaaacgcactggcacacacatatacacaggcacacatggatagatggacacacgtgtacacatacacacacacacagaaatacaaatgttcaggttttctaaaaaaaaaaaaattagagacgtgttgacttcatttttagcaaaaatcctgtcatgtatcttaaagtggattgaacccactatgtagcccaggctggcctccaaatgggcatccttctgcctcagtctcccgagggctaggataacaggagtatgccatcacacctggctaatagaaattttcaaaattgtttgtttgaaggtgactcttactatattgcctaactgatctccagttcgtgaaatcctcctgcctcagaaccaggactgtcaatataacccaccaagacaggccaacattcacaattgattgttagtttgtggtctgaatcaaggtcttatactgtagcccaggctagcccggaatacacgatatctccagtgcttcagatcctcagttctaactaagcatggccacatccatgtttaactgcaaatttgatgttaccatggtttggtttggtttggtttggtttggtttggtttggtttggttttttggccattttttttttctcatgctgaggccttgtgctctcaagttggggagacagcatggagggtagctgcaactgtaaccccagttccaggggacctgacaccctctggcctccacaagtattaggcacatctgtggtgcacagacatacaatcaggcaaaatattcatacacataaaataaaataatttaaaacaaaagcaaaaatcaggacctaagaaaaaaatctattcctgattcttttatgttttgtttgtattttatcaagacagggttgtttctctgtatagccctggctgtcttggaattcactctgtagaccaggctggcctcaaactcagaaatcctcctgcctttgccttccaagtgctggaattaaaggcatgcgccacc

SEQ ID NO: 88 - DNA, 1722 bp, Mus sp.
  source 1..1722: mol_type = other DNA
gacatgacccaaacggcccctggctgcaaggtaatatcggaagttgactaagaatggacgccccaccactgactgacccgccccctgagtctgagattggacttgtctctggatacagtcatactttgaggtactacaagttagaaactgttaggttactcagttcagtccatgacagtccaaccttctccatggttttccgatctcaggcccatggcgacctgccctgtcctgcagaaggagacactgttccgcacaggcgtccatgcttacagaatccctgctctgctctacctgaagaagcagaagaccctgctggcctttgcggaaaagcgagccagcaagacggatgagcacgcagagttgattgtcctgagaagaggaagctacaacgaagccaccaaccgtgtcaagtggcagcctgaggaagtggtgacccaagcccagctggaaggccaccgctccatgaatccatgtcccttgtatgacaagcaaacaaagaccctcttccttttcttcatcgctgtccctgggcgtgtatcagaacatcatcagctccacactaaggttaatgtcacacggctgtgctgtgtcagcagcactgaccatgggaggacctggagccccatccaggacctcacagagaccaccattggcagcactcatcaggaatgggccacatttgctgtgggtcctgggcattgtctgcagctgcggaacccagctgggagcctgctggtacctgcttatgcctaccggaaactgcaccctgctcagaagcctaccccctttgccttctgcttcatcagccttgaccatgggcacacatggaaactaggcaactttgtggctgaaaactcactggagtgccaggtggctgaggttggcactggagctcagaggatggtatatctcaatgctaggagcttcctgggagccagggtccaggcacaaagtcctaatgatggtctggatttccaggacaaccgggtagtgagtaagcttgtagagcccccccacgggtgtcatggaagtgtggttgccttccacaaccccatctctaagccacatgccttagacacatggcttctttatacacaccctacagactccaggaatagaaccaacctgggtgtgtacctaaaccagatgccactagatcccacagcctggtcagagcccaccctgctggccatgggcatctgtgcctactcagacttacagaacatggggcaaggccctgatggctccccacagtttgggtgtctgtatgaatcaggtaactatgaagagatcattttcctcatattcaccctgaagcaagctttccccactgtatttgatgcccagtgatctcagtgcacgtggcccaaagggcttccttgtgcttcaaaacacccatctctctttgcttccagcatcctctggactcttgagtccagctcttgggtaacttcctcaggaggatgcagagaatttggtctcttgactctctgcaggccttattgtttcagcctctggttctcttttcagcccagaaatcaaaggagcctggctttcctcagcctgttggcagggcaggtggggacagtatatatagaggctgccattctgcatgtcggttgtcactatgctagtttaacctgcctgtttccccatgcctagtgtttgaatgagtattaataaaatatccaacccagcccatttcttcctggaaaaaaa

SEQ ID NO: 89 - DNA, 3340 bp, Mus sp.
  source 1..3340: mol_type = other DNA
actgcgcggtgaaggggcgtggcctggccggggaggttgacacccagacgctgctctcagtcctctggcgcctgctccccagcgcattccttctgctcctgggatatttgtctcattactgccagttcttgcgcagcggtcactgggttcgtttcagcgtctgtggtttctgtcgctgttatccagtctccatcgccccagctcagcttcaggccttcttccgagactccacgggagagcccagagagcctccggagccgaagccatggaggaagtcccaccctactccctcagcagcaccctgttccagcaggaagaacagagtggggtgacctaccggatcccagccctgctgtaccttcctcccacccacaccttcctggcctttgcagagaagcggacctcagtcagagatgaggatgctgcctgcctggtgctcagacgagggctgatgaaggggcgctctgtacagtggggcccccaacggctactgatggaggccacattacctgggcatcgcaccatgaacccctgccctgtgtgggagaaaaatactggccgtgtgtacctgtttttcatctgtgtgcggggccatgttactgagaggtgccagattgtgtggggcaaaaatgccgcccgtctctgcttcctttgcagtgaagatgccggctgctcttggggtgaagtgaaagacttgaccgaggaggtcattggctcagaggtgaagcgctgggccacatttgctgtgggcccaggtcatggcatccagctacactcgggaaggctgatcatccccgcctatgcctactatgtctcacgttggtttctctgctttgcgtgttcagtcaagccccattccctgatgatctacagtgatgactttggagtcacatggcaccatggcaagttcattgagccccaggtgacaggggagtgccaagtggccgaagtggctgggacggctggtaaccctgtgctcactgcagtgcccgaacaccaagccgatttcgagcagaggcttttagtactgatagtggtggctgctttcagaagccaaccctgaacccacaactccatgagcctcgaaccggctgccaaggtagtgtagtgagcttccggcctttgaagatgccaaatacctatcaagactcaattggcaaaggtgctcccgctactcagaagtgccctctgctggacagtcctctggaggtggagaaaggagctgaaacaccatcagcaacatggctcttgtactcacatccaactagcaagaggaagaggattaacctaggcatctactacaaccggaaccccttggaggtgaactgctggtcccgcccgtggatcttgaaccgtgggcccagtggctactctgatctggctgttgtggaagaacaggacttggtggcgtgtttgtttgagtgtggggagaagaatgagtatgagcggattgacttctgtctgttttcagaccatgaggtcctgagctgtgaagactgtaccagccctagtagcgactaaagccaaatcaagacggatgagtgaggcccagcttcccacagaaaggaatggcagctacagccagggtaacagaggtctctgatgtctagagaaaactctaaaaactaataatctgctccttgaattttttcacttttcccttcaatgagcatggtgaaaattgtgccatatcttacataacgaggctcttgaactgggagtttgaatctcttctcttcccattaaaaggagaggccatgtgctcgcttcgcgttcgacaaagcctggattctgatcttgagtggaagccacaggcttgtcttttccaatggttcactgctcacctgagtattaggtgatgtgtaggtgccttggccagaagaaagatctgtgttgttgtatttttttaaatttatttatttactatatgtaagtacactgcagctgtcttcagacacaccagaagagggcgtcagatctcattagagatggttgtgagccaccatgtggttgctgggatttgaactcaggaccttcagaagagcagtcagtgctcttaactactgagccatctctcaagccccgcattgctgtatttttaataagaaaaatgcccttatccttccaataatgcctggagctgtacaaattctctgtcttagaagacttgagaaagcagaactgtaaggtcagatgctttctccagccttgatgctgtgttccaccttcccttcctcatccagaaaacagttactagggagaaaatgagaaacccatgccagctgcccttgatgatggttgataacggtgcttattgcttttgatgtcattacctctgttagagatgaatcagagtcagaggtccttagctgcatccacccatttccagggggacattctaacactgctgaacagtcagctaaaatgagagctgtgtgtcctagcctgattccaggttagtcatgatgcttcctggagctgggcttttatctaatcccaggagccatctaggggaggctcagagctagcaggtgatcttcctgagatggtttcaccgtgacaggtgaaccatgagcccttccaagcaaggccaaaggacaacattataggaaagatttctagtattaatatgccttttctctgtgtgtgtactgtcttgtagtgatgctatatagacaaatagatgatttcttattttttgtttgtttgtttgtttttttgtttttctgtagccctagctgtcctggaactcactttgtaaaccaggctggcctcgatctcagaaatccgcctgcctctgcctcccgagtgctgggattaaaggtgtgcaccaccacaccttaatgatgatcctataagtattcctaaaattatactagtaattattaactcctttataataggactgctattaaagccctcgctgatatgaaaactacagtgagaactctgccagtcttcacatgtcataattacttctgagatagaaagcaggcatttacaacttagaacacatttcttagagctgtaaaacaattaactagaggtcataaaagggaatgaaagatttattgtaggtgctaggacagaacataaaatattgactgggcttatctatatgaaacttcattgttaacttttacacaagaattatggtttttaactttcagtgaacctgcggagctagtgacagaagagaaatgtctagttagataactactcttaatggaaattcacataaacatctgttgccatcttctttttgaatttatgtttaaacttgtgaatgtttgaattagacactacgcgagcacatagaaaataaagaactaagcgtgaa

SEQ ID NO: 90 - DNA, 4608 bp, Mus sp.
  source 1..4608: mol_type = other DNA
ggacagtgtgcatcacggagcttgtggcccagactgtgcctggcagacccagaggacctaaggcttggctctagtggtggtcagcacagccctcggtggtctgcggagcctgatattgctttacgtaagggctgttctgctgtgcatctcctgtgtctgaagctattcgccatggagactgctggagctcccttctgcttccatgtggactccctggtaccttgctcctactggaaggttatggggcccacgcgtgttcccaggagaacggtgctcttccagagggaaaggacgggcctgacctaccgtgtgcctgcgttactctgtgtgcctcccaggcctactctgctggccttcgcggaacagcgacttagccctgatgactcccatgcccaccgcctggtgctacggaggggcacgctgaccaggggctcagtgcggtggggcactctgagtgtactggagactgcagtactggaggagcacaggtctatgaacccttgcccggtgctggatgagcactctggtaccatcttcctcttcttcattgccgtgctgggccacacaccggaggccgtgcaaatcgccactggcaagaacgctgctcgcctctgctgtgtgaccagctgtgacgctggcctcacctggggcagtgttcgagatctcactgaggaagccattggtgctgcattgcaggactgggccacctttgctgtgggtccgggccatggagttcagctgcgctcgggtcgcctgcttgttcctgcttacacctatcatgtggaccgacgggaatgttttggcaagatctgctggaccagtccccactccttggcattctacagtgatgatcatgggatctcctggcattgtggaggccttgtgcccaacctacgctctggagagtgccaactggctgcggtagatggagactttctctactgtaatgctcgaagccctctgggtaaccgtgtgcaggcactgagtgctgatgaaggcacgtccttcctaccaggggagctggtgcctacattggcagagacggctcgtggttgccagggtagcattgtgggcttcctagctccaccctcaatcgagcctcaggatgaccggtggacagggagtcctaggaacaccccacattccccatgcttcaatctcagagtacaggagtcttcgggggaaggtgccagaggtcttcttgaacgttggatgcccaggttgcctctctgctacccacagtcccggagcccagagaatcatggcctagagcctgggtcagatggagataagacatcctggactccggaatgtcctatgtcctctgattccatgcttcagagccccacatggctactatattcccacccagcagggcgtagagctcggctccacatgggaatctacctgagccgatcccccttggatccccacagctggacagagccctgggtgatctatgagggccccagtggctactctgaccttgcctttcttgggcctatgcctggggcatccctggtttttgcctgtctgtttgagagcgggaccaggacttcctatgaagacatttctttttgcttgttctcactggcggatgtcctggagaatgtgcccactggcttagagatgctaagtctcagggataaggctcaggggcattgctggccctcttgatggcctcaccctctcgtagccgcctggagaggaagggtagactatatagaggaggttaggggtaggtcagcatgatgctaggatggagagagctctgtcccctcgtggatggtggtggtgactcacccggggggccagctgctttctgagtgcaaatgagaaaaataaagagctgcgctgtgacttttctttccacatcaaagcttgggtgtcagtgctttagcttgatgctctgatcaccatgcaaatcttccaccggcgccttgctcagctttcatatcccaagggtgcctgggaggaaggcaacagggacagtggacatcactgcaccactttccacgaccctgtgtgccaacctcagccactttgaaacatgctgatgactgaggtctgttcactttcttaatttcaagcaggagaagcaggttggggagccagcctccccagctagaggggacagaacttgacttgagcaggggggtacctcctaggacctgctccatgtgcctacttctttacccttctctagagagggctcttgtcctgtcagagctgttttctcccttctcttgttttttctttttcaagactgtttctctgtgttagccctggctgtcctggatctcactctgtagatcaggctgaccttgagttcaaagctccatctgcctctacttctcacattactgtgattaaaggcatatactaccactgcctggtgcccttttgtatttcttattaaagtcctaatgtctgattataaaaacagtctgtgtgggctggagtgatggcttactcagtaaagcacttgccatggaatctgggcaatctgagtttcatttttagcatcctgtaaaaatcccaatttgatggtgtacttgtaatgtcagcatggagaggcagagataggtaagttccccaagactctttgaaccgacagcttggcctcactggcacattccaggtctcagtgagagaccctgcctcaaaatacaaagaaagagctgctgaagagtgggtcagagttgacctctgatctccggaagtatatgatacacacccgtgcatgcactcttccttacaaaataaaaagcaaaacaaaaccccaacaggtatatggccattttagaaaaattagaagatttagaaagctatacataaaaaaaaatgacctaaagaaaaatctttactgttctgggcactatccctatcaaaccactgtgttctttggccaagccttggggtggacactgttttgaggtgggtcctgttatctccactaggtagtggagttttgtgtcagactaactgggtcttaaagctgtctttaaggccatcaggagctactgacttgcctgcctcagcagagcatatcctgaaggtcggggttaagtctccttcccgagcgagttgccttccagtgggcccctggactcctaggtcctcagcgctcatcagctgccaaggactctgagggaatgtcctctgactgtggccccgaaaggtaggggagggggatgtgcttaggcttaggacagggtcctgtttcagtctgccttcactgttagtagcactgtgccacatggcacagactgggcgagctttaaaggaaggaggttgatattggttcccacttctggggatcatggttgagcagccttgtctgatgatggttgtcttgatggtagatcgtgaggtagttgatgaaggtatgacatggtgagaaactctgtgtgtgtgtgttattttctctgtgttctacctatacatctatctatgtatatatgtatctatctatctacctggaggctggagagatagcttagtggttaagaacatttgttgttcttgcatagtcctggatttaaattttcagcacccacatggcagctcacaacaacccataaatccagtttcagaggatccaacctctgatataccatgtcagccagagcagacacggctgaaggtggtttgatccccgtatggagaggtgacaattgggaagagagaaagatcaacttaaccatgcaaggaacaggaagttaaatactgaacagggaaggtaaaggcaggaagtagatgtagagggcaaatcaatgaaacccaaacatacccaaattacgctaaacacacactgacatgccaattaaaaggacaaattggctccactggcaaaaccaaaacagacactgaagatccaaacagtcacatgccaactaccgcggagggagacagacacagagaagaccgtgacagacacttggacactcttgagagtggatgtgcaggaagagagctctgccagtggagaagaaagcactcagaagaaagtgacagcagctgtaaatttgtattctgctaatgttatgttccaaagttgaaagcaaaattgtaccaattcataagaacaaacaggctgactctcagttgtgactgaacgtctctcagtaactgacggggcgagcaggccaaaggagagtcggctcagaagggtgcatagccacgccaaatcaaataagcaagtacaaccggcaggctctatttctagcacaaaggggtctgtgcctcattctgtgcttgggtcagagcttgggtctctcatttggatgtaagtggtgtagtggagaagcaggaaataatccggagcgcatattttgattttaacataagtgctgatttgggagggagttttgtcaaattgtgtttttacaatgtttttttttttttaaatgatgcttttttgtaaagtgtacaaatgtgatataagattggttctgctacattcagtttctataaaagtggttctaaaatattgtactgtcaatcatctcatgattattctactgtacacattactgactttgtatgtaataattaatattagaagaaaatataatttatttgaatataaaaaaaaaaaaaaaaaaa

SEQ ID NO: 91 - protein, 380 aa, synthetic construct
  REGION 1..380: note = Description of Artificial Sequence: Synthetic polypeptide
  MOD_RES 1: note = Ala, Arg, Asn, Asp, Gln, Glu, Gly, His, Leu, Lys, Met, Phe, Thr, Val, or not present
  MOD_RES 6: note = Phe, Trp, Tyr or Val
  MOD_RES 187: note = Arg, Ile or Lys
  MOD_RES 332: note = Ala, Cys, Ser or Val
XASLPXLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSXAYSDLQSMGTGPDGSPLFGCLYEANDYEEIVFLMFTLKQAFPAEYLPQ

SEQ ID NO: 92 - protein, 617 aa, synthetic construct
  REGION 1..617: note = Description of Artificial Sequence: Synthetic polypeptide
  MOD_RES 1: note = Ala, Arg, Asn, Asp, Gln, Glu, Gly, His, Leu, Lys, Met, Phe, Thr, Val, or not present
  MOD_RES 6: note = Phe, Trp, Tyr or Val
  MOD_RES 187: note = Arg, Ile or Lys
  MOD_RES 332: note = Ala, Cys, Ser or Val
XASLPXLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSXAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 93 - protein, 874 aa, synthetic construct
  REGION 1..874: note = Description of Artificial Sequence: Synthetic polypeptide
  MOD_RES 1: note = Ala, Arg, Asn, Asp, Gln, Glu, Gly, His, Leu, Lys, Met, Phe, Thr, Val, or not present
  MOD_RES 6: note = Phe, Trp, Tyr or Val
  MOD_RES 187: note = Arg, Ile or Lys
  MOD_RES 332: note = Ala, Cys, Ser or Val
XASLPXLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSXAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 94 - protein, 216 aa, synthetic construct
  REGION 1..216: note = Description of Artificial Sequence: Synthetic polypeptide
ELVLTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 95 - DNA, 648 bp, synthetic construct
  misc_feature 1..648: note = Description of Artificial Sequence: Synthetic polynucleotide
  source 1..648: mol_type = other DNA
gagctcgtgttgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaagcggccctcaggggtttctaatcgcttctctggctccaagtctggcaacacggcctccctgaccatctctgggctccaggctgaggacgaggctgattattactgcagctcatatacaagcagcagcactttggtattcggcggagggaccaagctgaccgtcctaggtcagcccaaggcggcgccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 96 - protein, 450 aa, synthetic construct
  REGION 1..450: note = Description of Artificial Sequence: Synthetic polypeptide
EVQLLESGGGLVQPGGSLRLSCTTSGFTFNTYAMSWVRQAPGKGLEWLSGINNNGRTAFYADSVKGRFTISRDNSKNTLYLQINSLRADDTAVYFCAKDVRFIAVPGDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 1350 bp, synthetic construct
  misc_feature 1..1350: note = Description of Artificial Sequence: Synthetic polynucleotide
  source 1..1350: mol_type = other DNA
gaggtgcagctgctcgagtcagggggaggcttggtacagccgggggggtccctgagactctcctgtacaacctctggattcacctttaacacgtatgccatgagttgggtccgccaggctccagggaaggggctggaatggctctcaggtattaataacaatggtcggactgcattctacgcagactccgtgaagggccgcttcaccatctccagagacaactccaaaaacacactttatctgcaaattaatagtctgagagcggacgacacggccgtttatttctgtgcgaaagatgtcagatttatcgcagtgcctggtgactcctggggccagggaaccctggtcaccgtctcctcagctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 98 - DNA, 1854 bp, synthetic construct
  misc_feature 1..1854: note = Description of Artificial Sequence: Synthetic polynucleotide
  source 1..1854: mol_type = other DNA
gacgcctctttaccctatttacagaaggagagcgtctttcagtccggcgctcacgcctataggatccccgctttactgtatttacccggtcagcagtctttactggctttcgccgagcagcgggcttccaagaaggacgagcacgctgagctgatcgtgttacgtaggggagactacgacgcccccacccatcaagttcaatggcaagctcaagaagtggtggctcaagctcggctcgatggccatcggagcatgaacccttgtcccctctacgacgcccaaaccggcactttatttctgttcttcatcgccatccccggtcaagttaccgagcagcaacagctgcagacccgggctaacgtgacaaggctgtgccaagttacctccaccgaccacggaaggacttggtcctcccctcgtgatctgaccgatgccgctatcggccccgcttaccgggagtggtccacctttgccgtgggacccggccattgtctgcagctgcatgatagggctcggtctttagtggtgcccgcttacgcctaccggaagctgcaccccaagcagcggcctatcccctccgctttttgttttttaagccatgaccatggtcgtacttgggctcgtggccattttgtggcccaagatactttagagtgccaagttgccgaggtggagactggtgagcagcgggtggtgactttaaatgcccggtcccatttaagggctagggtgcaagcccagtccaccaacgacggactggatttccaagaatcccagctggtgaagaagctcgtcgaacctcccccccaaggttgccaaggaagcgtgatctccttcccctcccctaggagcggacccggttcccccgctcagtggctgctctacacccatcccacccattcttggcagagggctgatttaggcgcctatttaaaccctcgtcctcccgctcccgaagcttggagcgagcccgtgctgctcgctaagggcagcgccgcctacagcgatttacagtccatgggaaccggacccgatggcagccctctgttcggctgtttatatgaggctaacgactacgaggagatcgtgtttctcatgttcactttaaagcaagcttttcccgctgagtatctgccccaaggtggaggcggcagcggcggcggcggctccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaccagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 99 - DNA, 2625 bp, synthetic construct
  misc_feature 1..2625: note = Description of Artificial Sequence: Synthetic polynucleotide
  source 1..2625: mol_type = other DNA
atggcctctttaccctatttacagaaggagagcgtctttcagtccggcgctcacgcctataggatccccgctttactgtatttacccggtcagcagtctttactggctttcgccgagcagcgggcttccaagaaggacgagcacgctgagctgatcgtgttacgtaggggagactacgacgcccccacccatcaagttcaatggcaagctcaagaagtggtggctcaagctcggctcgatggccatcggagcatgaacccttgtcccctctacgacgcccaaaccggcactttatttctgttcttcatcgccatccccggtcaagttaccgagcagcaacagctgcagacccgggctaacgtgacaaggctgtgccaagttacctccaccgaccacggaaggacttggtcctcccctcgtgatctgaccgatgccgctatcggccccgcttaccgggagtggtccacctttgccgtgggacccggccattgtctgcagctgcatgatagggctcggtctttagtggtgcccgcttacgcctaccggaagctgcaccccaagcagcggcctatcccctccgctttttgttttttaagccatgaccatggtcgtacttgggctcgtggccattttgtggcccaagatactttagagtgccaagttgccgaggtggagactggtgagcagcgggtggtgactttaaatgcccggtcccatttaagggctagggtgcaagcccagtccaccaacgacggactggatttccaagaatcccagctggtgaagaagctcgtcgaacctcccccccaaggttgccaaggaagcgtgatctccttcccctcccctaggagcggacccggttcccccgctcagtggctgctctacacccatcccacccattcttggcagagggctgatttaggcgcctatttaaaccctcgtcctcccgctcccgaagcttggagcgagcccgtgctgctcgctaagggcagctgcgcctacagcgatttacagtccatgggaaccggacccgatggcagccctctgttcggctgtttatatgaggctaacgactacgaggagatcgtgtttctcatgttcactttaaagcaagcttttcccgctgagtatctgccccaaggtggaggcggcagcggcggcggcggctccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggtggcggcagcggcggcggaggcagcggaggaggaggcagcgaggtgcagctggtggagtccggaggaggactggtgcagcccggaggatctttaaggctgagctgtgccgccagcggcttcaacatcaaggacacctacatccactgggtgaggcaagctcccggcaaaggactcgagtgggtggctcgtatctaccccaccaacggctatactcgttacgccgactccgtcaagggtcgtttcaccatttccgccgacacctccaagaacaccgcctatttacagatgaattctttacgggccgaagacacagctgtctactactgctcccggtggggcggagacggattctacgccatggactactggggacaaggtacactggtgacagtgtccagcggcggaggaggatctggcggcggcggaagcggcggtggcggtagcgatatccagatgacccagagcccttcctctttaagcgcttccgtgggcgatcgtgtcaccatcacttgtagggcctcccaagatgtgaacaccgctgtggcttggtaccagcagaagcccggcaaggctcccaagctgctgatctactccgccagctttctgtattccggagtgccttctcgtttcagcggctctcgtagcggcaccgacttcactttaaccatcagctctttacagcccgaggacttcgccacctactactgccagcagcattacaccacaccccccaccttcggacaaggtaccaaagtggagatcaagtga

SEQ ID NO: 100 - protein, 380 aa, synthetic construct
  REGION 1..380: note = Description of Artificial Sequence: Synthetic polypeptide
  MOD_RES 1: note = Ala, Arg, Asn, Asp, Gln, Glu, Gly, His, Leu, Lys, Met, Phe, Thr, Val, or not present
  MOD_RES 2: note = Ala or Lys
  MOD_RES 4: note = Asn or Leu
  MOD_RES 6: note = Phe, Trp, Tyr or Val
  MOD_RES 125: note = Ala, Cys, Ile, Ser or Val
  MOD_RES 187: note = Arg, Ile or Lys
  MOD_RES 196: note = Ala, Cys, Leu or Val
  MOD_RES 257: note = Glu or Lys
  MOD_RES 272: note = Cys or Val
  MOD_RES 325: note = Lys or Val
  MOD_RES 332: note = Ala, Cys, Ser or Val
  MOD_RES 352: note = Cys, Leu or Val
XXSXPXLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLXQVTSTDHGRTWSSPRDLTDAAIGPAYREWSTFAVGPGHCLQLHDRARSLVVPAYAYRKLHPXQRPIPSAFXFLSHDHGRTWARGHFVAQDTLECQVAEVETGEQRVVTLNARSHLRARVQAQSTNDGLDFQXSQLVKKLVEPPPQGXQGSVISFPSPRSGPGSPAQWLLYTHPTHSWQRADLGAYLNPRPPAPEAWSEPXLLAKGSXAYSDLQSMGTGPDGSPLFGXLYEANDYEEIVFLMFTLKQAFPAEYLPQ

SEQ ID NO: 101 - protein, 617 aa, synthetic construct
  REGION 1..617: note = Description of Artificial Sequence: Synthetic polypeptide
  MOD_RES 1: note = Ala, Arg, Asn, Asp, Gln, Glu, Gly, His, Leu, Lys, Met, Phe, Thr, Val, or not present
  MOD_RES 2: note = Ala or Lys
  MOD_RES 4: note = Asn or Leu
  MOD_RES 6: note = Phe, Trp, Tyr or Val
  MOD_RES 125: note = Ala, Cys, Ile, Ser or Val
  MOD_RES 187: note = Arg, Ile or Lys
  MOD_RES 196: note = Ala, Cys, Leu or Val
  MOD_RES 257: note = Glu or Lys
  MOD_RES 272: note = Cys or Val
  MOD_RES 325: note = Lys or Val
  MOD_RES 332: note = Ala, Cys, Ser or Val
  MOD_RES 352: note = Cys, Leu or Val
XXSXPXLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLXQVTSTDHGRTWSSPRDLTDAAIGPAYREWSTFAVGPGHCLQLHDRARSLVVPAYAYRKLHPXQRPIPSAFXFLSHDHGRTWARGHFVAQDTLECQVAEVETGEQRVVTLNARSHLRARVQAQSTNDGLDFQXSQLVKKLVEPPPQGXQGSVISFPSPRSGPGSPAQWLLYTHPTHSWQRADLGAYLNPRPPAPEAWSEPXLLAKGSXAYSDLQSMGTGPDGSPLFGXLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 102 - protein, 874 aa, synthetic construct
  REGION 1..874: note = Description of Artificial Sequence: Synthetic polypeptide
  MOD_RES 1: note = Ala, Arg, Asn, Asp, Gln, Glu, Gly, His, Leu, Lys, Met, Phe, Thr, Val, or not present
  MOD_RES 2: note = Ala or Lys
  MOD_RES 4: note = Asn or Leu
  MOD_RES 6: note = Phe, Trp, Tyr or Val
  MOD_RES 125: note = Ala, Cys, Ile, Ser or Val
  MOD_RES 187: note = Arg, Ile or Lys
  MOD_RES 196: note = Ala, Cys, Leu or Val
  MOD_RES 257: note = Glu or Lys
  MOD_RES 272: note = Cys or Val
  MOD_RES 325: note = Lys or Val
  MOD_RES 332: note = Ala, Cys, Ser or Val
  MOD_RES 352: note = Cys, Leu or Val
XXSXPXLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLXQVTSTDHGRTWSSPRDLTDAAIGPAYREWSTFAVGPGHCLQLHDRARSLVVPAYAYRKLHPXQRPIPSAFXFLSHDHGRTWARGHFVAQDTLECQVAEVETGEQRVVTLNARSHLRARVQAQSTNDGLDFQXSQLVKKLVEPPPQGXQGSVISFPSPRSGPGSPAQWLLYTHPTHSWQRADLGAYLNPRPPAPEAWSEPXLLAKGSXAYSDLQSMGTGPDGSPLFGXLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 103 - protein, 875 aa, synthetic construct
  REGION 1..875: note = Description of Artificial Sequence: Synthetic polypeptide
TVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYNGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 104 - DNA, 2625 bp, synthetic construct
  misc_feature 1..2625: note = Description of Artificial Sequence: Synthetic polynucleotide
  source 1..2625: mol_type = other DNA
acagtggaaaagtccgtggtgttcaaggccgagggcgagcacttcaccgaccagaaaggcaataccatcgtcggctctggcagcggcggcaccaccaagtactttagaatccccgccatgtgcaccaccagcaagggcaccattgtggtgttcgccgacgccagacacaacaccgccagcgatcagagcttcatcgataccgctgccgccagatctaccgatggcggcaagacctggaacaagaagatcgccatctacaacgaccgcgtgaacagcaagctgagcagagtgatggaccctacctgcatcgtggccaacatccagggcagagaaaccatcctggtcatggtcggaaagtggaacaacaacgataagacctggggcgcctacagagacaaggcccctgataccgattgggacctcgtgctgtacaagagcaccgatgacggcgtgaccttcagcaaggtggaaacaaacatccacgacatcgtgaccaagaacggcaccatctctgccatgctcggcggcgttggatctggcctgcaactgaatgatggcaagctggtgttccccgtgcagatggtccgaacaaagaatatcaccaccgtgctgaataccagcttcatctacagcaccgacggcatcacatggtccctgcctagcggctactgtgaaggctttggcagcgagaacaacatcatcgagttcaacgccagcctggtcaacaacatccggaacagcggcctgcggagaagcttcgagacaaaggacttcggaaagacgtggaccgagtttcctccaatggacaagaaggtggacaaccggaaccacggcgtgcagggcagcacaatcacaatccctagcggcaacaaactggtggccgctcactctagcgcccagaacaagaacaacgactacaccagaagcgacatcagcctgtacgcccacaacctgtacagcggcgaagtgaagctgatcgacgacttctaccccaaagtgggcaatgccagcggagccggctacagctgtctgagctaccggaaaaatgtggacaaagaaaccctgtacgtggtgtacgaggccaacggcagcatcgagtttcaggacctgagcagacatctgcccgtgatcaagagctacaacggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggaggcggaggatctggcggaggtggaagtggcggaggcggatctgaggtgcagctggttgaatctggcggaggactggttcagcctggcggatctctgagactgtcttgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgttctagatggggaggcgacggcttctacgccatggattattggggccagggcaccctggtcaccgtttcttctggcggaggaggatctggcggaggcggaagtggcggaggcggatctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgccagctttctgtactccggcgtgcccagcagattcagcggctctagaagcggcaccgacttcaccctgaccataagcagtctgcagcccgaggacttcgccacctactactgtcagcagcactacaccacacctccaacctttggccagggcaccaaggtggaaatcaag

SEQ ID NO: 105 - protein, 10 aa, synthetic construct
  REGION 1..10: note = Description of Artificial Sequence: Synthetic 10xHis tag
HHHHHHHHHH

SEQ ID NO: 106 - protein, 15 aa, synthetic construct
  REGION 1..15: note = Description of Artificial Sequence: Synthetic peptide
  REGION 1..15: note = MISC_FEATURE - This sequence may encompass 1-5 "Gly Gly Pro" repeating units
GGPGGPGGPGGPGGP

SEQ ID NO: 107 - protein, 25 aa, synthetic construct
  REGION 1..25: note = Description of Artificial Sequence: Synthetic peptide
  REGION 1..25: note = MISC_FEATURE - This sequence may encompass 1-5 "Gly Gly Gly Gly Ser" repeating units
GGGGSGGGGSGGGGSGGGGSGGGGS

SEQ ID NO: 108 - protein, 10 aa, synthetic construct
  REGION 1..10: note = Description of Artificial Sequence: Synthetic peptide
GGGGSGGGGS

SEQ ID NO: 109 - protein, 6 aa, synthetic construct
  REGION 1..6: note = Description of Artificial Sequence: Synthetic 6xHis tag
HHHHHH

(19) United States
(12) Patent Application Publication
(10) Pub. No.: US 2024/0344046 A1
(43) Pub. Date: Oct. 17, 2024

(54) RECOMBINANT HUMAN SIALIDASES, SIALIDASE FUSION PROTEINS, AND METHODS OF USING THE SAME

(71) Applicant: Palleon Pharmaceuticals Inc., Waltham, MA (US)

(72) Inventors: Li Peng, Lexington, MA (US); Lizhi Cao, Stoneham, MA (US); Lihui Xu, Chestnut Hill, MA (US)

(21) Appl. No.: 18/597,204

(22) Filed: Mar. 6, 2024

Related U.S. Application Data
(63) Continuation of application No. 16/958,914, filed on Jun. 29, 2020, now Pat. No. 11,965,188.
(60) Provisional application No. 62/755,279, filed on Nov. 2, 2018.
(60) Provisional application No. 62/613,363, filed on Jan. 3, 2018.

(51) Int. Cl. C12N 9/24; A61K 39/00; A61K 47/68; A61P 35/00; C07K 16/28; C07K 16/32

(52) U.S. Cl. CPC C12N 9/2402; A61K 47/6815; A61K 47/6855; A61P 35/00; C07K 16/2863; C07K 16/32; A61K 2039/505

(57) ABSTRACT

The invention relates generally to recombinant human sialidases and recombinant sialidase fusion proteins, wherein the sialidase optionally contains one or more mutations compared to wild-type human sialidase, e.g., a substitution, deletion, or addition of at least one amino acid. The invention also provides antibody conjugates including a sialidase and an antibody or a portion thereof. The invention further relates to methods of using the sialidase fusion proteins or antibody conjugates for treating cancer.

### CROSS REFERENCE TO RELATED APPLICATIONS

This application is continuation of U.S. patent application Ser. No. 16/958,914, filed on Jun. 29, 2020, which is a § 371 National Stage of International (PCT) Patent Application No. PCT/US2019/012207, filed Jan. 3, 2019, which claims the benefit of, and priority to, U.S. Provisional Patent Application No. 62/613,363, filed Jan. 3, 2018 and U.S. Provisional Patent Application No. 62/755,279, filed Nov. 2, 2018, the entire disclosure of each of which is hereby incorporated by reference for all purposes.

### SEQUENCE LISTING

The instant application contains a Sequence Listing which has been submitted electronically in XML format and is hereby incorporated by reference in its entirety. The XML copy, created on Mar. 5, 2024, is named PAL-009USC1_SL.xml and is 218.6 kilobytes in size.

### FIELD OF THE INVENTION

The invention relates generally to recombinant human sialidases and recombinant sialidase fusion proteins, and their use in the treatment of cancer.

### BACKGROUND

A growing body of evidence supports roles for glycans, and sialoglycans in particular, at various pathophysiological steps of tumor progression. Glycans regulate tumor proliferation, invasion, hematogenous metastasis and angiogenesis (Fuster et al. (2005) NAT. REV. CANCER 5 (7): 526-42). The sialylation of cell surface glycoconjugates is frequently altered in cancers, resulting in the expression of sialylated tumor-associated carbohydrate antigens. The expression of sialylated glycans by tumor cells is often associated with increased aggressiveness and metastatic potential of a tumor.

It has recently become apparent that Siglecs (sialic acid-binding immunoglobulin-like lectins), a family of sialic acid binding lectins, play a role in cancer immune suppression by binding to hypersialylated cancer cells and mediating the suppression of signals from activating NK cell receptors, thereby inhibiting NK cell-mediated killing of tumor cells (Jandus et al. (2014) J. CLIN. INVEST. 124:1810-1820; Läubli et al. (2014) PROC. NATL. ACAD. SCI. USA 111:14211-14216; Hudak et al. (2014) NAT. CHEM. BIOL. 10:69-75). Likewise, enzymatic removal of sialic acids by treatment with sialidase can enhance NK cell-mediated killing of tumor cells (Jandus, supra; Hudak, supra; Xiao et al. (2016) PROC. NATL. ACAD. SCI. USA 113 (37): 10304-9.)

Cancer immunotherapy with immune checkpoint inhibitors, including antibodies blocking the PD-1/PD-L1 pathway, has improved the outcome of many cancer patients. However, despite advances that have been made to date, many patients do not respond to currently available immune checkpoint inhibitors. Accordingly, there is still a need for effective interventions that overcome the immune suppressive tumor microenvironment and for treating cancers associated with hypersialylated cancer cells.

### SUMMARY OF THE INVENTION

The invention is based, in part, upon the discovery that it is possible to produce recombinant mutant forms of human sialidase enzymes and fusion proteins and/or antibody conjugates containing such enzymes that have suitable substrate specificities and activities to be useful in removing sialic acid and/or sialic acid containing molecules from the surface of cancer cells and/or removing sialic acid and/or sialic acid containing molecules from the tumor microenvironment, and/or reducing the concentration of sialic acid and/or sialic acid containing molecules in the tumor microenvironment.

In one aspect, the invention provides a recombinant mutant human sialidase comprising a substitution of at least one wild-type amino acid residue, wherein the substitution increases at least one of the (a) expression, (b) stability, and (c) activity of the sialidase, or a combination of (a) and (b), combination of (a) and (c), a combination of (b) and (c), or a combination of (a), (b) and (c).

In another aspect, the invention provides a recombinant mutant human sialidase enzyme comprising an N-terminus and a C-terminus and comprising: (a) a substitution of at least one wild-type cysteine residue; (b) a substitution of at least one wild-type amino acid residue, wherein the substitution increases the isoelectric point (pI) of the sialidase and/or decreases the hydrophobicity of the sialidase relative to a sialidase without the substitution; (c) a peptide at least two amino acid residues in length covalently associated with an N-terminal amino acid at the N-terminus of the sialidase; (d) a substitution of at least one wild-type amino acid residue, wherein the substitution increases hydrophobic interactions and/or hydrogen bonding between the N- and C-termini of the sialidase relative to a sialidase without the substitution; or (e) a substitution or deletion of an N-terminal methionine at the N-terminus of the sialidase; or a combination of any of the foregoing. For example, the recombinant mutant sialidase enzyme may comprise a combination of the above-identified features, namely (a), (b), (c), (d), and (c), and may include, for example, a combination selected from: (a) and (b); (a) and (c); (a) and (d); (a) and (c); (b) and (c); (b) and (d); (b) and (c); (c) and (d); (c) and (c); (d) and (c); (a) and (b) and (c); (b) and (c) and (d); (a) and (c) and (d); (a) and (b) and (d); (a) and (b) and (c); (a) and (c) and (c); (a) and (d) and (c); (b) and (c) and (c); (b) and (d) and (c); (c) and (d) and (c); (a) and (b) and (c) and (d); (a) and (b) and (c) and (c); (a) and (c) and (d) and (c); (b) and (c) and (d) and (c); and (a) and (b) and (c) and (d) and (c). In certain embodiments, the sialidase is selected from Neu1, Neu2, Neu3, and Neu4, e.g., the sialidase is Neu2.

In certain embodiments, the sialidase comprises a substitution of at least one wild-type cysteine residue, e.g., a free cysteine residue. The cysteine residue may, for example, be substituted by serine, isoleucine, valine, phenylalanine, leucine, or alanine. In certain embodiments, the sialidase comprises a substitution of a cysteine residue at a position corresponding to position 332 of wild-type human Neu2 (SEQ ID NO: 1), e.g., the cysteine residue at a position corresponding to position 332 of wild-type human Neu2 is substituted by alanine (C332A). In certain embodiments, the sialidase comprises a substitution of a cysteine residue at a position corresponding to position 352 of wild-type human Neu2 (SEQ ID NO: 1), e.g., the cysteine residue at a position corresponding to position 352 of wild-type human Neu2 is substituted by leucine (C352L). In certain embodiments, the sialidase comprises both the C332A and C352L substitutions. In certain embodiments, the sialidase contains an amino acid substitution at 2, 3, 4, 5, or 6 cysteines typically present in a human sialidase, e.g., Neu2 or Neu3.

In certain embodiments, the sialidase comprises a substitution of at least one wild-type amino acid residue, e.g., a solvent exposed wild-type amino acid residue, wherein the substitution increases the isoelectric point (pI) of the sialidase and/or decreases the hydrophobicity of the sialidase relative to a sialidase without the substitution. In certain embodiments, the wild-type amino acid is substituted by lysine, arginine, or histidine, e.g., the wild-type amino acid is substituted by lysine. In certain embodiments, the sialidase comprises a substitution of an alanine residue at a position corresponding to position 2 of wild-type human Neu2 (SEQ ID NO: 1), e.g., the alanine residue at a position corresponding to position 2 of wild-type human Neu2 is substituted by lysine (A2K).

In certain embodiments, the sialidase comprises a peptide at least two amino acid residues in length fused to the N-terminus of the sialidase, e.g., fused to an N-terminal amino acid residue of the sialidase, e.g., by a peptide bond. In certain embodiments, the peptide is between 2 amino acid residues and 20 amino acid residues in length. In certain embodiments, the peptide is at least two, three, four or five amino acid residues in length. In certain embodiments, the peptide comprises an amino acid sequence derived from wild-type mouse thymus Neu2 (SEQ ID NO: 2), e.g., in certain embodiments the peptide comprises EDLRP (SEQ ID NO: 3) or MEDLRP (SEQ ID NO: 4).

In certain embodiments, the sialidase comprises a substitution of at least one wild-type amino acid residue, wherein the substitution increases hydrophobic interactions and/or hydrogen bonding between the N- and C-termini of the sialidase relative to a sialidase without the substitution. For example, in certain embodiments, the sialidase comprises a substitution of a valine residue at a position corresponding to position 6 of wild-type human Neu2 (SEQ ID NO: 1), e.g., the valine residue at a position corresponding to position 6 of wild-type human Neu2 is substituted by tyrosine (V6Y).

In certain embodiments, the sialidase comprises a substitution or deletion of an N-terminal methionine at the N-terminus of the sialidase. For example, in certain embodiments, the sialidase comprises a substitution of a methionine residue at a position corresponding to position 1 of wild-type human Neu2 (SEQ ID NO: 1), e.g., the methionine at a position corresponding to position 1 of wild-type human Neu2 is substituted by alanine (MIA) or aspartic acid (MID).

In certain embodiments, the sialidase has a different substrate specificity than the corresponding wild-type sialidase. For example, in certain embodiments the sialidase can cleave α2,3, α2,6, and/or α2,8 linkages. In certain embodiments the sialidase can cleave α2,3 and α2,8 linkages.

In certain embodiments, the sialidase comprises SEQ ID NO: 5, SEQ ID NO: 6, SEQ ID NO: 36, SEQ ID NO: 37, SEQ ID NO: 38, SEQ ID NO: 39, SEQ ID NO: 70, SEQ ID NO: 71, SEQ ID NO: 72, or SEQ ID NO: 73.

In another aspect, the invention provides a fusion protein comprising: (a) a sialidase enzyme; and (b) an immunoglobulin Fc domain and/or an immunoglobulin antigen-binding domain; wherein the sialidase and the Fc domain and/or the antigen-binding domain are linked by a peptide bond or an amino acid linker. In certain embodiments, the sialidase is a human sialidase, e.g., a recombinant mutant human sialidase disclosed herein. In certain embodiments, the fusion protein further comprises a linker, for example, an amino acid linker, connecting the sialidase enzyme and the Fc domain and/or an antigen-binding domain. In certain embodiments, the immunoglobulin antigen-binding domain is associated (for example, covalently or non-covalently associated) with a second immunoglobulin antigen-binding domain to produce an antigen-binding site.

In certain embodiments, the immunoglobulin Fc domain is derived from a human IgG1, IgG2, IgG3, IgG4, IgA1, IgA2, IgD, IgE, or IgM Fc domain, e.g., the immunoglobulin Fc domain is derived from a human IgG1, IgG2, IgG3, or IgG4 Fc domain, e.g., the immunoglobulin Fc domain is derived from a human IgG1 Fc domain.

In certain embodiments, the immunoglobulin antigen-binding domain is derived from an antibody selected from trastuzumab, cetuximab, daratumumab, girentuximab, panitumumab, ofatumumab, and rituximab. In certain embodiments, the immunoglobulin antigen-binding domain is derived from trastuzumab.

In certain embodiments, the fusion protein comprises SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 63, SEQ ID NO: 74, SEQ ID NO: 75, SEQ ID NO: 76, SEQ ID NO: 77, SEQ ID NO: 78, or SEQ ID NO: 79, or an amino acid sequence that has at least 85%, 90%, 95%, 96%, 97%, 98%, or 99% sequence identity to SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 63, SEQ ID NO: 74, SEQ ID NO: 75, SEQ ID NO: 76, SEQ ID NO: 77, SEQ ID NO: 78, or SEQ ID NO: 79.

In another aspect, the invention provides an antibody conjugate comprising any of the foregoing fusion proteins. In certain embodiments, the antibody conjugate comprises a single sialidase. In other embodiments, the antibody conjugate comprises two sialidases, which can be the same or different. In certain embodiments the antibody conjugate comprises two identical sialidases. In certain embodiments, the antibody conjugate comprises a single antigen-binding site. In other embodiments, the antibody conjugate comprises two antigen-binding sites, which can be the same or different. In certain embodiments, the antibody conjugate comprises two identical antigen-binding sites.

In certain embodiments, the antibody conjugate has a molecular weight from about 135 kDa to about 165 kDa, or the antibody conjugate has a molecular weight from about 215 kDa to about 245 kDa.

In certain embodiments, the antibody conjugate comprises: (a) a first polypeptide comprising an immunoglobulin light chain; (b) a second polypeptide comprising an immunoglobulin heavy chain; and (c) a third polypeptide comprising an immunoglobulin Fc domain and a sialidase; wherein the first and second polypeptides are covalently linked together and the second and third polypeptides are covalently linked together, and wherein the first polypeptide and the second polypeptide together define an antigen-binding site. The third polypeptide may, for example, comprise the sialidase and the immunoglobulin Fc domain in an N- to C-terminal orientation. The first polypeptide may, for example, comprise SEQ ID NO: 49, the second polypeptide may, for example, comprise SEQ ID NO: 50, and/or the third polypeptide may, for example, comprise SEQ ID NO: 51, SEQ ID NO: 52, SEQ ID NO: 53, SEQ ID NO: 54, SEQ ID NO: 76, SEQ ID NO: 77, SEQ ID NO: 78, or SEQ ID NO: 79.

In certain embodiments, the antibody conjugate comprises: (a) a first polypeptide comprising a first immunoglobulin light chain; (b) a second polypeptide comprising a first immunoglobulin heavy chain and a first sialidase; (c) a third polypeptide comprising a second immunoglobulin heavy chain and a second sialidase; and (d) a fourth polypeptide comprising a second immunoglobulin light chain; wherein the first and second polypeptides are covalently linked together, the third and fourth polypeptides are covalently linked together, and the second and third polypeptides are covalently linked together, and wherein the first polypeptide and the second polypeptide together define a first antigen-binding site, and the third polypeptide and the fourth polypeptide together define a second antigen-binding site. The second and third polypeptides may, for example, comprise the first and second immunoglobulin heavy chain and the first and second sialidase, respectively, in an N- to C-terminal orientation.

In certain embodiments, the antibody conjugate comprises: (a) a first polypeptide comprising a first sialidase, a first immunoglobulin Fc domain, and a first single chain variable fragment (scFv); and (b) a second polypeptide comprising a second sialidase, a second immunoglobulin Fc domain, and an optional second single chain variable fragment (scFv); wherein the first and second polypeptides are covalently linked together, and wherein the first scFv defines a first antigen-binding site, and the second scFv, when present, defines a second antigen-binding site. The first polypeptide may, for example comprise the first sialidase, the first immunoglobulin Fc domain, and the first scFv in an N- to C-terminal orientation. The second polypeptide may, for example, comprise the second sialidase, the second immunoglobulin Fc domain, and the optional second scFv in an N- to C-terminal orientation. The first polypeptide may, for example, comprise SEQ ID NO: 43, SEQ ID NO: 44, SEQ ID NO: 45, SEQ ID NO: 46, SEQ ID NO: 47, SEQ ID NO: 48, SEQ ID NO: 74, or SEQ ID NO: 75, and/or the second polypeptide may, for example, comprise SEQ ID NO: 43, SEQ ID NO: 44, SEQ ID NO: 45, SEQ ID NO: 46, SEQ ID NO: 47, SEQ ID NO: 48, SEQ ID NO: 74, or SEQ ID NO: 75.

In another aspect, the invention provides an isolated nucleic acid comprising a nucleotide sequence encoding any of the foregoing recombinant mutant human sialidases, any of the foregoing fusion proteins, or at least a portion of any of the foregoing antibody conjugates. In another aspect, the invention provides an expression vector comprising any of the foregoing nucleic acids. In another aspect, the invention provides a host cell comprising any of the foregoing expression vectors.

In another aspect, the invention provides a pharmaceutical composition comprising any of the foregoing recombinant mutant human sialidases, any of the foregoing fusion proteins, or any of the foregoing antibody conjugates.

In another aspect, the invention provides a method of treating cancer in a subject in need thereof. The method comprises administering to the subject an effective amount of any of the foregoing sialidases, any of the foregoing fusion proteins, any of the foregoing antibody conjugates, or any of the foregoing pharmaceutical compositions. In certain embodiments, the cancer is an epithelial cancer, e.g., endometrial cancer, ovarian cancer, cervical cancer, vulvar cancer, uterine cancer, fallopian tube cancer, breast cancer, prostate cancer, lung cancer, pancreatic cancer, urinary cancer, bladder cancer, head and neck cancer, oral cancer and liver cancer.

In another aspect, the invention provides a method of increasing expression of HLA-DR, CD86, CD83, IFNγ, IL-1b, IL-6, TNFα, IL-17A, IL-2, or IL-6 in a cell or tissue. The method comprises contacting the cell or tissue with an effective amount of any of the foregoing sialidases, any of the foregoing fusion proteins, any of the foregoing antibody conjugates, or any of the foregoing pharmaceutical compositions. In certain embodiments, the cell is selected from a dendritic cell and a peripheral blood mononuclear cell (PBMC).

These and other aspects and features of the invention are described in the following detailed description and claims.

### DESCRIPTION OF THE DRAWINGS

The invention can be more completely understood with reference to the following drawings.

FIG. 1 depicts an SDS-PAGE gel showing recombinant human Neu1, Neu2, Neu3, and Salmonella typhimurium (St-sialidase) under non-reducing and reducing conditions. Monomer and dimer species are indicated.

FIG. 2 is a bar graph showing the enzymatic activity of recombinant human Neu1, Neu2, and Neu3.

FIG. 3 is a line graph showing enzymatic activity as a function of substrate concentration for recombinant human Neu2 and Neu3 at the indicated pH.

FIG. 4A is an SEC-HPLC trace of wild-type Neu2. FIG. 4B is an SEC-HPLC trace of Neu2-M38 (containing C332A and C352L substitutions). Quantities of monomer and aggregate species are indicated.

FIG. 5A is an SEC-HPLC trace of wild-type Neu2, FIG. 5B is an SEC-HPLC trace of Neu2-M62 (containing the A2K substitution), and FIG. 5C is an SEC-HPLC trace of Neu2-M71 (containing A2K and V325 substitutions). Quantities of monomer and aggregate species are indicated.

FIG. 6A is an SEC-HPLC trace of wild-type Neu2 and FIG. 6B is an SEC-HPLC trace of Neu2-M76 (which included MEDLRP (SEQ ID NO: 4) inserted at the N-terminus). Quantities of monomer and aggregate species are indicated.

FIG. 7A is a line graph showing the enzymatic activity as a function of substrate concentration for Neu2-M76 at the indicated pH. FIG. 7B depicts cleavage of α2-3, α2-6, or α2-8 substrate by Neu2-M76.

FIG. 8A is an SEC-HPLC trace of wild-type Neu2 and FIG. 8B is an SEC-HPLC trace of Neu2-M79 (containing the V6Y substitution). Quantities of monomer and aggregate species are indicated.

FIGS. 9A-9I depict schematic representations of certain antibody conjugate constructs containing a sialidase enzyme, e.g., a human sialidase enzyme, and an antigen binding site. For each antibody conjugate construct that contains more than one (e.g., two) sialidase, each sialidase may be the same or different. For each antibody conjugate construct that contains more than one (e.g., two) antigen binding site, each antigen binding site may be the same or different.

FIGS. 10A-C are schematic representations of fusion protein conjugates referred to as a Raptor antibody sialidase conjugate (FIG. 10A), a Janus antibody sialidase conjugate (FIG. 10B), and a Lobster antibody sialidase conjugate (FIG. 10C).

FIG. 11A is an SDS-PAGE gel showing antibody-sialidase conjugates (ASCs) made using Salmonella typhimurium sialidase (St-sialidase) and trastuzumab in the Raptor (left) and Janus (right) formats. FIG. 11B is an SEC-HPLC trace of trastuzumab (top), and ASCs made using St-sialidase and trastuzumab in the Janus (middle) and Raptor (bottom) formats.

FIG. 12A is a line graph (top) and a bar graph (bottom) showing the enzymatic activity for St-sialidase and ASCs made using St-sialidase and trastuzumab in the Raptor and Janus formats. FIG. 12B depicts binding to Her2 as determined by ForteBio Octet for trastuzumab (top), and ASCs made using St-sialidase and trastuzumab in the Raptor (middle) and Janus (bottom) formats.

FIGS. 13A-C show an SDS-PAGE gel depicting an ASC made using St-sialidase and trastuzumab in the Janus format (FIG. 13A), an SDS-PAGE gel depicting an ASC made using Neu2-M76 (which included MEDLRP (SEQ ID NO: 4) inserted at the N-terminus) and trastuzumab in the Janus format (FIG. 13B), and an SDS-PAGE gel depicting an ASC made using a Neu2-M85 (which included a deletion of M1 and V6Y and I187K mutations) and a scFv derived from trastuzumab in the Lobster format (FIG. 13C).

FIGS. 14A-C show line graphs depicting enzymatic activity as a function of substrate concentration for an ASC made using Neu2-M76 (which included MEDLRP (SEQ ID NO: 4) inserted at the N-terminus) and trastuzumab in the Janus format (FIG. 14A), wild-type Neu2 (FIG. 14B), and an ASC made using a Neu2-M85 (which included a deletion of M1 and V6Y and I187K mutations) and a scFv derived from trastuzumab in the Lobster format (FIG. 14C).

FIGS. 15A-D depict the testing of various configurations of antibody sialidase conjugates in a mouse syngeneic tumor model utilizing EMT6 mouse breast cancer cells engineered to express human Her2. Mice are treated via intraperitoneal injection of 10 mg/kg of each test article on the days marked with black triangles and tumor volume (mm³) recorded. Each line represents an individual mouse. Mice are treated with either trastuzumab (FIG. 15A), Raptor (FIG. 15B), Janus (FIG. 15C) or Lobster (FIG. 15D).

FIGS. 16A-D depict the testing of the Janus antibody sialidase conjugate in a mouse syngeneic tumor model utilizing EMT6 mouse breast cancer cells engineered to express human Her2. Mice were treated via intraperitoneal injection of 10 mg/kg of Janus on the days marked with black triangles and tumor volume (mm³) recorded. Mice were also treated on the same days as Janus with either anti-mouse NK1.1 (10 mg/kg) to deplete natural killer cells (FIG. 16A), liposomal clodronate (0.5 mg/mouse, three times a week for two weeks) to deplete macrophages (FIG. 16B), or anti-mouse CD8α (10 mg/kg) to deplete CD8+ T cells (FIG. 16C). Each line represents an individual mouse. FIG. 16D depicts the mean tumor volume with error bars of the indicated treatment groups from Example 8.

FIGS. 17A-B depict the testing of the Janus antibody sialidase conjugate in a mouse syngeneic orthotopic tumor model utilizing a second source of EMT6 mouse breast cancer cells engineered to express human Her2. Mice are treated via intraperitoneal injection of 10 mg/kg of each test article on the days marked with black triangles and tumor volume (mm³) recorded. Each line represents an individual mouse. Mice are treated (▾) with either vehicle, trastuzumab, Janus or Janus Loss of Function (FIG. 17A). FIG. 17B depicts the rechallenge experiment of either the three mice treated with Janus from FIG. 17A with complete regressions of the original EMT6-Her2 tumors (cured mice) or naïve mice. Cured mice were inoculated with either EMT6-Her2 cells or parental EMT6 cells on the left and right lower flank region. Naïve mice were inoculated with EMT6-Her2 cells.

FIGS. 18A-B depict the testing of the Janus antibody sialidase conjugate in a mouse syngeneic orthotopic tumor model in combination with anti-mouse PD1. Mice are treated via intraperitoneal injection of 10 mg/kg of either anti-mouse PD1 alone (FIG. 18A) or Janus and anti-mouse PD1 (10 mg/kg of each, FIG. 18B) on the days marked with black triangles (▾) and tumor volume (mm³) recorded. Each line represents an individual mouse.

FIG. 19 depicts the testing of various test articles in a mouse syngeneic tumor model injected with a B16 melanoma cell line expressing human Her2. Mice are treated via intraperitoneal injection of 10 mg/kg of either Janus, trastuzumab or a combination of anti-mouse PD1 and anti-mouse CTLA4 (10 mg/kg of each) on the days marked with black triangles (▾) and tumor volume (mm³) recorded. Each line represents an individual mouse.

FIG. 20 depicts binding of MAL II and PNA lectins to tumor cells, as assayed by FACS staining, following the indicated treatment. MAL II and PNA staining are indicative of cleavage of terminal sialic acids from the tumor cells, MAL II staining is expected to decrease upon loss of cell surface sialic acid and PNA staining is expected to increase with loss of cell surface sialic acid.

FIG. 21 depicts cell surface expression of dendritic cell (DC) activation markers HLA-DR, CD86 and CD83, as assayed by flow cytometry, following the indicated treatment. A indicates a Janus ASC made with St-sialidase and trastuzumab, B indicates a non-Her2 binding Janus ASC made with St-sialidase, and C indicates a Janus ASC made with a loss of function St-sialidase mutant and trastuzumab. *P≤0.05, **P≤0.01, ***P≤0.001, and *** P≤0.0001.

FIGS. 22A-D depict IFNγ (FIG. 22A), IL-1b (FIG. 22B), IL-6 (FIG. 22C), and TNFα (FIG. 22D) release following treatment with a Janus ASC including human Neu2 with ΔM1, V6Y, I187K, and C332A mutations and trastuzumab. Freshly isolated human peripheral blood mononuclear cells (PBMCs) were incubated with Janus at the indicated concentrations (shown in μg/ml) for 24 hours. PHA-L with IL-2 or LPS were used as positive controls to stimulate cytokine release. Trastuzumab (Tras) was used as a negative control.

FIGS. 23A-D depict IL-2 (FIG. 23A), IL-4 (FIG. 23B), IL-10 (FIG. 23C), and IL-13 (FIG. 23D) release following treatment with a Janus ASC including human Neu2 with ΔM1, V6Y, I187K, and C332A mutations and trastuzumab. Freshly isolated human peripheral blood mononuclear cells (PBMCs) were incubated with Janus at the indicated concentrations (shown in μg/ml) for 24 hours. PHA-L with IL-2 or LPS were used as positive controls to stimulate cytokine release. Trastuzumab (Tras) was used as a negative control.

FIG. 24 depicts relative expression of the indicated markers following addition of Janus and Janus LOF to the BioMAP VascHT29 co-culture tumor microenvironment model. The historical range of vehicle response is represented by the shaded area along the zero baseline. Values for each measurement are represented by the log of the ratio of test article to vehicle control. Analytes with a statistically meaningful value above historical ranges are annotated.

FIG. 25 depicts relative expression of the indicated markers following addition of Janus and pembrolizumab to the BioMAP VascHT29 co-culture tumor microenvironment model. The historical range of vehicle response is represented by the shaded area along the zero baseline. Values for each measurement are represented by the log of the ratio of test article to vehicle control. Analytes with a statistically meaningful value above historical ranges are annotated.

FIG. 26 depicts the testing of the Janus antibody sialidase conjugate in a mouse syngeneic tumor model utilizing EMT6 mouse breast cancer cells engineered to express human Her2. Mice were treated via intraperitoneal injection of 10 mg/kg of Janus, trastuzumab or a non-Her2 binding Janus versus vehicle on the days marked with black triangles (▾) and tumor volume (mm³) was recorded. Mean tumor volumes for each treatment group are shown. Tumor volumes for individual mice in each treatment group are shown. Complete Responses (CR, defined as regression below the limit of palpation at any point during the study) are shown as well.

FIG. 27 depicts the testing of human Lobster antibody sialidase conjugates in a mouse syngeneic tumor model utilizing EMT6 mouse breast cancer cells engineered to express human Her2. Mice were treated via intraperitoneal injection of 10 mg/kg of trastuzumab, human Lobster 1, human Lobster 2, and vehicle on the days marked with black triangles (▾) and tumor volume (mm³) was recorded. Graphs show the individual mice for the indicated treatments. Complete Responses (CR, defined as regression below the limit of palpation at any point during the study) are shown as well.

### DETAILED DESCRIPTION

Various features and aspects of the invention are discussed in more detail below. The invention provides a recombinant human sialidase that comprises at least one mutation relative to a wild-type human sialidase, e.g., a substitution, deletion, or addition (insertion) of at least one amino acid. The mutations, or combination of mutations, can improve the expression, activity or both the expression and activity of the sialidase to improve its use in cancer diagnosis and/or treatment.

The invention further relates to fusion proteins and/or antibody conjugates comprising a sialidase enzyme and an antibody or portion thereof, e.g., an immunoglobulin Fc domain and/or an antigen-binding domain. The sialidase enzyme portion of the fusion protein and/or antibody conjugate may comprise at least one mutation relative to a wild-type human sialidase.

The invention further relates to pharmaceutical compositions and methods of using fusion proteins and/or antibody conjugates to treat cancer, e.g., an epithelial cell cancer.

### I. Recombinant Human Sialidases

As used herein, the term “sialidase” refers to any enzyme, or a functional fragment thereof, that cleaves a terminal sialic acid residue from a substrate, for example, a glycoprotein or a glycolipid. The term sialidase includes variants having one or more amino acid substitutions, deletions, or insertions relative to a wild-type sialidase sequence, and/or fusion proteins or conjugates including a sialidase. Sialidases are also called neuraminidases, and, unless indicated otherwise, the two terms are used interchangeably herein. As used herein, the term “functional fragment” of a sialidase refers to fragment of a full-length sialidase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring sialidase. Sialidase enzymatic activity may be assayed by any method known in the art, including, for example, by measuring the release of sialic acid from the fluorogenic substrate 4-methylumbelliferyl-N-acetylneuraminic acid (4MU-NeuAc). In certain embodiments, the functional fragment comprises at least 100, 150, 200, 250, 300, 310, 320, 330, 340, 350, 360, or 370 consecutive amino acids present in a full-length, naturally occurring sialidase.

Four sialidases have been found in the human genome and are referred to as Neu1, Neu2, Neu3 and Neu4.

Human Neu1 is a lysosomal neuraminidase enzyme which functions in a complex with beta-galactosidase and cathepsin A. The amino acid sequence of human Neu1 is depicted in SEQ ID NO: 7, and a nucleotide sequence encoding human Neu1 is depicted in SEQ ID NO: 23.

Human Neu2 is a cytosolic sialidase enzyme. The amino acid sequence of human Neu2 is depicted in SEQ ID NO: 1, and a nucleotide sequence encoding human Neu2 is depicted in SEQ ID NO: 24.

Human Neu3 is a plasma membrane sialidase with an activity specific for gangliosides. Human Neu3 has two isoforms: isoform 1 and isoform 2. The amino acid sequence of human Neu3, isoform 1 is depicted in SEQ ID NO: 8, and a nucleotide sequence encoding human Neu3, isoform 1 is depicted in SEQ ID NO: 25. The amino acid sequence of human Neu3, isoform 2 is depicted in SEQ ID NO: 9, and a nucleotide sequence encoding human Neu3, isoform 2 is depicted in SEQ ID NO: 34.

Human Neu4 has two isoforms: isoform 1 is a peripheral membrane protein and isoform 2 localizes to the lysosome lumen. The amino acid sequence of human Neu4, isoform 1 is depicted in SEQ ID NO: 10, and a nucleotide sequence encoding human Neu4, isoform 1 is depicted in SEQ ID NO: 26. The amino acid sequence of human Neu4, isoform 2 is depicted in SEQ ID NO: 11, and a nucleotide sequence encoding human Neu4, isoform 2 is depicted in SEQ ID NO: 35.

Four sialidases have also been found in the mouse genome and are referred to as Neu1, Neu2, Neu3 and Neu4. The amino acid sequence of mouse Neu1 is depicted in SEQ ID NO: 83, and a nucleotide sequence encoding mouse Neu1 is depicted in SEQ ID NO: 87. The amino acid sequence of mouse Neu2 is depicted in SEQ ID NO: 84 and a nucleotide sequence encoding mouse Neu2 is depicted in SEQ ID NO: 88. The amino acid sequence of mouse Neu3 is depicted in SEQ ID NO: 85, and a nucleotide sequence encoding mouse Neu3 is depicted in SEQ ID NO: 89. The amino acid sequence of mouse Neu4 is depicted in SEQ ID NO: 86, and a nucleotide sequence encoding mouse Neu4 is depicted in SEQ ID NO: 90.

Exemplary prokaryotic sialidases include sialidases from Salmonella typhimurium and Vibrio cholera. The amino acid sequence of Salmonella typhimurium sialidase (St-sialidase) is depicted in SEQ ID NO: 30, and a nucleotide sequence encoding Salmonella typhimurium sialidase is depicted in SEQ ID NO: 80. The amino acid sequence of Vibrio cholera sialidase is depicted in SEQ ID NO: 81, and a nucleotide sequence encoding Vibrio cholera sialidase is depicted in SEQ ID NO: 82.

In certain embodiments, a recombinant mutant human sialidase has about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, about 100%, or more than 100% of the enzymatic activity of a corresponding (or template) wild-type human sialidase.

In certain embodiments, the recombinant mutant human sialidase has the same substrate specificity as the corresponding wild-type human sialidase. In other embodiments, the recombinant mutant human sialidase has a different substrate specificity than the corresponding wild-type human sialidase. For example, in certain embodiments the recombinant mutant human sialidase can cleave α2,3, α2,6, and/or α2,8 linkages. In certain embodiments the sialidase can cleave α2,3 and α2,8 linkages.

In certain embodiments, the expression yield of the recombinant mutant human sialidase in mammalian cells, e.g., HEK293 cells, CHO cells, murine myeloma cells (NSO, Sp2/0), or human fibrosarcoma cells (HT-1080), e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of the corresponding wild-type human sialidase.

In certain embodiments, the recombinant mutant human sialidase has about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, about 100%, or more than 100% of the enzymatic activity of a corresponding wild-type human sialidase, and the expression yield of the recombinant mutant human sialidase in mammalian cells, e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of a corresponding wild-type human sialidase.

a. Substitution of Cysteine Residues

In certain embodiments, the recombinant mutant human sialidase comprises a substitution of at least one cysteine (cys, C) residue. It has been discovered that certain cysteine residues in sialidases may inhibit expression of functional protein as a result of protein aggregation. Accordingly, in certain embodiments, the recombinant mutant human sialidase contains at least one mutation of a free cysteine (e.g., for Neu1 (SEQ ID NO: 7), C111, C117, C171, C183, C218, C240, C242, and C252; for Neu2 (SEQ ID NO: 1), C125, C196, C219, C272, C332, and C352; for Neu3 (SEQ ID NO: 8), C7, C90, C99, C106, C127, C136, C189, C194, C226, C242, C250, C273, C279, C295, C356, C365, C368, C384, C383, C394, and C415; and for Neu4 (SEQ ID NO: 10), C88, C125, C126, C186, C191, C211, C223, C239, C276, C437, C453, C480, and C481). Frec cysteines can be substituted with any amino acid. In certain embodiments, the free cysteine is substituted with serine (ser, S), isoleucine (iso, I), valine (val, V), phenylalanine (phe, F), leucine (leu, L), or alanine (ala, A). Exemplary cysteine substitutions in Neu2 include C125A, C125I, C125S, C125V, C196A, C196L, C196V, C272S, C272V, C332A, C332S, C332V, C352L, and C352V.

In certain embodiments, the recombinant mutant human sialidase comprises two or more cysteine substitutions. Exemplary double or triple substitutions in Neu2 include: C125S and C332S; C272V and C332A; C272V and C332S; C332A and C352L; C125S and C196L; C196L and C352L; C196L and C332A; C332A and C352L; and C196L, C332A and C352L.

In certain embodiments, the recombinant mutant human sialidase is a Neu2 sialidase and comprises the substitutions C322A and C352L (SEQ ID NO: 5).

In certain embodiments, the sialidase contains an amino acid substitution at 2, 3, 4, 5, or 6 cysteines typically present in a human sialidase, e.g., Neu2 or Neu3.

In certain embodiments, the recombinant mutant human sialidase comprises a substitution or combination of substitutions corresponding to a substitution or combination of substitutions listed in TABLE 2 herein below.

b. Substitutions of Residues to Increase pI and/or Decrease Hydrophobicity

The isoelectric point (pI) of a protein is the pH at which the net charge is zero. The pI also indicates the pH at which the protein is least soluble, which affects the ability to express and purify the protein. Generally, a protein has good solubility if its pI is greater than 2 units above the pH of the solution. Human Neu2 has a predicted pI of 7.5. Thus, human Neu2 is least soluble around neutral pH, which is undesirable because expression and physiological systems are at neutral pH. In contrast, the sialidase from Salmonella typhimurium (St-sialidase), which exhibits good solubility and recombinant expression, has a pI of 9.6. Accordingly, to increase expression of human Neu2 or the other human sialidases, a recombinant mutant human sialidase may be designed to contain one or more amino acid substitution(s) wherein the substitution(s) increase(s) the pI of the sialidase relative to a sialidase without the substitution. Additionally, decreasing the number of hydrophobic amino acids on the surface of a sialidase may improve expression of sialidase by, for example, reducing aggregation. Accordingly, to increase expression of human Neu2 or the other human sialidases, a recombinant mutant human sialidase may be designed to contain one or more amino acid substitution(s) wherein the substitution(s) decrease(s) the hydrophobicity of a surface of the sialidase relative to a sialidase without the substitution(s).

Accordingly, in certain embodiments, the recombinant mutant human sialidase comprises at least one amino acid substitution, wherein the substitution increases the isoelectric point (pI) of the sialidase and/or decreases the hydrophobicity of the sialidase relative to a sialidase without the substitution. This may be achieved by introducing one or more charged amino acids, for example, positively or negatively charged amino acids, into the recombinant sialidase. In certain embodiments, the amino acid substitution is to a charged amino acid, for example, a positively charged amino acid such as lysine (lys, K), histidine (his, H), or arginine (arg, R), or a negatively charged amino acid such as aspartic acid (asp, D) or glutamic acid (glu, E). In certain embodiments, the amino acid substitution is to a lysine residue. In certain embodiments, the substitution increases the pI of the sialidase to about 7.75, about 8, about 8.25, about 8.5, about 8.75, about 9, about 9.25, about 9.5, or about 9.75.

In certain embodiments, the amino acid substitution occurs at a surface exposed D or E amino acid, in a helix or loop, or in a position that has a K or R in the corresponding position of St-sialidase. In certain embodiments, the amino acid substitution occurs at an amino acid that is remote from the catalytic site or otherwise not involved in catalysis, an amino acid that is not conserved with the other human Neu proteins or with an St-Sialidase or Clostridium NanH, or an amino acid that is not located in a domain important for function (e.g., an Asp-box or beta strand).

Exemplary amino acid substitutions in Neu2 that increase the isoelectric point (pI) of the sialidase and/or decrease the hydrophobicity of the sialidase relative to a sialidase without the substitution include A2E, A2K, D215K, V325E, V325K, E257K, and E319K. In certain embodiments, the recombinant mutant human sialidase comprises two or more amino acid substitutions, including, for example, A2K and V325E, A2K and V325K, E257K and V325K, A2K and E257K, and E257K and A2K and V325K.

In certain embodiments, the recombinant mutant human sialidase comprises a substitution or combination of substitutions corresponding to a substitution or combination of substitutions listed in TABLE 3 herein below.

c. Addition of N-Terminal Peptides and N- or C-Terminal Substitutions

It has been discovered that the addition of a peptide sequence of two or more amino acids to the N-terminus of a human sialidase can improve expression and/or activity of the sialidase. In certain embodiments, the peptide is at least 2 amino acids in length, for example, from 2 to 20, from 2 to 10, from 2 to 5, or 2, 3, 4, 5, 6, 7, 8, 9, 10, 11, 12, 13, 14, 15, 16, 17, 18, 19, or 20 amino acids in length. In certain embodiments, the peptide may form, or have a propensity to form, an α-helix.

In mice, a Neu2 isoform (type B) found in thymus contains six amino acids not present in the canonical isoform of Neu2 found in skeletal muscle. In certain embodiments herein, the N-terminal six amino acids of the mouse thymus Neu2 isoform, MEDLRP (SEQ ID NO: 4), or variations thereof, can be added onto a human Nou, e.g., human Nou2. In certain embodiments, the recombinant mutant human sialidase comprises a peptide at least two amino acid residues in length covalently associated with an N-terminal amino acid of the sialidase. In certain embodiments the recombinant mutant human sialidase comprises the peptide MEDLRP (SEQ ID NO: 4) or EDLRP (SEQ ID NO: 3) covalently associated with an N-terminal amino acid of the sialidase. In certain embodiments, the sialidase may further comprise a cleavage site, e.g., a proteolytic cleavage site, located between the peptide, e.g., MEDLRP (SEQ ID NO: 4) or EDLRP (SEQ ID NO: 3), and the remainder of the sialidase. In certain embodiments, the peptide, e.g., MEDLRP (SEQ ID NO: 4) or EDLRP (SEQ ID NO: 3), may be post-translationally cleaved from the remainder of the sialidase.

Alternatively to, or in combination with, the N-terminal addition, 1-5 amino acids of the 12 amino acid N-terminal region of the recombinant mutant human sialidase may be removed, e.g., the N-terminal methionine can be removed. In certain embodiments, if the recombinant mutant human sialidase is Neu2, the N-terminal methionine can be removed, the first five amino acids (MASLP; SEQ ID NO: 12) can be removed, or the second through fourth amino acids (ASLP; SEQ ID NO: 13) can be removed.

In certain embodiments, 1-5 amino acids of the 12 amino acid N-terminal region of the recombinant mutant human sialidase are substituted with MEDLRP (SEQ ID NO: 4), EDLRP (SEQ ID NO: 3), or TVEKSVVF (SEQ ID NO: 14). For example, in certain embodiments, if the recombinant mutant human sialidase is Neu2, the amino acids MASLP (SEQ ID NO: 12), ASLP (SEQ ID NO: 13) or M are substituted with MEDLRP (SEQ ID NO: 4), EDLRP (SEQ ID NO: 3) or TVEKSVVF (SEQ ID NO: 14).

Human sialidases have a β-propeller structure, characterized by 6 blade-shaped β-sheets arranged toroidally around a central axis. Generally, hydrophobic interactions between the blades of a β-propeller, including between the N- and C-terminal blades, enhance stability. Accordingly, in order to increase expression of human Neu2 or the other human sialidases, a recombinant mutant human sialidase can be designed comprising an amino acid substitution that increases hydrophobic interactions and/or hydrogen bonding between the N- and C-terminal β-propeller blades of the sialidase.

Accordingly, in certain embodiments, the recombinant mutant human sialidase comprises a substitution of at least one wild-type amino acid residue, wherein the substitution increases hydrophobic interactions and/or hydrogen bonding between the N- and C-termini of the sialidase relative to a sialidase without the substitution. In certain embodiments, the wild-type amino acid is substituted with asparagine (asn, N), lysine (lys, K), tyrosine (tyr, Y), phenylalanine (phe, F), or tryptophan (trp, W). Exemplary substitutions in Neu2 that increase hydrophobic interactions and/or hydrogen bonding between the N- and C-termini include LAN, LAK, V6Y, L7N, LAN and L7N, L4N and V6Y and L7N, V12N, V12Y, V12L, V6Y, V6F, or V6W. In certain embodiments, the sialidase comprises the V6Y substitution.

In certain embodiments, the recombinant mutant human sialidase comprises a combination of the above substitutions. For example, a recombinant mutant human Neu2 sialidase can comprise the additional amino acids MEDLRP (SEQ ID NO: 4), EDLRP (SEQ ID NO: 3), or TVEKSVVF (SEQ ID NO: 14) at the N-terminus and, in combination, can comprise at least one LAN, LAK, V6Y, L7N, LAN and L7N, L4N and V6Y and L7N, V12N, V12Y, V12L, V6Y, V6F, or V6W substitution. In certain embodiments, the amino acids MASLP (SEQ ID NO: 12), ASLP (SEQ ID NO: 13) or M of a recombinant mutant human Neu2 sialidase are replaced with MEDLRP (SEQ ID NO: 4), EDLRP (SEQ ID NO: 3) or TVEKSVVF (SEQ ID NO: 14) and the recombinant mutant human Neu2 sialidase also comprises at least one L4N, LAK, V6Y, L7N, L4N and L7N, LAN and V6Y and L7N, V12N, V12Y, V12L, V6Y, V6F, or V6W substitution.

In certain embodiments, the recombinant mutant human sialidase comprises a substitution or combination of substitutions corresponding to a substitution or combination of substitutions listed in TABLES 4 or 5 herein below.

Additionally, in certain embodiments, the sialidase comprises a substitution or deletion of an N-terminal methionine at the N-terminus of the sialidase. For example, in certain embodiments, the sialidase comprises a substitution of a methionine residue at a position corresponding to position 1 of wild-type human Neu2 (SEQ ID NO: 1), e.g., the methionine at a position corresponding to position 1 of wild-type human Neu2 is substituted by alanine (MIA) or aspartic acid (MID). In other embodiments, the sialidase comprises a deletion of a methionine residue at a position corresponding to position 1 (ΔM1) of wild-type human Neu2 (SEQ ID NO: 1).

In certain embodiments, the recombinant mutant human sialidase comprises a substitution or combination of substitutions corresponding to a substitution or combination of substitutions listed in TABLE 6 herein below.

d. Other Substitutions and Combinations of Substitutions

The invention further provides a recombinant mutant human Neu2 sialidase comprising at least one of the following substitutions: I187K, A328E, K370N, or H210N. In certain embodiments, a recombinant mutant human Neu2 comprises the substitution of the amino acids GDYDAPTHQVQW (SEQ ID NO: 15) with the amino acids SMDQGSTW (SEQ ID NO: 16) or STDGGKTW (SEQ ID NO: 17). In certain embodiments, a recombinant mutant human Neu2 comprises the substitution of the amino acids PRPPAPEA (SEQ ID NO: 18) with the amino acids QTPLEAAC (SEQ ID NO: 19). In certain embodiments, a recombinant mutant human Neu2 comprises the substitution of the amino acids NPRPPAPEA (SEQ ID NO: 20) with the amino acids SQNDGES (SEQ ID NO: 21).

The invention further provides a recombinant mutant human Neu2 sialidase comprising at least one substitution at a position corresponding to V212, A213, Q214, D215, T216, L217, E218, C219, Q220, V221, A222, E223, V224, E225, or T225.

The invention further provides a recombinant mutant human Neu2 sialidase comprising a combination of any of the mutations contemplated herein. For example, the recombinant mutant sialidase enzyme may comprise a combination of 2, 3, 4, 5, 6, 7, 8, 9, 10, or more of the mutations contemplated herein. For example, the recombinant mutant sialidase enzyme may comprise a M1 deletion (ΔM1), MIA substitution, MID substitution, V6Y substitution, I187K substitution, C332A substitution, or a combination of any of the foregoing. For example, the recombinant mutant sialidase enzyme may comprise a combination of mutations selected from: M1A and V6Y; M1A and I187K; MIA and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; MIA, V6Y, and I187K; M1A, V6Y, and C332A; MIA, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant human sialidase comprises a substitution or combination of substitutions corresponding to a substitution or combination of substitutions listed in TABLE 7 herein below.

In certain embodiments, the recombinant mutant human sialidase comprises the amino acid sequence of SEQ ID NO: 5, SEQ ID NO: 6, SEQ ID NO: 36, SEQ ID NO: 37, SEQ ID NO: 38, SEQ ID NO: 39, SEQ ID NO: 70, SEQ ID NO: 71, SEQ ID NO: 72, or SEQ ID NO: 73, or an amino acid sequence that has at least 85%, 90%, 95%, 96%, 97%, 98%, or 99% sequence identity to SEQ ID NO: 5, SEQ ID NO: 6, SEQ ID NO: 36, SEQ ID NO: 37, SEQ ID NO: 38, SEQ ID NO: 39, SEQ ID NO: 70, SEQ ID NO: 71, SEQ ID NO: 72, or SEQ ID NO: 73.

In certain embodiments, the recombinant mutant human sialidase comprises the amino acid sequence of X₁X₂SX₃PX₄LQKESVFQSGAHAYRIPALLYLPGQQSLLAFAEQRASKKDEHAELIVLRRGDYD APTHQVQWQAQEVVAQARLDGHRSMNPCPLYDAQTGTLFLFFIAIPGQVTEQQQLQTRANVT RLX₅QVTSTDHGRTWSSPRDLTDAAIGPAYREWSTFAVGPGHCLQLHDRARSLVVPAYAYRKL HPX₆QRPIPSAFX₇FLSHDHGRTWARGHFVAQDTLECQVAEVETGEQRVVTLNARSHLRARVQ AQSTNDGLDFQX₈SQLVKKLVEPPPQGX₉QGSVISFPSPRSGPGSPAQWLLYTHPTHSWQRAD LGAYLNPRPPAPEAWSEPX₁₀LLAKGSX₁₁AYSDLQSMGTGPDGSPLFGX₁₂LYEANDYEEIVF LMFTLKQAFPAEYLPQ

(SEQ ID NO: 100), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Phe, Trp, Tyr or Val, X₅ is Ala, Cys, Ile, Ser, or Val, X₆ is Arg, Ile, or Lys, X₇ is Ala, Cys, Leu, or Val, X₈ is Glu or Lys, X₉ is Cys or Val, X₁₀ is Lys or Val, X₁₁ is Ala, Cys, Ser, or Val, and X₁₂ is Cys, Leu, or Val, and the sialidase comprises at least one mutation relative to wild-type human Neu2 (SEQ ID NO: 1).

In certain embodiments, the recombinant mutant human sialidase comprises the amino acid sequence of X₁ASLPX₂LQKESVFQSGAHAYRIPALLYLPGQQSLLAFAEQRASKKDEHAELIVLRRGDYDA PTHQVQWQAQEVVAQARLDGHRSMNPCPLYDAQTGTLFLFFIAIPGQVTEQQQLQTRANVTR LCQVTSTDHGRTWSSPRDLTDAAIGPAYREWSTFAVGPGHCLQLHDRARSLVVPAYAYRKLH PX₃QRPIPSAFCFLSHDHGRTWARGHFVAQDTLECQVAEVETGEQRVVTLNARSHLRARVQAQ STNDGLDFQESQLVKKLVEPPPQGCQGSVISFPSPRSGPGSPAQWLLYTHPTHSWQRADLGA YLNPRPPAPEAWSEPVLLAKGSX₄AYSDLQSMGTGPDGSPLFGCLYEANDYEEIVFLMFTLKQ AFPAEYLPQ

(SEQ ID NO: 91), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Arg, Ile, or Lys, and X₄ is Ala, Cys, Ser, or Val, and the sialidase comprises at least one mutation relative to wild-type human Neu2 (SEQ ID NO: 1). In certain embodiments, X₁ is Ala, Asp, Met, or not present, X₂ is Tyr or Val, X₃ is Ile or Lys, and X₄ is Ala or Cys.

In certain embodiments, the recombinant mutant human sialidase comprises a conservative substitution relative to a recombinant mutant human sialidase sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution: p matrix (e.g., the PAM 250 matrix).

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.:-G, Cost to open gap [Integer]: default=5 for nucleotides/11 for proteins;-E, Cost to extend gap [Integer]: default=2 for nucleotides/1 for proteins;-q, Penalty for nucleotide mismatch [Integer]: default=−3;-r, reward for nucleotide match [Integer]: default=1;-e, expect value [Real]: default=10; −W, wordsize [Integer]: default=11 for nucleotides/28 for megablast/3 for proteins;-y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

### II. Fusion Proteins/Antibody Conjugates

To promote the selective removal of sialic acids on hypersialylated cancer cells and/or in the tumor microenvironment, it may be helpful to target a sialidase as described herein to such a cell or to such a tumor microenvironment. Additionally, in order to promote the removal of sialic acid by a sialidase in a subject, it may be helpful to extend the plasma half-life of the sialidase in the subject. These can be achieved by including the sialidase in a fusion protein and/or antibody conjugate (e.g., a chemically conjugated conjugate).

Accordingly, the invention further provides fusion proteins comprising a sialidase enzyme, or a functional fragment thereof, and a portion or fragment of an antibody, such as an immunoglobulin Fc domain (also referred to herein as an Fc domain), or an immunoglobulin antigen-binding domain (also referred to herein as an antigen-binding domain). In certain embodiments, the sialidase and antibody or portion thereof (e.g., immunoglobulin Fc domain or antigen-binding domain) are linked by a peptide bond or an amino acid linker.

As used herein, unless otherwise indicated, the term “fusion protein” is understood to refer to a single polypeptide chain comprising amino acid sequences based upon two or more separate proteins or polypeptide chains, where the two amino acid sequences may be fused together directly or via an intervening linker sequence, e.g., via an intervening amino acid linker. A nucleotide sequence encoding a fusion protein can, for example, be created using conventional recombinant DNA technologies.

In certain embodiments, the fusion protein comprises a tag, such as a Strep tag (e.g., a Strep II tag), a His tag (e.g., a 10× His tag (SEQ ID NO: 105)), a myc tag, or a FLAG tag. The tag can be located on the C-terminus or the N-terminus of the fusion protein. In certain embodiments, a fusion protein comprises a sialidase portion joined to a polypeptide comprising an immunoglobulin heavy chain in an N- to C-terminal orientation, wherein the sialidase portion comprises an N-terminal addition of MEDLRP (SEQ ID NO: 4), and a Strep II Tag is located on the C-terminus of the immunoglobulin heavy chain or the N-terminus of the sialidase portion.

a. Sialidase Portion

The sialidase portion of the fusion protein described herein can be any sialidase, e.g., a fungal, bacterial, non-human mammalian or human sialidase. In certain embodiments, the sialidase portion is a recombinant human sialidase comprising at least one mutation relative to a wild-type human sialidase, e.g., a substitution, deletion, or addition of at least one amino acid, as described above.

In certain embodiments, the sialidase is any recombinant mutant human sialidase disclosed herein, or a functional fragment thereof.

In certain embodiments, the sialidase portion comprises a C332A and C352L mutation. In certain embodiments, the sialidase comprises an N-terminal addition of MEDLRP (SEQ ID NO: 4) or EDLRP (SEQ ID NO: 3). In certain embodiments, the sialidase portion comprises a LSHSLST (SEQ ID NO: 22) peptide on the N-terminus. In certain embodiments, the sialidase portion comprises an N-terminal addition of MEDLRP (SEQ ID NO: 4) and an A2K substitution. In certain embodiments, the sialidase portion comprises an N-terminal addition of MEDLRP (SEQ ID NO: 4) and a C332A substitution. In certain embodiments, the sialidase portion comprises an N-terminal addition of MEDLRP (SEQ ID NO: 4), a C332A substitution, and a C352L substitution.

In certain embodiments, the sialidase portion comprises a M1 deletion (ΔM1), M1A substitution, M1D substitution, V6Y substitution, I187K substitution, C332A substitution, or a combination of any of the foregoing. For example, the sialidase portion may comprise a combination of mutations selected from: MIA and V6Y; MIA and I187K; MIA and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; MIA, V6Y, and I187K; MIA, V6Y, and C332A; MIA, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; MIA, V6Y, I187K, and C332A; M1D, V6Y, I187K, and C332A; and ΔM1, V6Y, I187K, and C332A.

In certain embodiments, the sialidase portion comprises the amino acid sequence of SEQ ID NO: 5, SEQ ID NO: 6, SEQ ID NO: 36, SEQ ID NO: 37, SEQ ID NO: 38, SEQ ID NO: 39, SEQ ID NO: 70, SEQ ID NO: 71, SEQ ID NO: 72, or SEQ ID NO: 73, or an amino acid sequence that has at least 85%, 90%, 95%, 96%, 97%, 98%, or 99% sequence identity to SEQ ID NO: 5, SEQ ID NO: 6, SEQ ID NO: 36, SEQ ID NO: 37, SEQ ID NO: 38, SEQ ID NO: 39, SEQ ID NO: 70, SEQ ID NO: 71, SEQ ID NO: 72, or SEQ ID NO: 73.

b. Antibody Portion

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

In certain embodiments, the fusion protein comprises an immunoglobulin Fc domain. As used herein, unless otherwise indicated, the term “immunoglobulin Fc domain” refers to a fragment of an immunoglobulin heavy chain constant region which, either alone or in combination with a second immunoglobulin Fc domain, is capable of binding to an Fc receptor. An immunoglobulin Fc domain may include, e.g., immunoglobulin CH2 and CH3 domains. An immunoglobulin Fc domain may include, e.g., immunoglobulin CH2 and CH3 domains and an immunoglobulin hinge region. Boundaries between immunoglobulin hinge regions, CH2, and CH3 domains are well known in the art, and can be found, e.g., in the PROSITE database (available on the world wide web at prosite.expasy.org).

In certain embodiments, the immunoglobulin Fc domain is derived from a human IgG1, IgG2, IgG3, IgG4, IgA1, IgA2, IgD, IgE, and IgM Fc domain. A single amino acid substitution (S228P according to Kabat numbering; designated IgG4Pro) may be introduced to abolish the heterogeneity observed in recombinant IgG4 antibody. Sec Angal, S. et al. (1993) MOL. IMMUNOL. 30:105-108.

In certain embodiments, the immunoglobulin Fc domain is derived from a human IgG1 isotype or another isotype that elicits antibody-dependent cell-mediated cytotoxicity (ADCC) and/or complement mediated cytotoxicity (CDC). In certain embodiments, the immunoglobulin Fc domain is derived from a human IgG1 isotype (e.g., SEQ ID NO: 31 or SEQ ID NO: 69).

In certain embodiments, the immunoglobulin Fc domain is derived from a human IgG4 isotype or another isotype that elicits little or no antibody-dependent cell-mediated cytotoxicity (ADCC) and/or complement mediated cytotoxicity (CDC). In certain embodiments, the immunoglobulin Fc domain is derived from a human IgG4 isotype.

In certain embodiments, the immunoglobulin Fc domain comprises either a “knob” mutation, e.g., T366Y or a “hole” mutation, e.g., Y407T for heterodimerization with a second polypeptide (residue numbers according to EU numbering, Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

In certain embodiments, the fusion protein comprises an immunoglobulin antigen-binding domain. The inclusion of such a domain may improve targeting of a fusion protein to a sialylated cancer cell and/or to the tumor microenvironment. As used herein, unless otherwise indicated, the term “immunoglobulin antigen-binding domain” refers to a polypeptide that, alone or in combination with another immunoglobulin antigen-binding domain, defines an antigen-binding site. Exemplary immunoglobulin antigen-binding domains include, for example, immunoglobulin heavy chain variable region and an immunoglobulin light chain variable region, where the variable regions together define an antigen binding site.

The immunoglobulin antigen-binding domain and/or antigen binding site can be derived from an antibody selected from, for example, adecatumumab, ascrinvacumab, cixutumumab, conatumumab, daratumumab, drozitumab, duligotumab, durvalumab, dusigitumab, enfortumab, enoticumab, epratuxumab, figitumumab, ganitumab, glembatumumab, intetumumab, ipilimumab, iratumumab, icrucumab, lexatumumab, lucatumumab, mapatumumab, narnatumab, necitumumab, nesvacumab, ofatumumab, olaratumab, panitumumab, patritumab, pritumumab, radretumab, ramucirumab, rilotumumab, robatumumab, seribantumab, tarextumab, teprotumumab, tovetumab, vantictumab, vesencumab, votumumab, zalutumumab, flanvotumab, altumomab, anatumomab, arcitumomab, bectumomab, blinatumomab, detumomab, ibritumomab, minretumomab, mitumomab, moxctumomab, naptumomab, nofctumomab, pemtumomab, pintumomab, racotumomab, satumomab, solitomab, taplitumomab, tenatumomab, tositumomab, tremelimumab, abagovomab, atezolizumab, durvalumab, avelumab, igovomab, oregovomab, capromab, cdrecolomab, nacolomab, amatuximab, bavituximab, brentuximab, cetuximab, derlotuximab, dinutuximab, ensituximab, futuximab, girentuximab, indatuximab, isatuximab, margetuximab, rituximab, siltuximab, ublituximab, ecromeximab, abituzumab, alemtuzumab, bevacizumab, bivatuzumab, brontictuzumab, cantuzumab, cantuzumab, citatuzumab, clivatuzumab, dacctuzumab, demcizumab, dalotuzumab, denintuzumab, clotuzumab, emactuzumab, emibetuzumab, enoblituzumab, ctaracizumab, farletuzumab, ficlatuzumab, gemtuzumab, imgatuzumab, inotuzumab, labetuzumab, lifastuzumab, lintuzumab, lirilumab, lorvotuzumab, lumretuzumab, matuzumab, milatuzumab, moxctumomab, nimotuzumab, obinutuzumab, ocaratuzumab, otlertuzumab, onartuzumab, oportuzumab, parsatuzumab, pertuzumab, pidilizumab, pinatuzumab, polatuzumab, sibrotuzumab, simtuzumab, tacatuzumab, tigatuzumab, trastuzumab, tucotuzumab, urelumab, vandortuzumab, vanucizumab, veltuzumab, vorsetuzumab, sofituzumab, catumaxomab, ertumaxomab, depatuxizumab, ontuxizumab, blontuvetmab, tamtuvetmab, nivolumab, pembrolizumab, cpratuzumab, MEDI9447, urclumab, utomilumab, hu3F8, hu14.18-IL-2, 3F8/OKT3BsAb, lirilumab, BMS-986016 pidilizumab, AMP-224, AMP-514, BMS-936559, atezolizumab, and avelumab. In certain embodiments, the immunoglobulin antigen-binding domain can be derived from an antibody selected from trastuzumab, cetuximab, daratumumab, girentuximab, panitumumab, ofatumumab, and rituximab.

In certain embodiments, the immunoglobulin antigen-binding domain is derived from trastuzumab. The trastuzumab heavy chain amino acid sequence is depicted in SEQ ID NO: 40, and the trastuzumab light chain amino acid sequence is depicted in SEQ ID NO: 41. The amino acid sequence of an exemplary scFv derived from trastuzumab is depicted in SEQ ID NO: 42.

The immunoglobulin antigen-binding domain and/or antigen binding site can be derived from an antibody that binds a cancer antigen selected from, for example, adenosine A2a receptor (A2aR), A kinase anchor protein 4 (AKAP4), B melanoma antigen (BAGE), brother of the regulator of imprinted sites (BORIS), breakpoint cluster region Abelson tyrosine kinase (BCR/ABL), CA125, CAIX, CD19, CD20, CD22, CD30, CD33, CD52, CD73, CD137, carcinoembryonic antigen (CEA), CS1, cytotoxic T-lymphocyte-associated antigen 4 (CTLA-4), estrogen receptor binding site associated antigen 9 (EBAG9), epidermal growth factor (EGF), epidermal growth factor receptor (EGFR), EGF-like module receptor 2 (EMR2), epithelial cell adhesion molecule (EpCAM) (17-1A), FR-alpha, G antigen (GAGE), disialoganglioside GD2 (GD2), glycoprotein 100 (gp100), human epidermal growth factor receptor 2 (Her2), hepatocyte growth factor (HGF), human papillomavirus 16 (HPV-16), heat-shock protein 105 (HSP105), isocitrate dehydrogenase type 1 (IDH1), idiotype (NeuGcGM3), indolcamine-2,3-dioxygenase 1 (IDO1), IGF-1, IGFIR, IGGIK, killer cell immunoglobulin-like receptor (KIR), lymphocyte activation gene 3 (LAG-3), lymphocyte antigen 6 complex K (LY6K), Matrix-metalloproteinase-16 (MMP16), melanotransferrin (MF12), melanoma antigen 3 (MAGE-A3), melanoma antigen C2 (MAGE-C2), melanoma antigen D4 (MAGE-D4), melanoma antigen recognized by T-cells 1 (Melan-A/MART-1), N-methyl-N′-nitroso-guanidine human osteosarcoma transforming gene (MET), mucin 1 (MUC1), mucin 4 (MUC4), mucin 16 (MUC16), New York esophageal squamous cell carcinoma 1 (NY-ESO-1), prostatic acid phosphatase (PAP), programmed cell death receptor 1 (PD-1), programmed cell death receptor ligand 1 (PD-L1), phosphatidylserine, preferentially expressed antigen of melanoma (PRAME), prostate specific antigen (PSA), protein tyrosine kinase 7 (PTK7, also known as colon carcinoma kinase 4 (CCK4)), receptor tyrosine kinase orphan receptor 1 (ROR1), scatter factor receptor kinase, sialyl-Tn, sperm-associated antigen 9 (SPAG-9), synovial sarcoma X-chromosome breakpoint 1 (SSX1), survivin, telomerase, T-cell immunoglobulin domain and mucin domain-3 (TIM-3), vascular endothelial growth factor (VEGF) (e.g., VEGF-A), vascular endothelial growth factor Receptor 2 (VEGFR2), V-domain immunoglobulin-containing suppressor of T-cell activation (VISTA), Wilms' Tumor-1 (WT1), X chromosome antigen 1b (XAGE-1b), 5T4, Mesothelin, Glypican 3 (GPC3), Folate Receptor a (FRa), Prostate Specific Membrane Antigen (PSMA), cMET, CD38, B Cell Maturation Antigen (BCMA), CD123, CLDN6, CLDN9, LRRC15, PRLR (Prolactin Receptor), RING finger protein 43 (RNF43), Uroplakin-1 B (UPK1 B), tumor necrosis factor superfamily member 9 (TNFSF9), tumor necrosis factor receptor superfamily member 21 (TNFSRF21), bone morphogenetic protein receptor type-1B (BMPR1B), Kringle domain-containing transmembrane protein 2 (KREMEN2), Delta-like protein 3 (DLL3), Siglec7 and Siglec9. Additional exemplary cancer antigens include those found on cancer stem cells, e.g., SSEA3, SSEA4, TRA-1-60, TRA-1-81, SSEAI, CD133 (AC133), CD90 (Thy-1), CD326 (EpCAM), Cripto-1 (TDGF1), PODXL-1 (Podocalyxin-like protein 1), ABCG2, CD24, CD49f (Integrin a6), Notch2, CD146 (MCAM), CD10 (Neprilysin), CD117 (c-KIT), CD26 (DPP-4), CXCR4, CD34, CD271, CD13 (Alanine aminopeptidase), CD56 (NCAM), CD105 (Endoglin), LGR5, CD114 (CSF3R), CD54 (ICAM-1), CXCR1, 2, TIM-3 (HAVCR2), CD55 (DAF), DLL4 (Delta-like ligand 4), CD20 (MS4A1), and CD96.

The invention further provides antibody conjugates containing one or more of the fusion proteins disclosed herein. As used herein, unless otherwise indicated, the term “antibody conjugate” is understood to refer to an antibody, or a functional fragment thereof, that comprises antigen-binding activity and/or Fc receptor-binding activity, conjugated (e.g., covalently coupled) to an additional functional moiety. In certain embodiments, the antibody or functional antibody fragment is conjugated to a sialidase enzyme, e.g., a recombinant mutant human sialidase enzyme disclosed herein. In certain embodiments, an antibody conjugate comprises a single polypeptide chain. In certain embodiments, an antibody conjugate comprises two, three, four, or more polypeptide chains that are covalently or non-covalently associated together to produce a multimeric complex, e.g., a dimeric, trimeric or tetrameric complex.

TABLE 1 shows antibodies and antibody-drug conjugates suitable for use in accordance with the present invention, the antigen bound by the antibody or antibody-drug conjugate, and for certain antibodies, the type of cancer targeted by the antibody or antibody-drug conjugate.

TABLE 1

| Antibody or antibody- | | |
|---|---|---|
| drug conjugate | Cancer Antigen | Cancer Type |
| oregovomab | CA125 | |
| girentuximab | CAIX | |
| obinutuzumab | CD20 | |
| ofatumumab | CD20 | |
| rituximab | CD20 | |
| alemtuzumab | CD52 | |
| Ipilimumab | cytotoxic T-lymphocyte-associated | |
| | antigen 4 (CTLA-4) | |
| tremelimumab | CTLA-4 | |
| Cetuximab | epidermal growth factor receptor | |
| | (EGFR) | |
| necitumumab | EGFR | |
| panitumumab | EGFR | |
| zalutumumab | EGFR | |
| edrecolomab | epithelial cell adhesion molecule | |
| | (EpCAM) (17-1A) | |
| farletuzumab | FR-alpha | |
| Pertuzumab | human epidermal growth factor | |
| | receptor 2 (Her2) | |
| trastuzumab | Her2 | |
| rilotumumab | HGF | |
| figitumumab | IGF-1 | |
| Ganitumab | IGF1R | |
| durvalumab | IGG1K | |
| bavituximab | Phosphatidylserine | |
| onartuzumab | scatter factor receptor kinase | |
| bevacizumab | vascular endothelial growth factor-A | |
| | (VEGF-A) | |
| ramucirumab | vascular endothelial growth factor | |
| | Receptor 2 (VEGFR2) | |
| blinatumomab | CD19 | acute lymphoblastic leukemia |
| | | (ALL) |
| Rituximab; | CD20 | non-Hodgkin's lymphoma |
| ofatumumab, | | (NHL), chronic lymphocytic |
| ibritumomab (e.g., ⁹⁰Y- | | leukemia (CLL) |
| ibritumomab; | | B-cell NHL |
| tositumomab (e.g., ¹³¹I- | | pre-B ALL |
| tositumomab | | |
| brentuximab (e.g., | CD30 | Hodgkin's lymphoma |
| brentuximab vedotin | | |
| gemtuzumab (e.g., | CD33 | acute myelogenous leukemia |
| gemtuzumab ozogamicin | | (AML) |
| Alemtuzumab | CD52 | CLL |
| Ipilimumab | cytotoxic T-lymphocyte-associated | Unresectable or metastatic |
| | antigen 4 (CTLA-4) | melanoma |
| cetuximab; | epidermal growth factor receptor | colorectal cancer (CRC) |
| panitumumab | (EGFR) | Head and Neck |
| Catumaxomab | epithelial cell adhesion molecule | Malignant ascites |
| | (EpCAM) | |
| trastuzumab; | human epidermal growth factor | Breast |
| pertuzumab | receptor 2 (HER2) | |
| nivolumab, | programmed cell death receptor 1 | Metastatic melanoma, non- |
| pembrolizumab | (PD-1) | small cell lung cancer |
| | | (NSCLC) |
| Bevacizumab | vascular endothelial growth factor | Breast, Cervical |
| | (VEGF) | CRC, NSCLC |
| | | renal cell carcinoma (RCC), |
| | | Ovarian |
| | | Glioblastoma |
| Ramucirumab | vascular endothelial growth factor | Gastric |
| | receptor 2 (VEGF-R2) | NSCLC |
| Epratuzumab; | CD22 | acute lymphoblastic leukemia |
| moxetumomab; | | (ALL) |
| inotuzumab (e.g., | | |
| inotuzumab ozogamicin) | | |
| MEDI9447 | CD73 | Advanced solid tumors |
| Urelumab; | CD137 | Advanced solid tumors |
| utomilumab (PF- | | |
| 05082566) | | |
| Elotuzumab | CD2 subset 1 (CS1) | Multiple myeloma |
| Tremelimumab | cytotoxic T-lymphocyte-associated | Malignant mesothelioma |
| | antigen 4 (CTLA-4) | |
| Necitumumab | epidermal growth factor receptor | non-small cell lung cancer |
| | (EGFR) | (NSCLC) |
| dinutuximab, hu3F8; | disialoganglioside GD2 (GD2) | Neuroblastoma |
| hu14.18-IL-2; | | Retinoblastoma |
| 3F8/OKT3BsAb | | Melanoma other solid tumors |
| Racotumomab | Idiotype (NeuGcGM3) | NSCLC, Breast |
| | | Melanoma |
| Lirilumab | killer cell immunoglobulin-like | Lymphoma |
| | receptor (KIR) | |
| BMS-986016 | lymphocyte activation gene 3 | Breast, Hematological, |
| | (LAG-3) | Advanced solid tumors |
| Onartuzumab | N-methyl-N′-nitroso-guanidine | NSCLC |
| | human osteosarcoma transforming | |
| | gene (MET) | |
| abagovomab; | mucin 16 (MUC16) | Ovarian |
| oregovomab | | |
| pidilizumab; | programmed cell death receptor 1 | B-cell lymphoma |
| AMP-224; AMP-514 | (PD-1) | Melanoma, CRC |
| BMS-936559; | programmed cell death receptor | NSCLC, renal cell carcinoma |
| atezolizumab; | ligand 1 (PD-L1) | (RCC) |
| durvalumab; avelumab | | Bladder, Breast |
| | | Melanoma, squamous cell |
| | | carcinoma of the head and |
| | | neck (SCCHN) |
| naptumomab (e.g., | 5T4 | RCC, CRC |
| naptumomab estafenatox) | | Prostate |

c. Linker

In certain embodiments, the sialidase portion of the fusion protein can be linked or fused directly to the antibody portion (e.g., immunoglobulin Fc domain and/or immunoglobulin antigen-binding domain) of the fusion protein. In other embodiments, the sialidase portion can be covalently bound to the antibody portion by a linker.

The linker may couple, with one or more natural amino acids, the sialidase, or functional fragment thereof, and the antibody portions or fragments, where the amino acid (for example, a cysteine amino acid) may be introduced by site-directed mutagenesis. The linker may include one or more unnatural amino acids. It is contemplated that, in certain circumstances, a linker containing for example, one or more sulfhydryl reactive groups (e.g., a maleimide) may covalently link a cysteine in the sialidase portion or the antibody portion that is a naturally occurring cysteine residue or is the product of site-specific mutagenesis.

The linker may be a cleavable linker or a non-cleavable linker. Optionally or in addition, the linker may be a flexible linker or an inflexible linker.

The linker should be a length sufficiently long to allow the sialidase and the antibody portions to be linked without steric hindrance from one another and sufficiently short to retain the intended activity of the fusion protein. The linker preferably is sufficiently hydrophilic to avoid or minimize instability of the fusion protein. The linker preferably is sufficiently hydrophilic to avoid or minimize insolubility of the fusion protein. The linker should be sufficiently stable in vivo (e.g., it is not cleaved by serum, enzymes, etc.) to permit the fusion protein to be operative in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer Å in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker comprises a polypeptide linker that connects or fuses the sialidase portion of the fusion protein to the antibody portion (e.g., immunoglobulin Fc domain and/or immunoglobulin antigen-binding domain) of the fusion protein. For example, it is contemplated that a gene encoding a sialidase portion linked directly or indirectly (for example, via an amino acid containing linker) to an antibody portion can be created and expressed using conventional recombinant DNA technologies. For example, the amino terminus of a sialidase portion can be linked to the carboxy terminus of either the light or the heavy chain of an antibody portion. For example, for a Fab fragment, the amino terminus or carboxy terminus of the sialidase can be linked to the first constant domain of the heavy antibody chain (CH1). When a linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-10 amino acid residues. Exemplary linkers include glycine and serine-rich linkers, e.g., (GlyGlyPro)ₙ (SEQ ID NO: 106), or (GlyGlyGlyGlySer)ₙ (SEQ ID NO: 107), where n is 1-5. In certain embodiments, the linker is (Gly₄Ser)₂ (SEQ ID NO: 108). Additional exemplary linker sequences are disclosed, e.g., in George et al. (2003) PROTEIN ENGINEERING 15:871-879, and U.S. Pat. Nos. 5,482,858 and 5,525,491.

d. Antibody Conjugates

The invention further provides antibody conjugates comprising a fusion protein disclosed herein. The antibody conjugate may comprise a single polypeptide chain (i.e., a fusion protein disclosed herein) or, the antibody conjugate may comprise additional polypeptide chains (e.g., one, two, or three additional polypeptide chains). For example, an antibody conjugate may comprise a first polypeptide (fusion protein) comprising a recombinant mutant human sialidase enzyme and an immunoglobulin heavy chain, and a second polypeptide comprising an immunoglobulin light chain, where, for example, the immunoglobulin heavy and light chains together define a single antigen-binding site.

In certain embodiments, the antibody conjugate can include a single sialidase. In other embodiments, the antibody conjugate can include more than one (e.g., two) sialidases. If more than one sialidase is included, the sialidases can be the same or different. In certain embodiments, the antibody conjugate can include a single antigen-binding site. In other embodiments, the antibody conjugate can include more than one (e.g., two) antigen-binding sites. If two antigen-binding sites are used, they can be the same or different. In certain embodiments, the antibody conjugate comprises an immunoglobulin Fc fragment.

In certain embodiments, the antibody conjugate comprises one or two immunoglobulin heavy chains, or a functional fragment thereof. In certain embodiments, the antibody conjugate comprises one or two immunoglobulin light chains, or a functional fragment thereof. In certain embodiments, the antibody conjugate comprises a sialidase fused to the N- or C-terminus of an immunoglobulin heavy chain or an immunoglobulin light chain.

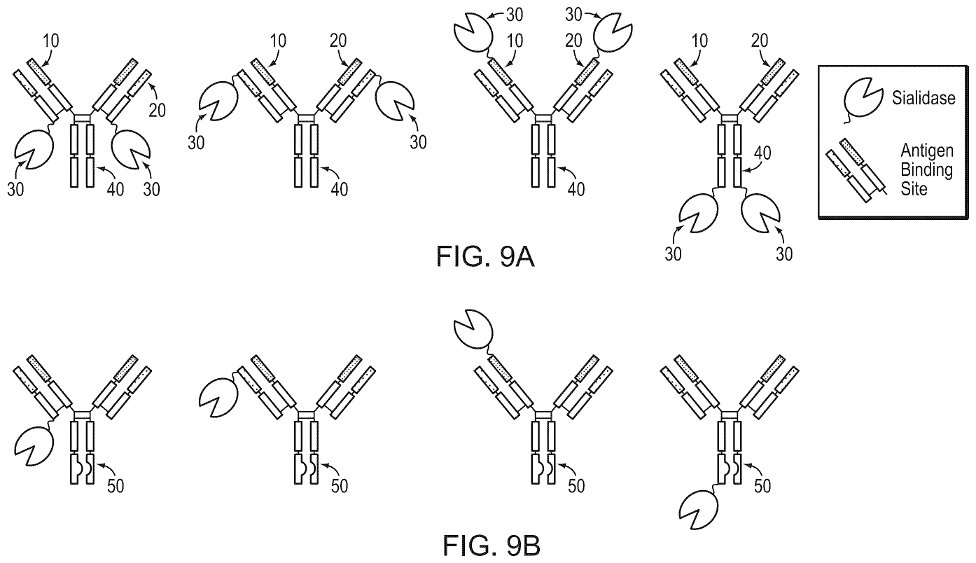

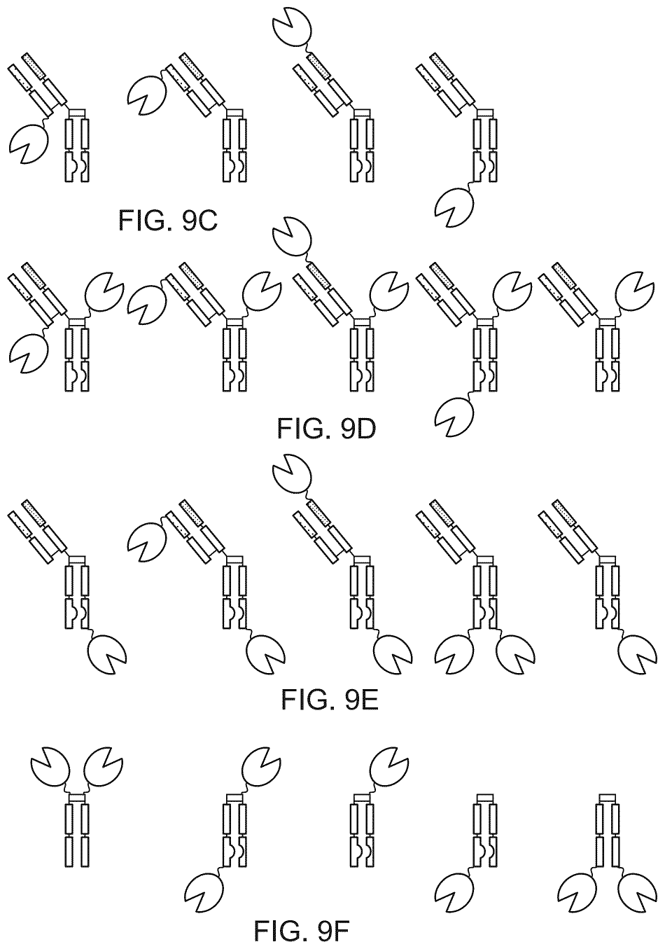

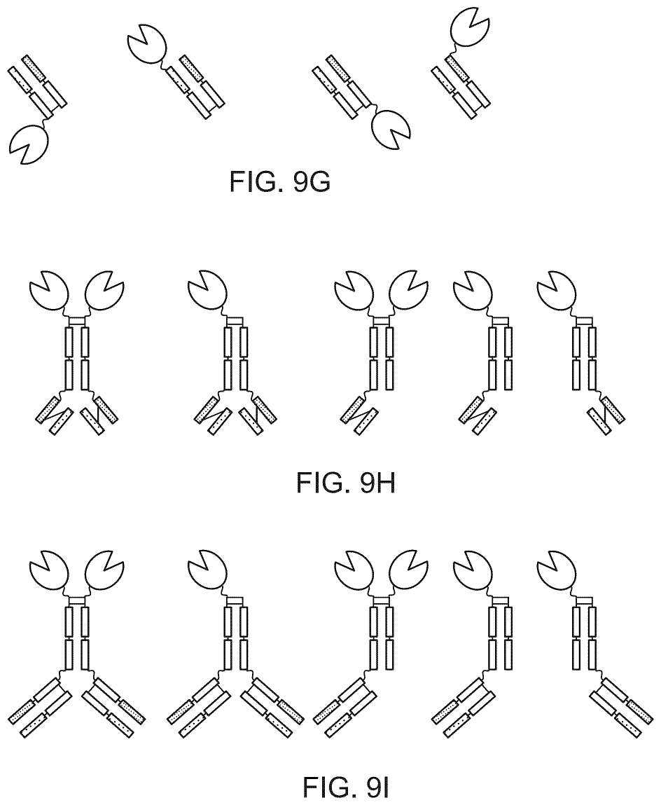

FIG. 9 depicts exemplary antibody conjugate constructs containing one or more sialidase enzymes. For example, in FIG. 9A, a first antigen-binding site is depicted as 10, a second antigen-binding site is depicted as 20, a sialidase is depicted as 30, and a Fab is depicted as 40. In each of the constructs depicted in FIGS. 9A-9I it is understood that the Fc may optionally be modified in some manner, e.g. using Knobs-into-Holes type technology, e.g., as depicted by 50 in FIG. 9B. Throughout FIG. 9 similar structures are depicted by similar schematic representations.

FIG. 9A depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first antigen-binding site as depicted as 10, and the third polypeptide and the fourth polypeptide together define a second antigen-binding site as depicted as 20. A sialidase enzyme as depicted as 30 can be conjugated to the N- or C-terminus of the first and second immunoglobulin light chain or the first and second immunoglobulin heavy chain.

FIG. 9B depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first antigen-binding site, and the third polypeptide and the fourth polypeptide together define a second antigen-binding site. A sialidase enzyme can be conjugated to the N- or C-terminus of the first immunoglobulin light chain or the first immunoglobulin heavy chain.

FIG. 9C depicts antibody conjugate constructs comprising a first polypeptide comprising an immunoglobulin light chain; a second polypeptide comprising an immunoglobulin heavy chain; and a third polypeptide comprising an immunoglobulin Fc domain. The first and second polypeptides can be covalently linked together and the second and third polypeptides can be covalently linked together. The covalent linkages can be disulfide bonds. In certain embodiments, the first polypeptide and the second polypeptide together define an antigen-binding site. A sialidase enzyme can be conjugated to the N- or C-terminus of the first immunoglobulin light chain or the first immunoglobulin heavy chain.

FIG. 9D depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the sialidase and the immunoglobulin Fc domain in an N- to C-terminal orientation. In certain embodiments, the first polypeptide and the second polypeptide together define an antigen-binding site. An optional second sialidase enzyme can be conjugated to the N- or C-terminus of the first immunoglobulin light chain or the first immunoglobulin heavy chain.

FIG. 9E depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the immunoglobulin Fc domain and the sialidase in an N- to C-terminal orientation. In certain embodiments, the first polypeptide and the second polypeptide together define an antigen-binding site. An optional second sialidase enzyme can be conjugated to the N- or C-terminus of the first immunoglobulin light chain or the first immunoglobulin heavy chain.

FIG. 9F depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N- or C-terminus of the first immunoglobulin Fc domain or to the N- or C-terminus of the second immunoglobulin Fc domain. An optional second sialidase enzyme can be conjugated to the N- or C-terminus of the first immunoglobulin Fc domain or to the N- or C-terminus of the second immunoglobulin Fc domain.

FIG. 9G depicts antibody conjugate constructs comprising a first polypeptide comprising an immunoglobulin light chain; and a second polypeptide comprising an immunoglobulin heavy chain variable region. The first and second polypeptides can be covalently linked together. The covalent linkages can be disulfide bonds. In certain embodiments, the first polypeptide and the second polypeptide together define an antigen-binding site. The sialidase enzyme can be conjugated to the N- or C-terminus of the immunoglobulin light chain or the immunoglobulin heavy chain variable region.

FIG. 9H depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. A single chain variable fragment (scFv) can be conjugated to the C-terminus of the first immunoglobulin Fc domain or the second immunoglobulin Fc domain. An optional second single chain variable fragment (scFv) can be conjugated to the C-terminus of the first immunoglobulin Fc domain or the second immunoglobulin Fc domain, respectively.

FIG. 9I depicts antibody conjugate constructs similar to those depicted in FIG. 9H except that each scFv is replaced with an immunoglobulin antigen binding fragment, e.g., an Fab. For example, FIG. 9I depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. An antibody fragment (Fab) can be conjugated or fused to the C-terminus of the first immunoglobulin Fc domain or the second immunoglobulin Fc domain. An optional second antibody fragment (Fab) can be conjugated or fused to the C-terminus of the second immunoglobulin Fc domain or the first immunoglobulin Fc domain, respectively. In the case of a fusion, the C terminus of the Fc domain is linked (either by a bond or an amino acid linker) to a first polypeptide chain defining an immunoglobulin antigen binding fragment. In the case of antibodies that have an antigen binding site defined by a single variable region, then this may be sufficient to impart binding affinity to a target antigen. In other instances, e.g., in the case of a human antibody, the first polypeptide chain defining an immunoglobulin antigen binding fragment can be conjugated (e.g., covalently conjugated, e.g., via a disulfide bond) to a second polypeptide chain defining an immunoglobulin antigen binding fragment, there the two antigen binding fragments together define an antigen binding site for binding the target antigen.

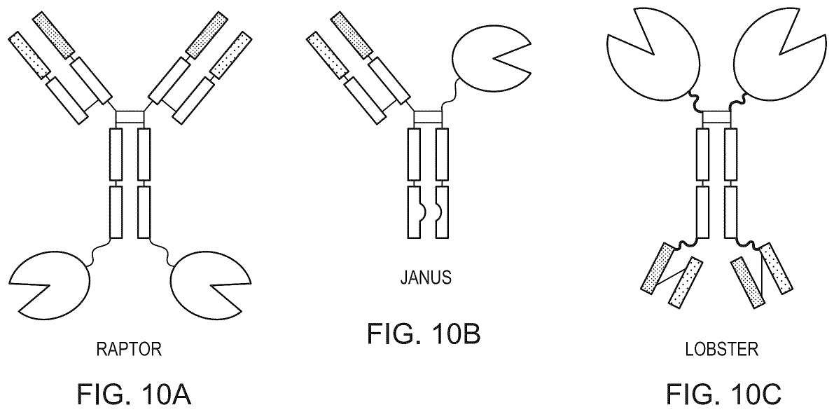

In certain embodiments, the antibody conjugate comprises a first polypeptide comprising a first immunoglobulin light chain; a second polypeptide comprising a first immunoglobulin heavy chain and a first sialidase; a third polypeptide comprising a second immunoglobulin heavy chain and a second sialidase; and a fourth polypeptide comprising a second immunoglobulin light chain. An example of this embodiment is shown in FIG. 10A. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first antigen-binding site, and the third polypeptide and the fourth polypeptide together define a second antigen-binding site. In certain embodiments, the second and third polypeptides comprise the first and second immunoglobulin heavy chain and the first and second sialidase, respectively, in an N- to C-terminal orientation. In certain embodiments, the second and third polypeptides comprise the first and second sialidase and the first and second immunoglobulin heavy chain, respectively, in an N- to C-terminal orientation.

In certain embodiments, the antibody conjugate comprises a first polypeptide comprising an immunoglobulin light chain; a second polypeptide comprising an immunoglobulin heavy chain; and a third polypeptide comprising an immunoglobulin Fc domain and a sialidase. An example of this embodiment is shown in FIG. 10B. The first and second polypeptides can be covalently linked together and the second and third polypeptides can be covalently linked together. The covalent linkages can be disulfide bonds. In certain embodiments, the first polypeptide and the second polypeptide together define an antigen-binding site. In certain embodiments, the third polypeptide comprises the sialidase and the immunoglobulin Fc domain in an N- to C-terminal orientation or the immunoglobulin Fc domain and the sialidase in an N- to C-terminal orientation.

In certain embodiments, the first polypeptide comprises the amino acid sequence of SEQ ID NO: 49, or an amino acid sequence that has at least 85%, 90%, 95%, 96%, 97%, 98%, or 99% sequence identity to SEQ ID NO: 49. In certain embodiments, the second polypeptide comprises the amino acid sequence of SEQ ID NO: 50 or an amino acid sequence that has at least 85%, 90%, 95%, 96%, 97%, 98%, or 99% sequence identity to SEQ ID NO: 50. In certain embodiments, the third polypeptide comprises the amino acid sequence of SEQ ID NO: 51, SEQ ID NO: 52, SEQ ID NO: 53, SEQ ID NO: 54, SEQ ID NO: 63, SEQ ID NO: 76, SEQ ID NO: 77, SEQ ID NO: 78, or SEQ ID NO: 79, or an amino acid sequence that has at least 85%, 90%, 95%, 96%, 97%, 98%, or 99% sequence identity to SEQ ID NO: 51, SEQ ID NO: 52, SEQ ID NO: 53, SEQ ID NO: 54, SEQ ID NO: 63, SEQ ID NO: 76, SEQ ID NO: 77, SEQ ID NO: 78, or SEQ ID NO: 79.

In certain embodiments, the third polypeptide comprises the amino acid sequence of X₁X₂SX₃PX₄LOKESVFQSGAHAYRIPALLYLPGQQSLLAFAEQRASKKDEHAELIVLRRGDYD APTHQVQWQAQEVVAQARLDGHRSMNPCPLYDAQTGTLFLFFIAIPGQVTEQQQLQTRANVT RLX₅QVTSTDHGRTWSSPRDLTDAAIGPAYREWSTFAVGPGHCLQLHDRARSLVVPAYAYRKL HPX₆QRPIPSAFX FLSHDHGRTWARGHFVAQDTLECQVAEVETGEQRVVTLNARSHLRARVQ AQSTNDGLDFQX₈SQLVKKLVEPPPQGX₉QGSVISFPSPRSGPGSPAQWLLYTHPTHSWQRAD LGAYLNPRPPAPEAWSEPX₁₀LLAKGSX₁₁AYSDLQSMGTGPDGSPLFGX₁₂LYEANDYEEIVF LMFTLKQAFPAEYLPQGGGGSGGGGSDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKE YKCKVSNKALPAPIEKTI SKAKGQPREPQVYTLPPSREEMTKNQVSLTCLVKGFYPSDIAVE WESNGQPENNYKTTPPVLDSDGSFFLTSKLTVDKSRWQQGNVFSCSVMHEALHNHYTQKSLS LSPGK

(SEQ ID NO: 101), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Phe, Trp, Tyr or Val, X₅ is Ala, Cys, Ile, Ser, or Val, X₆ is Arg, Ile, or Lys, X₇ is Ala, Cys, Leu, or Val, X₈ is Glu or Lys, X₉ is Cys or Val, X₁₀ is Lys or Val, X₁₁ is Ala, Cys, Ser, or Val, and X₁₂ is Cys, Leu, or Val.

In certain embodiments, the third polypeptide comprises the amino acid sequence of X₁ASLPX₂LQKESVFQSGAHAYRIPALLYLPGQQSLLAFAEQRASKKDEHAELIVLRRGDYDA PTHQVQWQAQEVVAQARLDGHRSMNPCPLYDAQTGTLFLFFIAIPGQVTEQQQLQTRANVTR LCQVTSTDHGRTWSSPRDLTDAAIGPAYREWSTFAVGPGHCLQLHDRARSLVVPAYAYRKLH PX₃QRPIPSAFCFLSHDHGRTWARGHFVAQDTLECQVAEVETGEQRVVTLNARSHLRARVQAQ STNDGLDFQESQLVKKLVEPPPQGCQGSVISFPSPRSGPGSPAQWLLYTHPTHSWQRADLGA YLNPRPPAPEAWSEPVLLAKGSX₄AYSDLQSMGTGPDGSPLFGCLYEANDYEEIVELMFTLKQ AFPAEYLPQGGGGSGGGGSDKTHTCPPCPAPELLGGPSVFLFPPKPKDTLMISRTPEVTCVV VDVSHEDPEVKFNWYVDGVEVHNAKTKPREEQYNSTYRVVSVLTVLHQDWLNGKEYKCKVSN KALPAPIEKTISKAKGQPREPQVYTLPPSREEMTKNQVSLTCLVKGFYPSDIAVEWESNGQP ENNYKTTPPVLDSDGSFFLTSKLTVDKSRWQQGNVFSCSVMHEALHNHYTQKSLSLSPGK

(SEQ ID NO: 92), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Arg, Ile, or Lys, and X₄ is Ala, Cys, Ser, or Val. In certain embodiments, X₁ is Ala, Asp, Met, or not present, X₂ is Tyr or Val, X₃ is Ile or Lys, and X₄ is Ala or Cys.

In certain embodiments, the first polypeptide comprises SEQ ID NO: 49, the second polypeptide comprises SEQ ID NO: 50, and the third polypeptide comprises SEQ ID NO: 51. In certain embodiments, the first polypeptide comprises SEQ ID NO: 49, the second polypeptide comprises SEQ ID NO: 50, and the third polypeptide comprises SEQ ID NO: 52. In certain embodiments, the first polypeptide comprises SEQ ID NO: 49, the second polypeptide comprises SEQ ID NO: 50, and the third polypeptide comprises SEQ ID NO: 53. In certain embodiments, the first polypeptide comprises SEQ ID NO: 49, the second polypeptide comprises SEQ ID NO: 50, and the third polypeptide comprises SEQ ID NO: 54. In certain embodiments, the first polypeptide comprises SEQ ID NO: 49, the second polypeptide comprises SEQ ID NO: 50, and the third polypeptide comprises SEQ ID NO: 63. In certain embodiments, the first polypeptide comprises SEQ ID NO: 49, the second polypeptide comprises SEQ ID NO: 50, and the third polypeptide comprises SEQ ID NO: 76. In certain embodiments, the first polypeptide comprises SEQ ID NO: 49, the second polypeptide comprises SEQ ID NO: 50, and the third polypeptide comprises SEQ ID NO: 77. In certain embodiments, the first polypeptide comprises SEQ ID NO: 49, the second polypeptide comprises SEQ ID NO: 50, and the third polypeptide comprises SEQ ID NO: 78. In certain embodiments, the first polypeptide comprises SEQ ID NO: 49, the second polypeptide comprises SEQ ID NO: 50, and the third polypeptide comprises SEQ ID NO: 79.

In certain embodiments, the antibody conjugate comprises a first polypeptide comprising a first sialidase, a first immunoglobulin Fc domain, and a first single chain variable fragment (scFv) (it is also understood that the scFv may be replaced by a first polypeptide chain of an immunoglobulin antigen binding fragment, e.g., Fab fragment); and a second polypeptide comprising a second sialidase, a second immunoglobulin Fc domain, and a second single chain variable fragment (scFv) (it is also understood that the scFv may be replaced by a second polypeptide chain of an immunoglobulin antigen binding fragment, e.g., Fab fragment). An example of this embodiment is shown in FIG. 10C. The first and second polypeptides can be covalently linked together. The covalent linkages can be disulfide bonds. In certain embodiments, the first scFv defines a first antigen-binding site, and the second scFv defines a second antigen-binding site. In certain embodiments, the first polypeptide comprises the first sialidase, the first immunoglobulin Fc domain, and the first scFv in an N- to C-terminal orientation. In certain embodiments, the first polypeptide comprises the first scFv, the first immunoglobulin Fc domain, and the first sialidase in an N- to C-terminal orientation. In certain embodiments, the second polypeptide comprises the second sialidase, the second immunoglobulin Fc domain, and the second scFv in an N- to C-terminal orientation. In certain embodiments, the second polypeptide comprises the second scFv, the second immunoglobulin Fc domain, and the second sialidase in an N- to C-terminal orientation.

In certain embodiments, the first polypeptide comprises the amino acid sequence of SEQ ID NO: 43, SEQ ID NO: 44, SEQ ID NO: 45, SEQ ID NO: 46, SEQ ID NO: 47, SEQ ID NO: 48, SEQ ID NO: 74, or SEQ ID NO: 75, or an amino acid sequence that has at least 85%, 90%, 95%, 96%, 97%, 98%, or 99% sequence identity to SEQ ID NO: 43, SEQ ID NO: 44, SEQ ID NO: 45, SEQ ID NO: 46, SEQ ID NO: 47, SEQ ID NO: 48, SEQ ID NO: 74, or SEQ ID NO: 75. In certain embodiments, the second polypeptide comprises the amino acid sequence of SEQ ID NO: 43, SEQ ID NO: 44, SEQ ID NO: 45, SEQ ID NO: 46, SEQ ID NO: 47, SEQ ID NO: 48, SEQ ID NO: 74, or SEQ ID NO: 75, or an amino acid sequence that has at least 85%, 90%, 95%, 96%, 97%, 98%, or 99% sequence identity to SEQ ID NO: 43, SEQ ID NO: 44, SEQ ID NO: 45, SEQ ID NO: 46, SEQ ID NO: 47, SEQ ID NO: 48, SEQ ID NO: 74, or SEQ ID NO: 75.

In certain embodiments, the first and/or second polypeptide comprises the amino acid sequence of X₁X-SX₃PX₄LOKESVFQSGAHAYRIPALLYLPGQQSLLAFAEQRASKKDEHAELIVLRRGDYD APTHQVQWQAQEVVAQARLDGHRSMNPCPLYDAQTGTLFLFFIAIPGQVTEQQQLQTRANVT RLX₅QVTSTDHGRTWSSPRDLTDAAIGPAYREWSTFAVGPGHCLQLHDRARSLVVPAYAYRKL HPX₆QRPIPSAFX₇FLSHDHGRTWARGHFVAQDTLECQVAEVETGEQRVVTLNARSHLRARVQ AQSTNDGLDFQX₈SQLVKKLVEPPPQGX₉QGSVISFPSPRSGPGSPAQWLLYTHPTHSWQRAD LGAYLNPRPPAPEAWSEPX₁₀LLAKGSX₁₁AYSDLQSMGTGPDGSPLFGX₁₂LYEANDYEEIVF LMFTLKQAFPAEYLPQGGGGSGGGGSDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKE YKCKVSNKALPAPIEKTISKAKGQPREPQVYTLPPSREEMTKNQVSLTCLVKGFYPSDIAVE WESNGQPENNYKTTPPVLDSDGSFFLYSKLTVDKSRWQQGNVFSCSVMHEALHNHYTOKSLS LSPGKGGGGGGGGSGGGGSEVOLVESGGGLVQPGGSLRLSCAASGFNIKDTYIHWVRQAPG KGLEWVARIYPTNGYTRYADSVKGRFTISADTSKNTAYLQMNSLRAEDTAVYYCSRWGGDGF YAMDYWGQGTLVTVSSGGGGSGGGGSGGGGSDIQMTQSPSSLSASVGDRVTITCRASQDVNT AVAWYQQKPGKAPKLLIYSASFLYSGVPSRFSGSRSGTDFTLTISSLOPEDFATYYCQQHYT TPPTFGQGTKVEIK

(SEQ ID NO: 102), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Phe, Trp, Tyr or Val, X₅ is Ala, Cys, Ile, Ser, or Val, X₆ is Arg, Ile, or Lys, X₇ is Ala, Cys, Leu, or Val, X₈ is Glu or Lys, X₉ is Cys or Val, X₁₀ is Lys or Val, X₁₁ is Ala, Cys, Ser, or Val, and X₁₂ is Cys, Leu, or Val.

In certain embodiments, the first and/or second polypeptide comprises the amino acid sequence of X₁ASLPX₂LOKESVFQSGAHAYRIPALLYLPGQQSLLAFAEQRASKKDEHAELIVLRRGDYDA PTHQVQWQAQEVVAQARLDGHRSMNPCPLYDAQTGTLFLFFIAIPGQVTEQQQLQTRANVTR LCQVTSTDHGRTWSSPRDLTDAAIGPAYREWSTFAVGPGHCLQLHDRARSLVVPAYAYRKLH PX₃QRPIPSAFCFLSHDHGRTWARGHFVAQDTLECQVAEVETGEQRVVTLNARSHLRARVQAQ STNDGLDFQESQLVKKLVEPPPQGCOGSVISFPSPRSGPGSPAQWLLYTHPTHSWQRADLGA YLNPRPPAPEAWSEPVLLAKGSX₄AYSDLQSMGTGPDGSPLFGCLYEANDYEEIVELMFTLKQ AFPAEYLPQGGGGSGGGGSDKTHTCPPCPAPELLGGPSVFLFPPKPKDTLMISRTPEVTCVV VDVSHEDPEVKFNWYVDGVEVHNAKTKPREEQYNSTYRVVSVLTVLHQDWLNGKEYKCKVSN KALPAPIEKTISKAKGQPREPQVYTLPPSREEMTKNQVSLTCLVKGFYPSDIAVEWESNGQP ENNYKTTPPVLDSDGSFFLYSKLTVDKSRWQQGNVFSCSVMHEALHNHYTQKSLSLSPGKGG GGSGGGGSGGGGSEVQLVESGGGLVQPGGSLRLSCAASGFNIKDTYIHWVRQAPGKGLEWVA RIYPTNGYTRYADSVKGRFTISADTSKNTAYLQMNSLRAEDTAVYYCSRWGGDGFYAMDYWG QGTLVTVSSGGGGGGGGSGGGGSDIQMTQSPSSLSASVGDRVTITCRASQDVNTAVAWYQQ KPGKAPKLLIYSASFLYSGVPSRFSGSRSGTDFTLTISSLOPEDFATYYCQQHYTTPPTFGQ GTKVEIK

(SEQ ID NO: 93), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Arg, Ile, or Lys, and X₄ is Ala, Cys, Ser, or Val. In certain embodiments, X₁ is Ala, Asp, Met, or not present, X₂ is Tyr or Val, X₃ is Ile or Lys, and X₄ is Ala or Cys.

In certain embodiments, the first and second polypeptide comprise SEQ ID NO: 43. In certain embodiments, the first and second polypeptide comprise SEQ ID NO: 44. In certain embodiments, the first and second polypeptide comprise SEQ ID NO: 45. In certain embodiments, the first and second polypeptide comprise SEQ ID NO: 46. In certain embodiments, the first and second polypeptide comprise SEQ ID NO: 47. In certain embodiments, the first and second polypeptide comprise SEQ ID NO: 48. In certain embodiments, the first and second polypeptide comprise SEQ ID NO: 74. In certain embodiments, the first and second polypeptide comprise SEQ ID NO: 75.

In certain embodiments, the antibody conjugate has a molecular weight from about 135 kDa to about 165 kDa, e.g., about 140 kDa. In other embodiments, the antibody conjugate has a molecular weight from about 215 kDa to about 245 kDa, e.g., about 230 kDa.

In certain embodiments, the antibody conjugate comprises two polypeptides that each comprise an immunoglobulin Fc domain, and the first polypeptide has either a “knob” mutation, e.g., T366Y, or a “hole” mutation, e.g., Y407T, for heterodimerization with the second polypeptide, and the second polypeptide has either a respective “knob” mutation, e.g., T366Y, or a “hole” mutation, e.g., Y407T, for heterodimerization with the first polypeptide (residue numbers according to EU numbering, Kabat, E. A., et al. (1991) supra). For example, in certain embodiments, the antibody comprises two polypeptides that each comprise an immunoglobulin Fc domain derived from human IgG1 Fc domain, and the first polypeptide comprises a Y407T mutation (e.g., the first polypeptide comprises SEQ ID NO: 32), and the second polypeptide comprises a T366Y mutation (e.g., the second polypeptide comprises SEQ ID NO: 33).

As used herein, the term “multispecific antibody” is understood to mean an antibody that specifically binds to at least two different antigens, i.e., an antibody that comprises at least two antigen-binding sites that bind to at least two different antigens. As used herein, the term “bispecific antibody” is understood to mean an antibody that specifically binds to two different antigens, i.e., an antibody that comprises two antigen-binding sites each of which bind to separate and distinct antigens. In other words, a first binding site binds a first antigen and a second binding site binds a second, different antigen. A multispecific or bispecific antibody may, for example, be a human or humanized antibody, and/or be a full length antibody or an antibody fragment (e.g., a F (ab′)₂ bispecific antibody).

The present invention encompasses antibody conjugates comprising antibody fragments, which may be generated by traditional means, such as enzymatic digestion, or by recombinant techniques. For a review of certain antibody fragments, see Hudson et al. (2003) supra.

In certain embodiments, the antibody conjugate or fusion protein can be covalently or non-covalently associated with a biological modifier, wherein the biological modifier can be used to enhance the solubility of the antibody, increase binding specificity, decrease immunogenicity or toxicity or modify the pharmacokinetic profile of the antibody. For example, the biological modifier can be used to increase the molecular weight of the antibody to increase its circulating half-life.

It is contemplated that the antibody conjugate or fusion protein may be covalently bound to one or more (for example, 2, 3, 4, 5, 6, 8, 9, 10 or more) biological modifiers that may comprise linear or branched polymers. Exemplary biological modifiers may include, for example, a variety of polymers, such as those described in U.S. Pat. No. 7,842,789. Particularly useful are polyalkylene ethers such as polyethylene glycol (PEG) and derivatives thereof (for example, alkoxy polyethylene glycol, for example, methoxypolyethylene glycol, ethoxypolyethylene glycol and the like); block copolymers of polyoxyethylene and polyoxypropylene (Pluronics); polymethacrylates; carbomers; and branched or unbranched polysaccharides which comprise the saccharide monomers such as D-mannose, D- and L-galactose, fucose, fructose, D-xylose, L-arabinose, and D-glucuronic acid.

In other embodiments, the biological modifier can be a hydrophilic polyvinyl polymer such as polyvinyl alcohol and polyvinylpyrrolidone (PVP)-type polymers. The biological modifier can be a functionalized polyvinylpyrrolidone, for example, carboxy or amine functionalized on one (or both) ends of the polymer (as available from PolymerSource). Alternatively, the biological modifier can include Poly N-(2-hydroxypropyl) methacrylamide (HPMA), or functionalized HPMA (amine, carboxy, etc.), Poly(N-isopropylacrylamide) or functionalized poly(N-isopropylacrylamide). Alternatively, the biological modifier can include Poly N-(2-hydroxypropyl) methacrylamide (HPMA), or functionalized HPMA (amine, carboxy, etc.), Poly(N-isopropylacrylamide) or functionalized poly(N-isopropylacrylamide). The modifier prior to conjugation need not be, but preferably is, water soluble, but the final conjugate should be water soluble.

In general, the biological modifier may have a molecular weight from about 2 kDa to about 5 kDa, from about 2 kDa to about 10 kDa, from about 2 kDa to about 20 kDa, from about 2 kDa to about 30 kDa, from about 2 kDa to about 40 kDa, from about 2 kDa to about 50 kDa, from about 2 kDa to about 60 kDa, from about 2 kDa to about 70 kDa, from about 2 kDa to about 80 kDa, from about 2 kDa to about 90 kDa, from about 2 kDa to about 100 kDa, from about 2 kDa to about 150 kDa, from about 5 kDa to about 10 kDa, from about 5 kDa to about 20 kDa, from about 5 kDa to about 30 kDa, from about 5 kDa to about 40 kDa, from about 5 kDa to about 50 kDa, from about 5 kDa to about 60 kDa, from about 5 kDa to about 70 kDa, from about 5 kDa to about 80 kDa, from about 5 kDa to about 90 kDa, from about 5 kDa to about 100 kDa, from about 5 kDa to about 150 kDa, from about 10 kDa to about 20 kDa, from about 10 kDa to about 30 kDa, from about 10 kDa to about 40 kDa, from about 10 kDa to about 50 kDa, from about 10 kDa to about 60 kDa, from about 10 kDa to about 70 kDa, from about 10 kDa to about 80 kDa, from about 10 kDa to about 90 kDa, from about 10 kDa to about 100 kDa, from about 10 kDa to about 150 kDa, from about 20 kDa to about 30 kDa, from about 20 kDa to about 40 kDa, from about 20 kDa to about 50 kDa, from about 20 kDa to about 60 kDa, from about 20 kDa to about 70 kDa, from about 20 kDa to about 80 kDa, from about 20 kDa to about 90 kDa, from about 20 kDa to about 100 kDa, from about 20 kDa to about 150 kDa, from about 30 kDa to about 40 kDa, from about 30 kDa to about 50 kDa, from about 30 kDa to about 60 kDa, from about 30 kDa to about 70 kDa, from about 30 kDa to about 80 kDa, from about 30 kDa to about 90 kDa, from about 30 kDa to about 100 kDa, from about 30 kDa to about 150 kDa, from about 40 kDa to about 50 kDa, from about 40 kDa to about 60 kDa, from about 40 kDa to about 70 kDa, from about 40 kDa to about 80 kDa, from about 40 kDa to about 90 kDa, from about 40 kDa to about 100 kDa, from about 40 kDa to about 150 kDa, from about 50 kDa to about 60 kDa, from about 50 kDa to about 70 kDa, from about 50 kDa to about 80 kDa, from about 50 kDa to about 90 kDa, from about 50 kDa to about 100 kDa, from about 50 kDa to about 150 kDa, from about 60 kDa to about 70 kDa, from about 60 kDa to about 80 kDa, from about 60 kDa to about 90 kDa, from about 60 kDa to about 100 kDa, from about 60 kDa to about 150 kDa, from about 70 kDa to about 80 kDa, from about 70 kDa to about 90 kDa, from about 70 kDa to about 100 kDa, from about 70 kDa to about 150 kDa, from about 80 kDa to about 90 kDa, from about 80 kDa to about 100 kDa, from about 80 kDa to about 150 kDa, from about 90 kDa to about 100 kDa, from about 90 kDa to about 150 kDa, or from about 100 kDa to about 150 kDa.

It is contemplated that the antibody conjugate or fusion protein is attached to about 10 or fewer polymer molecules (e.g., 9, 8, 7, 6, 5, 4, 3, 2, or 1), each polymer molecule having a molecular weight of at least about 20,000 D, or at least about 30,000 D, or at least about 40,000 D.

Although a variety of polymers can be used as biological modifiers, it is contemplated that the antibody conjugates or fusion proteins described herein may be attached to polyethylene glycol (PEG) polymers. In one embodiment, the antibody conjugate or fusion protein described herein is covalently attached to at least one PEG having an actual MW of at least about 20,000 D. In another embodiment, the antibody conjugate or fusion protein described herein is covalently attached to at least one PEG having an actual MW of at least about 30,000 D. In another embodiment, the antibody conjugate or fusion protein described herein is covalently attached to at least one PEG having an actual MW of at least about 40,000 D. In certain embodiments, the PEG is methoxyPEG (5000)-succinimidylpropionate (mPEG-SPA), methoxyPEG (5000)-succinimidylsuccinate (mPEG-SS). Such PEGS are commercially available from Nektar Therapeutics or SunBiowest.

Attachment sites on an antibody conjugate or fusion protein for a biological modifier include the N-terminal amino group and epsilon amino groups found on lysinc residues, as well as other amino, imino, carboxyl, sulfhydryl, hydroxyl or other hydrophilic groups. The polymer may be covalently bonded directly to the antibody conjugate or fusion protein with or without the known use of a multifunctional (ordinarily bifunctional) crosslinking agent using chemistries and used in the art. For example, sulfhydryl groups can be derivatized by coupling to maleimido-substituted PEG (e.g. alkoxy-PEG amine plus sulfosuccinimidyl 4-(N-maleimidomethyl)cyclohexane-1-carboxylate), or PEG-maleimide commercially available from Shearwater Polymers, Inc., Huntsville, Ala.).

### III. Methods of Making a Recombinant Human Sialidase, Fusion Protein, or Antibody Conjugate

Methods for producing recombinant human sialidases, fusion proteins, e.g., those disclosed herein, antibodies, or antibody conjugates, e.g., those disclosed herein, are known in the art. For example, DNA molecules encoding light chain variable regions and/or heavy chain variable regions can be synthesized chemically or by recombinant DNA methodologies. For example, the sequences of the antibodies can be cloned from hybridomas by conventional hybridization techniques or polymerase chain reaction (PCR) techniques, using the appropriate synthetic nucleic acid primers. The resulting DNA molecules encoding the variable regions of interest can be ligated to other appropriate nucleotide sequences, including, for example, constant region coding sequences, and expression control sequences, to produce conventional gene expression constructs (i.e., expression vectors) encoding the desired antibodies. Production of defined gene constructs is within routine skill in the art.

Nucleic acids encoding desired recombinant human sialidases, fusion proteins, and/or antibody conjugates can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

The host cells express a recombinant human sialidase or a fusion protein and/or antibody conjugate comprising a sialidase and V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments involving fusion proteins and/or antibody conjugates, a host cell is transfected with a single vector expressing a polypeptide expressing a sialidase and an entire, or part of, a heavy chain (e.g., a heavy chain variable region) or a sialidase and a light chain (e.g., a light chain variable region), or a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain, wherein in (a) or in (b), the polypeptide may also comprise a sialidase. In some embodiments, a host cell is co-transfected with more than one expression vector (e.g., one expression vector expressing a polypeptide comprising an entire, or part of, a heavy chain or heavy chain variable region, optionally comprising a sialidase fused thereto, and another expression vector expressing a polypeptide comprising an entire, or part of, a light chain or light chain variable region, optionally comprising a sialidase fused thereto).

A polypeptide comprising a sialidase or a fusion protein, e.g., a fusion protein comprising an immunoglobulin heavy chain variable region or light chain variable region, can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

In embodiments in which a fusion protein and/or antibody conjugate is produced, a sialidase fused to a monoclonal antibody, Fc domain, or an antigen-binding domain of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The sialidase will be fused to one or more of the chains. The intact fusion protein and/or antibody conjugate can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

In certain embodiments, in order to express a protein, e.g., a recombinant human sialidase, as a secreted protein, a native N-terminal signal sequence of the protein is replaced, e.g., with MDMRVPAQLLGLLLLWLPGARC (SEQ ID NO: 28). In certain embodiments, to express a protein, e.g., a recombinant human sialidase, as a secreted protein, an N-terminal signal sequence, e.g., MDMRVPAQLLGLLLLWLPGARC (SEQ ID NO: 28), is added. Additional exemplary N-terminal signal sequences include signal sequences from interleukin-2, CD-5, IgG kappa light chain, trypsinogen, serum albumin, and prolactin. In certain embodiments, in order to express a protein, e.g., a recombinant human sialidase, as a secreted protein, a C terminal lysosomal signal motif, e.g., YGTL (SEQ ID NO: 29) is removed.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. Scc, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332:323-327; Verhocyen et al. (1988) SCIENCE 239:1534-1536; and Winter (1998) FEBS LETT 430:92-94.

In an approach called “SUPERHUMANIZATION™,” human CDR sequences are chosen from human germline genes, based on the structural similarity of the human CDRs to those of the mouse antibody to be humanized. Sec, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” Sec, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains can be produced. Sec, e.g., U.S. Pat. No. 6,706,477 (Zauderer); U.S. Pat. No. 6,800,442 (Zauderer); and 6,872,518 (Zauderer). Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and 5,869,619 (Studnicka).

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human monoclonal antibodies can also be prepared and optimized from phage display libraries by techniques referenced in, e.g., Knappik et al., J. MOL. BIOL. 296:57-86, 2000; and Krebs et al., J. IMMUNOL. METH. 254:67-84 2001).

The present invention encompasses fusion proteins comprising antibody fragments, which may be generated by traditional means, such as enzymatic digestion, or by recombinant techniques. For a review of certain antibody fragments, see Hudson et al. (2003) NAT. MED. 9:129-134.

Various techniques have been developed for the production of antibody fragments. Traditionally, these fragments were derived via proteolytic digestion of intact antibodies (see, e.g., Morimoto et al. (1992) JOURNAL OF BIOCHEMICAL AND BIOPHYSICAL METHODS 24:107-117; and Brennan et al. (1985) SCIENCE 229:81). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F (ab′) 2 fragments (Carter et al. (1992) BIO/TECHNOLOGY 10:163-167). According to another approach, F (ab′) 2 fragments can be isolated directly from recombinant host cell culture. Fab and F (ab′) 2 fragments with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). Sec U.S. Pat. Nos. 5,571,894 and 5,587,458.

Methods for making bispecific antibodies are known in the art. See Milstein and Cuello (1983) NATURE 305:537, International (PCT) Publication No. WO93/08829, and Traunecker et al. (1991) EMBO J., 10:3655. For further details of generating bispecific antibodies see, for example, Suresh et al. (1986) METHODS ENZYMOL. 121:210. Bispecific antibodies include cross-linked or “heteroconjugate” or “heterodimer” antibodies. For example, one of the antibodies in the heterodimer can be coupled to avidin, the other to biotin. Heterodimer antibodies may be made using any convenient cross-linking method. Suitable cross-linking agents are well known in the art, and are disclosed in U.S. Pat. No. 4,676,980, along with a number of cross-linking techniques.

Examples of heterodimeric or asymmetric IgG-like molecules include but are not limited to those obtained with the following technologies or using the following formats: Triomab/Quadroma, Knobs-into-Holes, CrossMabs, electrostatically-matched antibodies, LUZ-Y, Strand Exchange Engineered Domain body, Biclonic and DuoBody.

Advantages of using antibody fragments (e.g., F (ab) and F (ab′) 2 fragments) include the elimination of non-specific binding between Fc portions of antibodies and Fc receptors on cells (such as macrophages, dendritic cells, neutrophils, NK cells and B cells). In addition, they may be able to penetrate tissues more efficiently due to their smaller size.

Heterodimeric antibodies, or asymmetric antibodies, allow for greater flexibility and new formats for attaching a variety of drugs to the antibody arms. One of the general formats for creating a heterodimeric antibody is the “knobs-into-holes” format. This format is specific to the heavy chain part of the constant region in antibodies. The “knobs” part is engineered by replacing a small amino acid with a larger one, which fits into a “hole”, which is engineered by replacing a large amino acid with a smaller one. What connects the “knobs” to the “holes” are the disulfide bonds between each chain. The “knobs-into-holes” shape facilitates antibody dependent cell mediated cytotoxicity. Single chain variable fragments (scFv) are connected to the variable domain of the heavy and light chain via a short linker peptide. The linker is rich in glycine, which gives it more flexibility, and serine/threonine, which gives it specificity. Two different scFv fragments can be connected together, via a hinge region, to the constant domain of the heavy chain or the constant domain of the light chain. This gives the antibody bispecificity, allowing for the binding specificities of two different antigens. The “knobs-into-holes” format enhances heterodimer formation but doesn't suppress homodimer formation.

Several approaches to support heterodimerization have been described, for example in International (PCT) Publication Nos. WO96/27011, WO98/050431, WO2007/110205, WO2007/147901, WO2009/089004, WO2010/129304, WO2011/90754, WO2011/143545, WO2012/058768, WO2013/157954, and WO2013/096291, and European Patent Publication No. EP1870459. Typically, in the approaches known in the art, the CH₃ domain of the first heavy chain and the CH₃ domain of the second heavy chain are both engineered in a complementary manner so that the heavy chain comprising one engineered CH₃ domain can no longer homodimerize with another heavy chain of the same structure (e.g. a CH₃-engineered first heavy chain can no longer homodimerize with another CH₃-engineered first heavy chain; and a CH₃-engineered second heavy chain can no longer homodimerize with another CH₃-engineered second heavy chain). Thereby the heavy chain comprising one engineered CH₃ domain is forced to heterodimerize with another heavy chain comprising the CH₃ domain, which is engineered in a complementary manner. As a result, the CH₃ domain of the first heavy chain and the CH₃ domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g., for steric reasons).

### IV. Pharmaceutical Compositions

For therapeutic use, a recombinant human sialidase or a fusion protein and/or antibody conjugate thereof preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1:10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D (−)-3-hydroxybutyric acid. Sustained release compositions may also include liposomes that can be prepared by any of several methods known in the art.

Pharmaceutical compositions containing a recombinant human sialidase, a recombinant human sialidase fusion protein, or an antibody conjugate disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a recombinant human sialidase, a recombinant human sialidase fusion protein, or an antibody conjugate disclosed herein is administered by IV infusion. In certain embodiments, a recombinant human sialidase, a recombinant human sialidase fusion protein, or an antibody conjugate disclosed herein is administered by intratumoral injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, a recombinant human sialidase or fusion protein and/or antibody conjugate thereof, is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the recombinant human sialidase or fusion protein and/or antibody conjugate thereof, and the disease being treated. Exemplary dosing frequencies are once per day, once per week and once every two weeks. A preferred route of administration is parenteral, e.g., intravenous infusion. In certain embodiments, a recombinant human sialidase or a fusion protein and/or antibody conjugate thereof is lyophilized, and then reconstituted in buffered saline, at the time of administration.

### V. Therapeutic Uses

The compositions and methods disclosed herein can be used to treat various forms of cancer in a subject or inhibit cancer growth in a subject. The invention provides a method of treating a cancer in a subject. The method comprises administering to the subject an effective amount of a recombinant human sialidase or a fusion protein and/or antibody conjugate thereof, e.g., a recombinant human sialidase, fusion protein, or antibody conjugate disclosed herein, either alone or in a combination with another therapeutic agent to treat the cancer in the subject. The term “effective amount” as used herein refers to the amount of an active agent (e.g., recombinant human sialidase or fusion protein thereof according to the present invention) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments the cancer is an epithelial cancer, e.g., an epithelial cancer that upregulates the expression of sialylated glycans. Exemplary epithelial cancers include, but are not limited to, endometrial cancer, colon cancer, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer and liver cancer. Epithelial cancers also include carcinomas, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In certain embodiments, the cancer is breast cancer. In certain embodiments, the cancer is an adenocarcinoma. In certain embodiments, the cancer is a metastatic cancer. In certain embodiments, the cancer is a refractory cancer.

In certain embodiments, the cancer is resistant to or non-responsive to treatment with an antibody, e.g., an antibody with ADCC activity, e.g., trastuzumab.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a combination of therapeutics of different classes.

In certain embodiments, a method or composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP U.S. Pat. No. 1,537,878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), duvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, a method or composition described herein is administered in combination with (i) a PD-1 or PD-L1 inhibitor, e.g., a PD-1 or PD-L1 inhibitor disclosed herein, and (ii) CTLA-4 inhibitor, e.g., a CTLA-4 inhibitor disclosed herein.

In certain embodiments, a method or composition described herein is administered in combination with an IDO inhibitor. Exemplary IDO inhibitors include 1-methyl-D-tryptophan (known as indoximod), epacadostat (INCB24360), navoximod (GDC-0919), and BMS-986205.

Exemplary cytotoxic agents that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, ctoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

The invention also provides a method of increasing the expression of HLA-DR, CD86, CD83, IFNγ, IL-1b, IL-6, TNFα, IL-17A, IL-2, or IL-6 in a cell or tissue. The method comprises contacting the cell or tissue with an effective amount of a recombinant human sialidase or a fusion protein and/or antibody conjugate thereof, e.g., a recombinant human sialidase, fusion protein, or antibody conjugate disclosed herein. In certain embodiments, the cell is selected from a dendritic cell and a peripheral blood mononuclear cell (PBMC).

In certain embodiments, expression of HLA-DR, CD86, CD83, IFNγ, IL-1b, IL-6, TNFα, IL-17A, IL-2, or IL-6 in the cell or tissue is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the recombinant human sialidase, fusion protein, or antibody conjugate. Gene expression may be measured by any suitable method known in the art, for example, by ELISA, or by Luminex multiplex assays, as described in Example 13 herein.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This example describes the construction of recombinant human sialidases (Neu1, Neu2, and Neu3) with substitutions of cysteine residues to enhance expression and/or reduce aggregation.

The human sialidases Neu1, Neu2, Neu3 (isoform 1), and Neu4 (isoform 1) were expressed as secreted proteins with a 10×His tag (SEQ ID NO: 105). To express Neu1 as a secreted protein, the native N terminal signal peptide (MTGERPSTALPDRRWGPRILGFWGGCRVWVFAAIFLLLSLAASWSKA; SEQ ID NO: 27) was replaced by MDMRVPAQLLGLLLLWLPGARC (SEQ ID NO: 28), and the C terminal lysosomal signal motif (YGTL; SEQ ID NO: 29) was removed. To express Neu2, Neu3, and Neu4 as secreted proteins, the N terminal signal peptide MDMRVPAQLLGLLLLWLPGARC (SEQ ID NO: 28) was added to each.

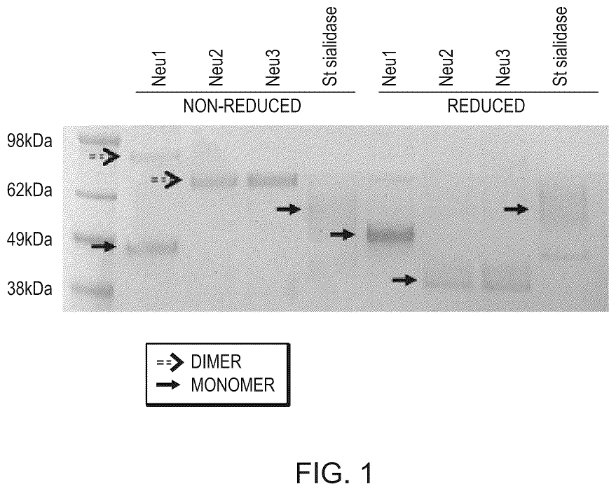

Sialidases were expressed in a 200 mL transfection of HEK293F human cells in 24-well plates using the pCEP4 mammalian expression vector with an N-terminal 6×His tag (SEQ ID NO: 109). Sialidases were purified using Ni-NTA columns, quantified with a UV-Vis spectrophotometer (NanoDrop), and examined by SDS-PAGE as shown in FIG. 1. Neu1 expressed well, with a yield of ˜3 μg/ml, and was present primarily in a monomeric form. Neu2 and Neu3 expression each gave yields of ˜0.15 μg/mL and each were present primarily in a dimeric form. Neu4 had no detectable expression yield as measured by NanoDrop. Bacterial sialidase from Salmonella typhimurium (St-sialidase; SEQ ID NO: 30), which was used as a positive control for expression, gave a comparable yield to Neu1, and was present primarily in a monomeric form.

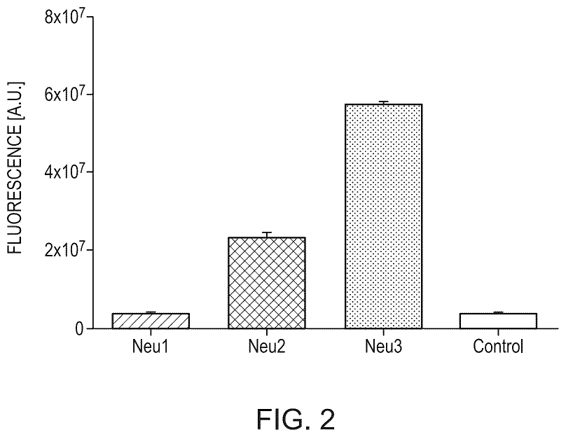

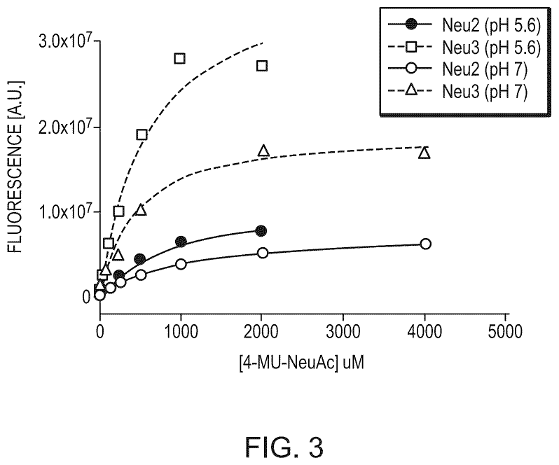

The activity of the recombinantly expressed sialidases was assayed by measuring the release of sialic acid from the fluorogenic substrate 4-methylumbelliferyl-N-acetylneuraminic acid (4MU-NeuAc). As shown in FIG. 2, Neu1 has no detectable activity above a no-enzyme control, which is consistent with previous reports indicating that Neu1 is inactive unless it is in complex with beta-galactosidase and protective protein/cathepsin A (PPCA). Neu2 and Neu3 were active. An enzyme kinetics assay was performed with Neu2 and Neu3. A fixed concentration of enzyme at 1 nM was incubated with fluorogenic substrate 4MU-NeuAc at concentrations ranging from 4000 μM to 7.8 μM. Assays were conducted at both acidic (pH 5.6) and neutral (pH 7) conditions. As shown in FIG. 3, both Neu2 and Neu3 were active at acidic and neutral conditions and showed enzyme kinetics that were comparable to those previously reported.

Most of the recombinantly expressed sialidases ran as aggregates or dimers on a non-reducing SDS-PAGE gel. Subsequent treatment with the reducing agent dithiothreitol (DTT) resulted in a monomeric form of the enzyme that ran at 42 kDa on a reducing SDS-PAGE gel (FIG. 1). Thus, free cysteine residues in the sialidases may cause aggregation, dimerization, and/or low expression. Accordingly, each of the six free cysteine residues of Neu2 (125, 196, 219, 272, 332, 352) were substituted with the amino acids S, I, V, F, L, or A, using site-directed mutagenesis. The resulting mutant sialidases were expressed in 24-well plates as secreted proteins with an N-terminal human Fc tag in Expi293F cells using the pCEP4 mammalian expression vector. Expression was assayed using a ForteBio Octet with anti-human Fc sensors and Western blot and enzymatic activity was assayed using the fluorogenic substrate 4MU-NeuAc as described above. Expression and activity levels for the mutant sialidases are shown in TABLE 2.

In TABLE 2, enzymatic activity is indicated as “++,” which denotes activity comparable to wild-type Neu2, “+,” which denotes activity lower than wild-type Neu2, or “−,” which denotes no detectable activity, and expression is indicated as “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 2

| Identifier | Mutation(s) | Activity | Expression |
|---|---|---|---|
| Neu2 | Wild-type Neu2 | ++ | + |
| Neu2-M1 | C125A | ++ | + |
| Neu2-M2 | C125I | ++ | + |
| Neu2-M3 | C125S | ++ | + |
| Neu2-M4 | C125V | ++ | + |
| Neu2-M5 | C196A | ++ | + |
| Neu2-M6 | C196L | ++ | + |
| Neu2-M7 | C196F | − | − |
| Neu2-M8 | C196S | − | − |
| Neu2-M9 | C196V | ++ | + |
| Neu2- | C219A | − | +++ |
| M10 | | | |
| Neu2- | C219N | − | +++ |
| M11 | | | |
| Neu2- | C219S | − | − |
| M12 | | | |
| Neu2- | C219V | − | − |
| M13 | | | |
| Neu2- | C219D | − | +++ |
| M14 | | | |
| Neu2- | C219I | − | + |
| M15 | | | |
| Neu2- | C219L | − | + |
| M16 | | | |
| Neu2- | C219Q | − | +++ |
| M17 | | | |
| Neu2- | C219M | − | +++ |
| M18 | | | |
| Neu2- | C219T | − | +++ |
| M19 | | | |
| Neu2- | C272S | + | + |
| M20 | | | |
| Neu2- | C272V | ++ | + |
| M21 | | | |
| Neu2- | C332A | ++ | + |
| M22 | | | |
| Neu2- | C332S | ++ | + |
| M23 | | | |
| Neu2- | C332V | ++ | + |
| M24 | | | |
| Neu2- | C352L | ++ | + |
| M25 | | | |
| Neu2- | C352S | − | − |
| M26 | | | |
| Neu2- | C352V | ++ | + |
| M27 | | | |
| Neu2- | C196S + 219S + 332S | − | +++ |
| M28 | | | |
| Neu2- | C125S + C196S + C272S + C352S + | − | − |
| M29 | C332S | | |
| Neu2- | C125S + C196S + C219S + C272S + | − | − |
| M30 | C352S + C332S | | |
| Neu2- | C125S + C332S | ++ | + |
| M31 | | | |
| Neu2- | C196A + C219A | − | + |
| M32 | | | |
| Neu2- | C196V + C219V | − | − |
| M33 | | | |
| Neu2- | C196L + C219N | − | − |
| M34 | | | |
| Neu2- | C196L + C219A | − | + |
| M35 | | | |
| Neu2- | C272V + C332A | ++ | + |
| M36 | | | |
| Neu2- | C272V + C332S | ++ | + |
| M37 | | | |
| Neu2- | C332A + C352L | + | ++ |
| M38 | | | |
| Neu2- | C125S + C196L | ++ | + |
| M39 | | | |
| Neu2- | C196L + C219N + C332S | − | + |
| M40 | | | |
| Neu2- | C196L + C352L | ++ | + |
| M41 | | | |
| Neu2- | C196L + C219N + C332A | − | +++ |
| M42 | | | |
| Neu2- | C196L + C272V + C352L | − | + |
| M43 | | | |
| Neu2- | C272V + C332A + C352L | − | +++ |
| M44 | | | |
| Neu2- | C196L + C272V + C352L + C332A | − | + |
| M45 | | | |
| Neu2- | C196L + C272V + C352L + C332S | − | + |
| M46 | | | |
| Neu2- | C196L + C332S | + | − |
| M47 | | | |
| Neu2- | C196L + C332A | ++ | + |
| M48 | | | |
| Neu2- | C125S + C196L + C272V + C352L + | − | + |
| M49 | C332A | | |
| Neu2- | C125S + C196L + C272V + C352L + | − | + |
| M50 | C332S | | |
| Neu2- | C196L + C332A + C352L | + | + |
| M51 | | | |
| Neu2- | C125S + C272V + C332A + C352L | − | + |
| M52 | | | |
| Neu2- | C272V + C332A + C352L + K45A | − | + |
| M53 | | | |
| Neu2- | C196L + C219T + C332S | − | ++ |
| M54 | | | |
| Neu2- | C196L + C219T + C332A | − | +++ |
| M55 | | | |
| Neu2- | C125S + C196L + C219T + C272V + | − | +++ |
| M56 | C352L + C332A | | |
| Neu2- | C125S + C196L + C219T + C272V + | − | +++ |
| M57 | C352L + C332S | | |
| Neu2- | C196L + C219N + C332A | − | + |
| M58 | | | |
| Neu2- | C219N + C332A | − | +++ |
| M59 | | | |
| Neu2- | C219A + C332A | − | +++ |
| M60 | | | |
| Neu2- | C196L + C219A + C332A | − | +++ |
| M61 | | | |

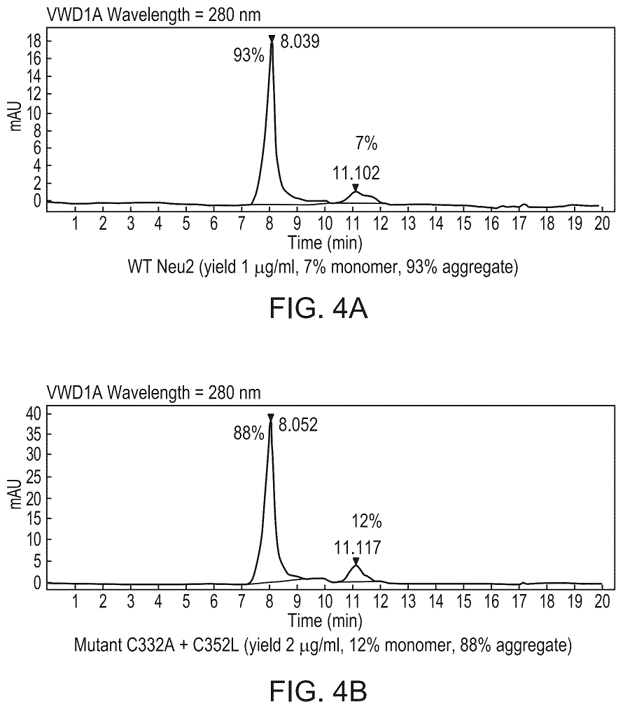

As seen in TABLE 2, mutation of cysteine 219 greatly enhances expression, but negatively effects enzymatic activity. This may be due to the effects of the cysteine 219 mutation on the neighboring amino acid glutamate 218, which is believed to be a critical catalytic residue that acts a nucleophile for catalysis. Individual mutations of the other five cysteines (125, 196, 272, 332, and 352) had minimal impact on expression. However, through extensive combinatorial mutagenesis, a mutant sialidase with both the C332A and C352L substitutions (Neu2-M38) was identified that had improved expression and maintained enzymatic activity (although with reduced enzymatic activity relative to wild-type). To confirm these results, Neu2-M38 was expressed in a 100 mL transfection in shaking flasks and purified with a protein A column. Neu2-M38 had 2 fold higher expression than wild-type Neu2 under the same conditions and improved monomer content (12% vs 7%) as characterized by SEC-HPLC (FIGS. 4A and 4B). Together, these results show that mutating free cysteine residues in a human sialidase can be advantageous for producing secreted recombinant human sialidases and improving expression of recombinant human sialidases.

### Example 2

This example demonstrates that engineering surface exposed residues of a human sialidase can increase the isoelectric point (pI) of the sialidase and/or reduce the hydrophobicity of a surface on the sialidase to improve solubility and/or decrease protein aggregation.

Human Neu2 has a predicted pI of 7.5, as compared to pI of 9.6 of the Salmonella typhimurium sialidase (St-sialidase). Additionally, an analysis of the surface hydrophobicity of Neu2 using the available crystal structure revealed a large exposed hydrophobic patch on the surface of Neu2, primarily including the N-terminal amino acids of Neu2, e.g., A2, as well as V325. These features may be suboptimal for protein stability and solubility in neutral aqueous conditions, possibly as a result of aggregation due to inter-molecular hydrophobic interactions.

Surface residues of Neu2 were chosen as candidates for substitutions to increase solubility and/or expression, according to the following criteria: surface exposed D or E residues; hydrophobic residues contributing to surface hydrophobic patches; residues not involved in catalysis; residues not well conserved between human Neu 1, 3, 4, St-sialidase; and residues at positions that have a homologous K or R in other sialidases. Using these criteria, the acidic amino acids E72, D215, and E257 in Neu2 were mutated to lysine to increase pI, and the hydrophobic amino acids A2 and V325 in Neu2 were mutated to lysine or glutamate to reduce the hydrophobicity of the predicted Neu2 surface hydrophobic patch. The resulting mutant sialidases were expressed in 24-well plates as secreted proteins with a human Fc tag in Expi293F cells using the pCEP4 mammalian expression vector and assayed for expression and activity as described above in Example 1.

Expression and activity levels for the mutant sialidases are shown in TABLE 3. In TABLE 3, enzymatic activity is indicated as “++,” which denotes activity comparable to wild-type Neu2, “+,” which denotes activity lower than wild-type Neu2, or “−,” which denotes no detectable activity, and expression is indicated as “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 3

| Identifier | Mutation(s) | Activity | Expression |
|---|---|---|---|
| Neu2 | Wild-type Neu2 | ++ | + |
| Neu2-M62 | A2K | ++ | ++ |
| Neu2-M63 | E72K | + | − |
| Neu2-M64 | D215K | + | + |
| Neu2-M65 | E257K | + | ++ |
| Neu2-M66 | E319K | − | ++ |
| Neu2-M67 | V325K | + | ++ |
| Neu2-M68 | A2K + E257K | + | +++ |
| Neu2-M69 | A2K + V325E | + | + |
| Neu2-M70 | A2E + V325K | + | − |
| Neu2-M71 | A2K + V325K | + | +++ |
| Neu2-M72 | E257K + V325K | + | ++ |
| Neu2-M73 | E257K + A2K + V325K | − | +++ |

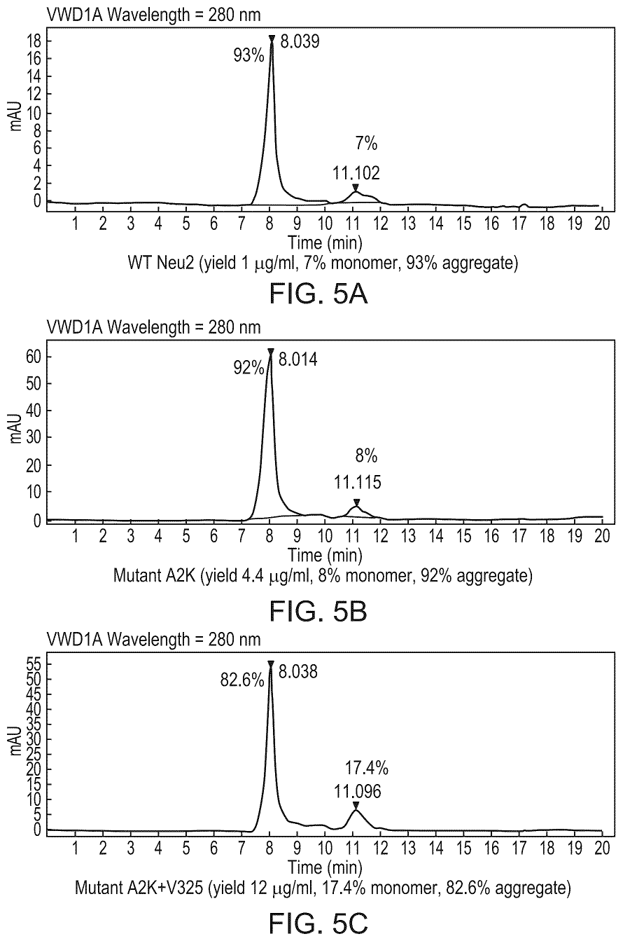

As seen in TABLE 3, the Neu2-M62 (A2K), Neu2-M68 (A2K+E257K), and Neu2-M71 (A2K+V325K) mutant sialidases showed improved expression and comparable or reduced enzymatic activity compared to wild-type Neu2. To confirm these results, the Neu2-M62 and Neu2-M71 mutant sialidases were expressed in a 100 mL transfection in shaking flasks and purified with a protein A column. Neu2-M62 had ˜4.4 fold higher expression than wild-type Neu2 (4.4 μg/mL vs 1 μg/mL) and similar monomer content (8% vs 7%) as characterized by SEC-HPLC (FIGS. 5A and 5B). Neu2-M71 had ˜12 fold higher expression than wild-type Neu2 (12 μg/mL vs 1 μg/mL) and improved monomer content (17% vs 7%) as characterized by SEC-HPLC (FIGS. 5A and 5C), but had no enzymatic activity.

Together, these results show that mutating surface exposed residues in a human sialidase can increase the isoelectric point (pI) of the sialidase and/or reduce the hydrophobicity of a surface on the sialidase to improve solubility, decrease protein aggregation, and/or improve expression of recombinant human sialidase.

### Example 3

This Example demonstrates that the addition of a short peptide to the N-terminus of a human sialidase can increase expression and/or activity of the sialidase.

Using homology-based engineering, we grafted variants of an N-terminal sequence (MEDLRP; SEQ ID NO: 4) from mouse thymus Neu2 onto human Neu2 by overlapping PCR. The resulting mutant sialidases were expressed in 24-well plates as secreted proteins with a human Fc tag in Expi293F cells using the pCEP4 mammalian expression vector and assayed for expression and activity as described in Example 1.

Expression and activity levels for the mutant sialidases are shown in TABLE 4. In TABLE 4, enzymatic activity is indicated as “+++,” which denotes activity greater than wild-type Neu2, “++,” which denotes activity comparable to wild-type Neu2, “+,” which denotes activity lower than wild-type Neu2, or “−,” which denotes no activity, and expression is indicated as “+++,” which denotes expression >6 fold higher than wild-type Neu2, “++,” which denotes expression 2-5 fold higher than wild-type Neu2, or “+,” which denotes expression comparable to wild-type Neu2.

TABLE 4

| Identifier | Mutation(s) | Activity | Expression |
|---|---|---|---|
| Neu2 | Wild-type Neu2 | ++ | + |
| Neu2- | Substitute M at the N-terminus of | ++ | + |
| M74 | Neu2 with EDLRP (SEQ ID NO: 3) | | |
| Neu2- | Substitute M at the N-terminus of | +++ | +++ |
| M75 | Neu2 with MEDLRP (SEQ ID NO: 4) | | |
| Neu2- | Insert MEDLRP (SEQ ID NO: 4) at | +++ | +++ |
| M76 | the N-terminus of Neu2 | | |
| Neu2- | Substitute MASLP (SEQ ID NO: 12) | +++ | +++ |
| M77 | at the N-terminus of Neu2 with | | |
| | MEDLRP (SEQ ID NO: 4) | | |

As shown in TABLE 4, all variants tested that included the MEDLRP (SEQ ID NO: 4) N-terminal sequence had both increased expression and activity relative to wild-type Neu2.

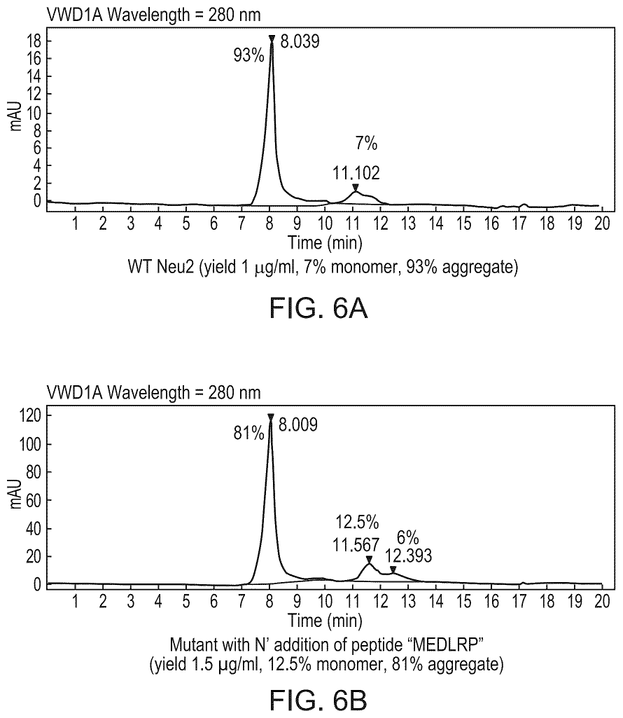

To confirm these results, the mutant Neu2-M76 (which included MEDLRP (SEQ ID NO: 4) inserted at the N-terminus) was expressed in a 100 mL transfection in shaking flasks and purified with a protein A column. Compared to the 24-well format, Neu2-M76 only showed a modest improvement in expression, ˜1.5 fold higher than that of wild-type Neu2 (1.5 μg/mL vs 1 μg/mL) with improved monomer content (12.5% vs 7%) as characterized by SEC-HPLC (FIGS. 6A and 6B).

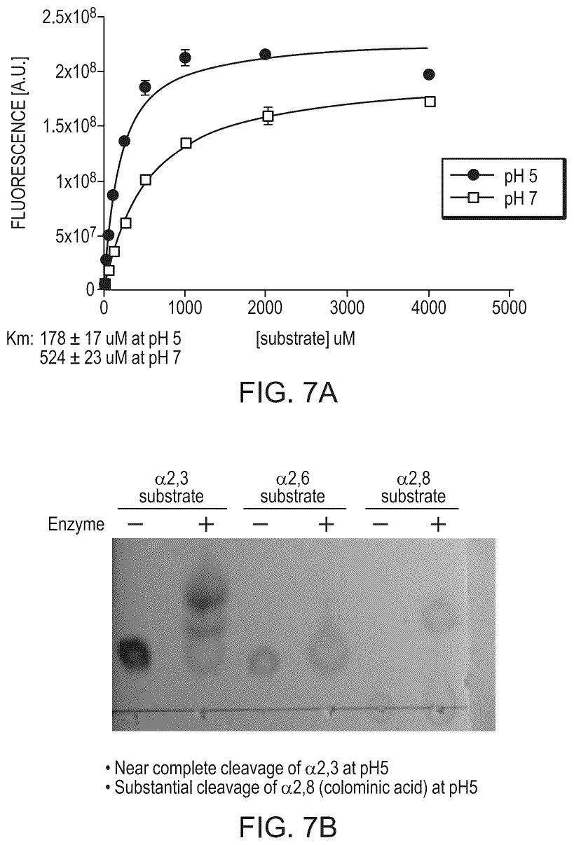

Enzyme kinetics measurements were carried out with purified Neu2-M76. A fixed concentration of Neu2-M76 at 100 nM was incubated with fluorogenic substrate 4MU-NeuAc at concentrations ranging from 4000 μM to 7.8 μM. As shown in FIG. 7A, this variant had a KM of ˜ 175 μM, 5 fold more potent than that of wild-type Neu2 (KM of ˜867 μM). Additionally, as shown in FIG. 7B, Neu2-M76 also had an altered substrate specificity relative to wild-type Neu2, as it cleaved sialic acid with α2,8 linkages (colominic acid) at pH 5, while wild-type Neu2 had no such activity.

Together, these results show that the addition of a short peptide to the N-terminus of a human sialidase can increase expression, increase activity, and/or modify the substrate specificity of the sialidase.

### Example 4

This Example demonstrates that mutating residues in the N- or C-terminus of a human sialidase to increase hydrophobic interactions and/or hydrogen bonding between the N- and C-termini can increase stability and/or expression of the sialidase.

Based on the crystal structure of Neu2, residues LA, V6, L7, and L12 were mutated to promote hydrophobic interactions or hydrogen bonding between the N- and C-termini of Neu2. The resulting mutant sialidases were expressed in 24-well plates as secreted proteins with a human Fc tag in Expi293F cells using the pCEP4 mammalian expression vector and assayed for expression and activity as described in Example 1.

Expression and activity levels for the mutant sialidases are shown in TABLE 5. In TABLE 5, enzymatic activity is indicated as “++,” which denotes activity comparable to wild-type Neu2, “+,” which denotes activity lower than wild-type Neu2, or “−,” which denotes no detectable activity, and expression is indicated as “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 5

| Identifier | Mutation(s) | Activity | Expression |
|---|---|---|---|
| Neu2 | Wild-type Neu2 | ++ | + |
| Neu2-M78 | L4N | ++ | + |
| Neu2-M79 | V6Y | ++ | +++ |
| Neu2-M80 | L7N | + | + |
| Neu2-M81 | L4N + L7N | − | − |
| Neu2-M82 | V12N | − | + |
| Neu2-M83 | V6F | + | +++ |
| Neu2-M84 | V6W | + | ++ |

As shown in TABLE 5, the V6Y substitution (Neu2-M79) resulted in improved expression and enzymatic activity compared to wild-type Neu2.

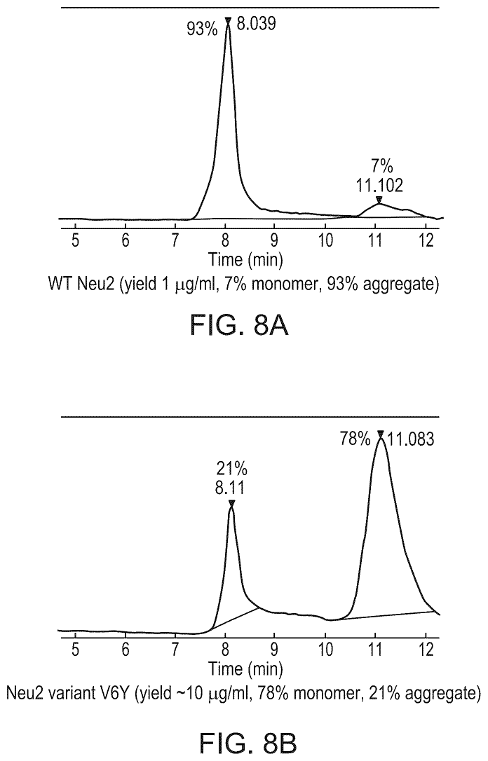

To confirm these results, Neu2-M79 was expressed in a 100 mL transfection in shaking flasks and purified with a protein A column. Neu2-M79 had ˜ 10 fold higher expression than wild-type Neu2 (10 μg/mL vs 1 μg/mL), substantially improved monomer content (78% vs 7%) as characterized by SEC-HPLC (FIGS. 8A and 8B), and was as active as wild-type Neu2.

Together, these results show that mutating residues in the N- or C-termini of a human sialidase to increase hydrophobic interactions and/or hydrogen bonding between the N- and C-termini can increase stability and/or expression of the sialidase.

### Example 5

This Example demonstrates that mutating the N-terminal methionine of a human sialidase can increase stability and/or expression of the sialidase.

The first residue (M1) of human Neu2 was deleted or mutated to R, H, K, D, E, S, T, N, Q, G, P, A, V, L, F, and Y. All mutations were tested in combination with V6Y and I187K substitutions. The resulting mutant sialidases were expressed in shaking flasks as secreted proteins with a human Fc tag in Expi293F cells using the pCEP4 mammalian expression vector. The proteins were purified with a protein A column, quantified by Nanodrop, and characterized for enzymatic activity as described in Example 1.

Expression and activity levels for the mutant sialidases are shown in TABLE 6. In TABLE 6, enzymatic activity is indicated as “++,” which denotes activity comparable to wild-type Neu2, “+,” which denotes activity lower than wild-type Neu2, or “−,” which denotes no detectable activity, and expression is indicated as “+++,” which denotes expression >6 fold higher than wild-type Neu2, “++,” which denotes expression 2-5 fold higher than wild-type Neu2, “+,” which denotes expression comparable to wild-type Neu2, or “,” which denotes no detectable expression.

TABLE 6

| Identifier | Mutation(s) | Activity | Expression |
|---|---|---|---|
| Neu2 | None-Wild-type Neu2 | ++ | + |
| Neu2-M85 | Deletion of M1, V6Y, I187K | + | +++ |
| Neu2-M86 | M1R, V6Y, I187K | + | +++ |
| Neu2-M87 | M1H, V6Y, I187K | +++ | ++ |
| Neu2-M88 | M1K, V6Y, I187K | + | +++ |
| Neu2-M89 | M1D, V6Y, I187K | +++ | +++ |
| Neu2-M90 | M1E, V6Y, I187K | − | +++ |
| Neu2-M91 | M1S, V6Y, I187K | − | − |
| Neu2-M92 | M1T, V6Y, I187K | ++ | ++ |
| Neu2-M93 | M1N, V6Y, I187K | ++ | +++ |
| Neu2-M94 | M1Q, V6Y, I187K | + | ++ |
| Neu2-M95 | M1G, V6Y, I187K | + | +++ |
| Neu2-M96 | M1P, V6Y, I187K | − | − |
| Neu2-M97 | M1A, V6Y, I187K | ++ | +++ |
| Neu2-M98 | M1V, V6Y, I187K | + | +++ |
| Neu2-M99 | M1L, V6Y, I187K | +++ | +++ |
| Neu2-M100 | M1F, V6Y, I187K | +++ | ++ |
| Neu2-M101 | M1Y, V6Y, I187K | + | + |

As shown in TABLE 6, deletion of M1 or mutation of M1 to R, H, K, D, E, T, N, Q, G, A, V, L, or F in combination with V6Y and I187K substitutions increased expression of the sialidase, with the M1H, M1D, MIL, and MIF mutations resulting in increased expression and enzymatic activity. Together, these results show that mutating the N-terminal methionine of a human sialidase can increase stability and/or expression of the sialidase.

### Example 6

This Example describes mutations and combinations of mutations that can increase stability and/or expression of the sialidase.

Human Neu2 was mutated as shown in TABLE 7. The resulting mutant sialidases were expressed in shaking flasks as secreted proteins with a human Fc tag in Expi293F cells using the pCEP4 mammalian expression vector. The proteins were purified with a protein A column, quantified by Nanodrop, and characterized for enzymatic activity as described in Example 1.

Expression and activity levels for the mutant sialidases are shown in TABLE 7. In TABLE 7, enzymatic activity is indicated as “++,” which denotes activity comparable to wild-type Neu2, “+,” which denotes activity lower than wild-type Neu2, or “−,” which denotes no detectable activity, and expression is indicated as “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 7

| Identifier | Mutation(s) | Activity | Expression |
|---|---|---|---|
| Neu2-M102 | M1D, V6Y, I187K, C332A | +++ | +++ |
| Neu2-M103 | V6Y, I187K, C332A | +++ | ++ |

### Example 7

This Example describes the construction and expression of antibody-sialidase genetic fusion proteins, and antibody sialidase conjugates (ASCs) containing the fusion proteins, with bacterial and mutated human sialidases.

The architecture for three types of ASCs used in this Example is depicted in FIG. 10. The first type of ASC, referred to as “Raptor,” includes an antibody (with two heavy chains and two light chains) with a sialidase fused at the C-terminus of each heavy chain of the antibody. The second type of ASC, referred to as “Janus,” contains one antibody arm (with one heavy chain and one light chain), and one sialidase-Fc fusion with a sialidase fused at the N-terminus of the Fc. Each Fc domain polypeptide in the Janus ASC contains either the “knob” (T366Y) or “hole” (Y407T) mutation for heterodimerization (residue numbers according to EU numbering, Kabat, E. A., et al. (1991) supra). The third type of ASC, referred to as “Lobster,” contains two Fc domain polypeptides each with a sialidase fused at the N-terminus of the Fc and a scFv fused at the C-terminus of the Fc.

The following ASCs were expressed and characterized for purity using SDS-PAGE and SEC-HPLC, and assayed for enzymatic using 4MU-NeuAc as described in Example 1: (i) a Janus ASC including Salmonella typhimurium sialidase (St-sialidase) and trastuzumab (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 55, encoded by nucleotide sequence SEQ ID NO: 56); (ii) a Raptor ASC including St-sialidase and trastuzumab (including first and fourth polypeptide chains with amino acid sequence SEQ ID NO: 67, encoded by nucleotide sequence SEQ ID NO: 68, and second and third polypeptide chains with amino acid sequence SEQ ID NO: 59, encoded by nucleotide sequence SEQ ID NO: 60); and (iii) a Janus ASC including St-sialidase with two loss of function mutations, D100V and G231V, and trastuzumab (“Janus-LOF,” including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 61, encoded by nucleotide sequence SEQ ID NO: 62).

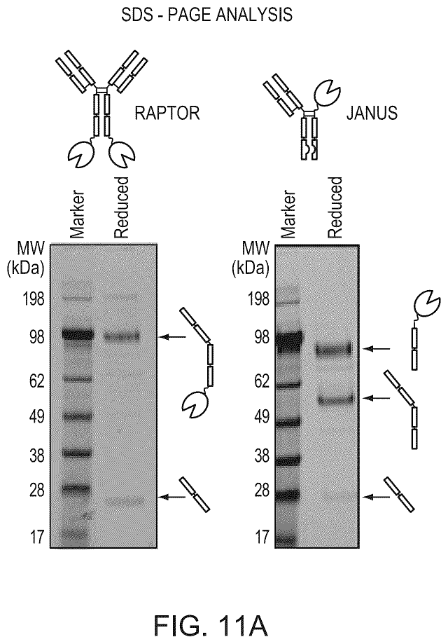

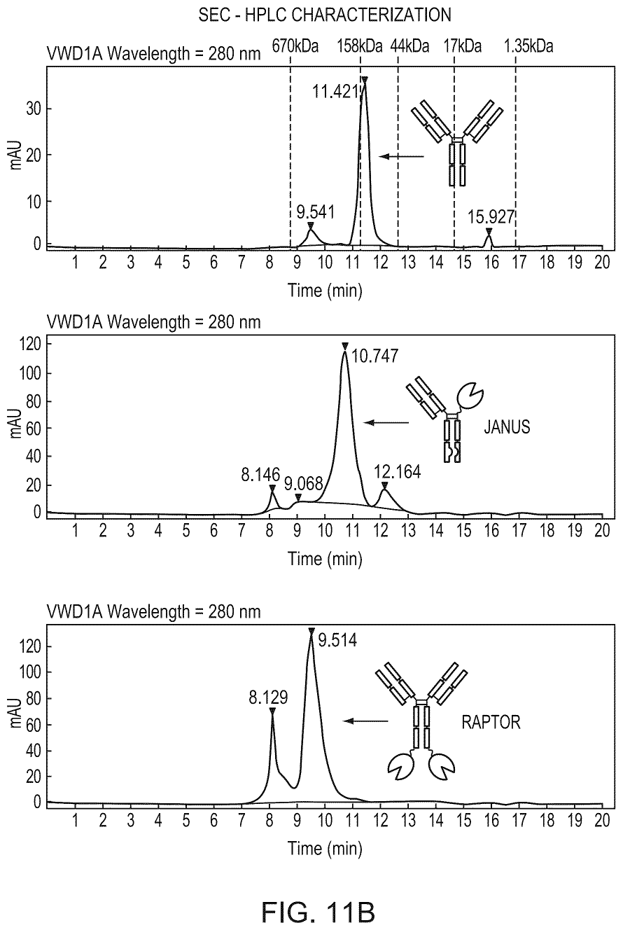

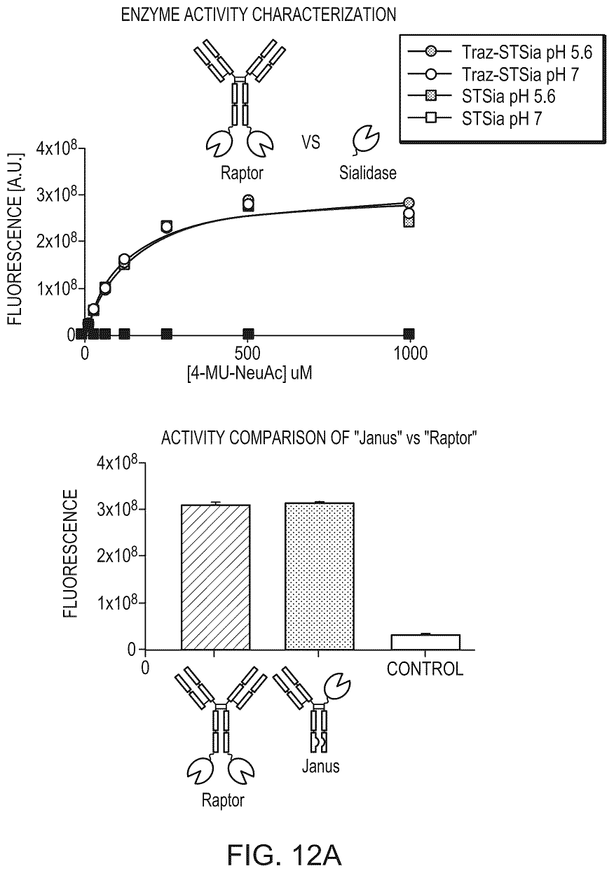

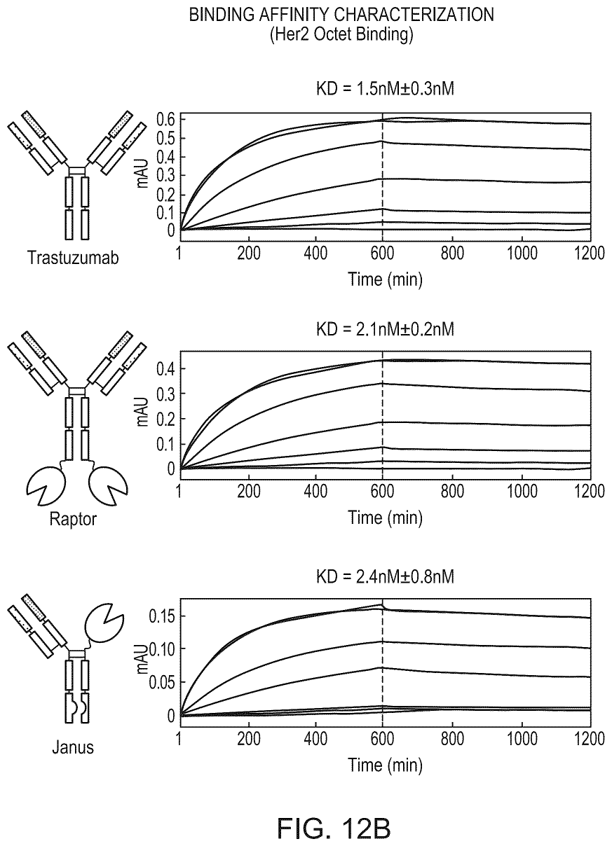

The ASCs were tested for antigen (Her2) binding by using ForteBio Octet with the ASC captured on anti-Fc sensors with dipping into serial dilutions of His-tagged Her2 (50 to 0.78 nM at 1:2 dilutions). The ASCs had good expression with a yield of 30 μg/mL and high purity, were as active as unconjugated St-sialidase, and bound to Her2 with comparable binding affinities to trastuzumab (FIGS. 11 and 12). Janus-LOF mutant abolished the sialidase activity as expected, and expressed well with similar biochemical characteristics as the parent Janus ASC.

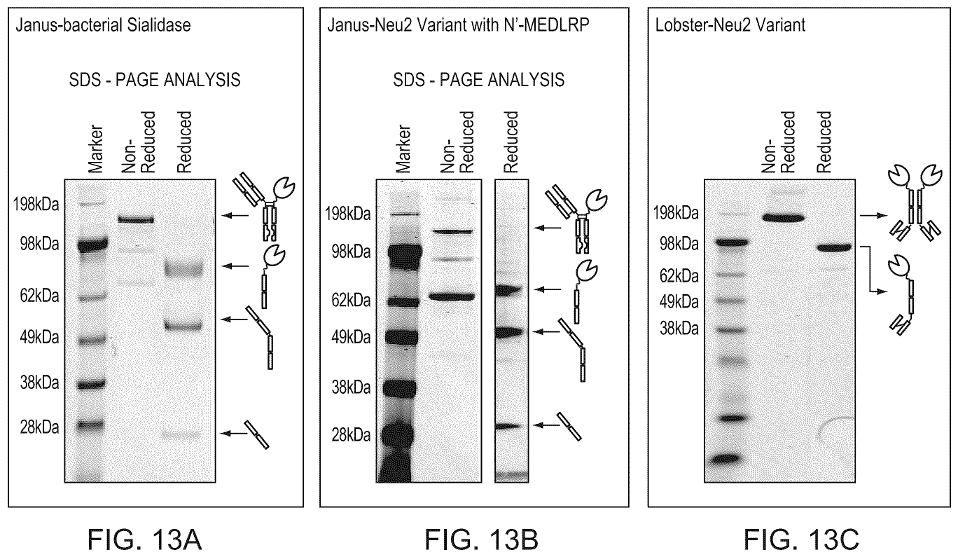

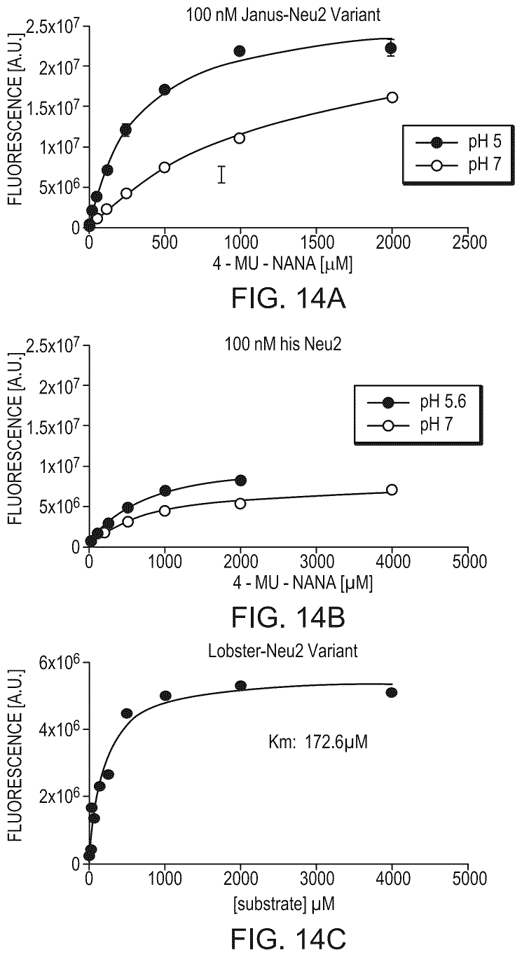

A Janus ASC was made using Neu2-M76 (which included MEDLRP (SEQ ID NO: 4) inserted at the N-terminus) and trastuzumab. This Janus ASC (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 63, encoded by nucleotide sequence SEQ ID NO: 64) was expressed and characterized for purity using SDS-PAGE and enzymatic activity using 4MU-NeuAc as described in Example 1. The Janus ASC had an expression yield of ˜5 μg/mL with good purity after purification (FIG. 13), and showed improved activity compared to a Janus ASC wild-type conjugated Neu2 (FIG. 14).

Additionally a Lobster ASC was made using Neu2-M85 (which included a deletion of M1 and mutations V6Y and I187K) and a scFv derived from trastuzumab. This Lobster ASC (including first and second polypeptide chains with amino acid sequence SEQ ID NO: 65, encoded by nucleotide sequence SEQ ID NO: 66) was expressed and characterized for purity using SDS-PAGE and enzymatic activity using 4MU-NeuAc as described in Example 1. The Lobster ASC had an expression yield of ˜5 μg/mL with good purity after purification (FIG. 13), and a Km of 172.6 μM (FIG. 14).

### Example 8

This Example describes the in vivo administration of antibody sialidase conjugates (ASCs) containing bacterial sialidases.

The following ASCs were made and tested in this Example: (i) a Janus ASC including Salmonella typhimurium sialidase (St-sialidase) and trastuzumab (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 55, encoded by nucleotide sequence SEQ ID NO: 56); (ii) a Raptor ASC including St-sialidase and trastuzumab (including first and fourth polypeptide chains with amino acid sequence SEQ ID NO: 67, encoded by nucleotide sequence SEQ ID NO: 68, and second and third polypeptide chains with amino acid sequence SEQ ID NO: 59, encoded by nucleotide sequence SEQ ID NO: 60); and (iii) a Lobster ASC including St-sialidase and an scFv derived from trastuzumab (including first and second polypeptide chains with amino acid sequence SEQ ID NO: 103, encoded by nucleotide sequence SEQ ID NO: 104). ASCs were made as described in Example 7.

These ASCs were compared to trastuzumab in a mouse syngeneic tumor model injected with a murine breast cancer cell line expressing human Her2 (EMT6-hHer2 cells). Female BALB/c mice, 6-8 weeks of age, were inoculated subcutaneously in the right lower flank region with EMT6-Her2 tumor cells (5×10⁵) in 0.1 ml of PBS for tumor development. Mice were randomly allocated to 8 groups when tumors reached 50-100 mm³, mean ˜75-100 mm³. Treatment groups are described in TABLE 8 with dosing schedule indicated post randomization. Anti-mouse NK1.1 (Clone: PK136; BioXcell, 621717N1), anti-mouse CD8a (Clone: 53-6.7; BioXcell, BE0004-1) and liposomal clodronate (FormuMax Scientific, Inc.) were included in treatment groups as indicated.

TABLE 8

| | | | | Dose | | |
|---|---|---|---|---|---|---|
| | Animal | | Dose | volume | | Schedule |
| Group | No. | Treatment | (mg/kg) | (μL/g) | Route | (Days) |
| 1 | 8 | Vehicle (PBS) | NA | 10 | i.p. | 0, 3, 7, 10, 14, 17 |
| 2 | 8 | Trastuzumab | 10 | 10 | i.p. | 0, 3, 7, 10, 14, 17 |
| 3 | 8 | Raptor | 10 | 10 | i.p. | 0, 3, 7, 10, 14, 17 |
| 4 | 8 | Janus | 10 | 10 | i.p. | 0, 3, 7, 10, 14, 17 |
| 5 | 8 | Lobster | 10 | 10 | i.p. | 0, 3, 7, 10, 14, 17 |
| 6 | 8 | Janus | 10 | 10 | i.p. | 0, 3, 7, 10, 14, 17 |
| | | anti-mouse NK1.1 | 10 | 10 | i.p. | 0, 3, 7, 10, 14, 17 |
| | | (Clone: PK136) | | | | |
| 7 | 8 | Janus | 10 | 10 | i.p. | 0, 3, 7, 10, 14, 17 |
| | | anti-mouse CD8α | 10 | 10 | i.p. | 0, 3, 7, 10, 14, 17 |
| | | (Clone: 53-6.7) | | | | |
| 8 | 8 | Janus | 10 | 10 | i.p. | 0, 3, 7, 10, 14, 17 |
| | | liposomal clodronate | 0.5 mg/ | 100 μL/ | i.p. | TIW × 2 wks |
| | | | mouse | mouse | | |

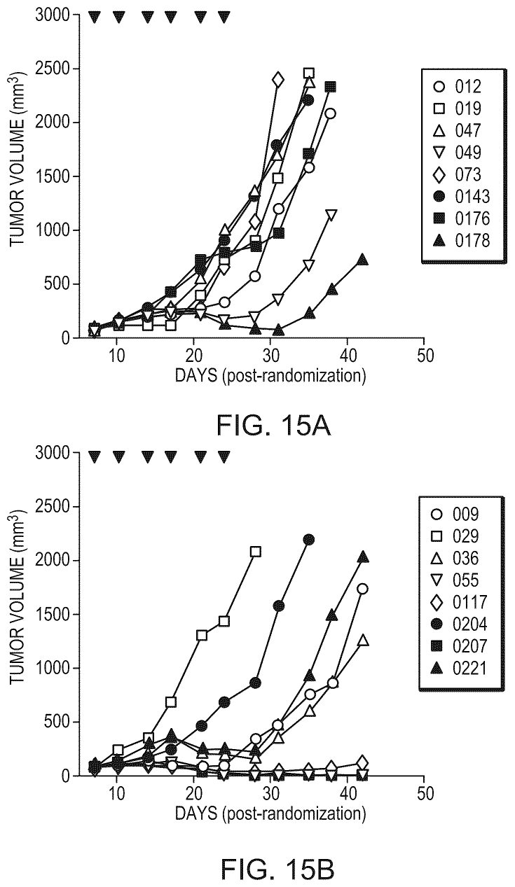

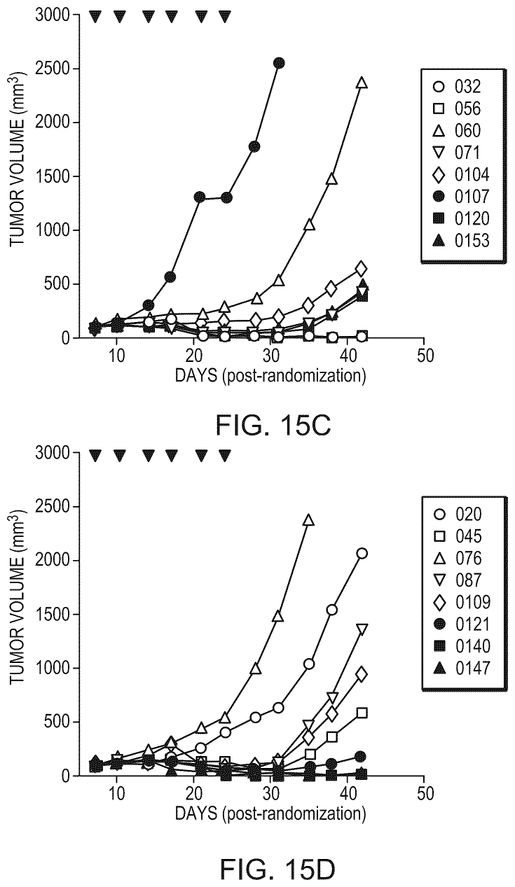

The results from for treatment with trastuzumab, and Raptor, Janus and Lobster ASCs are shown in FIGS. 15A, 15B, 15C and 15D respectively. As can be seen, trastuzumab resulted in no complete responses in eight individual mice as treated (defined as regression below the limit of palpation at any point for the duration of the study, FIG. 15A). This is in contrast to Raptor which demonstrated 2 out of 8 animals with a complete response (FIG. 15B), Janus which demonstrated 3 out of 8 animals with a complete response (FIG. 15C) and Lobster which demonstrated 2 out of 8 animals with a complete response (FIG. 15D).

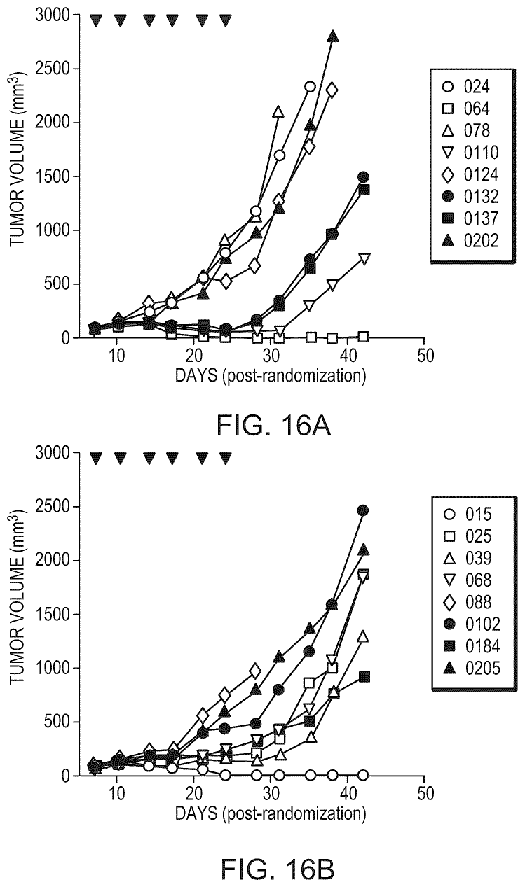

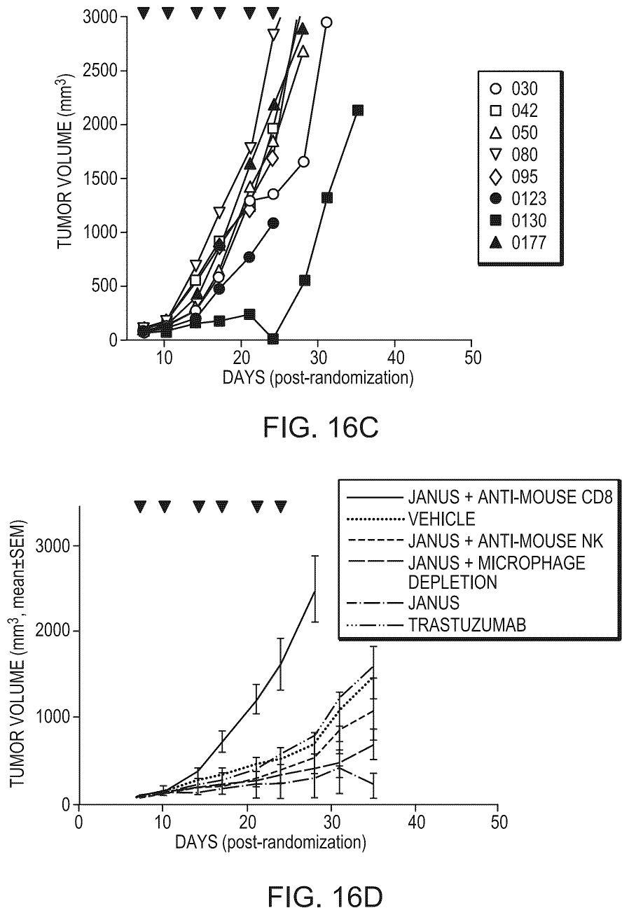

The results of administration of Janus with NK depletion (anti-mouse NK1.1), macrophage depletion (liposomal clodronate) and CD8 T cell depletion (anti-mouse CD8a) are shown in FIG. 16. As can be seen, compared to Janus treatment alone (FIG. 15C), where there was a complete response in 3 out of 8 animals, NK depletion reduced the number of complete responses to 1 out of 8 animals (FIG. 16A). Macrophage depletion also reduced the number of complete responses to 1 out of 8 animals (FIG. 16B). CD8 T cell depletion completely reversed the effects of Janus, with no animals showing a complete response (FIG. 16C). FIG. 16D shows the mean tumor volume for vehicle, Janus alone, trastuzumab alone and Janus with NK, macrophage and CD8 T cell depletions. These results demonstrate that innate immunity (NK and macrophage dependent) as well as adaptive immunity (CD8 T cells) contribute to in vivo ASC activity.

### Example 9

This Example describes the in vivo administration of antibody sialidase conjugates (ASCs) with bacterial sialidases.

The following ASCs were made and tested in this Example: (i) a Janus ASC including Salmonella typhimurium sialidase (St-sialidase) and trastuzumab (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 55, encoded by nucleotide sequence SEQ ID NO: 56); and (ii) a Janus ASC including St-sialidase with two loss of function mutations, D100V and G231V, and trastuzumab (“Janus-LOF,” including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 61, encoded by nucleotide sequence SEQ ID NO: 62). ASCs were made as described in Example 7.

These ASCs were tested in a mouse syngeneic orthotopic tumor model injected with an independent EMT6 cell line expressing human Her2 (EMT6-hHer2 cells as described in D'Amico et al. (2016) ANNALS OF ONCOLOGY, Volume 27, Issue suppl_8, 41P. Female BALB/c mice, 6-8 weeks of age, were inoculated via intra mammary implantation with EMT6-Her2 tumor cells (5×10⁶). Mice were randomly allocated to 6 groups when tumors reached approximately 250 mm³. The treatment groups are described in TABLE 9 with dosing schedule indicated post randomization. Anti-mouse PD1 was obtained from BioXcell (RMP1-14, Cat. #665418F1). Janus and Janus Loss of Function (Janus LOF) are described above in Example 7.

TABLE 9

| | | | | Dose | | |
|---|---|---|---|---|---|---|
| | Animal | | Dose | volume | | Schedule |
| Group | No. | Treatment | (mg/kg) | (μL/g) | Route | (Days) |
| 1 | 6 | Vehicle (PBS) | NA | 10 | i.p. | 0, 3, 7, 10 |
| 2 | 6 | Trastuzumab | 10 | 10 | i.p. | 0, 3, 7, 10 |
| 3 | 6 | Janus | 10 | 10 | i.p. | 0, 3, 7, 10 |
| 4 | 6 | Janus Loss of | 10 | 10 | i.p. | 0, 3, 7, 10 |
| | | Function (LOF) | | | | |
| 5 | 6 | anti-mouse PD1 | 10 | 10 | i.p. | 0, 3, 7, 10 |
| 6 | 6 | Janus | 10 | 10 | i.p. | 0, 3, 7, 10 |
| | | anti-mouse PD1 | 10 | 10 | i.p. | 0, 3, 7, 10 |

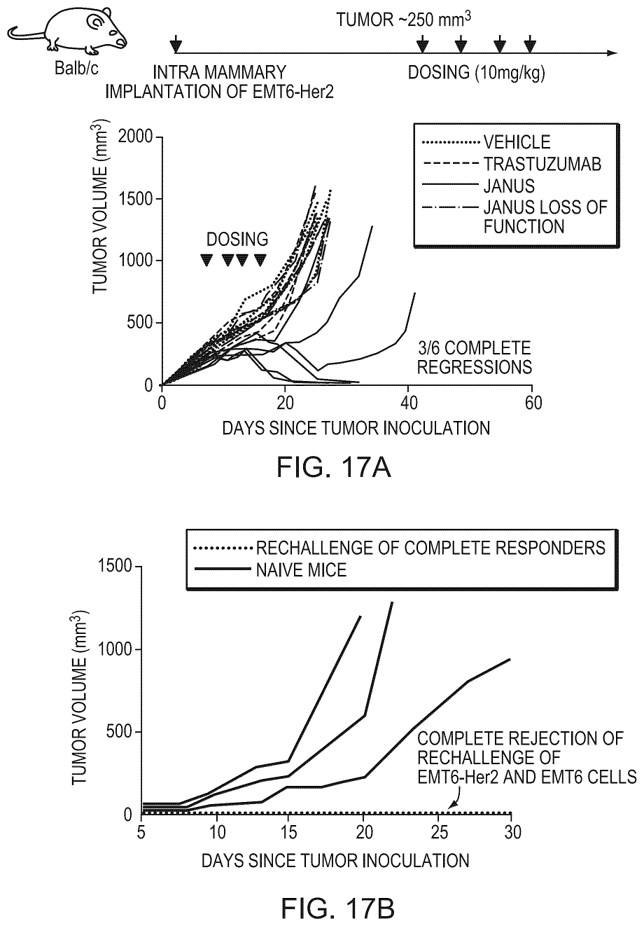

The results for Groups 1 through 4 (vehicle, trastuzumab, Janus and Janus LOF) are shown in FIG. 17A. As can be seen, 3 out of 6 animals treated with Janus had a complete regression of tumor growth. Notably, Janus LOF and trastuzumab were both comparable to vehicle treated animals.

The 3 mice with a complete regression (“cured mice”) were rechallenged with either the same EMT6-Her2 cells used originally or parental EMT6 cells (lacking engineered human Her2 expression). EMT6 cells and EMT6-Her2 cells were inoculated subcutaneously in the right or left lower flank region respectively (5×10⁵) in 0.1 ml of PBS for tumor development of all three cured mice. EMT6-Her2 cells were also inoculated subcutaneously into naïve mice as a control. As can be seen in FIG. 17B, neither EMT6-Her2 cells nor parental EMT6 cells resulted in tumor growth in the cured mice while EMT6-Her2 cells developed into tumors as expected in the naïve mice. These results suggest that the antibody sialidase conjugates of the present invention are capable of inducing long term memory against tumors. In addition, the long term memory is towards the tumor cell and is independent of the originally targeted cancer antigen (Her2 in this case).

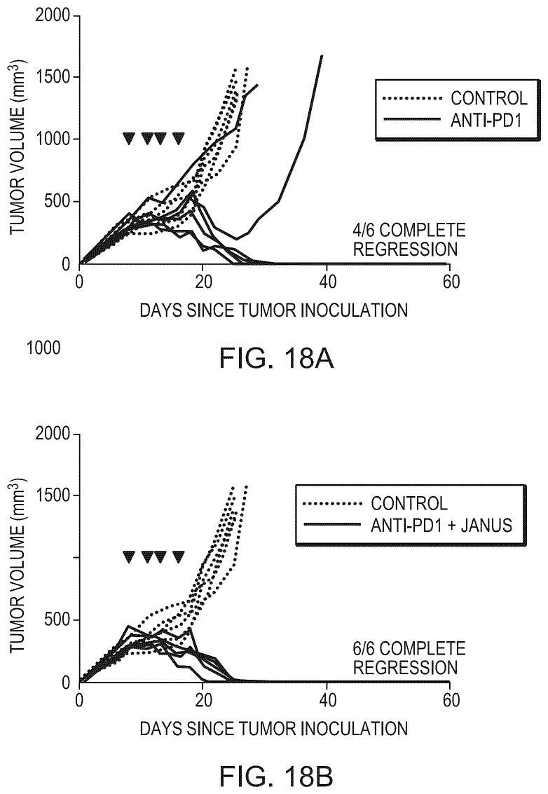

The results for Groups 1, 5 and 6 (vehicle, anti-mouse PD1 and anti-mouse PD1 combined with Janus) are shown in FIG. 18A and FIG. 18B. While anti-mouse PD1 had good activity with 4 out of 6 mice demonstrating complete regressions (similar to Janus alone with 3 out of 6 mice demonstrating complete regression, see FIG. 17A), the combination of anti-mouse PD1 with Janus demonstrated complete regression of tumor growth in all 6 mice (FIG. 18B). There was no body weight loss in any of the animals given this combination.

### Example 10

This Example describes the in vivo administration of antibody sialidase conjugates (ASCs) with bacterial sialidases.

A Janus ASC including Salmonella typhimurium sialidase (St-sialidase) and trastuzumab (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 55, encoded by nucleotide sequence SEQ ID NO: 56) was made as described in Example 7.

The ASC was tested in a mouse syngeneic tumor model injected with a B16 melanoma cell line expressing human Her2 (B16D5-Her2, Surana et al. CANCER IMMUNOL RES, 2 (11): 1103-1112). Female C57BL/6 mice, 6-8 weeks of age, were inoculated subcutaneously in the right lower flank region with B16D5-Her2 tumor cells (5×10⁵). Mice were randomly allocated to 3 groups when tumors reached approximately 50 to 100 mm3. Treatment groups are described in TABLE 10 with dosing schedule indicated post randomization. Anti-mouse PD1, obtained from BioXcell (RMP1-14, Cat. No. 665418F1) and anti-mouse CTLA4, obtained from BioXcell (9D9, Cat. #BE0164), and were used in combination.

TABLE 10

| | | | | Dose | | |
|---|---|---|---|---|---|---|
| | No. of | | Dose | volume | | Schedule |
| Group | Animal | Treatment | (mg/kg) | (uL/g) | Route | (Days) |
| 1 | 6 | Janus | NA | 10 | i.p. | 0, 3, 7, 10 |
| 2 | 6 | Trastuzumab | 10 | 10 | i.p. | 0, 3, 7, 10 |
| 3 | 6 | anti-mouse CTLA4 | 10 | 10 | i.p. | 0, 3, 7, 10 |
| | | anti-mouse PD1 | 10 | 10 | i.p. | 0, 3, 7, 10 |

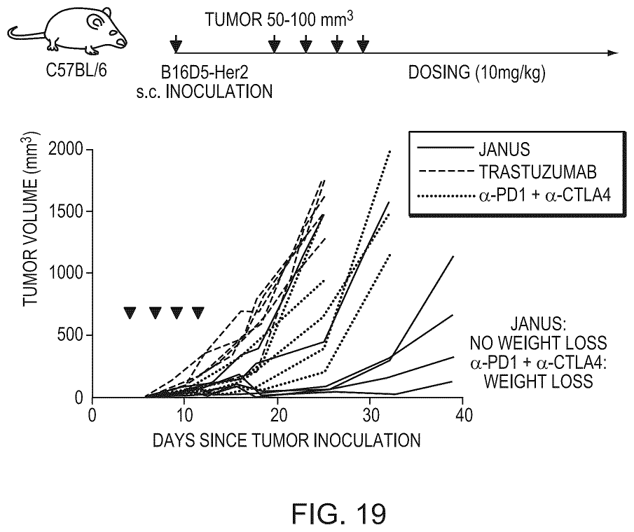

The B16 melanoma mouse model is considered a difficult tumor model to treat with immuno-oncology approaches. A comparison of Janus to a combination of anti-mouse PD1 and anti-mouse CTLA4 was carried out. The results are shown in FIG. 19. Anti-mouse PD1 combined with anti-mouse CTLA4 had an impact on B16D5-Her2 tumor growth, but this combination also demonstrated significant weight loss in the treated animals. By comparison, Janus demonstrated a more robust anti-tumor activity with no significant weight loss. Trastuzumab alone demonstrated marginal activity in this model.

### Example 11

This example describes targeted cleavage of terminal sialic acids from tumor cells by antibody sialidase conjugates (ASCs).

The following ASCs were made and tested in this Example: (i) a Raptor ASC including St-sialidase and trastuzumab (including first and fourth polypeptide chains with amino acid sequence SEQ ID NO: 67, encoded by nucleotide sequence SEQ ID NO: 68, and second and third polypeptide chains with amino acid sequence SEQ ID NO: 59, encoded by nucleotide sequence SEQ ID NO: 60); (ii) a Janus ASC including Salmonella typhimurium sialidase (St-sialidase) and trastuzumab (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 55, encoded by nucleotide sequence SEQ ID NO: 56); (iii) a Lobster ASC including St-sialidase and an scFv derived from trastuzumab (including first and second polypeptide chains with amino acid sequence SEQ ID NO: 103, encoded by nucleotide sequence SEQ ID NO: 104); (iv) a Janus ASC including St-sialidase with two loss of function mutations, D100V and G231V, and trastuzumab (“Janus-LOF,” including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 61, encoded by nucleotide sequence SEQ ID NO: 62); and (v) a non-Her2 binding Janus ASC including St-sialidase and an antibody recognizing respiratory syncytial virus F protein (“Janus non-Her2”; including a first polypeptide chain with amino acid sequence SEQ ID NO: 94, encoded by nucleotide sequence SEQ ID NO: 95, a second polypeptide chain with amino acid sequence SEQ ID NO: 96, encoded by nucleotide sequence SEQ ID NO: 97, and a third polypeptide chain with amino acid sequence SEQ ID NO: 55, encoded by nucleotide sequence SEQ ID NO: 56). ASCs were made as described in Example 7.

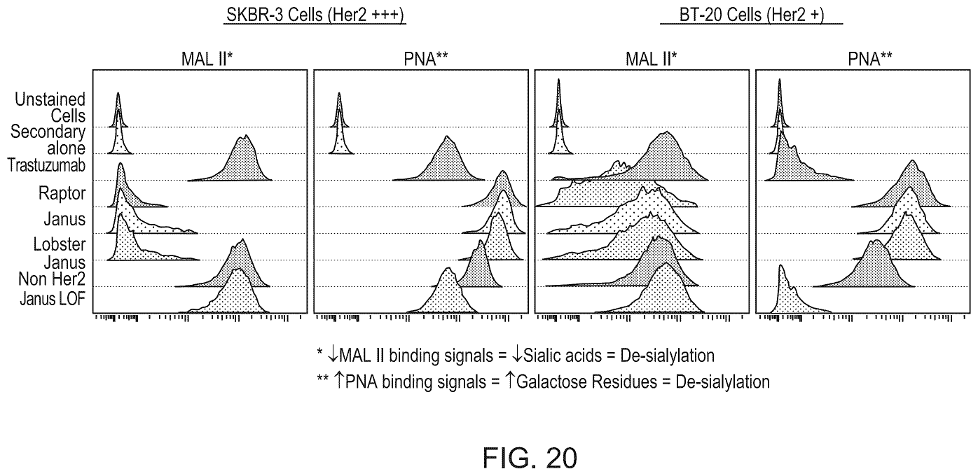

SKBR-3 cells (Her2+++) or BT-20 cells (Her2+) were incubated with trastuzumab or the indicated ASCs, and FACS staining with MAL II and PNA was used to measure the degree of sialic acid removal (see, FIG. 20). MAL II is a lectin with high affinity for sialic acid, and therefore MAL II staining was expected to decrease upon loss of cell surface sialic acid following cleavage by an ASC. PNA is a lectin with high affinity for terminal galactose residues and therefore PNA staining was expected to increase with loss of cell surface galactose coupled sialic acid following cleavage by an ASC. Compared to trastuzumab, treatment with Janus, Raptor and Lobster ASCs decreased sialic acid levels on cancer cells with high or low levels of Her2 (SKBR-3 and BT-20 cells, respectively). No sialic acid cleavage was observed for the Janus LOF construct and substantially reduced sialic acid cleavage was observed for the Janus non-Her2 binding construct.

### Example 12

This example describes a reduction in cancer-cell mediated inhibition of dendritic cell (DC) activation by antibody sialidase conjugates (ASCs).

DCs play a major role in initiating and sustaining an immune response. They seck antigens in tissues (including tumor sites). Once DCs encounter antigens, they mature, activate, and move to draining lymph nodes for presentation of the processed antigen to T cells. This process of DC activation can be inhibited by the interaction of hypersialylated proteins on cancer cells with Siglecs on the surface of DCs. Desialylation of hypersialylated proteins on cancer cells by an ASC can potentially reduce this inhibition and result in increased activation of DCs.

To test this, SKBR-3 cells (which express high levels of Her2) were initially incubated with either: (i) a Janus ASC including Salmonella typhimurium sialidase (St-sialidase) and trastuzumab (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 55, encoded by nucleotide sequence SEQ ID NO: 56); (ii) a Janus ASC including St-sialidase with two loss of function mutations, D100V and G231V, and trastuzumab (“Janus-LOF,” including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 61, encoded by nucleotide sequence SEQ ID NO: 62); or (iii) a non-Her2 binding Janus ASC including St-sialidase and an antibody recognizing respiratory syncytial virus F protein (“Janus non-Her2”; including a first polypeptide chain with amino acid sequence SEQ ID NO: 94, encoded by nucleotide sequence SEQ ID NO: 95, a second polypeptide chain with amino acid sequence SEQ ID NO: 96, encoded by nucleotide sequence SEQ ID NO: 97, and a third polypeptide chain with amino acid sequence SEQ ID NO: 55, encoded by nucleotide sequence SEQ ID NO: 56). ASCs were made as described in Example 7. Cells were then washed and co-cultured with DCs for 16 hours in presence or absence of lipopolysaccharide (LPS; a DC activation signal). DC surface activation markers were assessed by flow cytometry.

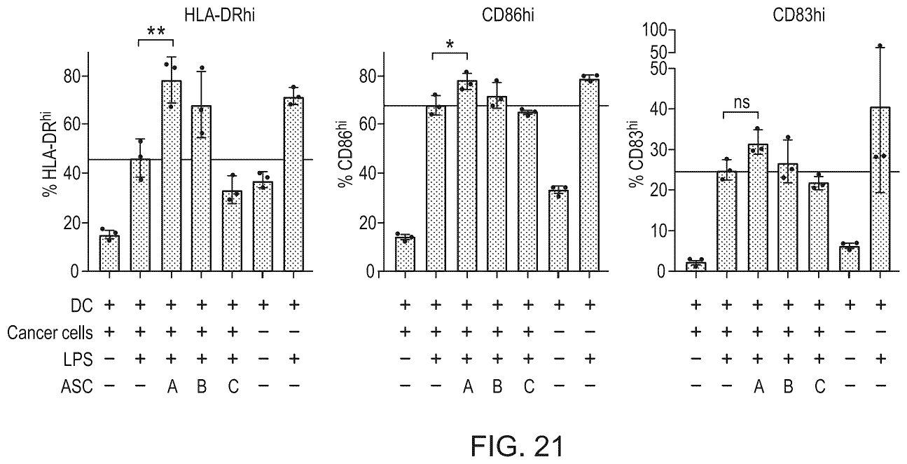

As can be seen in FIG. 21, co-culture of DCs with LPS increased levels of the surface activation markers HLA-DR, CD86 and CD83. However, this increase was reduced in the presence of SKBR-3 cancer cells, reflecting inhibition of DC activation by the cancer cells. Treatment of the SKBR-3 cell line with Janus blocked SKBR-3-mediated inhibition of DCs, as evidenced by increased cell surface expression of HLA-DR, CD86 and CD83 on the DCs. The effect was reduced for Janus non-Her2 and completely absent for Janus LOF versions of the ASC, indicating that both active and targeted sialidase activity in the ASC is required for an optimal effect.

These results demonstrate that targeted desialylation of cancer cells by ASCs can reduce cancer-cell mediated inhibition of dendritic cell (DC) activation. Accordingly, treatment with ASCs may be an effective strategy for enhancing immunogenicity of tumor antigens by enhancing their presentation by DCs.

### Example 13

This Example describes induction of proinflammatory cytokines in peripheral blood mononuclear cells (PBMCs) by an antibody sialidase conjugate (ASC) with a human sialidase.

A Janus ASC was constructed that includes Neu2 with M1D, V6Y, I187K, and C332A mutations and trastuzumab (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 54, encoded by nucleotide sequence SEQ ID NO: 98)

Human PBMCs were freshly isolated, washed two times, and plated in culture medium (RPMI 1640+L-glut, 10% FBS, 1% P/S) at 250,000 PBMCs per well. PBMCs were incubated in quadruplicate with the Janus ASC at 2.5× serial dilutions, with a maximum concentration of 100 μg/ml. After 24 hours at 37° C., cells were removed by centrifugation and supernatants collected for cytokine measurement using Luminex multiplex assays according to manufacturer's instruction.

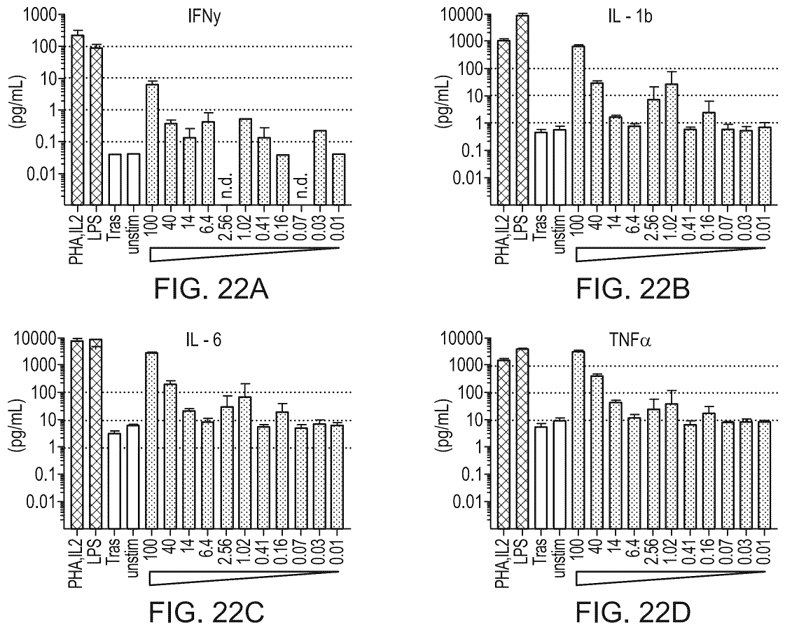

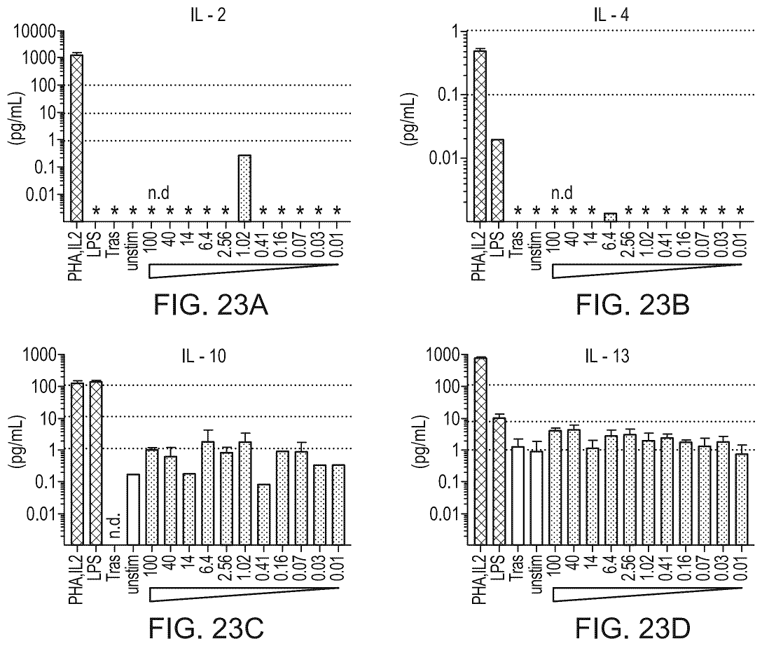

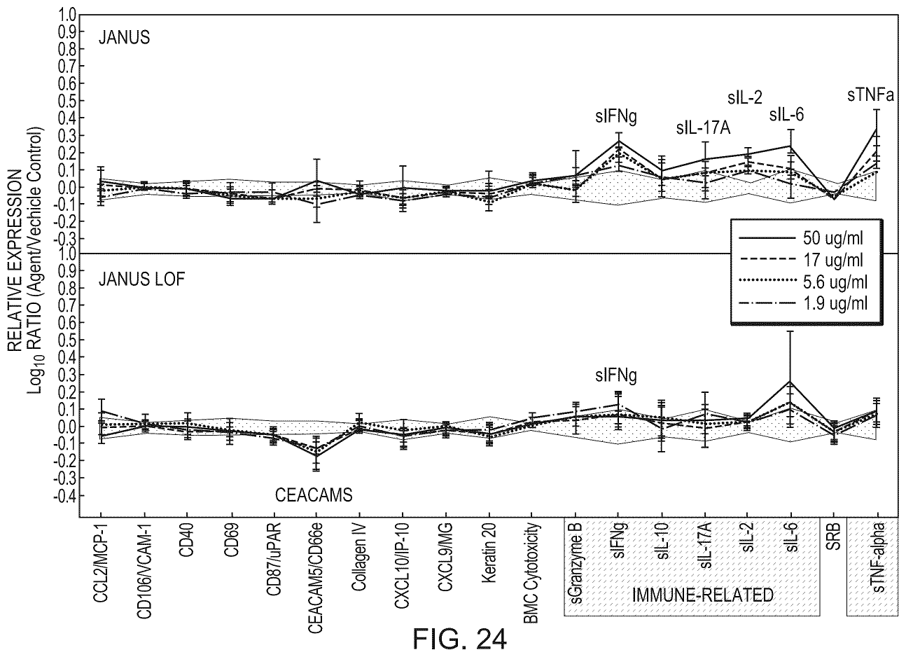

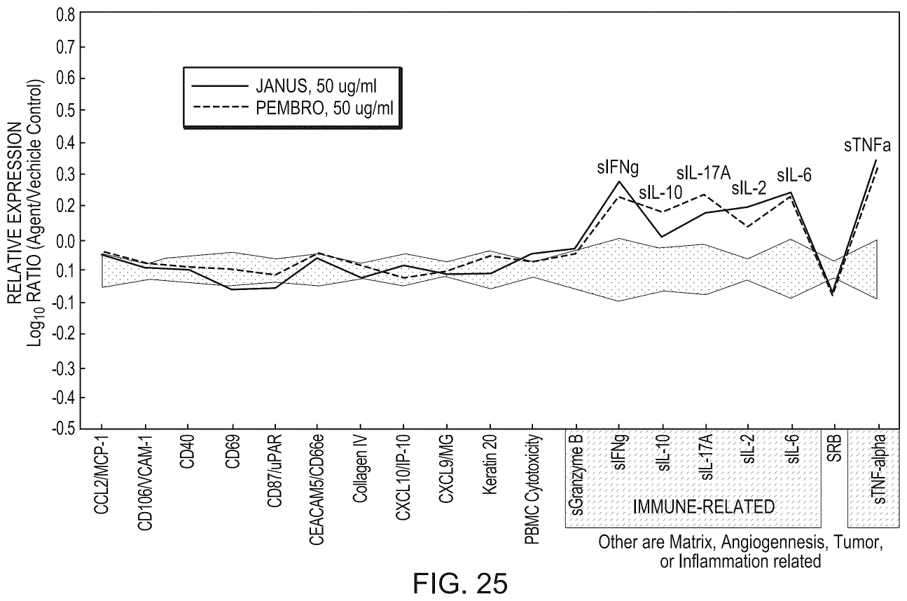

As positive controls, phytohemagglutinin-L (PHA-L; 5 μg/mL) with IL-2 (10 U/mL) or LPS (10 ng/mL) were used to stimulate cytokine release. As a negative control, trastuzumab (10 μg/mL) was used. As seen FIG. 22, the human Janus ASC induced proinflammatory cytokine secretion in PBMCs (e.g., INFγ, IL-1b, IL-6 and TNFα). FIG. 23 depicts the effects of the human Janus ASC on PBMC secretion of proinflammatory cytokine IL-2, antiinflammatory cytokines IL-4 and IL-10, and pro- and antiinflammatory cytokine IL-13.

These results demonstrate that ASCs can induce secretion of proinflammatory cytokines in PBMCs.

### Example 14

This example describes increased immune-related activities following addition of antibody sialidase conjugates (ASCs) to a host-tumor microenvironment model system.

BioMAP Oncology Panels (Eurofins, Fremont, CA) are a complex co-culture of tumor cell lines and early passage primary human cells (endothelial cells/fibroblasts and PBMCs) that mimic cancer-induced immune suppression. In certain circumstances, BioMAP results have correlated with clinical outcomes. For example, pembrolizumab has been shown to increase immune response in the model, while IDO inhibitors had no effect.

The following constructs were tested in a blinded study using the BioMAP VascHT29 co-culture system: (i) a Janus ASC including Salmonella typhimurium sialidase (St-sialidase) and trastuzumab (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 55, encoded by nucleotide sequence SEQ ID NO: 56); (ii) a Janus ASC including St-sialidase with two loss of function mutations, D100V and G231V, and trastuzumab (“Janus-LOF,” including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 61, encoded by nucleotide sequence SEQ ID NO: 62); (iii) isotype control; (iv) trastuzumab; or (v) pembrolizumab. All ASCs were made as described in Example 7.

Test reagents were tested at a range of concentrations (50, 17, 5.6 and 1.9 μg/ml) for 48 hours. Following exposure, a number of parameters were analyzed as depicted in FIG. 24 and FIG. 25. In both figures, the historical range of vehicle response is represented by the shaded area along the zero baseline. Values for each measurement are represented by the log of the ratio of test article to vehicle control. Analytes with a statistically meaningful value above historical ranges are annotated.

FIG. 24 demonstrates that Janus is not cytotoxic at the concentrations tested in this study. Janus demonstrated a dose dependent increase in a number of immune-related activities, including increased soluble IL-17A, IL-6, IL-2, and IFNγ as well as inflammation-related activities as seen by increased TNFα. Janus LOF is not cytotoxic at the concentrations tested in this study and demonstrated modest immune-related activities with increased IFNγ and tumor-related activities with decreased CEACAM5. FIG. 25 is a comparison of Janus to pembrolizumab. As can be seen in FIG. 25, Janus has a similar activity to pembrolizumab in this tumor microenvironment model.

### Example 15

This Example describes the in vivo administration of antibody sialidase conjugates (ASCs) containing bacterial sialidases.

The following ASCs were made and tested in this Example: (i) a Janus ASC including Salmonella typhimurium sialidase (St-sialidase) and trastuzumab (including a first polypeptide chain with amino acid sequence SEQ ID NO: 67, encoded by nucleotide sequence SEQ ID NO: 68, a second polypeptide chain with amino acid sequence SEQ ID NO: 57, encoded by nucleotide sequence SEQ ID NO: 58, and a third polypeptide chain with amino acid sequence SEQ ID NO: 55, encoded by nucleotide sequence SEQ ID NO: 56); and (ii) a non-Her2 binding Janus ASC including St-sialidase and an antibody recognizing respiratory syncytial virus F protein (“Janus non-Her2”; including a first polypeptide chain with amino acid sequence SEQ ID NO: 94, encoded by nucleotide sequence SEQ ID NO: 95, a second polypeptide chain with amino acid sequence SEQ ID NO: 96, encoded by nucleotide sequence SEQ ID NO: 97, and a third polypeptide chain with amino acid sequence SEQ ID NO: 55, encoded by nucleotide sequence SEQ ID NO: 56). ASCs were made as described in Example 7.

The ASCs were compared to trastuzumab in a mouse syngeneic tumor model. Female BALB/c mice, 6-8 weeks of age, were inoculated subcutaneously in the right lower flank region with a murine breast cancer cell line expressing human Her2 (EMT6-hHer2 cells; 5×10⁵ cells) in 0.1 ml of PBS for tumor development. Mice were randomly allocated to 4 groups of 8 animals each when tumors reached 50-100 mm³, mean ˜75-100 mm³.

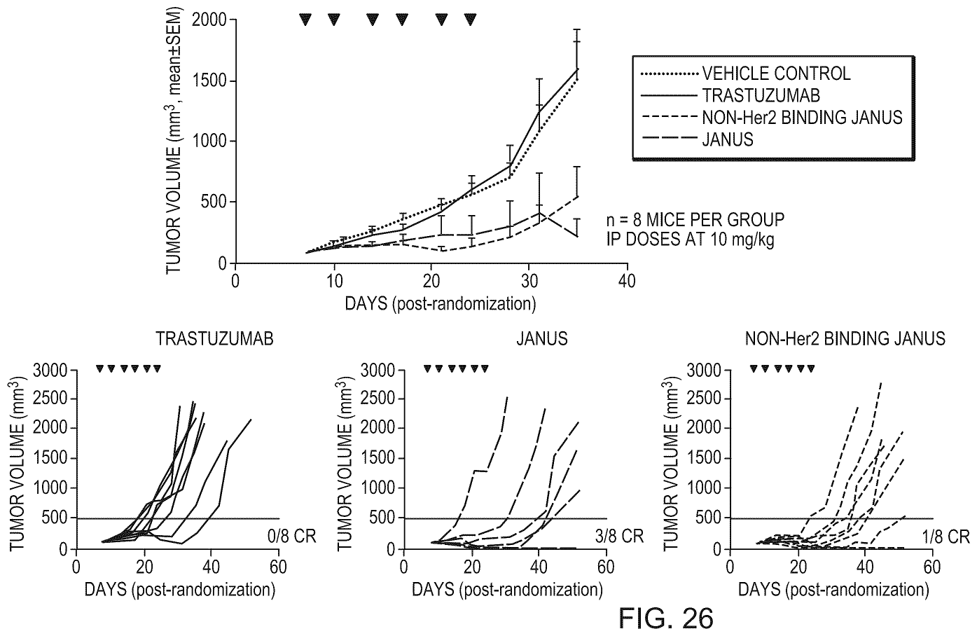

Mice were treated via intraperitoneal injection of 10 mg/kg of Janus, trastuzumab or non-Her2 binding Janus and tumor volume (mm³) was recorded. FIG. 26 shows mean tumor volumes for each treatment group. FIG. 26 shows tumor volumes for individual mice in each treatment group. Complete Responses (CR, defined as regression below the limit of palpation at any point during the study) are shown as well. Trastuzumab and vehicle control demonstrated similar tumor growth curves and no CRs. In contrast, Janus demonstrated reduced tumor growth compared to vehicle with 3 out of 8 mice demonstrating CR. Non-Her2 binding Janus demonstrated a reduced tumor growth compared to vehicle with 1 out of 8 mice demonstrating CR. These results show that ASCs may be active towards a tumor with low expression levels of the tumor antigen targeted by the ASC. Additionally, these results suggest that a non-targeted ASC, e.g., a sialidase-Fc fusion protein, may be active towards a tumor lacking a specific tumor-associated antigen.

### Example 16

This Example describes the in vivo administration of antibody sialidase conjugates (ASCs) containing human sialidases.

The following ASCs were made and tested in this Example: (i) a Lobster ASC including Neu2 with ΔM1, V6Y and I187K mutations and an scFv derived from trastuzumab (including first and second polypeptide chains with amino acid sequence SEQ ID NO: 65, encoded by nucleotide sequence SEQ ID NO: 66, and referred to as “Lobster 1” in this example); and (ii) a Lobster ASC including Neu2 with V6Y and I187K mutations and an scFv derived from trastuzumab (including first and second polypeptide chains with amino acid sequence SEQ ID NO: 74, encoded by nucleotide sequence SEQ ID NO: 99, and referred to as “Lobster 2” in this example). ASCs were made as described in Example 7.

These ASCs were compared to trastuzumab in a mouse syngeneic tumor model injected with a murine breast cancer cell line expressing human Her2 (EMT6-hHer2 cells). Female BALB/c mice, 6-8 weeks of age, were inoculated subcutaneously in the right lower flank region with EMT6-Her2 tumor cells (5×10⁵) in 0.1 ml of PBS for tumor development. Mice were randomly allocated to 4 groups of 5 animals each when tumors reached 50-100 mm³, mean ˜75-100 mm³.

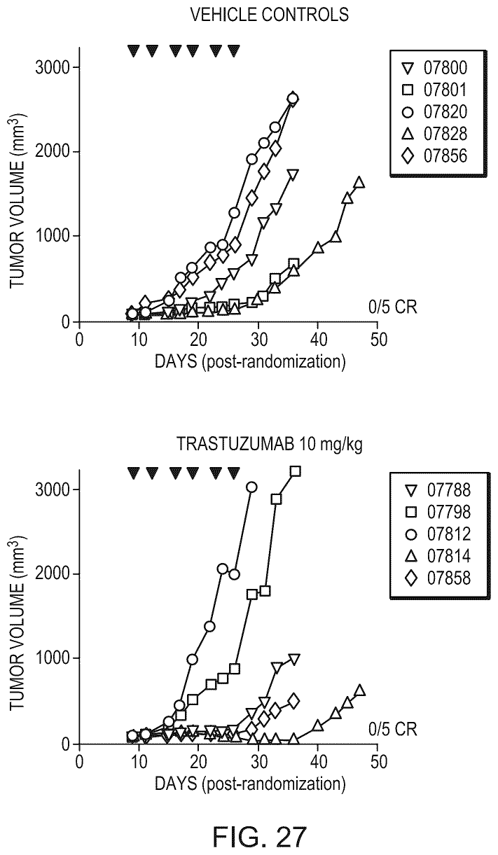

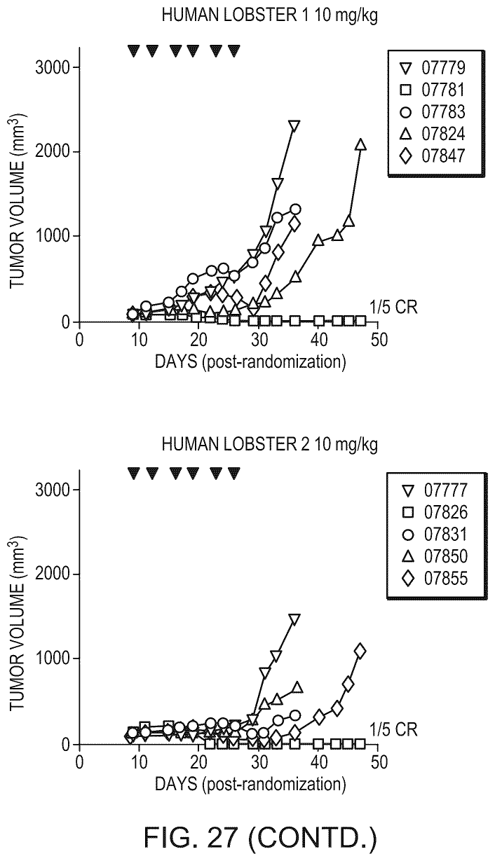

Mice were treated via intraperitoneal injection of 10 mg/kg of either human Lobster 1, human Lobster 2, or trastuzumab and tumor volume (mm³) was recorded. FIG. 27 shows mean tumor volumes for the individual mice for the indicated treatments. Complete Responses (CR, defined as regression below the limit of palpation at any point during the study) are shown. Trastuzumab and vehicle control demonstrated no CRs. In contrast, both human Lobster 1 and human Lobster 2 demonstrated a reduced tumor growth compared to vehicle with 1 out of 5 mice in both groups demonstrating CR. This example demonstrates that human sialidase based ASCs demonstrate efficacy in an in vivo tumor model.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING
SEQ ID NO: 1:
MASLPVLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPIQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSCAYSDLQSM
GTGPDGSPLFGCLYEANDYEEIVFLMFTLKQAFPAEYLPQ
SEQ ID NO: 2:
MEDLRPMATCPVLQKETLFRTGVHAYRIPALLYLKKQKTLLAFAEKRASKTDEHAELIVLRRGSYNEATNRVKWQPEEVVTQAQL
EGHRSMNPCPLYDKQTKTLFLFFIAVPGRVSEHHQLHTKVNVTRLCCVSSTDHGRTWSPIQDLTETTIGSTHQEWATFAVGPGHC
LQLRNPAGSLLVPAYAYRKLHPAQKPTPFAFCFISLDHGHTWKLGNFVAENSLECQVAEVGTGAQRMVYLNARSFLGARVQAQSP
NDGLDFQDNRVVSKLVEPPHGCHGSVVAFHNPISKPHALDTWLLYTHPTDSRNRTNLGVYLNQMPLDPTAWSEPTLLAMGICAYS
DLQNMGQGPDGSPQFGCLYESGNYEEIIFLIFTLKQAFPTVFDAQ
SEQ ID NO: 3:
EDLRP
SEQ ID NO: 4:
MEDLRP
SEQ ID NO: 5:
MASLPVLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPIQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGLLYEANDYEEIVFLMFTLKQAFPAEYLPQ
SEQ ID NO: 6:
MEDLRPMASLPVLQKESVFQSGAHAYRIPALLYLPGQQSLLAFAEQRASKKDEHAELIVLRRGDYDAPTHQVQWQAQEVVAQARL
DGHRSMNPCPLYDAQTGTLFLFFIAIPGQVTEQQQLQTRANVTRLCQVTSTDHGRTWSSPRDLTDAAIGPAYREWSTFAVGPGHC
LQLHDRARSLVVPAYAYRKLHPIQRPIPSAFCFLSHDHGRTWARGHFVAQDTLECQVAEVETGEQRVVTLNARSHLRARVQAQST
NDGLDFQESQLVKKLVEPPPQGCQGSVISFPSPRSGPGSPAQWLLYTHPTHSWQRADLGAYLNPRPPAPEAWSEPVLLAKGSCAY
SDLQSMGTGPDGSPLFGCLYEANDYEEIVFLMFTLKQAFPAEYLPQ
SEQ ID NO: 7:
ENDFGLVQPLVTMEQLLWVSGRQIGSVDTFRIPLITATPRGTLLAFAEARKVISSSDEGAKFIALRRSMDQGSTWSPTAFIVNDG
DVPDGLNLGAVVSDVETGVVFLFYSLCAHKAGCQVASTMLVWSKDDGVSWSTPRNLSLDIGTEVFAPGPGSGIQKQREPRKGRLI
VCGHGTLERDGVFCLLSDDHGASWRYGSGVSGIPYGQPKQENDFNPDECQPYELPDGSVVINARNQNNYHCHCRIVLRSYDACDT
LRPRDVTFDPELVDPVVAAGAVVTSSGIVFFSNPAHPEFRVNLTLRWSFSNGTSWRKETVQLWPGPSGYSSLATLEGSMDGEEQA
PQLYVLYEKGRNHYTESISVAKISV
SEQ ID NO: 8:
MEEVTTCSFNSPLFRQEDDRGITYRIPALLYIPPTHTFLAFAEKRSTRRDEDALHLVLRRGLRIGQLVQWGPLKPLMEATLPGHR
TMNPCPVWEQKSGCVFLFFICVRGHVTERQQIVSGRNAARLCFIYSQDAGCSWSEVRDLTEEVIGSELKHWATFAVGPGHGIQLQ
SGRLVIPAYTYYIPSWFFCFQLPCKTRPHSLMIYSDDLGVTWHHGRLIRPMVTVECEVAEVTGRAGHPVLYCSARTPNRCRAEAL
STDHGEGFQRLALSRQLCEPPHGCQGSVVSFRPLEIPHRCQDSSSKDAPTIQQSSPGSSLRLEEEAGTPSESWLLYSHPTSRKQR
VDLGIYLNQTPLEAACWSRPWILHCGPCGYSDLAALEEEGLFGCLFECGTKQECEQIAFRLFTHREILSHLQGDCTSPGRNPSQF
KSN
SEQ ID NO: 9:
MRPADLPPRPMEESPASSSAPTETEEPGSSAEVMEEVTTCSFNSPLFRQEDDRGITYRIPALLYIPPTHTFLAFAEKRSTRRDED
ALHLVLRRGLRIGQLVQWGPLKPLMEATLPGHRTMNPCPVWEQKSGCVFLFFICVRGHVTERQQIVSGRNAARLCFIYSQDAGCS
WSEVRDLTEEVIGSELKHWATFAVGPGHGIQLQSGRLVIPAYTYYIPSWFFCFQLPCKTRPHSLMIYSDDLGVTWHHGRLIRPMV
TVECEVAEVTGRAGHPVLYCSARTPNRCRAEALSTDHGEGFQRLALSRQLCEPPHGCQGSVVSFRPLEIPHRCQDSSSKDAPTIQ
QSSPGSSLRLEEEAGTPSESWLLYSHPTSRKQRVDLGIYLNQTPLEAACWSRPWILHCGPCGYSDLAALEEEGLFGCLFECGTKQ
ECEQIAFRLFTHREILSHLQGDCTSPGRNPSQFKSN
SEQ ID NO: 10:
MGVPRTPSRTVLFERERTGLTYRVPSLLPVPPGPTLLAFVEQRLSPDDSHAHRLVLRRGTLAGGSVRWGALHVLGTAALAEHRSM
NPCPVHDAGTGTVFLFFIAVLGHTPEAVQIATGRNAARLCCVASRDAGLSWGSARDLTEEAIGGAVQDWATFAVGPGHGVQLPSG
RLLVPAYTYRVDRRECFGKICRTSPHSFAFYSDDHGRTWRCGGLVPNLRSGECQLAAVDGGQAGSFLYCNARSPLGSRVQALSTD
EGTSFLPAERVASLPETAWGCQGSIVGFPAPAPNRPRDDSWSVGPGSPLQPPLLGPGVHEPPEEAAVDPRGGQVPGGPFSRLQPR
GDGPRQPGPRPGVSGDVGSWTLALPMPFAAPPQSPTWLLYSHPVGRRARLHMGIRLSQSPLDPRSWTEPWVIYEGPSGYSDLASI
GPAPEGGLVFACLYESGARTSYDEISFCTFSLREVLENVPASPKPPNLGDKPRGCCWPS
SEQ ID NO: 11:
MMSSAAFPRWLSMGVPRTPSRTVLFERERTGLTYRVPSLLPVPPGPTLLAFVEQRLSPDDSHAHRLVLRRGTLAGGSVRWGALHV
LGTAALAEHRSMNPCPVHDAGTGTVFLFFIAVLGHTPEAVQIATGRNAARLCCVASRDAGLSWGSARDLTEEAIGGAVQDWATFA
VGPGHGVQLPSGRLLVPAYTYRVDRRECFGKICRTSPHSFAFYSDDHGRTWRCGGLVPNLRSGECQLAAVDGGQAGSFLYCNARS
PLGSRVQALSTDEGTSFLPAERVASLPETAWGCQGSIVGFPAPAPNRPRDDSWSVGPGSPLQPPLLGPGVHEPPEEAAVDPRGGQ
VPGGPFSRLQPRGDGPRQPGPRPGVSGDVGSWTLALPMPFAAPPQSPTWLLYSHPVGRRARLHMGIRLSQSPLDPRSWTEPWVIY
EGPSGYSDLASIGPAPEGGLVFACLYESGARTSYDEISFCTFSLREVLENVPASPKPPNLGDKPRGCCWPS
SEQ ID NO: 12:
MASLP
SEQ ID NO: 13:
ASLP
SEQ ID NO: 14:
TVEKSVVF
SEQ ID NO: 15:
GDYDAPTHQVQW
SEQ ID NO: 16:
SMDQGSTW
SEQ ID NO: 17:
STDGGKTW
SEQ ID NO: 18:
PRPPAPEA
SEQ ID NO: 19:
QTPLEAAC
SEQ ID NO: 20:
NPRPPAPEA
SEQ ID NO: 21:
SQNDGES
SEQ ID NO: 22:
LSHSLST
SEQ ID NO: 23:
GAGAACGACTTTGGACTGGTGCAGCCTCTGGTCACCATGGAACAGCTGCTGTGGGTTTCCGGCAGACAGATCGGCAGCGTGGACA
CCTTCAGAATCCCTCTGATCACCGCCACACCTAGAGGCACCCTGCTGGCCTTTGCCGAGGCCAGAAAGATGAGCAGCTCTGACGA
GGGCGCCAAGTTTATTGCCCTGAGGCGGTCTATGGACCAGGGCTCTACATGGTCCCCTACCGCCTTCATCGTGAACGATGGCGAC
GTGCCCGATGGCCTGAATCTGGGAGCTGTGGTGTCCGATGTGGAAACCGGCGTGGTGTTCCTGTTCTACAGCCTGTGTGCCCACA
AGGCCGGTTGTCAGGTGGCCAGCACAATGCTCGTGTGGTCCAAGGACGACGGCGTGTCCTGGTCTACCCCTAGAAACCTGAGCCT
GGACATCGGCACCGAAGTGTTTGCTCCAGGACCTGGCTCTGGCATCCAGAAGCAGAGAGAGCCCAGAAAGGGCAGACTGATCGTG
TGTGGCCACGGCACCCTTGAGAGAGATGGCGTTTTCTGCCTGCTGAGCGACGATCATGGCGCCTCTTGGAGATACGGCAGCGGAG
TGTCTGGAATCCCTTACGGCCAGCCTAAGCAAGAGAACGATTTCAACCCCGACGAGTGCCAGCCTTACGAGCTGCCTGATGGCAG
CGTCGTGATCAACGCCCGGAACCAGAACAACTACCACTGCCACTGCCGGATCGTGCTGAGAAGCTACGACGCCTGCGATACCCTG
CGGCCTAGAGATGTGACCTTCGATCCTGAGCTGGTGGACCCTGTTGTTGCCGCTGGTGCCGTCGTGACATCTAGCGGCATCGTGT
TCTTCAGCAACCCTGCTCACCCCGAGTTCAGAGTGAATCTGACCCTGCGGTGGTCCTTCAGCAATGGCACAAGCTGGCGGAAAGA
AACCGTGCAGCTTTGGCCTGGACCTAGCGGCTACTCTTCTCTGGCTACACTGGAAGGCAGCATGGACGGCGAAGAACAGGCCCCT
CAGCTGTACGTGCTGTACGAGAAGGGCAGAAACCACTACACCGAGAGCATCAGCGTGGCCAAGATCAGCGTT
SEQ ID NO: 24:
ATGGCCAGCCTGCCTGTGCTGCAGAAAGAAAGCGTGTTCCAGTCTGGCGCCCACGCCTACAGAATTCCCGCTCTGCTGTATCTGC
CAGGCCAGCAGTCTCTGCTGGCTTTCGCTGAACAGCGGGCCAGCAAGAAGGATGAGCACGCCGAACTGATCGTGCTGCGGAGAGG
CGATTACGACGCCCCTACACATCAGGTGCAGTGGCAGGCTCAAGAGGTGGTGGCTCAGGCTAGACTGGACGGCCACAGATCTATG
AACCCCTGTCCTCTGTACGATGCCCAGACCGGCACACTGTTTCTGTTCTTTATCGCTATCCCCGGCCAAGTGACCGAGCAGCAGC
AGCTGCAGACAAGAGCCAACGTGACCAGACTGTGTCAAGTGACCTCCACCGACCACGGCAGAACCTGGTCTAGCCCTAGAGATCT
GACCGACGCCGCCATCGGACCTGCCTATAGAGAGTGGTCCACCTTCGCCGTTGGACCTGGACACTGTCTCCAGCTGCACGACAGG
GCTAGATCTCTGGTGGTGCCTGCCTACGCCTATAGAAAGCTGCACCCCATCCAGCGGCCTATTCCTAGCGCCTTCTGCTTTCTGA
GCCACGATCACGGCAGGACATGGGCCAGAGGACATTTCGTGGCCCAGGACACACTGGAATGCCAGGTGGCCGAAGTGGAAACCGG
CGAGCAGAGAGTCGTGACCCTGAACGCCAGATCTCACCTGAGAGCCAGAGTGCAGGCCCAGAGCACAAACGACGGCCTGGATTTC
CAAGAGAGCCAGCTGGTCAAGAAACTGGTGGAACCTCCTCCACAGGGCTGTCAGGGAAGCGTGATCAGCTTTCCATCTCCTAGAA
GCGGCCCTGGCTCTCCTGCTCAGTGGCTGCTGTATACACACCCCACACACAGCTGGCAGAGAGCCGATCTGGGCGCCTACCTGAA
TCCTAGACCTCCTGCTCCTGAGGCTTGGAGCGAACCTGTTCTGCTGGCCAAGGGCAGCTGTGCCTACAGCGATCTGCAGTCTATG
GGCACAGGCCCTGATGGCAGCCCTCTGTTTGGCTGTCTGTACGAGGCCAACGACTACGAAGAGATCGTGTTCCTGATGTTCACCC
TGAAGCAGGCCTTTCCAGCCGAGTACCTGCCTCAA
SEQ ID NO: 25:
ATGGAGGAAGTGACCACCTGTAGCTTCAACAGCCCTCTGTTCCGGCAAGAGGACGACCGGGGCATCACCTACAGAATCCCTGCTC
TGCTGTACATCCCTCCTACACACACCTTTCTGGCCTTCGCCGAGAAGCGGAGCACCAGACGAGATGAAGATGCCCTGCACCTGGT
GCTGAGAAGAGGCCTGAGAATCGGACAGCTGGTGCAGTGGGGACCTCTGAAGCCTCTGATGGAAGCCACACTGCCCGGCCACAGA
ACCATGAATCCTTGTCCTGTGTGGGAGCAGAAAAGCGGCTGCGTGTTCCTGTTCTTCATCTGCGTGCGGGGCCACGTGACCGAGA
GACAGCAAATCGTGTCCGGCAGAAACGCCGCCAGACTGTGCTTCATCTACAGCCAGGATGCCGGCTGCTCTTGGAGCGAAGTTCG
GGATCTGACCGAAGAAGTGATCGGCAGCGAGCTGAAGCACTGGGCCACATTTGCTGTTGGCCCTGGCCACGGAATCCAGCTGCAA
TCTGGCAGACTGGTCATCCCCGCCTACACCTACTATATCCCCAGCTGGTTCTTCTGCTTCCAACTGCCTTGCAAGACCCGGCCTC
ACAGCCTGATGATCTACAGCGACGATCTGGGCGTGACATGGCACCACGGCAGACTGATCAGACCCATGGTCACCGTGGAATGCGA
GGTGGCCGAAGTGACAGGCAGAGCTGGACACCCTGTGCTGTACTGCTCTGCCAGAACACCCAACCGGTGTAGAGCCGAGGCTCTG
TCTACAGATCACGGCGAGGGCTTTCAGAGACTGGCCCTCTCTAGACAGCTGTGCGAACCTCCTCATGGCTGTCAGGGCAGCGTGG
TGTCCTTCAGACCTCTGGAAATCCCTCACCGGTGCCAGGACAGCAGCTCTAAGGATGCCCCTACCATCCAGCAGTCTAGCCCTGG
CAGCAGCCTGAGACTGGAAGAGGAAGCCGGAACACCTAGCGAGAGCTGGCTGCTGTACTCTCACCCCACCAGCAGAAAGCAGAGA
GTGGACCTGGGCATCTACCTGAATCAGACCCCTCTGGAAGCCGCCTGTTGGAGCAGACCTTGGATTCTGCACTGTGGCCCTTGCG
GCTACTCTGATCTGGCCGCTCTGGAAGAAGAGGGCCTGTTCGGCTGCCTGTTTGAGTGCGGCACAAAGCAAGAGTGCGAGCAGAT
CGCCTTCCGGCTGTTCACCCACAGAGAGATCCTGAGCCATCTGCAGGGCGACTGCACAAGCCCAGGCAGAAATCCCAGCCAGTTC
AAGAGCAAC
SEQ ID NO: 26:
ATGGGCGTGCCCAGAACACCCAGCAGAACCGTGCTGTTCGAGAGAGAGAGGACCGGCCTGACCTACAGAGTGCCTTCTCTGCTGC
CTGTGCCTCCTGGACCTACACTGCTGGCCTTCGTGGAACAGAGACTGAGCCCCGATGATTCTCACGCCCACAGACTGGTGCTGAG
AAGAGGAACACTGGCTGGCGGCTCTGTTAGATGGGGAGCACTGCATGTGCTGGGCACAGCTGCTCTTGCCGAGCACAGATCCATG
AATCCCTGTCCTGTGCACGACGCCGGAACCGGCACAGTGTTTCTGTTCTTTATCGCCGTGCTGGGCCACACACCTGAGGCCGTTC
AAATTGCCACCGGCAGAAATGCCGCCAGACTGTGTTGTGTGGCCTCCAGAGATGCCGGCCTGTCTTGGGGATCTGCCAGAGATCT
GACCGAGGAAGCCATTGGCGGAGCCGTTCAGGATTGGGCCACATTTGCTGTTGGACCTGGACACGGCGTGCAGCTGCCAAGTGGT
AGACTGCTGGTGCCTGCCTACACATACAGAGTGGATCGGAGAGAGTGCTTCGGAAAGATCTGCCGGACAAGCCCTCACAGCTTCG
CCTTCTACTCCGACGATCACGGCCGGACTTGGAGATGTGGTGGCCTGGTGCCTAATCTGAGAAGCGGCGAATGTCAACTGGCCGC
CGTTGATGGTGGACAGGCTGGCAGCTTCCTGTACTGCAACGCCAGATCTCCTCTGGGCTCTAGAGTGCAGGCCCTGTCTACCGAT
GAGGGCACCAGTTTTCTGCCCGCCGAAAGAGTTGCCTCTCTGCCTGAAACAGCCTGGGGCTGTCAGGGCTCTATCGTGGGATTTC
CTGCTCCTGCTCCAAACAGACCCCGGGACGATTCTTGGAGTGTCGGCCCTGGATCTCCACTGCAGCCTCCATTGCTTGGACCAGG
CGTTCACGAGCCACCTGAAGAGGCTGCCGTTGATCCTAGAGGCGGACAAGTTCCTGGCGGCCCTTTTAGCAGACTGCAGCCAAGA
GGCGACGGCCCTAGACAACCTGGACCAAGACCTGGCGTCAGCGGAGATGTTGGCTCTTGGACACTGGCCCTGCCTATGCCTTTTG
CCGCTCCTCCTCAGTCTCCTACCTGGCTGCTGTACTCTCACCCTGTTGGCAGACGGGCCAGACTGCACATGGGCATCAGACTGTC
TCAGAGCCCTCTGGACCCCAGAAGCTGGACAGAGCCTTGGGTCATCTATGAGGGCCCTAGCGGCTACAGCGATCTGGCCTCTATT
GGCCCAGCTCCTGAAGGCGGACTGGTGTTCGCTTGTCTGTATGAGAGCGGCGCCAGAACCAGCTACGACGAGATCAGCTTCTGCA
CCTTCAGCCTGCGCGAGGTGCTGGAAAATGTGCCCGCCTCTCCTAAGCCTCCTAACCTGGGCGATAAGCCTAGAGGCTGTTGCTG
GCCATCT
SEQ ID NO: 27:
MTGERPSTALPDRRWGPRILGFWGGCRVWVFAAIFLLLSLAASWSKA
SEQ ID NO: 28:
MDMRVPAQLLGLLLLWLPGARC
SEQ ID NO: 29:
YGTL
SEQ ID NO: 30:
MTVEKSVVFKAEGEHFTDQKGNTIVGSGSGGTTKYFRIPAMCTTSKGTIVVFADARHNTASDQSFIDTAAARSTDGGKTWNKKIA
IYNDRVNSKLSRVMDPTCIVANIQGRETILVMVGKWNNNDKTWGAYRDKAPDTDWDLVLYKSTDDGVTFSKVETNIHDIVTKNGT
ISAMLGGVGSGLQLNDGKLVFPVQMVRTKNITTVLNTSFIYSTDGITWSLPSGYCEGFGSENNIIEFNASLVNNIRNSGLRRSFE
TKDFGKTWTEFPPMDKKVDNRNHGVQGSTITIPSGNKLVAAHSSAQNKNNDYTRSDISLYAHNLYSGEVKLIDDFYPKVGNASGA
GYSCLSYRKNVDKETLYVVYEANGSIEFQDLSRHLPVIKSYN
SEQ ID NO: 31:
EPKSCDKTHTCPPCPAPELLGGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLSPGK
SEQ ID NO: 32:
DKTHTCPPCPAPELLGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTCLVKGFYPSDIAVEWESNGQPENN
YKTTPPVLDSDGSFELTSKLTVDKSRWQQGNVFSCSVMHEALHNHYTQKSLSLSPGK
SEQ ID NO: 33:
EPKSCDKTHTCPPCPAPELLGGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTKNQVSLYCLVKGFYPSDIAVEWESNG
QPENNYKTTPPVLDSDGSFFLYSKLTVDKSRWQQGNVFSCSVMHEALHNHYTQKSLSLSPGK
SEQ ID NO: 34:
ATGAGACCTGCGGACCTGCCCCCGCGCCCCATGGAAGAATCCCCGGCGTCCAGCTCTGCCCCGACAGAGACGGAGGAGCCGGGGT
CCAGTGCAGAGGTCATGGAAGAAGTGACAACATGCTCCTTCAACAGCCCTCTGTTCCGGCAGGAAGATGACAGAGGGATTACCTA
CCGGATCCCAGCCCTGCTCTACATACCCCCCACCCACACCTTCCTGGCCTTTGCAGAGAAGCGTTCTACGAGGAGAGATGAGGAT
GCTCTCCACCTGGTGCTGAGGCGAGGGTTGAGGATTGGGCAGTTGGTACAGTGGGGGCCCCTGAAGCCACTGATGGAAGCCACAC
TACCGGGGCATCGGACCATGAACCCCTGTCCTGTATGGGAGCAGAAGAGTGGTTGTGTGTTCCTGTTCTTCATCTGTGTGCGGGG
CCATGTCACAGAGCGTCAACAGATTGTGTCAGGCAGGAATGCTGCCCGCCTTTGCTTCATCTACAGTCAGGATGCTGGATGTTCA
TGGAGTGAGGTGAGGGACTTGACTGAGGAGGTCATTGGCTCAGAGCTGAAGCACTGGGCCACATTTGCTGTGGGCCCAGGTCATG
GCATCCAGCTGCAGTCAGGGAGACTGGTCATCCCTGCGTATACCTACTACATCCCTTCCTGGTTCTTTTGCTTCCAGCTACCATG
TAAAACCAGGCCTCATTCTCTGATGATCTACAGTGATGACCTAGGGGTCACATGGCACCATGGTAGACTCATTAGGCCCATGGTT
ACAGTAGAATGTGAAGTGGCAGAGGTGACTGGGAGGGCTGGCCACCCTGTGCTATATTGCAGTGCCCGGACACCAAACAGGTGCC
GGGCAGAGGCGCTCAGCACTGACCATGGTGAAGGCTTTCAGAGACTGGCCCTGAGTCGACAGCTCTGTGAGCCCCCACATGGTTG
CCAAGGGAGTGTGGTAAGTTTCCGGCCCCTGGAGATCCCACATAGGTGCCAGGACTCTAGCAGCAAAGATGCACCCACCATTCAG
CAGAGCTCTCCAGGCAGTTCACTGAGGCTGGAGGAGGAAGCTGGAACACCGTCAGAATCATGGCTCTTGTACTCACACCCAACCA
GTAGGAAACAGAGGGTTGACCTAGGTATCTATCTCAACCAGACCCCCTTGGAGGCTGCCTGCTGGTCCCGCCCCTGGATCTTGCA
CTGTGGGCCCTGTGGCTACTCTGATCTGGCTGCTCTGGAGGAGGAGGGCTTGTTTGGGTGTTTGTTTGAATGTGGGACCAAGCAA
GAGTGTGAGCAGATTGCCTTCCGCCTGTTTACACACCGGGAGATCCTGAGTCACCTGCAGGGGGACTGCACCAGCCCTGGTAGGA
ACCCAAGCCAATTCAAAAGCAAT
SEQ ID NO: 35:
ATGATGAGCTCTGCAGCCTTCCCAAGGTGGCTGAGCATGGGGGTCCCTCGTACCCCTTCACGGACAGTGCTCTTCGAGCGGGAGA
GGACGGGCCTGACCTACCGCGTGCCCTCGCTGCTCCCCGTGCCCCCCGGGCCCACCCTGCTGGCCTTTGTGGAGCAGCGGCTCAG
CCCTGACGACTCCCACGCCCACCGCCTGGTGCTGAGGAGGGGCACGCTGGCCGGGGGCTCCGTGCGGTGGGGTGCCCTGCACGTG
CTGGGGACAGCAGCCCTGGCGGAGCACCGGTCCATGAACCCCTGCCCTGTGCACGATGCTGGCACGGGCACCGTCTTCCTCTTCT
TCATCGCGGTGCTGGGCCACACGCCTGAGGCCGTGCAGATCGCCACGGGAAGGAACGCCGCGCGCCTCTGCTGTGTGGCCAGCCG
TGACGCCGGCCTCTCGTGGGGCAGCGCCCGGGACCTCACCGAGGAGGCCATCGGTGGTGCCGTGCAGGACTGGGCCACATTCGCT
GTGGGTCCCGGCCACGGTGTGCAGCTGCCCTCAGGCCGCCTGCTGGTACCCGCCTACACCTACCGCGTGGACCGCCGAGAGTGTT
TTGGCAAGATCTGCCGGACCAGCCCTCACTCCTTCGCCTTCTACAGCGATGACCACGGCCGCACCTGGCGCTGTGGAGGCCTCGT
GCCCAACCTGCGCTCAGGCGAGTGCCAGCTGGCAGCGGTGGACGGTGGGCAGGCCGGCAGCTTCCTCTACTGCAATGCCCGGAGC
CCACTGGGCAGCCGTGTGCAGGCGCTCAGCACTGACGAGGGCACCTCCTTCCTGCCCGCAGAGCGCGTGGCTTCCCTGCCCGAGA
CTGCCTGGGGCTGCCAGGGCAGCATCGTGGGCTTCCCAGCCCCCGCCCCCAACAGGCCACGGGATGACAGTTGGTCAGTGGGCCC
CGGGAGTCCCCTCCAGCCTCCACTCCTCGGTCCTGGAGTCCACGAACCCCCAGAGGAGGCTGCTGTAGACCCCCGTGGAGGCCAG
GTGCCTGGTGGGCCCTTCAGCCGTCTGCAGCCTCGGGGGGATGGCCCCAGGCAGCCTGGCCCCAGGCCTGGGGTCAGTGGGGATG
TGGGGTCCTGGACCCTGGCACTCCCCATGCCCTTTGCTGCCCCGCCCCAGAGCCCCACGTGGCTGCTGTACTCCCACCCAGTGGG
GCGCAGGGCTCGGCTACACATGGGTATCCGCCTGAGCCAGTCCCCGCTGGACCCGCGCAGCTGGACAGAGCCCTGGGTGATCTAC
GAGGGCCCCAGCGGCTACTCCGACCTGGCGTCCATCGGGCCGGCCCCTGAGGGGGGCCTGGTTTTTGCCTGCCTGTACGAGAGCG
GGGCCAGGACCTCCTATGATGAGATTTCCTTTTGTACATTCTCCCTGCGTGAGGTCCTGGAGAACGTGCCCGCCAGCCCCAAACC
GCCCAACCTTGGGGACAAGCCTCGGGGGTGCTGCTGGCCCTCC
SEQ ID NO: 36:
ASLPYLQKESVFQSGAHAYRIPALLYLPGQQSLLAFAEQRASKKDEHAELIVLRRGDYDAPTHQVQWQAQEVVAQARLDGHRSMN
PCPLYDAQTGTLFLFFIAIPGQVTEQQQLQTRANVTRLCQVTSTDHGRTWSSPRDLTDAAIGPAYREWSTFAVGPGHCLQLHDRA
RSLVVPAYAYRKLHPKQRPIPSAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEAWSEPVLLAKGSCAYSDLQSMG
TGPDGSPLFGCLYEANDYEEIVFLMFTLKQAFPAEYLPQ
SEQ ID NO: 37:
ASLPYLQKESVFQSGAHAYRIPALLYLPGQQSLLAFAEQRASKKDEHAELIVLRRGDYDAPTHQVQWQAQEVVAQARLDGHRSMN
PCPLYDAQTGTLFLFFIAIPGQVTEQQQLQTRANVTRLCQVTSTDHGRTWSSPRDLTDAAIGPAYREWSTFAVGPGHCLQLHDRA
RSLVVPAYAYRKLHPKQRPIPSAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEAWSEPVLLAKGSAAYSDLQSMG
TGPDGSPLFGCLYEANDYEEIVFLMFTLKQAFPAEYLPQ
SEQ ID NO: 38:
AASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGCLYEANDYEEIVFLMFTLKQAFPAEYLPQ
SEQ ID NO: 39:
DASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGCLYEANDYEEIVFLMFTLKQAFPAEYLPQ
SEQ ID NO: 40:
EVQLVESGGGLVQPGGSLRLSCAASGFNIKDTYIHWVRQAPGKGLEWVARIYPTNGYTRYADSVKGRFTISADTSKNTAYLQMNS
LRAEDTAVYYCSRWGGDGFYAMDYWGQGTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLYCLVKGFYPSDIAVEWESNGQPENNYKTTPPVLDSDGSFFLYSKLTVDKSRWQQGNV
FSCSVMHEALHNHYTQKSLSLSPGK
SEQ ID NO: 41:
DIQMTQSPSSLSASVGDRVTITCRASQDVNTAVAWYQQKPGKAPKLLIYSASFLYSGVPSRFSGSRSGTDFTLTISSLQPEDFAT
YYCQQHYTTPPTFGQGTKVEIKRTVAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
SEQ ID NO: 42:
EVQLVESGGGLVQPGGSLRLSCAASGFNIKDTYIHWVRQAPGKGLEWVARIYPTNGYTRYADSVKGRFTISADTSKNTAYLQMNS
LRAEDTAVYYCSRWGGDGFYAMDYWGQGTLVTVSSGGGGSGGGGSGGGGSDIQMTQSPSSLSASVGDRVTITCRASQDVNTAVAW
YQQKPGKAPKLLIYSASFLYSGVPSRFSGSRSGTDFTLTISSLQPEDFATYYCQQHYTTPPTFGQGTKVEIK
SEQ ID NO: 43:
ASLPYLQKESVFQSGAHAYRIPALLYLPGQQSLLAFAEQRASKKDEHAELIVLRRGDYDAPTHQVQWQAQEVVAQARLDGHRSMN
PCPLYDAQTGTLFLFFIAIPGQVTEQQQLQTRANVTRLCQVTSTDHGRTWSSPRDLTDAAIGPAYREWSTFAVGPGHCLQLHDRA
RSLVVPAYAYRKLHPKQRPIPSAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEAWSEPVLLAKGSCAYSDLQSMG
TGPDGSPLFGCLYEANDYEEIVFLMFTLKQAFPAEYLPQGGGGSGGGGSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKLTVDKSRWQQGNVFSCS
VMHEALHNHYTQKSLSLSPGKGGGGSGGGGSGGGGSEVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSRWGGDGFYAMDYWGQGTLVTVSSGGGGSGGGGSGGGG
SDIQMTQSPSSLSASVGDRVTITCRASQDVNTAVAWYQQKPGKAPKLLIYSASFLYSGVPSRFSGSRSGTDFTLTISSLQPEDFA
TYYCQQHYTTPPTFGQGTKVEIK
SEQ ID NO: 44:
ASLPYLQKESVFQSGAHAYRIPALLYLPGQQSLLAFAEQRASKKDEHAELIVLRRGDYDAPTHQVQWQAQEVVAQARLDGHRSMN
PCPLYDAQTGTLFLFFIAIPGQVTEQQQLQTRANVTRLCQVTSTDHGRTWSSPRDLTDAAIGPAYREWSTFAVGPGHCLQLHDRA
RSLVVPAYAYRKLHPKQRPIPSAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEAWSEPVLLAKGSAAYSDLQSMG
TGPDGSPLFGCLYEANDYEEIVFLMFTLKQAFPAEYLPQGGGGSGGGGSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKLTVDKSRWQQGNVFSCS
VMHEALHNHYTQKSLSLSPGKGGGGSGGGGSGGGGSEVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSRWGGDGFYAMDYWGQGTLVTVSSGGGGSGGGGSGGGG
SDIQMTQSPSSLSASVGDRVTITCRASQDVNTAVAWYQQKPGKAPKLLIYSASFLYSGVPSRFSGSRSGTDFTLTISSLQPEDFA
TYYCQQHYTTPPTFGQGTKVEIK
SEQ ID NO: 45:
AASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSGAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGKGGGGSGGGGSGGGGSEVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYCSRWGGDGFYAMDYWGQGTLVTVSSGGGGSGGGGSGGG
GSDIQMTQSPSSLSASVGDRVTITCRASQDVNTAVAWYQQKPGKAPKLLIYSASFLYSGVPSRFSGSRSGTDFTLTISSLQPEDF
ATYYCQQHYTTPPTFGQGTKVEIK
SEQ ID NO: 46:
DASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSCAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGKGGGGSGGGGSGGGGSEVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYCSRWGGDGFYAMDYWGQGTLVTVSSGGGGSGGGGSGGG
GSDIQMTQSPSSLSASVGDRVTITCRASQDVNTAVAWYQQKPGKAPKLLIYSASFLYSGVPSRFSGSRSGTDFTLTISSLQPEDF
ATYYCQQHYTTPPTFGQGTKVEIK
SEQ ID NO: 47:
AASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGKGGGGSGGGGSGGGGSEVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYCSRWGGDGFYAMDYWGQGTLVTVSSGGGGSGGGGSGGG
GSDIQMTQSPSSLSASVGDRVTITCRASQDVNTAVAWYQQKPGKAPKLLIYSASFLYSGVPSRFSGSRSGTDFTLTISSLQPEDF
ATYYCQQHYTTPPTFGQGTKVEIK
SEQ ID NO: 48:
DASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGKGGGGSGGGGSGGGGSEVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYCSRWGGDGFYAMDYWGQGTLVTVSSGGGGSGGGGSGGG
GSDIQMTQSPSSLSASVGDRVTITCRASQDVNTAVAWYQQKPGKAPKLLIYSASFLYSGVPSRFSGSRSGTDFTLTISSLQPEDF
ATYYCQQHYTTPPTFGQGTKVEIK
SEQ ID NO: 49:
DIQMTQSPSSLSASVGDRVTITCRASQDVNTAVAWYQQKPGKAPKLLIYSASFLYSGVPSRFSGSRSGTDFTLTISSLQPEDFAT
YYCQQHYTTPPTFGQGTKVEIKRTVAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
SEQ ID NO: 50:
EVQLVESGGGLVQPGGSLRLSCAASGFNIKDTYIHWVRQAPGKGLEWVARIYPTNGYTRYADSVKGRFTISADTSKNTAYLQMNS
LRAEDTAVYYCSRWGGDGFYAMDYWGQGTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLYCLVKGFYPSDIAVEWESNGQPENNYKTTPPVLDSDGSFFLYSKLTVDKSRWQQGNV
FSCSVMHEALHNHYTQKSLSLSPGK
SEQ ID NO: 51:
ASLPYLQKESVFQSGAHAYRIPALLYLPGQQSLLAFAEQRASKKDEHAELIVLRRGDYDAPTHQVQWQAQEVVAQARLDGHRSMN
PCPLYDAQTGTLFLFFIAIPGQVTEQQQLQTRANVTRLCQVTSTDHGRTWSSPRDLTDAAIGPAYREWSTFAVGPGHCLQLHDRA
RSLVVPAYAYRKLHPKQRPIPSAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEAWSEPVLLAKGSCAYSDLQSMG
TGPDGSPLFGCLYEANDYEEIVFLMFTLKQAFPAEYLPQGGGGSGGGGSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLTSKLTVDKSRWQQGNVFSCS
VMHEALHNHYTQKSLSLSPGK
SEQ ID NO: 52:
ASLPYLQKESVFQSGAHAYRIPALLYLPGQQSLLAFAEQRASKKDEHAELIVLRRGDYDAPTHQVQWQAQEVVAQARLDGHRSMN
PCPLYDAQTGTLFLFFIAIPGQVTEQQQLQTRANVTRLCQVTSTDHGRTWSSPRDLTDAAIGPAYREWSTFAVGPGHCLQLHDRA
RSLVVPAYAYRKLHPKQRPIPSAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEAWSEPVLLAKGSAAYSDLQSMG
TGPDGSPLFGCLYEANDYEEIVFLMFTLKQAFPAEYLPQGGGGSGGGGSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLTSKLTVDKSRWQQGNVFSCS
VMHEALHNHYTQKSLSLSPGK
SEQ ID NO: 53:
AASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLTSKLTVDKSRWQQGNVFSC
SVMHEALHNHYTQKSLSLSPGK
SEQ ID NO: 54:
DASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLTSKLTVDKSRWQQGNVFSC
SVMHEALHNHYTQKSLSLSPGK
SEQ ID NO: 55
TVEKSVVFKAEGEHFTDQKGNTIVGSGSGGTTKYFRIPAMCTTSKGTIVVFADARHNTASDQSFIDTAAARSTDGGKTWNKKIAI
YNDRVNSKLSRVMDPTCIVANIQGRETILVMVGKWNNNDKTWGAYRDKAPDTDWDLVLYKSTDDGVTFSKVETNIHDIVTKNGTI
SAMLGGVGSGLQLNDGKLVFPVQMVRTKNITTVLNTSFIYSTDGITWSLPSGYCEGFGSENNIIEFNASLVNNIRNSGLRRSFET
KDFGKTWTEFPPMDKKVDNRNHGVQGSTITIPSGNKLVAAHSSAQNKNNDYTRSDISLYAHNLYSGEVKLIDDFYPKVGNASGAG
YSCLSYRKNVDKETLYVVYEANGSIEFQDLSRHLPVIKSYNGGGGSGGGGSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLTSKLTVDKSRWQQGNVFS
CSVMHEALHNHYTQKSLSLSPGK
SEQ ID NO: 56
ACAGTGGAAAAGTCCGTGGTGTTCAAGGCCGAGGGCGAGCACTTCACCGACCAGAAAGGCAATACCATCGTCGGCTCTGGCAGCG
GCGGCACCACCAAGTACTTTAGAATCCCCGCCATGTGCACCACCAGCAAGGGCACCATTGTGGTGTTCGCCGACGCCAGACACAA
CACCGCCAGCGATCAGAGCTTCATCGATACCGCTGCCGCCAGATCTACCGATGGCGGCAAGACCTGGAACAAGAAGATCGCCATC
TACAACGACCGCGTGAACAGCAAGCTGAGCAGAGTGATGGACCCTACCTGCATCGTGGCCAACATCCAGGGCAGAGAAACCATCC
TGGTCATGGTCGGAAAGTGGAACAACAACGATAAGACCTGGGGCGCCTACAGAGACAAGGCCCCTGATACCGATTGGGACCTCGT
GCTGTACAAGAGCACCGATGACGGCGTGACCTTCAGCAAGGTGGAAACAAACATCCACGACATCGTGACCAAGAACGGCACCATC
TCTGCCATGCTCGGCGGCGTTGGATCTGGCCTGCAACTGAATGATGGCAAGCTGGTGTTCCCCGTGCAGATGGTCCGAACAAAGA
ATATCACCACCGTGCTGAATACCAGCTTCATCTACAGCACCGACGGCATCACATGGTCCCTGCCTAGCGGCTACTGTGAAGGCTT
TGGCAGCGAGAACAACATCATCGAGTTCAACGCCAGCCTGGTCAACAACATCCGGAACAGCGGCCTGCGGAGAAGCTTCGAGACA
AAGGACTTCGGAAAGACGTGGACCGAGTTTCCTCCAATGGACAAGAAGGTGGACAACCGGAACCACGGCGTGCAGGGCAGCACAA
TCACAATCCCTAGCGGCAACAAACTGGTGGCCGCTCACTCTAGCGCCCAGAACAAGAACAACGACTACACCAGAAGCGACATCAG
CCTGTACGCCCACAACCTGTACAGCGGCGAAGTGAAGCTGATCGACGACTTCTACCCCAAAGTGGGCAATGCCAGCGGAGCCGGC
TACAGCTGTCTGAGCTACCGGAAAAATGTGGACAAAGAAACCCTGTACGTGGTGTACGAGGCCAACGGCAGCATCGAGTTTCAGG
ACCTGAGCAGACATCTGCCCGTGATCAAGAGCTACAACggcggaggtggaagtggcggaggcggatccgacaaaactcacacatg
cccaccgtgcccagcacctgaactcctggggggaccgtcagtcttcctcttccccccaaaacccaaggacaccctcatgatctcc
cggacccctgaggtcacatgcgtggtggtggacgtgagccacgaagaccctgaggtcaagttcaactggtacgtggacggcgtgg
aggtgcataatgccaagacaaagccgcgggaggagcagtacaacagcacgtaccgtgtggtcagcgtcctcaccgtcctgcacca
ggactggctgaatggcaaggagtacaagtgcaaggtctccaacaaagccctcccagcccccatcgagaaaaccatctccaaagcc
aaagggcagccccgagaaccacaggtctacaccctgcccccatcccgggaggagatgaccaagaaccaggtcagcctgacctgcc
tggtcaaaggcttctatcccagcgacatcgccgtggagtgggagagcaatgggcagccggagaacaactacaagaccacgcctcc
cgtgctggactccgacggctccttcttcctcactagcaagctcaccgtggacaagagcaggtggcagcaggggaacgtcttctca
tgctccgtgatgcatgaggctctgcacaaccactacacgcagaagagcctctccctgtctccgggtaaa
SEQ ID NO: 57
EVQLVESGGGLVQPGGSLRLSCAASGFNIKDTYIHWVRQAPGKGLEWVARIYPTNGYTRYADSVKGRFTISADTSKNTAYLQMNS
LRAEDTAVYYCSRWGGDGFYAMDYWGQGTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLYCLVKGFYPSDIAVEWESNGQPENNYKTTPPVLDSDGSFFLYSKLTVDKSRWQQGNV
FSCSVMHEALHNHYTQKSLSLSPGK
SEQ ID NO: 58
GAGGTGCAGCTGGTTGAATCTGGCGGAGGACTGGTTCAGCCTGGCGGATCTCTGAGACTGTCTTGTGCCGCCAGCGGCTTCAACA
TCAAGGACACCTACATCCACTGGGTCCGACAGGCCCCTGGCAAAGGACTTGAATGGGTCGCCAGAATCTACCCCACCAACGGCTA
CACCAGATACGCCGACTCTGTGAAGGGCAGATTCACCATCAGCGCCGACACCAGCAAGAACACCGCCTACCTGCAGATGAACAGC
CTGAGAGCCGAGGACACCGCCGTGTACTACTGTTCTAGATGGGGAGGCGACGGCTTCTACGCCATGGATTATTGGGGCCAGGGCA
CCCTGGTCACCGTTTCTTCTGCtagcACCAAGGGCCCATCcGTCTTCCCCCTGGCACCCTCCTCCAAGAGCACCTCTGGGGGCAC
AGCGGCCCTGGGCTGCCTGGTCAAGGACTACTTCCCCGAACCGGTGACGGTGTCcTGGAACTCAGGCGCtCTGACCAGCGGCGTG
CACACCTTCCCGGCTGTCCTACAGTCCTCAGGACTCTACTCCCTCAGCAGCGTGGTGACCGTGCCCTCCAGCAGCTTGGGCACCC
AGACCTACATCTGCAACGTGAATCACAAGCCCAGCAACACCAAGGTGGACAAGAAAGTTGAGCCCAAATCTTGTGACAAAACTCA
CACATGCCCACCGTGCCCAGCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGTCAGCGTCCTCACCGTCCT
GCACCAGGACTGGCTGAATGGCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTcTACACCCTGCCCCCATCCCGGGAGGAGATGACCAAGAACCAGGTCAGCCTGt
acTGCCTGGTCAAAGGCTTCTATCCCAGCGACATCGCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGTCTCCGGGTAAA
SEQ ID NO: 59
EVQLVESGGGLVQPGGSLRLSCAASGFNIKDTYIHWVRQAPGKGLEWVARIYPTNGYTRYADSVKGRFTISADTSKNTAYLQMNS
LRAEDTAVYYCSRWGGDGFYAMDYWGQGTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGKGGGGSGGGGSGGGGSTVEKSVVFKAEGEHFTDQKGNTIVGSGSGGTTKYFRIPAMCTTSK
GTIVVFADARHNTASDQSFIDTAAARSTDGGKTWNKKIAIYNDRVNSKLSRVMDPTCIVANIQGRETILVMVGKWNNNDKTWGAY
RDKAPDTDWDLVLYKSTDDGVTFSKVETNIHDIVTKNGTISAMLGGVGSGLQLNDGKLVFPVQMVRTKNITTVLNTSFIYSTDGI
TWSLPSGYCEGFGSENNIIEFNASLVNNIRNSGLRRSFETKDFGKTWTEFPPMDKKVDNRNHGVQGSTITIPSGNKLVAAHSSAQ
NKNNDYTRSDISLYAHNLYSGEVKLIDDFYPKVGNASGAGYSCLSYRKNVDKETLYVVYEANGSIEFQDLSRHLPVIKSYN
SEQ ID NO: 60
gaggtgcagctggttgaatctggcggaggactggttcagcctggcggatctctgagactgtcttgtgccgccagcggcttcaaca
tcaaggacacctacatccactgggtccgacaggcccctggcaaaggacttgaatgggtcgccagaatctaccccaccaacggcta
caccagatacgccgactctgtgaagggcagattcaccatcagcgccgacaccagcaagaacaccgcctacctgcagatgaacagc
ctgagagccgaggacaccgccgtgtactactgttctagatggggaggcgacggcttctacgccatggattattggggccagggca
ccctggtcaccgtttcttctgctagcaccaagggcccatccgtcttccccctggcaccctcctccaagagcacctctgggggcac
agcggccctgggctgcctggtcaaggactacttccccgaaccggtgacggtgtcctggaactcaggcgctctgaccagcggcgtg
cacaccttcccggctgtcctacagtcctcaggactctactccctcagcagcgtggtgaccgtgccctccagcagcttgggcaccc
agacctacatctgcaacgtgaatcacaagcccagcaacaccaaggtggacaagaaagttgagcccaaatcttgtgacaaaactca
cacatgcccaccgtgcccagcacctgaactcctggggggaccgtcagtcttcctcttccccccaaaacccaaggacaccctcatg
atctcccggacccctgaggtcacatgcgtggtggtggacgtgagccacgaagaccctgaggtcaagttcaactggtacgtggacg
gcgtggaggtgcataatgccaagacaaagccgcgggaggagcagtacaacagcacgtaccgtgtggtcagcgtcctcaccgtcct
gcaccaggactggctgaatggcaaggagtacaagtgcaaggtctccaacaaagccctcccagcccccatcgagaaaaccatctcc
aaagccaaagggcagccccgagaaccacaggtctacaccctgcccccatcccgggaggagatgaccaagaaccaggtcagcctga
cctgcctggtcaaaggcttctatcccagcgacatcgccgtggagtgggagagcaatgggcagccggagaacaactacaagaccac
gcctcccgtgctggactccgacggctccttcttcctctatagcaagctcaccgtggacaagagcaggtggcagcaggggaacgtc
ttctcatgctccgtgatgcatgaggctctgcacaaccactacacgcagaagagcctctccctgtctccgggtaaaggtggcggag
gatctggcggaggtggaagcggcggaggcggatctacagtggaaaagtccgtggtgttcaaggccgagggcgagcacttcaccga
ccagaaaggcaataccatcgtcggctctggcagcggcggcaccaccaagtactttagaatccccgccatgtgcaccaccagcaag
ggcaccattgtggtgttcgccgacgccagacacaacaccgccagcgatcagagcttcatcgataccgctgccgccagaagtacag
acggcggcaagacctggaacaagaagatcgccatctacaacgaccgcgtgaacagcaagctgagcagagtgatggaccctacctg
catcgtggccaacatccagggcagagaaaccatcctggtcatggtcggaaagtggaacaacaacgataagacctggggcgcctac
agagacaaggcccctgataccgattgggacctcgtgctgtataagagcaccgacgacggcgtgaccttcagcaaggtggaaacaa
acatccacgacatcgtgaccaagaacggcaccatctctgccatgctcggcggcgttggatctggcctgcaactgaatgatggcaa
gctggtgttccccgtgcagatggtccgaacaaagaacatcaccaccgtgctgaataccagcttcatctactccaccgacggcatc
acatggtccctgcctagcggctactgtgaaggctttggcagcgagaacaacatcatcgagttcaacgccagcctggtcaacaaca
tccggaacagcggcctgcggagaagcttcgagacaaaggacttcggaaagacgtggaccgagtttcctccaatggacaagaaggt
ggacaaccggaaccacggcgtgcagggcagcacaatcacaatccctagcggcaacaaactggtggccgctcactctagcgcccag
aacaagaacaacgattacaccagaagcgacatcagcctgtacgcccacaacctgtactccggcgaagtgaagctgatcgacgact
tctaccccaaagtgggcaatgccagcggagccggctacagctgtctgagctaccggaaaaatgtggacaaagaaaccctgtacgt
ggtgtacgaggccaacggcagcatcgagtttcaggacctgagcagacatctgcccgtgatcaagagctacaat
SEQ ID NO: 61
TVEKSVVFKAEGEHFTDQKGNTIVGSGSGGTTKYFRIPAMCTTSKGTIVVFADARHNTASDQSFIDTAAARSTDGGKTWNKKIAI
YNDRVNSKLSRVMVPTCIVANIQGRETILVMVGKWNNNDKTWGAYRDKAPDTDWDLVLYKSTDDGVTFSKVETNIHDIVTKNGTI
SAMLGGVGSGLQLNDGKLVFPVQMVRTKNITTVLNTSFIYSTDGITWSLPSGYCEGFGSVNNIIEFNASLVNNIRNSGLRRSFET
KDFGKTWTEFPPMDKKVDNRNHGVQGSTITIPSGNKLVAAHSSAQNKNNDYTRSDISLYAHNLYSGEVKLIDDFYPKVGNASGAG
YSCLSYRKNVDKETLYVVYEANGSIEFQDLSRHLPVIKSYNGGGGSGGGGSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLTSKLTVDKSRWQQGNVFS
CSVMHEALHNHYTQKSLSLSPGK
SEQ ID NO: 62
acagtggaaaagtccgtggtgttcaaggccgagggcgagcacttcaccgaccagaaaggcaataccatcgtcggctctggcagcg
gcggcaccaccaagtactttagaatccccgccatgtgcaccaccagcaagggcaccattgtggtgttcgccgacgccagacacaa
caccgccagcgatcagagcttcatcgataccgctgccgccagaagtacagacggcggcaagacctggaacaagaagatcgccatc
tacaacgaccgcgtgaacagcaagctgagcagagtgatggtccctacctgcatcgtggccaacatccagggcagagaaaccatcc
tggtcatggtcggaaagtggaacaacaacgataagacctggggcgcctacagagacaaggcccctgataccgattgggacctcgt
gctgtataagagcaccgacgacggcgtgaccttcagcaaggtggaaacaaacatccacgacatcgtgaccaagaacggcaccatc
tctgccatgctcggcggcgttggatctggcctgcaactgaatgatggcaagctggtgttccccgtgcagatggtccgaacaaaga
acatcaccaccgtgctgaataccagcttcatctactccaccgacggcatcacatggtccctgcctagcggctactgtgaaggctt
tggcagcgtgaacaacatcatcgagttcaacgccagcctggtcaacaacatccggaacagcggcctgcggagaagcttcgagaca
aaggacttcggaaagacgtggaccgagtttcctccaatggacaagaaggtggacaaccggaaccacggcgtgcagggcagcacaa
tcacaatccctagcggcaacaaactggtggccgctcactctagcgcccagaacaagaacaacgattacaccagaagcgacatcag
cctgtacgcccacaacctgtactccggcgaagtgaagctgatcgacgacttctaccccaaagtgggcaatgccagcggagccggc
tacagctgtctgagctaccggaaaaatgtggacaaagaaaccctgtacgtggtgtacgaggccaacggcagcatcgagtttcagg
acctgagcagacatctgcccgtgatcaagagctacaatggcggaggtggaagtggcggaggcggatccgacaaaactcacacatg
cccaccgtgcccagcacctgaactcctggggggaccgtcagtcttcctcttccccccaaaacccaaggacaccctcatgatctcc
cggacccctgaggtcacatgcgtggtggtggacgtgagccacgaagaccctgaggtcaagttcaactggtacgtggacggcgtgg
aggtgcataatgccaagacaaagccgcgggaggagcagtacaacagcacgtaccgtgtggtcagcgtcctcaccgtcctgcacca
ggactggctgaatggcaaggagtacaagtgcaaggtctccaacaaagccctcccagcccccatcgagaaaaccatctccaaagcc
aaagggcagccccgagaaccacaggtctacaccctgcccccatcccgggaggagatgaccaagaaccaggtcagcctgacctgcc
tggtcaaaggcttctatcccagcgacatcgccgtggagtgggagagcaatgggcagccggagaacaactacaagaccacgcctcc
cgtgctggactccgacggctccttcttcctcactagcaagctcaccgtggacaagagcaggtggcagcaggggaacgtcttctca
tgctccgtgatgcatgaggctctgcacaaccactacacgcagaagagcctctccctgtctccgggtaaa
SEQ ID NO: 63
MEDLRPMASLPVLQKESVFQSGAHAYRIPALLYLPGQQSLLAFAEQRASKKDEHAELIVLRRGDYDAPTHQVQWQAQEVVAQARL
DGHRSMNPCPLYDAQTGTLFLFFIAIPGQVTEQQQLQTRANVTRLCQVTSTDHGRTWSSPRDLTDAAIGPAYREWSTFAVGPGHC
LQLHDRARSLVVPAYAYRKLHPIQRPIPSAFCFLSHDHGRTWARGHFVAQDTLECQVAEVETGEQRVVTLNARSHLRARVQAQST
NDGLDFQESQLVKKLVEPPPQGCQGSVISFPSPRSGPGSPAQWLLYTHPTHSWQRADLGAYLNPRPPAPEAWSEPVLLAKGSCAY
SDLQSMGTGPDGSPLFGCLYEANDYEEIVFLMFTLKQAFPAEYLPQ
SEQ ID NO: 64
ATGGAAGATCTCAGGCCCATGGCATCTCTGCCTGTGCTGCAGAAAGAAAGCGTGTTCCAGTCTGGCGCCCACGCCTACAGAATTC
CCGCTCTGCTGTATCTGCCAGGCCAGCAGTCTCTGCTGGCTTTCGCTGAACAGCGGGCCAGCAAGAAGGATGAGCACGCCGAACT
GATCGTGCTGCGGAGAGGCGATTACGACGCCCCTACACATCAGGTGCAGTGGCAGGCTCAAGAGGTGGTGGCTCAGGCTAGACTG
GACGGCCACAGATCTATGAACCCCTGTCCTCTGTACGATGCCCAGACCGGCACACTGTTTCTGTTCTTTATCGCTATCCCCGGCC
AAGTGACCGAGCAGCAGCAGCTGCAGACAAGAGCCAACGTGACCAGACTGTGTCAAGTGACCTCCACCGACCACGGCAGAACCTG
GTCTAGCCCTAGAGATCTGACCGACGCCGCCATCGGACCTGCCTATAGAGAGTGGTCCACCTTCGCCGTTGGACCTGGACACTGT
CTCCAGCTGCACGACAGGGCTAGATCTCTGGTGGTGCCTGCCTACGCCTATAGAAAGCTGCACCCCATCCAGCGGCCTATTCCTA
GCGCCTTCTGCTTTCTGAGCCACGATCACGGCAGGACATGGGCCAGAGGACATTTCGTGGCCCAGGACACACTGGAATGCCAGGT
GGCCGAAGTGGAAACCGGCGAGCAGAGAGTCGTGACCCTGAACGCCAGATCTCACCTGAGAGCCAGAGTGCAGGCCCAGAGCACA
AACGACGGCCTGGATTTCCAAGAGAGCCAGCTGGTCAAGAAACTGGTGGAACCTCCTCCACAGGGCTGTCAGGGAAGCGTGATCA
GCTTTCCATCTCCTAGAAGCGGCCCTGGCTCTCCTGCTCAGTGGCTGCTGTATACACACCCCACACACAGCTGGCACACAGCCGA
TCTGGGCGCCTACCTGAATCCTAGACCTCCTGCTCCTGAGGCTTGGAGCGAACCTGTTCTGCTGGCCAAGGGCAGCTGTGCCTAC
AGCGATCTGCAGTCTATGGGCACAGGCCCTGATGGCAGCCCTCTGTTTGGCTGTCTGTACGAGGCCAACGACTACGAAGAGATCG
TGTTCCTGATGTTCACCCTGAAGCAGGCCTTTCCAGCCGAGTACCTGCCTCAA
SEQ ID NO: 65
ASLPYLQKESVFQSGAHAYRIPALLYLPGQQSLLAFAEQRASKKDEHAELIVLRRGDYDAPTHQVQWQAQEVVAQARLDGHRSMN
PCPLYDAQTGTLFLFFIAIPGQVTEQQQLQTRANVTRLCQVTSTDHGRTWSSPRDLTDAAIGPAYREWSTFAVGPGHCLQLHDRA
RSLVVPAYAYRKLHPKQRPIPSAFCFLSHDHGRTWARGHFVAQDTLECQVAEVETGEQRVVTLNARSHLRARVQAQSTNDGLDFQ
ESQLVKKLVEPPPQGCQGSVISFPSPRSGPGSPAQWLLYTHPTHSWQRADLGAYLNPRPPAPEAWSEPVLLAKGSCAYSDLQSMG
TGPDGSPLFGCLYEANDYEEIVFLMFTLKQAFPAEYLPQGGGGSGGGGSDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKLTVDKSRWQQGNVFSCS
VMHEALHNHYTQKSLSLSPGKGGGGSGGGGSGGGGSEVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSRWGGDGFYAMDYWGQGTLVTVSSGGGGSGGGGSGGGG
SDIQMTQSPSSLSASVGDRVTITCRASQDVNTAVAWYQQKPGKAPKLLIYSASFLYSGVPSRFSGSRSGTDFTLTISSLQPEDFA
TYYCQQHYTTPPTFGQGTKVEIK
SEQ ID NO: 66
GCATCTCTGCCTTACCTGCAGAAAGAAAGCGTGTTCCAGTCTGGCGCCCACGCCTACAGAATTCCCGCTCTGCTGTATCTGCCAG
GCCAGCAGTCTCTGCTGGCTTTCGCTGAACAGCGGGCCAGCAAGAAGGATGAGCACGCCGAACTGATCGTGCTGCGGAGAGGCGA
TTACGACGCCCCTACACATCAGGTGCAGTGGCAGGCTCAAGAGGTGGTGGCTCAGGCTAGACTGGACGGCCACAGATCTATGAAC
CCCTGTCCTCTGTACGATGCCCAGACCGGCACACTGTTTCTGTTCTTTATCGCTATCCCCGGCCAAGTGACCGAGCAGCAGCAGC
TGCAGACAAGAGCCAACGTGACCAGACTGTGTCAAGTGACCTCCACCGACCACGGCAGAACCTGGTCTAGCCCTAGAGATCTGAC
CGACGCCGCCATCGGACCTGCCTATAGAGAGTGGTCCACCTTCGCCGTTGGACCTGGACACTGTCTCCAGCTGCACGACAGGGCT
AGATCTCTGGTGGTGCCTGCCTACGCCTATAGAAAGCTGCACCCCAAACAGCGGCCTATTCCTAGCGCCTTCTGCTTTCTGAGCC
ACGATCACGGCAGGACATGGGCCAGAGGACATTTCGTGGCCCAGGACACACTGGAATGCCAGGTGGCCGAAGTGGAAACCGGCGA
GCAGAGAGTCGTGACCCTGAACGCCAGATCTCACCTGAGAGCCAGAGTGCAGGCCCAGAGCACAAACGACGGCCTGGATTTCCAA
GAGAGCCAGCTGGTCAAGAAACTGGTGGAACCTCCTCCACAGGGCTGTCAGGGAAGCGTGATCAGCTTTCCATCTCCTAGAAGCG
GCCCTGGCTCTCCTGCTCAGTGGCTGCTGTATACACACCCCACACACAGCTGGCAGAGAGCCGATCTGGGCGCCTACCTGAATCC
TAGACCTCCTGCTCCTGAGGCTTGGAGCGAACCTGTTCTGCTGGCCAAGGGCAGCTGTGCCTACAGCGATCTGCAGTCTATGGGC
ACAGGCCCTGATGGCAGCCCTCTGTTTGGCTGTCTGTACGAGGCCAACGACTACGAAGAGATCGTGTTCCTGATGTTCACCCTGA
AGCAGGCCTTTCCAGCCGAGTACCTGCCTCAAGGCGGAGGTGGAAGTGGCGGAGGCGGATCcGACAAAACTCACACATGCCCACC
GTGCCCAGCACCTGAACTCCTGGGGGGACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTACGTGGACGGCGTGGAGGTGC
ATAATGCCAAGACAAAGCCGCGGGAGGAGCAGTACAACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAGCCCCCATCGAGAAAACCATCTCCAAAGCCAAAGGG
CAGCCCCGAGAACCACAGGTcTACACCCTGCCCCCATCCCGGGAGGAGATGACCAAGAACCAGGTCAGCCTGACCTGCCTGGTCA
AAGGCTTCTATCCCAGCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAACGTCTTCTCATGCTCC
GTGATGCATGAGGCTCTGCACAACCACTACACGCAGAAGAGCCTCTCCCTGTCTCCGGGTAAAGGAGGCGGAGGATCTGGCGGAG
GTGGAAGTGGCGGAGGCGGATCTGAGGTGCAGCTGGTTGAATCTGGCGGAGGACTGGTTCAGCCTGGCGGATCTCTGAGACTGTC
TTGTGCCGCCAGCGGCTTCAACATCAAGGACACCTACATCCACTGGGTCCGACAGGCCCCTGGCAAAGGACTTGAATGGGTCGCC
AGAATCTACCCCACCAACGGCTACACCAGATACGCCGACTCTGTGAAGGGCAGATTCACCATCAGCGCCGACACCAGCAAGAACA
CCGCCTACCTGCAGATGAACAGCCTGAGAGCCGAGGACACCGCCGTGTACTACTGTTCTAGATGGGGAGGCGACGGCTTCTACGC
CATGGATTATTGGGGCCAGGGCACCCTGGTCACCGTTTCTTCTGGCGGAGGAGGATCTGGCGGAGGCGGAAGTGGCGGAGGCGGA
TCTGACATCCAGATGACACAGAGCCCTAGCAGCCTGTCTGCCAGCGTGGGAGACAGAGTGACCATCACCTGTAGAGCCAGCCAGG
ACGTGAACACAGCCGTGGCTTGGTATCAGCAGAAGCCTGGCAAGGCCCCTAAGCTGCTGATCTACAGCGCCAGCTTTCTGTACTC
CGGCGTGCCCAGCAGATTCAGCGGCTCTAGAAGCGGCACCGACTTCACCCTGACCATAAGCAGTCTGCAGCCCGAGGACTTCGCC
ACCTACTACTGTCAGCAGCACTACACCACACCTCCAACCTTTGGCCAGGGCACCAAGGTGGAAATCAG
SEQ ID NO: 67
DIQMTQSPSSLSASVGDRVTITCRASQDVNTAVAWYQQKPGKAPKLLIYSASFLYSGVPSRFSGSRSGTDFTLTISSLQPEDFAT
YYCQQHYTTPPTFGQGTKVEIKRTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC
SEQ ID NO: 68
GACATCCAGATGACACAGAGCCCTAGCAGCCTGTCTGCCAGCGTGGGAGACAGAGTGACCATCACCTGTAGAGCCAGCCAGGACG
TGAACACAGCCGTGGCTTGGTATCAGCAGAAGCCTGGCAAGGCCCCTAAGCTGCTGATCTACAGCGCCAGCTTTCTGTACTCCGG
CGTGCCCAGCAGATTCAGCGGCTCTAGAAGCGGCACCGACTTCACCCTGACCATAAGCAGTCTGCAGCCCGAGGACTTCGCCACC
TACTACTGTCAGCAGCACTACACCACACCTCCAACCTTTGGCCAGGGCACCAAGGTGGAAATCAAGCGTACGGTGGCTGCACCAT
CTGTCTTCATCTTCCCGCCATCTGATGAGCAGTTGAAATCTGGAACTGCCTCTGTTGTGTGCCTGCTGAATAACTTCTATCCCAG
AGAGGCCAAAGTACAGTGGAAGGTGGATAACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACACAAAGTCTACGCCTGCGAAGTCACCCATC
AGGGCCTGAGCTCGCCCGTCACAAAGAGCTTCAACAGGGGAGAGTGT
SEQ ID NO: 69
DKTHTCPPCPAPELLGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 70:
MASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSCAYSDLQSM
GTGPDGSPLFGCLYEANDYEEIVFLMFTLKQAFPAEYLPQ
SEQ ID NO: 71:
MASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGCLYEANDYEEIVFLMFTLKQAFPAEYLPQ
SEQ ID NO: 72:
AASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSCAYSDLQSM
GTGPDGSPLFGCLYEANDYEEIVFLMFTLKQAFPAEYLPQ
SEQ ID NO: 73:
DASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSCAYSDLQSM
GTGPDGSPLFGCLYEANDYEEIVFLMFTLKQAFPAEYLPQ
SEQ ID NO: 74:
MASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSCAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGKGGGGSGGGGSGGGGSEVQLVESGGGLVQPGGSLRLSCARSGFNIKDTYIHWVRQAPGKGLEWV
ARIYPTNGYTRYADSVKGRFTISADTSKNTAYLQMNSLRAEDTAVYYCSRWGGDGFYAMDYWGQGTLVTVSSGGGGSGGGGSGGG
GSDIQMTQSPSSLSASVGDRVTITCRASQDVNTAVAWYQQKPGKAPKLLIYSASFLYSGVPSRFSGSRSGTDFTLTISSLQPEDF
ATYYCQQHYTTPPTFGQGTKVEIK
SEQ ID NO: 75:
MASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGKGGGGSGGGGSGGGGSEVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYCSRWGGDGFYAMDYWGQGTLVTVSSGGGGSGGGGSGGG
GSDIQMTQSPSSLSASVGDRVTITCRASQDVNTAVAWYQQKPGKAPKLLIYSASFLYSGVPSRFSGSRSGTDFTLTISSLQPEDF
ATYYCQQHYTTPPTFGQGTKVEIK
SEQ ID NO: 76:
AASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSCAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLTSKLTVDKSRWQQGNVFSC
SVMHEALHNHYTQKSLSLSPGK
SEQ ID NO: 77:
DASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSCAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLTSKLTVDKSRWQQGNVFSC
SVMHEALHNHYTQKSLSLSPGK
SEQ ID NO: 78:
MASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSCAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLTSKLTVDKSRWQQGNVFSC
SVMHEALHNHYTQKSLSLSPGK
SEQ ID NO: 79:
MASLPYLQKESVFQSGAHAYRIPALLYLPGQQSLLAFAEQRASKKDEHAELIVLRRGDYDAPTHQVQWQAQEVVAQARLDGHRSM
NPCPLYDAQTGTLFLFFIAIPGQVTEQQQLQTRANVTRLCQVTSTDHGRTWSSPRDLTDAAIGPAYREWSTFAVGPGHCLQLHDR
ARSLVVPAYAYRKLHPKQRPIPSAFCFLSHDHGRTWARGHFVAQDTLECQVAEVETGEQRVVTLNARSHLRARVQAQSTNDGLDF
QESQLVKKLVEPPPQGCQGSVISFPSPRSGPGSPAQWLLYTHPTHSWQRADLGAYLNPRPPAPEAWSEPVLLAKGSAAYSDLQSM
GTGPDGSPLFGCLYEANDYEEIVFLMFTLKQAFPAEYLPQGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLTSKLTVDKSRWQQGNVFSC
SVMHEALHNHYTQKSLSLSPGK
SEQ ID NO: 80:
ACAGTGGAAAAGTCCGTGGTGTTCAAGGCCGAGGGCGAGCACTTCACCGACCAGAAAGGCAATACCATCGTCGGCTCTGGCAGCG
GCGGCACCACCAAGTACTTTAGAATCCCCGCCATGTGCACCACCAGCAAGGGCACCATTGTGGTGTTCGCCGACGCCAGACACAA
CACCGCCAGCGATCAGAGCTTCATCGATACCGCTGCCGCCAGATCTACCGATGGCGGCAAGACCTGGAACAAGAAGATCGCCATC
TACAACGACCGCGTGAACAGCAAGCTGAGCAGAGTGATGGACCCTACCTGCATCGTGGCCAACATCCAGGGCAGAGAAACCATCC
TGGTCATGGTCGGAAAGTGGAACAACAACGATAAGACCTGGGGCGCCTACAGAGACAAGGCCCCTGATACCGATTGGGACCTCGT
GCTGTACAAGAGCACCGATGACGGCGTGACCTTCAGCAAGGTGGAAACAAACATCCACGACATCGTGACCAAGAACGGCACCATC
TCTGCCATGCTCGGCGGCGTTGGATCTGGCCTGCAACTGAATGATGGCAAGCTGGTGTTCCCCGTGCAGATGGTCCGAACAAAGA
ATATCACCACCGTGCTGAATACCAGCTTCATCTACAGCACCGACGGCATCACATGGTCCCTGCCTAGCGGCTACTGTGAAGGCTT
TGGCAGCGAGAACAACATCATCGAGTTCAACGCCAGCCTGGTCAACAACATCCGGAACAGCGGCCTGCGGAGAAGCTTCGAGACA
AAGGACTTCGGAAAGACGTGGACCGAGTTTCCTCCAATGGACAAGAAGGTGGACAACCGGAACCACGGCGTGCAGGGCAGCACAA
TCACAATCCCTAGCGGCAACAAACTGGTGGCCGCTCACTCTAGCGCCCAGAACAAGAACAACGACTACACCAGAAGCGACATCAG
CCTGTACGCCCACAACCTGTACAGCGGCGAAGTGAAGCTGATCGACGACTTCTACCCCAAAGTGGGCAATGCCAGCGGAGCCGGC
TACAGCTGTCTGAGCTACCGGAAAAATGTGGACAAAGAAACCCTGTACGTGGTGTACGAGGCCAACGGCAGCATCGAGTTTCAGG
ACCTGAGCAGACATCTGCCCGTGATCAAGAGCTACAAC
SEQ ID NO: 81:
MRFKNVKKTALMLAMFGMATSSNAALFDYNATGDTEFDSPAKQGWMQDNTNNGSGVLTNADGMPAWLVQGIGGRAQWTYSLSTNQ
HAQASSFGWRMTTEMKVLSGGMITNYYANGTQRVLPIISLDSSGNLVVEFEGQTGRTVLATGTAATEYHKFELVFLPGSNPSASF
YFDGKLIRDNIQPTASKQNMIVWGNGSSNTDGVAAYRDIKFEIQGDVIFRGPDRIPSIVASSVTPGVVTAFAEKRVGGGDPGALS
NTNDIITRTSRDGGITWDTELNLTEQINVSDEFDFSDPRPIYDPSSNTVLVSYARWPTDAAQNGDRIKPWMPNGIFYSVYDVASG
NWQAPIDVTDQVKERSFQIAGWGGSELYRRNTSLNSQQDWQSNAKIRIVDGAANQIQVADGSRKYVVTLSIDESGGLVANLNGVS
APIILQSEHAKVHSFHDYELQYSALNHTTTLFVDGQQITTWAGEVSQENNIQFGNADAQIDGRLHVQKIVLTQQGHNLVEFDAFY
LAQQTPEVEKDLEKLGWTKIKTGNTMSLYGNASVNPGPGHGITLTRQQNISGSQNGRLIYPAIVLDRFFLNVMSIYSDDGGSNWQ
TGSTLPIPFRWKSSSILETLEPSEADMVELQNGDLLLTARLDFNQIVNGVNYSPRQQFLSKDGGITWSLLEANNANVFSNISTGT
VDASITRFEQSDGSHFLLFTNPQGNPAGTNGRQNLGLWFSFDEGVTWKGPIQLVNGASAYSDIYQLDSENAIVIVETDNSNMRIL
RMPITLLKQKLTLSQN
SEQ ID NO: 82:
TTGTCAATCAAGATGACTTCACAACGAAGAAGAGCATCGATTCACAAGGAAACAGATTCTAATATAAAGGGAGTAGATATGCGTT
TCAAAAACGTAAAGAAAACCGCTTTAATGCTTGCAATGTTCGGTATGGCGACAAGCTCAAACGCCGCACTTTTTGACTATAACGC
AACGGGTGACACTGAGTTTGACAGTCCAGCCAAACAGGGATGGATGCAAGACAACACGAATAATGGCAGCGGCGTTTTAACCAAT
GCAGATGGAATGCCCGCTTGGTTGGTGCAAGGTATTGGAGGGAGAGCTCAATGGACATATTCTCTCTCTACTAATCAACATGCCC
AAGCATCAAGTTTCGGTTGGCGAATGACGACAGAAATGAAAGTGCTCAGTGGTGGAATGATCACAAACTACTACGCCAACGGCAC
TCAGCGTGTCTTACCCATCATTTCATTAGATAGCAGTGGTAACTTAGTTGTTGAGTTTGAAGGGCAAACTGGACGCACCGTTTTG
GCAACCGGCACAGCAGCAACGGAATATCATAAATTTGAATTGGTATTCCTTCCTGGAAGTAACCCATCCGCTAGCTTTTACTTCG
ATGGCAAACTCATTCGTGACAACATCCAGCCGACTGCATCAAAACAAAATATGATCGTATGGGGGAATGGCTCATCAAATACGGA
TGGTGTCGCCGCTTATCGTGATATTAAGTTTGAAATTCAAGGCGACGTCATCTTCAGAGGCCCAGACCGTATACCGTCCATTGTA
GCAAGTAGCGTAACACCAGGGGTGGTAACCGCATTTGCAGAGAAACGTGTGGGGGGAGGAGATCCCGGTGCTCTGAGTAATACCA
ATGACATAATCACTCGTACCTCACGAGATGGCGGTATAACTTGGGATACCGAGCTCAACCTCACTGAGCAAATCAATGTCAGTGA
TGAGTTTGATTTCTCCGATCCTCGGCCTATCTATGATCCTTCCTCCAATACGGTTCTTGTCTCTTATGCTCGATGGCCGACCGAT
GCCGCTCAAAACGGAGATCGAATAAAACCATGGATGCCAAACGGTATTTTTTACAGCGTCTATGATGTTGCATCAGGGAACTGGC
AAGCGCCTATCGATGTTACCGATCAGGTGAAAGAACGCAGTTTCCAAATCGCTGGTTGGGGTGGTTCAGAGCTGTATCGCCGAAA
TACCAGCCTAAATAGCCAGCAAGACTGGCAATCAAACGCTAAGATCCGAATTGTTGATGGTGCAGCGAACCAGATACAAGTTGCC
GATGGTAGCCGAAAATATGTTGTCACACTGAGTATTGATGAATCAGGTGGTCTAGTCGCTAATCTAAACGGTGTTAGTGCTCCGA
TTATCCTGCAATCTGAACACGCAAAGGTACACTCTTTCCATGACTACGAACTTCAATATTCGGCGTTAAACCACACCACAACGTT
ATTCGTGGATGGTCAGCAAATCACAACTTGGGCTGGCGAAGTATCGCAGGAGAACAACATTCAGTTTGGTAATGCGGATGCCCAA
ATTGACGGCAGACTGCATGTGCAAAAAATTGTTCTCACACAGCAAGGCCATAACCTCGTGGAGTTTGATGCTTTCTATTTAGCAC
AGCAAACCCCTGAAGTAGAGAAAGACCTTGAAAAGCTTGGTTGGACAAAAATTAAAACGGGCAACACCATGAGTTTGTATGGAAA
TGCCAGTGTCAACCCAGGACCGGGTCATGGCATCACCCTTACTCGACAACAAAATATCAGTGGCAGCCAAAACGGCCGCTTGATC
TACCCAGCGATTGTGCTTGATCGTTTCTTCTTGAACGTCATGTCTATTTACAGTGATGATGGCGGTTCAAACTGGCAAACCGGTT
CAACACTCCCTATCCCCTTTCGCTGGAAGAGTTCGAGTATCCTAGAAACTCTCGAACCTAGTGAAGCTGATATGGTTGAACTCCA
AAACGGTGATCTACTCCTTACTGCACGCCTTGATTTTAACCAAATCGTTAATGGTGTGAACTATAGCCCACGCCAGCAATTTTTG
AGTAAAGATGGTGGAATCACGTGGAGCCTACTTGAGGCTAACAACGCTAACGTCTTTAGCAATATCAGTACTGGTACCGTTGATG
CTTCTATTACTCGGTTCGAGCAAAGTGACGGTAGCCATTTCTTACTCTTTACTAACCCACAAGGAAACCCTGCGGGGACAAATGG
CAGGCAAAATCTAGGCTTATGGTTTAGCTTCGATGAAGGGGTGACATGGAAAGGACCAATTCAACTTGTTAATGGTGCATCGGCA
TATTCTGATATTTATCAATTGGATTCGGAAAATGCGATTGTCATTGTTGAAACGGATAATTCAAATATGCGAATTCTTCGTATGC
CTATCACATTGCTAAAACAGAAGCTGACCTTATCGCAAAACTAA
SEQ ID NO: 83:
MVGADPTRPRGPLSYWAGRRGQGLAAIFLLLVSAAESEARAEDDFSLVQPLVTMEQLLWVSGKQIGSVDTFRIPLITATPRGTLL
AFAEARKKSASDEGAKFIAMRRSTDQGSTWSSTAFIVDDGEASDGLNLGAVVNDVDTGIVFLIYTLCAHKVNCQVASTMLVWSKD
DGISWSPPRNLSVDIGTEMFAPGPGSGIQKQREPGKGRLIVCGHGTLERDGVFCLLSDDHGASWHYGTGVSGIPFGQPKHDHDFN
PDECQPYELPDGSVIINARNQNNYHCRCRIVLRSYDACDTLRPRDVTFDPELVDPVVAAGALATSSGIVFFSNPAHPEFRVNLTL
RWSFSNGTSWLKERVQVWPGPSGYSSLTALENSTDGKKQPPQLFVLYEKGLNRYTESISMVKISVYGTL
SEQ ID NO: 84:
MTVQPSPWFSDLRPMATCPVLQKETLFRTGVHAYRIPALLYLKKQKTLLAFAEKRASKTDEHAELIVLRRGSYNEATNRVKWQPE
EVVTQAQLEGHRSMNPCPLYDKQTKTLFLFFIAVPGRVSEHHQLHTKVNVTRLCCVSSTDHGRTWSP1QDLTETTIGSTHQEWAT
FAVGPGHCLQLRNPAGSLLVPAYAYRKLHPAQKPTPFAFCFISLDHGHTWKLGNFVAENSLECQVAEVGTGAQRMVYLNARSFLG
ARVQAQSPNDGLDFQDNRVVSKLVEPPHGCHGSVVAFHNPISKPHALDTWLLYTHPTDSRNRTNLGVYLNQMPLDPTAWSEPTLL
AMGICAYSDLQNMGQGPDGSPQFGCLYESGNYEEIIFLIFTLKQAFPTVFDAQ
SEQ ID NO: 85:
MEEVPPYSLSSTLFQQEEQSGVTYRIPALLYLPPTHTFLAFAEKRTSVRDEDAACLVLRRGLMKGRSVQWGPQRLLMEATLPGHR
TMNPCPVWEKNTGRVYLFFICVRGHVTERCQIVWGKNAARLCFLCSEDAGCSWGEVKDLTEEVIGSEVKRWATFAVGPGHGIQLH
SGRLIIPAYAYYVSRWFLCFACSVKPHSLMIYSDDFGVTWHHGKFIEPQVTGECQVAEVAGTAGNPVLYCSARTPSRFRAEAFST
DSGGCFQKPTLNPQLHEPRTGCQGSVVSFRPLKMPNTYQDSIGKGAPATQKCPLLDSPLEVEKGAETPSATWLLYSHPTSKRKRI
NLGIYYNRNPLEVNCWSRPWILNRGPSGYSDLAVVEEQDLVACLFECGEKNEYERIDFCLFSDHEVLSCEDCTSPSSD
SEQ ID NO: 86:
METAGAPFCFHVDSLVPCSYWKVMGPTRVPRRTVLFQRERTGLTYRVPALLCVPPRPTLLAFAEQRLSPDDSHAHRLVLRRGTLT
RGSVRWGTLSVLETAVLEEHRSMNPCPVLDEHSGTIFLFFIAVLGHTPEAVQIATGKNAARLCCVTSCDAGLTWGSVRDLTEEAI
GAALQDWATFAVGPGHGVQLRSGRLLVPAYTYHVDRRECFGKICWTSPHSLAFYSDDHGISWHCGGLVPNLRSGECQLAAVDGDF
LYCNARSPLGNRVQALSADEGTSFLPGELVPTLAETARGCQGSIVGFLAPPSIEPQDDRWTGSPRNTPHSPCFNLRVQESSGEGA
RGLLERWMPRLPLCYPQSRSPENHGLEPGSDGDKTSWTPECPMSSDSMLQSPTWLLYSHPAGRRARLHMGIYLSRSPLDPHSWTE
PWVIYEGPSGYSDLAFLGPMPGASLVFACLFESGTRTSYEDISFCLFSLADVLENVPTGLEMLSLRDKAQGHCWPS
SEQ ID NO: 87:
GGGTCACATGCTGATGGACTAATTGGAGTCGCGGCAGCGCGGGCTGCGGCCCCCAAGGGGAGGGGTCGGAGTGACGTGCGCGCTT
TTAAAGGGCCGAGGTCAGCTGACGGCTTGCCACCGGTGACCAGTTCCTGGACAGGGATCGCCGGGAGCTATGGTGGGGGCAGACC
CGACCAGACCCCGGGGACCGCTGAGCTATTGGGCGGGCCGTCGGGGTCAGGGGCTCGCAGCGATCTTCCTGCTCCTGGTGTCCGC
GGCGGAATCCGAGGCCAGGGCAGAGGATGACTTCAGCCTGGTGCAGCCGCTGGTGACCATGGAGCAGCTGCTGTGGGTGAGCGGG
AAGCAGATCGGCTCTGTAGACACTTTCCGCATCCCGCTCATCACAGCCACCCCTCGGGGCACGCTCCTGGCCTTCGCTGAGGCCA
GGAAAAAATCTGCATCCGATGAGGGGGCCAAGTTCATCGCCATGAGGAGGTCCACGGACCAGGGTAGCACGTGGTCCTCTACAGC
CTTCATCGTAGACGATGGGGAGGCCTCCGATGGCCTGAACCTGGGCGCTGTGGTGAACGATGTAGACACAGGGATAGTGTTCCTT
ATCTATACCCTCTGTGCTCACAAGGTCAACTGCCAGGTGGCCTCTACCATGTTGGTTTGGAGTAAGGACGACGGCATTTCCTGGA
GCCCACCCCGGAATCTCTCTGTGGATATTGGCACAGAGATGTTTGCCCCTGGACCTGGCTCAGGCATTCAGAAACAGCGGGAGCC
TGGGAAGGGCCGGCTCATTGTGTGTGGACACGGGACGCTGGAGCGAGATGGGGTCTTCTGTCTCCTCAGTGATGACCACGGTGCC
TCCTGGCACTACGGCACTGGAGTGAGCGGCATTCCCTTTGGCCAGCCCAAACACGATCACGATTTCAACCCCGACGAGTGCCAGC
CCTACGAGCTTCCAGATGGCTCGGTCATCATCAACGCCCGGAACCAGAATAACTACCATTGCCGCTGCAGGATCGTCCTCCGCAG
CTATGACGCCTGTGACACCCTCAGGCCCCGGGATGTGACCTTCGACCCTGAGCTCGTGGACCCTGTGGTAGCTGCAGGAGCACTA
GCCACCAGCTCCGGCATTGTCTTCTTCTCCAATCCAGCCCACCCTGAGTTCCGAGTGAACCTGACCCTGCGCTGGAGTTTCAGCA
ATGGTACATCCTGGCAGAAGGAGAGGGTCCAGGTGTGGCCGGGACCCAGCGGCTACTCGTCCCTGACAGCCCTGGAAAACAGCAC
GGATGGAAAGAAGCAGCCCCCGCAGCTGTTCGTTCTGTACGAGAAAGGCCTGAACCGGTACACCGAGAGCATCTCCATGGTCAAA
ATCAGCGTCTACGGCACGCTCTGAGCCCCGTGCCCAAAGGACACCAAGTCCTGGTCGCTGACTTCACAGCTCTCTGGACCATCTG
CAGAGGGTGCCTGAAACACAGCTCTTCCTCTGAACTCTGACCTTTTGCAACTTCTCATCAACAGGGAAGTCTCTTCGTTATGACT
TAACACCCAGCTTCCTCTCGGGGCAGGAAGTCCCTCCGTCACCAAGAGCACTTTTTTCCAGTATGCTGGGGATGGCCCCTGTCCA
TTCTCTTCCAGGACAACGGAGCTGTGCCTTTCTGGGACAGGATGGGGGAGGGGCTCCCCCTGGAGAGATGAACAGATACGAACTC
AGGGAACTGAGAAGGCCCGGTGTCCTAGGGTACAAAGGCAGGTACTAGATGTGATTGCTGAAAGTCCCCAGGGCAGAGTGTCCTT
TCAGAGCAAGGATAAGCACACCTACGTGTGCACCTTTGATTATTTATGAATCGAAATATTTGTAACTTAAAATTTTTGATGCAGA
AAAAGCGTTTGTGGAGTCTGTGGTTCTGTCTGCTCACGCCTTCCCAATTGCCTCCTGGAGAGACAGGAAGGCAGCTGGAAGAGGA
GCCGATGTACTTACTGGGAAGCAGAAACCCCTAGATTCCATCCTGGCTGCTGCTGTTTGCAAGTGTCAAAGATGGGGGGGCGTGT
TTATATTTTATATTTCTAAGATGGGGTGGCATAGGAAATAGGGAACAGATGTGTAAAACCAGATGGGAAGGACAGTCTGTGAGAA
AGGAGCAAGCAGTTGCTGCAGGTGTGGGAGAGCAAAGCCCTTCTCCACGTGGAAAGAGCCCAGATGGACGCTAAGCATGTTGGGC
ACCTGTAACCCCGCACTCGCTGGACTGACGGTGTAGCTCAGTGGTGGAGCTAGTACTTGGAACGCCTAAGACTCTGGGTTCAGTC
CTTGGGGGGGGGGGTATGTGTTTATTGAGAGGAAGGTGTACGTACTGTAGGTCAGAGGACAGCTTACTGGAGTTGTCTCTCTCCT
TCACGCTGTGAGTCCTGTGGAATGACCTCAGGTGTCAGAGTTGGGGGCAGGTGCCTTTGCCAGCTGAGCCATCTTGCTGTCTCTG
CTTTAATTTAAAAAAAAAAAAAAAAAAGAATATTAAGGTCTGAGGGATTCGGGCTGCGTTCATTTCAATTAGAGGGTCATATTTC
TTTTGACATTTCTTCTCTAAGAAATGTTAAGATCATTTGTTCTGTGTGATAGAGGTATAGCTCCATTGTATGTCAGCAGTGAGGG
ATCCTGTGCATTTTATCCAGAGTTTGTACGGTGTTCTAGGGGCTGCTAGTGCAGCCCAGTGCTAAACACTTCAGCATGCACAAGG
CCTCAATCAGTGCATGCATGTGCACACACACACAGACACACACGTACACACTGACACAGGTACACAAATACACACTGGCCCACAT
GTACACATCGACTCACAGGTACACAGACCCACTTTGACACACATATACACAGACACAAACGCACTGGCACACACATATACACAGG
CACACATGGATAGATGGACACACGTGTACACATACACACACACACAGAAATACAAATGTTCAGGTTTTCTAAAAAAAAAAAAATT
AGAGACGTGTTGACTTCATTTTTAGCAAAAATCCTGTCATGTATCTTAAAGTGGATTGAACCCACTATGTAGCCCAGGCTGGCCT
CCAAATGGGCATCCTTCTGCCTCAGTCTCCCGAGGGCTAGGATAACAGGAGTATGCCATCACACCTGGCTAATAGAAATTTTCAA
AATTGTTTGTTTGAAGGTGACTCTTACTATATTGCCTAACTGATCTCCAGTTCGTGAAATCCTCCTGCCTCAGAACCAGGACTGT
CAATATAACCCACCAAGACAGGCCAACATTCACAATTGATTGTTAGTTTGTGGTCTGAATCAAGGTCTTATACTGTAGCCCAGGC
TAGCCCGGAATACACGATATCTCCAGTGCTTCAGATCCTCAGTTCTAACTAAGCATGGCCACATCCATGTTTAACTGCAAATTTG
ATGTTACCATGGTTTGGTTTGGTTTGGTTTGGTTTGGTTTGGTTTGGTTTGGTTTTTTGGCCATTTTTTTTTTCTCATGCTGAGG
CCTTGTGCTCTCAAGTTGGGGAGACAGCATGGAGGGTAGCTGCAACTGTAACCCCAGTTCCAGGGGACCTGACACCCTCTGGCCT
CCACAAGTATTAGGCACATCTGTGGTGCACAGACATACAATCAGGCAAAATATTCATACACATAAAATAAAATAATTTAAAACAA
AAGCAAAAATCAGGACCTAAGAAAAAAATCTATTCCTGATTCTTTTATGTTTTGTTTGTATTTTATCAAGACAGGGTTGTTTCTC
TGTATAGCCCTGGCTGTCTTGGAATTCACTCTGTAGACCAGGCTGGCCTCAAACTCAGAAATCCTCCTGCCTTTGCCTTCCAAGT
GCTGGAATTAAAGGCATGCGCCACC
SEQ ID NO: 88:
GACATGACCCAAACGGCCCCTGGCTGCAAGGTAATATCGGAAGTTGACTAAGAATGGACGCCCCACCACTGACTGACCCGCCCCC
TGAGTCTGAGATTGGACTTGTCTCTGGATACAGTCATACTTTGAGGTACTACAAGTTAGAAACTGTTAGGTTACTCAGTTCAGTC
CATGACAGTCCAACCTTCTCCATGGTTTTCCGATCTCAGGCCCATGGCGACCTGCCCTGTCCTGCAGAAGGAGACACTGTTCCGC
ACAGGCGTCCATGCTTACAGAATCCCTGCTCTGCTCTACCTGAAGAAGCAGAAGACCCTGCTGGCCTTTGCGGAAAAGCGAGCCA
GCAAGACGGATGAGCACGCAGAGTTGATTGTCCTGAGAAGAGGAAGCTACAACGAAGCCACCAACCGTGTCAAGTGGCAGCCTGA
GGAAGTGGTGACCCAAGCCCAGCTGGAAGGCCACCGCTCCATGAATCCATGTCCCTTGTATGACAAGCAAACAAAGACCCTCTTC
CTTTTCTTCATCGCTGTCCCTGGGCGTGTATCAGAACATCATCAGCTCCACACTAAGGTTAATGTCACACGGCTGTGCTGTGTCA
GCAGCACTGACCATGGGAGGACCTGGAGCCCCATCCAGGACCTCACAGAGACCACCATTGGCAGCACTCATCAGGAATGGGCCAC
ATTTGCTGTGGGTCCTGGGCATTGTCTGCAGCTGCGGAACCCAGCTGGGAGCCTGCTGGTACCTGCTTATGCCTACCGGAAACTG
CACCCTGCTCAGAAGCCTACCCCCTTTGCCTTCTGCTTCATCAGCCTTGACCATGGGCACACATGGAAACTAGGCAACTTTGTGG
CTGAAAACTCACTGGAGTGCCAGGTGGCTGAGGTTGGCACTGGAGCTCAGAGGATGGTATATCTCAATGCTAGGAGCTTCCTGGG
AGCCAGGGTCCAGGCACAAAGTCCTAATGATGGTCTGGATTTCCAGGACAACCGGGTAGTGAGTAAGCTTGTAGAGCCCCCCCAC
GGGTGTCATGGAAGTGTGGTTGCCTTCCACAACCCCATCTCTAAGCCACATGCCTTAGACACATGGCTTCTTTATACACACCCTA
CAGACTCCAGGAATAGAACCAACCTGGGTGTGTACCTAAACCAGATGCCACTAGATCCCACAGCCTGGTCAGAGCCCACCCTGCT
GGCCATGGGCATCTGTGCCTACTCAGACTTACAGAACATGGGGCAAGGCCCTGATGGCTCCCCACAGTTTGGGTGTCTGTATGAA
TCAGGTAACTATGAAGAGATCATTTTCCTCATATTCACCCTGAAGCAAGCTTTCCCCACTGTATTTGATGCCCAGTGATCTCAGT
GCACGTGGCCCAAAGGGCTTCCTTGTGCTTCAAAACACCCATCTCTCTTTGCTTCCAGCATCCTCTGGACTCTTGAGTCCAGCTC
TTGGGTAACTTCCTCAGGAGGATGCAGAGAATTTGGTCTCTTGACTCTCTGCAGGCCTTATTGTTTCAGCCTCTGGTTCTCTTTT
CAGCCCAGAAATCAAAGGAG1CCTGGCTTTCCTCAGCCTGTTGGCAGGGCAGGTGGGGACAGTATATATAGAGGCTGCCATTCTG
CATGTCGGTTGTCACTATGCTAGTTTAACCTGCCTGTTTCCCCATGCCTAGTGTTTGAATGAGTATTAATAAAATATCCAACCCA
GCCCATTTCTTCCTGGAAAAAAA
SEQ ID NO: 89:
ACTGCGCGGTGAAGGGGCGTGGCCTGGCCGGGGAGGTTGACACCCAGACGCTGCTCTCAGTCCTCTGGCGCCTGCTCCCCAGCGC
ATTCCTTCTGCTCCTGGGATATTTGTCTCATTACTGCCAGTTCTTGCGCAGCGGTCACTGGGTTCGTTTCAGCGTCTGTGGTTTC
TGTCGCTGTTATCCAGTCTCCATCGCCCCAGCTCAGCTTCAGGCCTTCTTCCGAGACTCCACGGGAGAGCCCAGAGAGCCTCCGG
AGCCGAAGCCATGGAGGAAGTCCCACCCTACTCCCTCAGCAGCACCCTGTTCCAGCAGGAAGAACAGAGTGGGGTGACCTACCGG
ATCCCAGCCCTGCTGTACCTTCCTCCCACCCACACCTTCCTGGCCTTTGCAGAGAAGCGGACCTCAGTCAGAGATGAGGATGCTG
CCTGCCTGGTGCTCAGACGAGGGCTGATGAAGGGGCGCTCTGTACAGTGGGGCCCCCAACGGCTACTGATGGAGGCCACATTACC
TGGGCATCGCACCATGAACCCCTGCCCTGTGTGGGAGAAAAATACTGGCCGTGTGTACCTGTTTTTCATCTGTGTGCGGGGCCAT
GTTACTGAGAGGTGCCAGATTGTGTGGGGCAAAAATGCCGCCCGTCTCTGCTTCCTTTGCAGTGAAGATGCCGGCTGCTCTTGGG
GTGAAGTGAAAGACTTGACCGAGGAGGTCATTGGCTCAGAGGTGAAGCGCTGGGCCACATTTGCTGTGGGCCCAGGTCATGGCAT
CCAGCTACACTCGGGAAGGCTGATCATCCCCGCCTATGCCTACTATGTCTCACGTTGGTTTCTCTGCTTTGCGTGTTCAGTCAAG
CCCCATTCCCTGATGATCTACAGTGATGACTTTGGAGTCACATGGCACCATGGCAAGTTCATTGAGCCCCAGGTGACAGGGGAGT
GCCAAGTGGCCGAAGTGGCTGGGACGGCTGGTAACCCTGTGCTCACTGCAGTGCCCGAACACCAAGCCGATTTCGAGCAGAGGCT
TTTAGTACTGATAGTGGTGGCTGCTTTCAGAAGCCAACCCTGAACCCACAACTCCATGAGCCTCGAACCGGCTGCCAAGGTAGTG
TAGTGAGCTTCCGGCCTTTGAAGATGCCAAATACCTATCAAGACTCAATTGGCAAAGGTGCTCCCGCTACTCAGAAGTGCCCTCT
GCTGGACAGTCCTCTGGAGGTGGAGAAAGGAGCTGAAACACCATCAGCAACATGGCTCTTGTACTCACATCCAACTAGCAAGAGG
AAGAGGATTAACCTAGGCATCTACTACAACCGGAACCCCTTGGAGGTGAACTGCTGGTCCCGCCCGTGGATCTTGAACCGTGGGC
CCAGTGGCTACTCTGATCTGGCTGTTGTGGAAGAACAGGACTTGGTGGCGTGTTTGTTTGAGTGTGGGGAGAAGAATGAGTATGA
GCGGATTGACTTCTGTCTGTTTTCAGACCATGAGGTCCTGAGCTGTGAAGACTGTACCAGCCCTAGTAGCGACTAAAGCCAAATC
AAGACGGATGAGTGAGGCCCAGCTTCCCACAGAAAGGAATGGCAGCTACAGCCAGGGTAACAGAGGTCTCTGATGTCTAGAGAAA
ACTCTAAAAACTAATAATCTGCTCCTTGAATTTTTTCACTTTTCCCTTCAATGAGCATGGTGAAAATTGTGCCATATCTTACATA
ACGAGGCTCTTGAACTGGGAGTTTGAATCTCTTCTCTTCCCATTAAAAGGAGAGGCCATGTGCTCGCTTCGCGTTCGACAAAGCC
TGGATTCTGATCTTGAGTGGAAGCCACAGGCTTGTCTTTTCCAATGGTTCACTGCTCACCTGAGTATTAGGTGATGTGTAGGTGC
CTTGGCCAGAAGAAAGATCTGTGTTGTTGTATTTTTTTAAATTTATTTATTTACTATATGTAAGTACACTGCAGCTGTCTTCAGA
CACACCAGAAGAGGGCGTCAGATCTCATTAGAGATGGTTGTGAGCCACCATGTGGTTGCTGGGATTTGAACTCAGGACCTTCAGA
AGAGCAGTCAGTGCTCTTAACTACTGAGCCATCTCTCAAGCCCCGCATTGCTGTATTTTTAATAAGAAAAATGCCCTTATCCTTC
CAATAATGCCTGGAGCTGTACAAATTCTCTGTCTTAGAAGACTTGAGAAAGCAGAACTGTAAGGTCAGATGCTTTCTCCAGCCTT
GATGCTGTGTTCCACCTTCCCTTCCTCATCCAGAAAACAGTTACTAGGGAGAAAATGAGAAACCCATGCCAGCTGCCCTTGATGA
TGGTTGATAACGGTGCTTATTGCTTTTGATGTCATTACCTCTGTTAGAGATGAATCAGAGTCAGAGGTCCTTAGCTGCATCCACC
CATTTCCAGGGGGACATTCTAACACTGCTGAACAGTCAGCTAAAATGAGAGCTGTGTGTCCTAGCCTGATTCCAGGTTAGTCATG
ATGCTTCCTGGAGCTGGGCTTTTATCTAATCCCAGGAGCCATCTAGGGGAGGCTCAGAGCTAGCAGGTGATCTTCCTGAGATGGT
TTCACCGTGACAGGTGAACCATGAGCCCTTCCAAGCAAGGCCAAAGGACAACATTATAGGAAAGATTTCTAGTATTAATATGCCT
TTTCTCTGTGTGTGTACTGTCTTGTAGTGATGCTATATAGACAAATAGATGATTTCTTATTTTTTGTTTGTTTGTTTGTTTTTTT
GTTTTTCTGTAGCCCTAGCTGTCCTGGAACTCACTTTGTAAACCAGGCTGGCCTCGATCTCAGAAATCCGCCTGCCTCTGCCTCC
CGAGTGCTGGGATTAAAGGTGTGCACCACCACACCTTAATGATGATCCTATAAGTATTCCTAAAATTATACTAGTAATTATTAAC
TCCTTTATAATAGGACTGCTATTAAAGCCCTCGCTGATATGAAAACTACAGTGAGAACTCTGCCAGTCTTCACATGTCATAATTA
CTTCTGAGATAGAAAGCAGGCATTTACAACTTAGAACACATTTCTTAGAGCTGTAAAACAATTAACTAGAGGTCATAAAAGGGAA
TGAAAGATTTATTGTAGGTGCTAGGACAGAACATAAAATATTGACTGGGCTTATCTATATGAAACTTCATTGTTAACTTTTACAC
AAGAATTATGGTTTTTAACTTTCAGTGAACCTGCGGAGCTAGTGACAGAAGAGAAATGTCTAGTTAGATAACTACTCTTAATGGA
AATTCACATAAACATCTGTTGCCATCTTCTTTTTGAATTTATGTTTAAACTTGTGAATGTTTGAATTAGACACTACGCGAGCACA
TAGAAAATAAAGAACTAAGCGTGAA
SEQ ID NO: 90:
GGACAGTGTGCATCACGGAGCTTGTGGCCCAGACTGTGCCTGGCAGACCCAGAGGACCTAAGGCTTGGCTCTAGTGGTGGTCAGC
ACAGCCCTCGGTGGTCTGCGGAGCCTGATATTGCTTTACGTAAGGGCTGTTCTGCTGTGCATCTCCTGTGTCTGAAGCTATTCGC
CATGGAGACTGCTGGAGCTCCCTTCTGCTTCCATGTGGACTCCCTGGTACCTTGCTCCTACTGGAAGGTTATGGGGCCCACGCGT
GTTCCCAGGAGAACGGTGCTCTTCCAGAGGGAAAGGACGGGCCTGACCTACCGTGTGCCTGCGTTACTCTGTGTGCCTCCCAGGC
CTACTCTGCTGGCCTTCGCGGAACAGCGACTTAGCCCTGATGACTCCCATGCCCACCGCCTGGTGCTACGGAGGGGCACGCTGAC
CAGGGGCTCAGTGCGGTGGGGCACTCTGAGTGTACTGGAGACTGCAGTACTGGAGGAGCACAGGTCTATGAACCCTTGCCCGGTG
CTGGATGAGCACTCTGGTACCATCTTCCTCTTCTTCATTGCCGTGCTGGGCCACACACCGGAGGCCGTGCAAATCGCCACTGGCA
AGAACGCTGCTCGCCTCTGCTGTGTGACCAGCTGTGACGCTGGCCTCACCTGGGGCAGTGTTCGAGATCTCACTGAGGAAGCCAT
TGGTGCTGCATTGCAGGACTGGGCCACCTTTGCTGTGGGTCCGGGCCATGGAGTTCAGCTGCGCTCGGGTCGCCTGCTTGTTCCT
GCTTACACCTATCATGTGGACCGACGGGAATGTTTTGGCAAGATCTGCTGGACCAGTCCCCACTCCTTGGCATTCTACAGTGATG
ATCATGGGATCTCCTGGCATTGTGGAGGCCTTGTGCCCAACCTACGCTCTGGAGAGTGCCAACTGGCTGCGGTAGATGGAGACTT
TCTCTACTGTAATGCTCGAAGCCCTCTGGGTAACCGTGTGCAGGCACTGAGTGCTGATGAAGGCACGTCCTTCCTACCAGGGGAG
CTGGTGCCTACATTGGCAGAGACGGCTCGTGGTTGCCAGGGTAGCATTGTGGGCTTCCTAGCTCCACCCTCAATCGAGCCTCAGG
ATGACCGGTGGACAGGGAGTCCTAGGAACACCCCACATTCCCCATGCTTCAATCTCAGAGTACAGGAGTCTTCGGGGGAAGGTGC
CAGAGGTCTTCTTGAACGTTGGATGCCCAGGTTGCCTCTCTGCTACCCACAGTCCCGGAGCCCAGAGAATCATGGCCTAGAGCCT
GGGTCAGATGGAGATAAGACATCCTGGACTCCGGAATGTCCTATGTCCTCTGATTCCATGCTTCAGAGCCCCACATGGCTACTAT
ATTCCCACCCAGCAGGGCGTAGAGCTCGGCTCCACATGGGAATCTACCTGAGCCGATCCCCCTTGGATCCCCACAGCTGGACAGA
GCCCTGGGTGATCTATGAGGGCCCCAGTGGCTACTCTGACCTTGCCTTTCTTGGGCCTATGCCTGGGGCATCCCTGGTTTTTGCC
TGTCTGTTTGAGAGCGGGACCAGGACTTCCTATGAAGACATTTCTTTTTGCTTGTTCTCACTGGCGGATGTCCTGGAGAATGTGC
CCACTGGCTTAGAGATGCTAAGTCTCAGGGATAAGGCTCAGGGGCATTGCTGGCCCTCTTGATGGCCTCACCCTCTCGTAGCCGC
CTGGAGAGGAAGGGTAGACTATATAGAGGAGGTTAGGGGTAGGTCAGCATGATGCTAGGATGGAGAGAGCTCTGTCCCCTCGTGG
ATGGTGGTGGTGACTCACCCGGGGGGCCAGCTGCTTTCTGAGTGCAAATGAGAAAAATAAAGAGCTGCGCTGTGACTTTTCTTTC
CACATCAAAGCTTGGGTGTCAGTGCTTTAGCTTGATGCTCTGATCACCATGCAAATCTTCCACCGGCGCCTTGCTCAGCTTTCAT
ATCCCAAGGGTGCCTGGGAGGAAGGCAACAGGGACAGTGGACATCACTGCACCACTTTCCACGACCCTGTGTGCCAACCTCAGCC
ACTTTGAAACATGCTGATGACTGAGGTCTGTTCACTTTCTTAATTTCAAGCAGGAGAAGCAGGTTGGGGAGCCAGCCTCCCCAGC
TAGAGGGGACAGAACTTGACTTGAGCAGGGGGGTACCTCCTAGGACCTGCTCCATGTGCCTACTTCTTTACCCTTCTCTAGAGAG
GGCTCTTGTCCTGTCAGAGCTGTTTTCTCCCTTCTCTTGTTTTTTCTTTTTCAAGACTGTTTCTCTGTGTTAGCCCTGGCTGTCC
TGGATCTCACTCTGTAGATCAGGCTGACCTTGAGTTCAAAGCTCCATCTGCCTCTACTTCTCACATTACTGTGATTAAAGGCATA
TACTACCACTGCCTGGTGCCCTTTTGTATTTCTTATTAAAGTCCTAATGTCTGATTATAAAAACAGTCTGTGTGGGCTGGAGTGA
TGGCTTACTCAGTAAAGCACTTGCCATGGAATCTGGGCAATCTGAGTTTCATTTTTAGCATCCTGTAAAAATCCCAATTTGATGG
TGTACTTGTAATGTCAGCATGGAGAGGCAGAGATAGGTAAGTTCCCCAAGACTCTTTGAACCGACAGCTTGGCCTCACTGGCACA
TTCCAGGTCTCAGTGAGAGACCCTGCCTCAAAATACAAAGAAAGAGCTGCTGAAGAGTGGGTCAGAGTTGACCTCTGATCTCCGG
AAGTATATGATACACACCCGTGCATGCACTCTTCCTTACAAAATAAAAAGCAAAACAAAACCCCAACAGGTATATGGCCATTTTA
GAAAAATTAGAAGATTTAGAAAGCTATACATAAAAAAAAATGACCTAAAGAAAAATCTTTACTGTTCTGGGCACTATCCCTATCA
AACCACTGTGTTCTTTGGCCAAGCCTTGGGGTGGACACTGTTTTGAGGTGGGTCCTGTTATCTCCACTAGGTAGTGGAGTTTTGT
GTCAGACTAACTGGGTCTTAAAGCTGTCTTTAAGGCCATCAGGAGCTACTGACTTGCCTGCCTCAGCAGAGCATATCCTGAAGGT
CGGGGTTAAGTCTCCTTCCCGAGCGAGTTGCCTTCCAGTGGGCCCCTGGACTCCTAGGTCCTCAGCGCTCATCAGCTGCCAAGGA
CTCTGAGGGAATGTCCTCTGACTGTGGCCCCGAAAGGTAGGGGAGGGGGATGTGCTTAGGCTTAGGACAGGGTCCTGTTTCAGTC
TGCCTTCACTGTTAGTAGCACTGTGCCACATGGCACAGACTGGGCGAGCTTTAAAGGAAGGAGGTTGATATTGGTTCCCACTTCT
GGGGATCATGGTTGAGCAGCCTTGTCTGATGATGGTTGTCTTGATGGTAGATCGTGAGGTAGTTGATGAAGGTATGACATGGTGA
GAAACTCTGTGTGTGTGTGTTATTTTCTCTGTGTTCTACCTATACATCTATCTATGTATATATGTATCTATCTATCTACCTGGAG
GCTGGAGAGATAGCTTAGTGGTTAAGAACATTTGTTGTTCTTGCATAGTCCTGGATTTAAATTTTCAGCACCCACATGGCAGCTC
ACAACAACCCATAAATCCAGTTTCAGAGGATCCAACCTCTGATATACCATGTCAGCCAGAGCAGACACGGCTGAAGGTGGTTTGA
TCCCCGTATGGAGAGGTGACAATTGGGAAGAGAGAAAGATCAACTTAACCATGCAAGGAACAGGAAGTTAAATACTGAACAGGGA
AGGTAAAGGCAGGAAGTAGATGTAGAGGGCAAATCAATGAAACCCAAACATACCCAAATTACGCTAAACACACACTGACATGCCA
ATTAAAAGGACAAATTGGCTCCACTGGCAAAACCAAAACAGACACTGAAGATCCAAACAGTCACATGCCAACTACCGCGGAGGGA
GACAGACACAGAGAAGACCGTGACAGACACTTGGACACTCTTGAGAGTGGATGTGCAGGAAGAGAGCTCTGCCAGTGGAGAAGAA
AGCACTCAGAAGAAAGTGACAGCAGCTGTAAATTTGTATTCTGCTAATGTTATGTTCCAAAGTTGAAAGCAAAATTGTACCAATT
CATAAGAACAAACAGGCTGACTCTCAGTTGTGACTGAACGTCTCTCAGTAACTGACGGGGCGAGCAGGCCAAAGGAGAGTCGGCT
CAGAAGGGTGCATAGCCACGCCAAATCAAATAAGCAAGTACAACCGGCAGGCTCTATTTCTAGCACAAAGGGGTCTGTGCCTCAT
TCTGTGCTTGGGTCAGAGCTTGGGTCTCTCATTTGGATGTAAGTGGTGTAGTGGAGAAGCAGGAAATAATCCGGAGCGCATATTT
TGATTTTAACATAAGTGCTGATTTGGGAGGGAGTTTTGTCAAATTGTGTTTTTACAATGTTTTTTTTTTTTTAAATGATGCTTTT
TTGTAAAGTGTACAAATGTGATATAAGATTGGTTCTGCTACATTCAGTTTCTATAAAAGTGGTTCTAAAATATTGTACTGTCAAT
CATCTCATGATTATTCTACTGTACACATTACTGACTTTGTATGTAATAATTAATATTAGAAGAAAATATAATTTATTTGAATATA
AAA
SEQ ID NO: 91:
X₁ASLPX₂LQKESVFQSGAHAYRIPALLYLPGQQSLLAFAEQRASKKDEHAELIVLRRGDYDAPTHQVQWQAQEVVAQARLDGHR
SMNPCPLYDAQTGTLFLFFIAIPGQVTEQQQLQTRANVTRLCQVTSTDHGRTWSSPRDLTDAAIGPAYREWSTFAVGPGHCLQLH
DRARSLVVPAYAYRKLHPX₃QRPIPSAFCFLSHDHGRTWARGHFVAQDTLECQVAEVETGEQRVVTLNARSHLRARVQAQSTNDG
LDFQESQLVKKLVEPPPQGCQGSVISFPSPRSGPGSPAQWLLYTHPTHSWQRADLGAYLNPRPPAPEAWSEPVLLAKGSX₄AYSD
LQSMGTGPDGSPLFGCLYEANDYEEIVFLMFTLKQAFPAEYLPQ
SEQ ID NO: 92:
X₁ASLPX₂LQKESVFQSGAHAYRIPALLYLPGQQSLLAFAEQRASKKDEHAELIVLRRGDYDAPTHQVQWQAQEVVAQARLDGHR
SMNPCPLYDAQTGTLFLFFIAIPGQVTEQQQLQTRANVTRLCQVTSTDHGRTWSSPRDLTDAAIGPAYREWSTFAVGPGHCLQLH
DRARSLVVPAYAYRKLHPX₃QRPIPSAFCFLSHDHGRTWARGHFVAQDTLECQVAEVETGEQRVVTLNARSHLRARVQAQSTNDG
LDFQESQLVKKLVEPPPQGCQGSVISFPSPRSGPGSPAQWLLYTHPTHSWQRADLGAYLNPRPPAPEAWSEPVLLAKGSX₄AYSD
LQSMGTGPDGSPLFGCLYEANDYEEIVFLMFTLKQAFPAEYLPQGGGGSGGGGSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSFFLTSKLTVDKSRWQQGN
VFSCSVMHEALHNHYTQKSLSLSPGK
SEQ ID NO: 93:
X₁ASLPX₂LQKESVFQSGAHAYRIPALLYLPGQQSLLAFAEQRASKKDEHAELIVLRRGDYDAPTHQVQWQAQEVVAQARLDGHR
SMNPCPLYDAQTGTLFLFFIAIPGQVTEQQQLQTRANVTRLCQVTSTDHGRTWSSPRDLTDAAIGPAYREWSTFAVGPGHCLQLH
DRARSLVVPAYAYRKLHPX₃QRPIPSAFCFLSHDHGRTWARGHFVAQDTLECQVAEVETGEQRVVTLNARSHLRARVQAQSTNDG
LDFQESQLVKKLVEPPPQGCQGSVISFPSPRSGPGSPAQWLLYTHPTHSWQRADLGAYLNPRPPAPEAWSEPVLLAKGSX₄AYSD
LQSMGTGPDGSPLFGCLYEANDYEEIVFLMFTLKQAFPAEYLPQGGGGSGGGGSDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGKGGGGSGGGGSGGGGSEVQLVESGGGLVQPGGSLRLSCAASGFNIKDTYIHWVRQAPGKG
LEWVARIYPTNGYTRYADSVKGRFTISADTSKNTAYLQMNSLRAEDTAVYYCSRWGGDGFYAMDYWGQGTLVTVSSGGGGSGGGG
SGGGGSDIQMTQSPSSLSASVGDRVTITCRASQDVNTAVAWYQQKPGKAPKLLIYSASFLYSGVPSRFSGSRSGTDFTLTISSLQ
PEDFATYYCQQHYTTPPTFGQGTKVEIK
SEQ ID NO: 94:
ELVLTQPASVSGSPGQSITISCTGTSSDVGGYNYVSWYQQHPGKAPKLMIYDVSKRPSGVSNRFSGSKSGNTASLTISGLQAEDE
ADYYCSSYTSSSTLVFGGGTKLTVLGQPKAAPSVTLFPPSSEELQANKATLVCLISDFYPGAVTVAWKADSSPVKAGVETTTPSK
QSNNKYAASSYLSLTPEQWKSHRSYSCQVTHEGSTVEKTVAPTECS
SEQ ID NO: 95:
GAGCTCGTGTTGACTCAGCCTGCCTCCGTGTCTGGGTCTCCTGGACAGTCGATCACCATCTCCTGCACTGGAACCAGCAGTGACG
TTGGTGGTTATAACTATGTCTCCTGGTACCAACAGCACCCAGGCAAAGCCCCCAAACTCATGATTTATGATGTCAGTAAGCGGCC
CTCAGGGGTTTCTAATCGCTTCTCTGGCTCCAAGTCTGGCAACACGGCCTCCCTGACCATCTCTGGGCTCCAGGCTGAGGACGAG
GCTGATTATTACTGCAGCTCATATACAAGCAGCAGCACTTTGGTATTCGGCGGAGGGACCAAGCTGACCGTCCTAGGTCAGCCCA
AGGCGGCGCCCTCGGTCACTCTGTTCCCGCCCTCCTCTGAGGAGCTTCAAGCCAACAAGGCCACACTGGTGTGTCTCATAAGTGA
CTTCTACCCGGGAGCCGTGACAGTGGCCTGGAAGGCAGATAGCAGCCCCGTCAAGGCGGGAGTGGAGACCACCACACCCTCCAAA
CAAAGCAACAACAAGTACGCGGCCAGCAGCTACCTGAGCCTGACGCCTGAGCAGTGGAAGTCCCACAGAAGCTACAGCTGCCAGG
TCACGCATGAAGGGAGCACCGTGGAGAAGACAGTGGCCCCTACAGAATGTTCA
SEQ ID NO: 96:
EVQLLESGGGLVQPGGSLRLSCTTSGFTFNTYAMSWVRQAPGKGLEWLSGINNNGRTAFYADSVKGRFTISRDNSKNTLYLQINS
LRADDTAVYFCAKDVRFIAVPGDSWGQGTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLYCLVKGFYPSDIAVEWESNGQPENNYKTTPPVLDSDGSFFLYSKLTVDKSRWQQGNV
FSCSVMHEALHNHYTQKSLSLSPGK
SEQ ID NO: 97:
GAGGTGCAGCTGCTCGAGTCAGGGGGAGGCTTGGTACAGCCGGGGGGGTCCCTGAGACTCTCCTGTACAACCTCTGGATTCACCT
TTAACACGTATGCCATGAGTTGGGTCCGCCAGGCTCCAGGGAAGGGGCTGGAATGGCTCTCAGGTATTAATAACAATGGTCGGAC
TGCATTCTACGCAGACTCCGTGAAGGGCCGCTTCACCATCTCCAGAGACAACTCCAAAAACACACTTTATCTGCAAATTAATAGT
CTGAGAGCGGACGACACGGCCGTTTATTTCTGTGCGAAAGATGTCAGATTTATCGCAGTGCCTGGTGACTCCTGGGGCCAGGGAA
CCCTGGTCACCGTCTCCTCAGCTAGCACCAAGGGCCCATCCGTCTTCCCCCTGGCACCCTCCTCCAAGAGCACCTCTGGGGGCAC
AGCGGCCCTGGGCTGCCTGGTCAAGGACTACTTCCCCGAACCGGTGACGGTGTCCTGGAACTCAGGCGCTCTGACCAGCGGCGTG
CACACCTTCCCGGCTGTCCTACAGTCCTCAGGACTCTACTCCCTCAGCAGCGTGGTGACCGTGCCCTCCAGCAGCTTGGGCACCC
AGACCTACATCTGCAACGTGAATCACAAGCCCAGCAACACCAAGGTGGACAAGAAAGTTGAGCCCAAATCTTGTGACAAAACTCA
CACATGCCCACCGTGCCCAGCACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGTCAGCGTCCTCACCGTCCT
GCACCAGGACTGGCTGAATGGCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTCTACACCCTGCCCCCATCCCGGGAGGAGATGACCAAGAACCAGGTCAGCCTGT
ACTGCCTGGTCAAAGGCTTCTATCCCAGCGACATCGCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGTCTCCGGGTAAA
SEQ ID NO: 98:
GACGCCTCTTTACCCTATTTACAGAAGGAGAGCGTCTTTCAGTCCGGCGCTCACGCCTATAGGATCCCCGCTTTACTGTATTTAC
CCGGTCAGCAGTCTTTACTGGCTTTCGCCGAGCAGCGGGCTTCCAAGAAGGACGAGCACGCTGAGCTGATCGTGTTACGTAGGGG
AGACTACGACGCCCCCACCCATCAAGTTCAATGGCAAGCTCAAGAAGTGGTGGCTCAAGCTCGGCTCGATGGCCATCGGAGCATG
AACCCTTGTCCCCTCTACGACGCCCAAACCGGCACTTTATTTCTGTTCTTCATCGCCATCCCCGGTCAAGTTACCGAGCAGCAAC
AGCTGCAGACCCGGGCTAACGTGACAAGGCTGTGCCAAGTTACCTCCACCGACCACGGAAGGACTTGGTCCTCCCCTCGTGATCT
GACCGATGCCGCTATCGGCCCCGCTTACCGGGAGTGGTCCACCTTTGCCGTGGGACCCGGCCATTGTCTGCAGCTGCATGATAGG
GCTCGGTCTTTAGTGGTGCCCGCTTACGCCTACCGGAAGCTGCACCCCAAGCAGCGGCCTATCCCCTCCGCTTTTTGTTTTTTAA
GCCATGACCATGGTCGTACTTGGGCTCGTGGCCATTTTGTGGCCCAAGATACTTTAGAGTGCCAAGTTGCCGAGGTGGAGACTGG
TGAGCAGCGGGTGGTGACTTTAAATGCCCGGTCCCATTTAAGGGCTAGGGTGCAAGCCCAGTCCACCAACGACGGACTGGATTTC
CAAGAATCCCAGCTGGTGAAGAAGCTCGTCGAACCTCCCCCCCAAGGTTGCCAAGGAAGCGTGATCTCCTTCCCCTCCCCTAGGA
GCGGACCCGGTTCCCCCGCTCAGTGGCTGCTCTACACCCATCCCACCCATTCTTGGCAGAGGGCTGATTTAGGCGCCTATTTAAA
CCCTCGTCCTCCCGCTCCCGAAGCTTGGAGCGAGCCCGTGCTGCTCGCTAAGGGCAGCGCCGCCTACAGCGATTTACAGTCCATG
GGAACCGGACCCGATGGCAGCCCTCTGTTCGGCTGTTTATATGAGGCTAACGACTACGAGGAGATCGTGTTTCTCATGTTCACTT
TAAAGCAAGCTTTTCCCGCTGAGTATCTGCCCCAAGGTGGAGGCGGCAGCGGCGGCGGCGGCTCCGACAAAACTCACACATGCCC
ACCGTGCCCAGCACCTGAACTCCTGGGGGGACCGTCAGTCTTCCTCTTCCCCCCAAAACCCAAGGACACCCTCATGATCTCCCGG
ACCCCTGAGGTCACATGCGTGGTGGTGGACGTGAGCCACGAAGACCCTGAGGTCAAGTTCAACTGGTACGTGGACGGCGTGGAGG
TGCATAATGCCAAGACAAAGCCGCGGGAGGAGCAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAGCCCCCATCGAGAAAACCATCTCCAAAGCCAAA
GGGCAGCCCCGAGAACCACAGGTGTACACCCTGCCCCCATCCCGGGAGGAGATGACCAAGAACCAGGTCAGCCTGACCTGCCTGG
TCAAAGGCTTCTATCCCAGCGACATCGCCGTGGAGTGGGAGAGCAATGGGCAGCCGGAGAACAACTACAAGACCACGCCTCCCGT
GCTGGACTCCGACGGCTCCTTCTTCCTCACCAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTAAATGA
SEQ ID NO: 99:
ATGGCCTCTTTACCCTATTTACAGAAGGAGAGCGTCTTTCAGTCCGGCGCTCACGCCTATAGGATCCCCGCTTTACTGTATTTAC
CCGGTCAGCAGTCTTTACTGGCTTTCGCCGAGCAGCGGGCTTCCAAGAAGGACGAGCACGCTGAGCTGATCGTGTTACGTAGGGG
AGACTACGACGCCCCCACCCATCAAGTTCAATGGCAAGCTCAAGAAGTGGTGGCTCAAGCTCGGCTCGATGGCCATCGGAGCATG
AACCCTTGTCCCCTCTACGACGCCCAAACCGGCACTTTATTTCTGTTCTTCATCGCCATCCCCGGTCAAGTTACCGAGCAGCAAC
AGCTGCAGACCCGGGCTAACGTGACAAGGCTGTGCCAAGTTACCTCCACCGACCACGGAAGGACTTGGTCCTCCCCTCGTGATCT
GACCGATGCCGCTATCGGCCCCGCTTACCGGGAGTGGTCCACCTTTGCCGTGGGACCCGGCCATTGTCTGCAGCTGCATGATAGG
GCTCGGTCTTTAGTGGTGCCCGCTTACGCCTACCGGAAGCTGCACCCCAAGCAGCGGCCTATCCCCTCCGCTTTTTGTTTTTTAA
GCCATGACCATGGTCGTACTTGGGCTCGTGGCCATTTTGTGGCCCAAGATACTTTAGAGTGCCAAGTTGCCGAGGTGGAGACTGG
TGAGCAGCGGGTGGTGACTTTAAATGCCCGGTCCCATTTAAGGGCTAGGGTGCAAGCCCAGTCCACCAACGACGGACTGGATTTC
CAAGAATCCCAGCTGGTGAAGAAGCTCGTCGAACCTCCCCCCCAAGGTTGCCAAGGAAGCGTGATCTCCTTCCCCTCCCCTAGGA
GCGGACCCGGTTCCCCCGCTCAGTGGCTGCTCTACACCCATCCCACCCATTCTTGGCAGAGGGCTGATTTAGGCGCCTATTTAAA
CCCTCGTCCTCCCGCTCCCGAAGCTTGGAGCGAGCCCGTGCTGCTCGCTAAGGGCAGCTGCGCCTACAGCGATTTACAGTCCATG
GGAACCGGACCCGATGGCAGCCCTCTGTTCGGCTGTTTATATGAGGCTAACGACTACGAGGAGATCGTGTTTCTCATGTTCACTT
TAAAGCAAGCTTTTCCCGCTGAGTATCTGCCCCAAGGTGGAGGCGGCAGCGGCGGCGGCGGCTCCGACAAAACTCACACATGCCC
ACCGTGCCCAGCACCTGAACTCCTGGGGGGACCGTCAGTCTTCCTCTTCCCCCCAAAACCCAAGGACACCCTCATGATCTCCCGG
ACCCCTGAGGTCACATGCGTGGTGGTGGACGTGAGCCACGAAGACCCTGAGGTCAAGTTCAACTGGTACGTGGACGGCGTGGAGG
TGCATAATGCCAAGACAAAGCCGCGGGAGGAGCAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAGCCCCCATCGAGAAAACCATCTCCAAAGCCAAA
GGGCAGCCCCGAGAACCACAGGTGTACACCCTGCCCCCATCCCGGGAGGAGATGACCAAGAACCAGGTCAGCCTGACCTGCCTGG
TCAAAGGCTTCTATCCCAGCGACATCGCCGTGGAGTGGGAGAGCAATGGGCAGCCGGAGAACAACTACAAGACCACGCCTCCCGT
GCTGGACTCCGACGGCTCCTTCTTCCTCTACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTAAAGGAGGTGGCGGCAGCGGCG
GCGGAGGCAGCGGAGGAGGAGGCAGCGAGGTGCAGCTGGTGGAGTCCGGAGGAGGACTGGTGCAGCCCGGAGGATCTTTAAGGCT
GAGCTGTGCCGCCAGCGGCTTCAACATCAAGGACACCTACATCCACTGGGTGAGGCAAGCTCCCGGCAAAGGACTCGAGTGGGTG
GCTCGTATCTACCCCACCAACGGCTATACTCGTTACGCCGACTCCGTCAAGGGTCGTTTCACCATTTCCGCCGACACCTCCAAGA
ACACCGCCTATTTACAGATGAATTCTTTACGGGCCGAAGACACAGCTGTCTACTACTGCTCCCGGTGGGGCGGAGACGGATTCTA
CGCCATGGACTACTGGGGACAAGGTACACTGGTGACAGTGTCCAGCGGCGGAGGAGGATCTGGCGGCGGCGGAAGCGGCGGTGGC
GGTAGCGATATCCAGATGACCCAGAGCCCTTCCTCTTTAAGCGCTTCCGTGGGCGATCGTGTCACCATCACTTGTAGGGCCTCCC
AAGATGTGAACACCGCTGTGGCTTGGTACCAGCAGAAGCCCGGCAAGGCTCCCAAGCTGCTGATCTACTCCGCCAGCTTTCTGTA
TTCCGGAGTGCCTTCTCGTTTCAGCGGCTCTCGTAGCGGCACCGACTTCACTTTAACCATCAGCTCTTTACAGCCCGAGGACTTC
GCCACCTACTACTGCCAGCAGCATTACACCACACCCCCCACCTTCGGACAAGGTACCAAAGTGGAGATCAAGTGA
SEQ ID NO: 100:
X₁X₂SX₃PX₄LQKESVFQSGAHAYRIPALLYLPGQQSLLAFAEQRASKKDEHAELIVLRRGDYDAPTHQVQWQAQEVVAQARLDG
HRSMNPCPLYDAQTGTLFLFFIAIPGQVTEQQQLQTRANVTRLX₅QVTSTDHGRTWSSPRDLTDAAIGPAYREWSTFAVGPGHCL
QLHDRARSLVVPAYAYRKLHPX₆QRPIPSAFX₇FLSHDHGRTWARGHFVAQDTLECQVAEVETGEQRVVTLNARSHLRARVQAQS
TNDGLDFQX₈SQLVKKLVEPPPQGX₉QGSVISFPSPRSGPGSPAQWLLYTHPTHSWQRADLGAYLNPRPPAPEAWSEPX₁₀LLAK
GSX₁₁AYSDLQSMGTGPDGSPLFGX₁₂LYEANDYEEIVFLMFTLKQAFPAEYLPQ
SEQ ID NO: 101:
X₁X₂SX₃PX₄LQKESVFQSGAHAYRIPALLYLPGQQSLLAFAEQRASKKDEHAELIVLRRGDYDAPTHQVQWQAQEVVAQARLDG
HRSMNPCPLYDAQTGTLFLFFIAIPGQVTEQQQLQTRANVTRLX₅QVTSTDHGRTWSSPRDLTDAAIGPAYREWSTFAVGPGHCL
QLHDRARSLVVPAYAYRKLHPX₆QRPIPSAFX₇FLSHDHGRTWARGHFVAQDTLECQVAEVETGEQRVVTLNARSHLRARVQAQS
TNDGLDFQX₈SQLVKKLVEPPPQGX₉QGSVISFPSPRSGPGSPAQWLLYTHPTHSWQRADLGAYLNPRPPAPEAWSEPX₁₀LLAK
GSX₁₁AYSDLQSMGTGPDGSPLFGX₁₂LYEANDYEEIVFLMFTLKQAFPAEYLPQGGGGSGGGGSDKTHTCPPCPAPELLGGPSV
FLFPPKPKDTLMISRTPEVTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNYKTTPPVLDSDGSFFLTSKL
TVDKSRWQQGNVFSCSVMHEALHNHYTQKSLSLSPGK
SEQ ID NO: 102:
X₁X₂SX₃PX₄LQKESVFQSGAHAYRIPALLYLPGQQSLLAFAEQRASKKDEHAELIVLRRGDYDAPTHQVQWQAQEVVAQARLDG
HRSMNPCPLYDAQTGTLFLFFIAIPGQVTEQQQLQTRANVTRLX₅QVTSTDHGRTWSSPRDLTDAAIGPAYREWSTFAVGPGHCL
QLHDRARSLVVPAYAYRKLHPX₆QRPIPSAFX₇FLSHDHGRTWARGHFVAQDTLECQVAEVETGEQRVVTLNARSHLRARVQAQS
TNDGLDFQX₈SQLVKKLVEPPPQGX₉QGSVISFPSPRSGPGSPAQWLLYTHPTHSWQRADLGAYLNPRPPAPEAWSEPX₁₀LLAK
GSX₁₁AYSDLQSMGTGPDGSPLFGX₁₂LYEANDYEEIVFLMFTLKQAFPAEYLPQGGGGSGGGGSDKTHTCPPCPAPELLGGPSVF
LFPPKPKDTLMISRTPEVTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGKGGGGSGGGGSGGGGSEVQLVESGGGLVQPGGSLRLSCAASGFNIKDTYI
HWVRQAPGKGLEWVARIYPTNGYTRYADSVKGRFTISADTSKNTAYLQMNSLRAEDTAVYYCSRWGGDGFYAMDYWGQGTLVTVS
SGGGGSGGGGSGGGGSDIQMTQSPSSLSASVGDRVTITCRASQDVNTAVAWYQQKPGKAPKLLIYSASFLYSGVPSRFSGSRSGT
DFTLTISSLQPEDFATYYCQQHYTTPPTFGQGTKVEIK
SEQ ID NO: 103:
TVEKSVVFKAEGEHFTDQKGNTIVGSGSGGTTKYFRIPAMCTTSKGTIVVFADARHNTASDQSFIDTAAARSTDGGKTWNKKIAI
YNDRVNSKLSRVMDPTCIVANIQGRETILVMVGKWNNNDKTWGAYRDKAPDTDWDLVLYKSTDDGVTFSKVETNIHDIVTKNGTI
SAMLGGVGSGLQLNDGKLVFPVQMVRTKNITTVLNTSFIYSTDGITWSLPSGYCEGFGSENNIIEFNASLVNNIRNSGLRRSFET
KDFGKTWTEFPPMDKKVDNRNHGVQGSTITIPSGNKLVAAHSSAQNKNNDYTRSDISLYAHNLYSGEVKLIDDFYPKVGNASGAG
YSCLSYRKNVDKETLYVVYEANGSIEFQDLSRHLPVIKSYNGGGGSGGGGSDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGKGGGGSGGGGSGGGGSEVQLVESGGGLVQPGGSLRLSCAASGFNIKDTYIHWVRQAPGKGLEW
VARIYPTNGYTRYADSVKGRFTISADTSKNTAYLQMNSLRAEDTAVYYCSRWGGDGFYAMDYWGQGTLVTVSSGGGGSGGGGSGG
GGSDIQMTQSPSSLSASVGDRVTITCRASQDVNTAVAWYQQKPGKAPKLLIYSASFLYSGVPSRFSGSRSGTDFTLTISSLQPED
FATYYCQQHYTTPPTFGQGTKVEIK
SEQ ID NO: 104:
ACAGTGGAAAAGTCCGTGGTGTTCAAGGCCGAGGGCGAGCACTTCACCGACCAGAAAGGCAATACCATCGTCGGCTCTGGCAGCG
GCGGCACCACCAAGTACTTTAGAATCCCCGCCATGTGCACCACCAGCAAGGGCACCATTGTGGTGTTCGCCGACGCCAGACACAA
CACCGCCAGCGATCAGAGCTTCATCGATACCGCTGCCGCCAGATCTACCGATGGCGGCAAGACCTGGAACAAGAAGATCGCCATC
TACAACGACCGCGTGAACAGCAAGCTGAGCAGAGTGATGGACCCTACCTGCATCGTGGCCAACATCCAGGGCAGAGAAACCATCC
TGGTCATGGTCGGAAAGTGGAACAACAACGATAAGACCTGGGGCGCCTACAGAGACAAGGCCCCTGATACCGATTGGGACCTCGT
GCTGTACAAGAGCACCGATGACGGCGTGACCTTCAGCAAGGTGGAAACAAACATCCACGACATCGTGACCAAGAACGGCACCATC
TCTGCCATGCTCGGCGGCGTTGGATCTGGCCTGCAACTGAATGATGGCAAGCTGGTGTTCCCCGTGCAGATGGTCCGAACAAAGA
ATATCACCACCGTGCTGAATACCAGCTTCATCTACAGCACCGACGGCATCACATGGTCCCTGCCTAGCGGCTACTGTGAAGGCTT
TGGCAGCGAGAACAACATCATCGAGTTCAACGCCAGCCTGGTCAACAACATCCGGAACAGCGGCCTGCGGAGAAGCTTCGAGACA
AAGGACTTCGGAAAGACGTGGACCGAGTTTCCTCCAATGGACAAGAAGGTGGACAACCGGAACCACGGCGTGCAGGGCAGCACAA
TCACAATCCCTAGCGGCAACAAACTGGTGGCCGCTCACTCTAGCGCCCAGAACAAGAACAACGACTACACCAGAAGCGACATCAG
CCTGTACGCCCACAACCTGTACAGCGGCGAAGTGAAGCTGATCGACGACTTCTACCCCAAAGTGGGCAATGCCAGCGGAGCCGGC
TACAGCTGTCTGAGCTACCGGAAAAATGTGGACAAAGAAACCCTGTACGTGGTGTACGAGGCCAACGGCAGCATCGAGTTTCAGG
ACCTGAGCAGACATCTGCCCGTGATCAAGAGCTACAACGGCGGAGGTGGAAGTGGCGGAGGCGGATCcGACAAAACTCACACATG
CCCACCGTGCCCAGCACCTGAACTCCTGGGGGGACCGTCAGTCTTCCTCTTCCCCCCAAAACCCAAGGACACCCTCATGATCTCC
CGGACCCCTGAGGTCACATGCGTGGTGGTGGACGTGAGCCACGAAGACCCTGAGGTCAAGTTCAACTGGTACGTGGACGGCGTGG
AGGTGCATAATGCCAAGACAAAGCCGCGGGAGGAGCAGTACAACAGCACGTACCGTGTGGTCAGCGTCCTCACCGTCCTGCACCA
GGACTGGCTGAATGGCAAGGAGTACAAGTGCAAGGTCTCCAACAAAGCCCTCCCAGCCCCCATCGAGAAAACCATCTCCAAAGCC
AAAGGGCAGCCCCGAGAACCACAGGTcTACACCCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTAAAGGAGGCGGAGGATCTG
GCGGAGGTGGAAGTGGCGGAGGCGGATCTGAGGTGCAGCTGGTTGAATCTGGCGGAGGACTGGTTCAGCCTGGCGGATCTCTGAG
ACTGTCTTGTGCCGCCAGCGGCTTCAACATCAAGGACACCTACATCCACTGGGTCCGACAGGCCCCTGGCAAAGGACTTGAATGG
GTCGCCAGAATCTACCCCACCAACGGCTACACCAGATACGCCGACTCTGTGAAGGGCAGATTCACCATCAGCGCCGACACCAGCA
AGAACACCGCCTACCTGCAGATGAACAGCCTGAGAGCCGAGGACACCGCCGTGTACTACTGTTCTAGATGGGGAGGCGACGGCTT
CTACGCCATGGATTATTGGGGCCAGGGCACCCTGGTCACCGTTTCTTCTGGCGGAGGAGGATCTGGCGGAGGCGGAAGTGGCGGA
GGCGGATCTGACATCCAGATGACACAGAGCCCTAGCAGCCTGTCTGCCAGCGTGGGAGACAGAGTGACCATCACCTGTAGAGCCA
GCCAGGACGTGAACACAGCCGTGGCTTGGTATCAGCAGAAGCCTGGCAAGGCCCCTAAGCTGCTGATCTACAGCGCCAGCTTTCT
GTACTCCGGCGTGCCCAGCAGATTCAGCGGCTCTAGAAGCGGCACCGACTTCACCCTGACCATAAGCAGTCTGCAGCCCGAGGAC
TTCGCCACCTACTACTGTCAGCAGCACTACACCACACCTCCAACCTTTGGCCAGGGCACCAAGGTGGAAATCAAG

1.-89. (canceled)

90. A recombinant mutant human sialidase enzyme comprising an amino acid sequence having at least 90% sequence identity to SEQ ID NO: 1; a substitution of the methionine residue at position 1 of SEQ ID NO: 1; and sialidase enzymatic activity.

91. The sialidase of claim 90, wherein the methionine residue at position 1 is substituted by arginine (M1R), histidine (M1H), lysine (M1K), aspartic acid (M1D), threonine (M1T), asparagine (M1N), glutamine (M1Q), glycine (M1G), alanine (M1A), valine (M1V), leucine (M1L), phenylalanine (M1F), or tyrosine (M1Y).

92. The sialidase of claim 91, wherein the methionine residue at position 1 is substituted by histidine (M1H).

93. The sialidase of claim 91, wherein the methionine residue at position 1 is substituted by leucine (MIL).

94. The sialidase of claim 91, wherein the methionine residue at position 1 is substituted by phenylalanine (M1F).

95. The sialidase of claim 91, wherein the methionine residue at position 1 is substituted by asparagine (MIN).

96. The sialidase of claim 91, wherein the methionine residue at position 1 is substituted by alanine (MIA).

97. The sialidase of claim 91, wherein the methionine residue at position 1 is substituted by threonine (MIT).

98. The sialidase of claim 90, further comprising one or more of (a) a substitution of the valine residue at position 6 of SEQ ID NO: 1; (b) a substitution of the isoleucine residue at position 187 of SEQ ID NO: 1 with a lysine (I187K); and (c) a substitution of the cysteine residue at position 332.

99. The sialidase of claim 98, wherein the valine residue at position 6 is substituted by tyrosine (V6Y) and/or the cysteine residue at position 332 is substituted by alanine (C332A).

100. A fusion protein comprising:
(a) the sialidase enzyme of claim 90; and
(b) an immunoglobulin Fc domain and/or an immunoglobulin antigen-binding domain; wherein the sialidase and the immunoglobulin Fc domain and/or the immunoglobulin antigen-binding domain are linked by a peptide bond or an amino acid linker.

101. An antibody conjugate comprising the fusion protein of claim 100.

102. The antibody conjugate of claim 101, wherein the antibody conjugate comprises:
(a) a first polypeptide comprising an immunoglobulin light chain;
(b) a second polypeptide comprising an immunoglobulin heavy chain; and
(c) a third polypeptide comprising the immunoglobulin Fc domain and the sialidase; wherein the first and second polypeptides are covalently linked together and the second and third polypeptides are linked together, and wherein the first polypeptide and the second polypeptide together define the immunoglobulin antigen-binding domain.

103. An isolated nucleic acid comprising a nucleotide sequence encoding the recombinant mutant human sialidase of claim 90.

104. An expression vector comprising the nucleic acid of claim 103.

105. A host cell comprising the expression vector of claim 104.

106. A pharmaceutical composition comprising the recombinant mutant human sialidase of claim 90.

107. A method of treating cancer in a subject in need thereof, the method comprising administering to the subject an effective amount of the recombinant mutant human sialidase of claim 90.

108. A method of treating cancer in a subject in need thereof, the method comprising administering to the subject an effective amount of the antibody conjugate of claim 102.

109. A method of increasing expression of HLA-DR, CD86, CD83, IFNγ, IL-1b, IL-6, TNFα, IL-17A, IL-2, or IL-6 in a cell or tissue, the method comprising contacting the cell or tissue with an effective amount of the recombinant mutant human sialidase of claim 90.
